(19) United States
(12) Patent Application Publication
(10) Pub. No.: US 2024/0002824 A1
(43) Pub. Date: Jan. 4, 2024

(54) PROTEIN COMPOSITIONS AND METHODS OF PRODUCTION

(71) Applicant: Clara Foods Co., Daly City, CA (US)

(72) Inventors: Logan HURST, Daly City, CA (US); Weixi ZHONG, Daly City, CA (US); Charles Albert TINDELL, Daly City, CA (US); Lauren KOLYER, Daly City, CA (US); Ranjan PATNAIK, Daly City, CA (US)

(21) Appl. No.: 18/344,773

(22) Filed: Jun. 29, 2023

Related U.S. Application Data
(60) Provisional application No. 63/356,972, filed on Jun. 29, 2022.

(51) Int. Cl. C12N 9/26; C12N 1/16

(52) U.S. Cl. CPC C12N 9/2431; C07K 2319/035; C12N 1/165; C12Y 302/01026

(57) ABSTRACT

Provided are systems and methods for recombinant proteins in microorganisms engineered to use alternate carbon sources.

### CROSS-REFERENCE TO RELATED APPLICATIONS

This application claims priority to and benefit of U.S. Provisional Application No. 63/356,972, filed Jun. 29, 2022; which is herein incorporated by reference in its entirety.

### SEQUENCE LISTING

The instant application contains a Sequence Listing which has been submitted in XML format via Patent Center and is hereby incorporated by reference in its entirety. Said XML copy, created on Jun. 28, 2023, is named 56025US_CRF_sequencelisting.xml and is 425,213 bytes in size.

### BACKGROUND

A significant expense in commercial recombinant protein production is due to the cost of the carbon (e.g., sugar) fed to the recombinant organism during fermentation. This expense may be reduced by feeding the recombinant organisms less expensive carbon sources. Unfortunately, many recombinant organisms are unable to metabolize these less expensive carbon sources. Thus, there is a need to create recombinant organisms which are able to metabolize these less expensive carbon sources when used for commercial recombinant protein production.

### SUMMARY

An aspect of the present disclosure is a fusion protein comprising a catalytic domain of a heterologous glycosyl hydrolase. In some embodiments, the fusion protein further comprises a and an anchoring domain of a glycosylphosphatidylinositol (GPI)-anchored protein, wherein the anchoring domain comprises at least about 200 amino acids and/or at least about 30% of the residues in the anchoring domain are serines or threonines.

Another aspect of the present disclosure is an engineered host cell comprising: an integrated coding sequence of a fusion protein comprising a catalytic domain of a heterologous glycosyl hydrolase; and an integrated coding sequence of a heterologous protein of interest (POI); wherein the engineered host cell does not endogenously express the glycosyl hydrolase and the POI; and wherein the glycosyl hydrolase is anchored on the surface of the engineered host cell.

In some embodiments, the glycosyl hydrolase is an invertase selected from: S. cerevisiae, Kluyveromyces lactis, Cyberlindnera jadinii, Oryza sativa japonica (rice), Oryza sativa japonica (rice), Arabidopsis thaliana, Arabidopsis thaliana, Arabidopsis thaliana, Rattus norvegicus (rat), Oryctolagus cuniculus (Rabbit), and Homo sapiens.

In some embodiments, the invertase is encoded by the SUC2 gene.

In some embodiments, the invertase is encoded by the MAL1 gene.

In some embodiments, the invertase is encoded by a gene selected from: invertase (INV1), cytosolic invertase 1 (CINV1), CIN2, CINV1, INVA, INVE, and sucrase-isomaltase (SI) gene.

In some embodiments, the fusion protein is surface-displayed on the engineered host cell; wherein the surface-displayed fusion protein comprises a catalytic domain of the glycosyl hydrolase and an anchoring domain of a glycosylphosphatidylinositol (GPI)-anchored protein, wherein the anchoring domain comprises at least about 200 amino acids and/or at least about 30% of the residues in the anchoring domain are serines or threonines.

In some embodiments, the anchoring domain comprises at least about 225 amino acids, at least about 250 amino acids, at least about 275 amino acids, at least about 300 amino acids, at least about 325 amino acids, at least about 350 amino acids, at least about 375 amino acids, or at least about 400 amino acids.

In some embodiments, at least about 35% of the residues in the anchoring domain are serines or threonines, at least about 40% of the residues in the anchoring domain are serines or threonines, at least about 45% of the residues in the anchoring domain are serines or threonines, or at least about 50% of the residues in the anchoring domain are serines or threonines.

In some embodiments, the serines or threonines in the anchoring domain are capable of being O-mannosylated.

In some embodiments, a fusion protein having an anchoring domain comprising at least about 325 amino acids provides greater glycosyl hydrolase activity relative to a fusion protein having an anchoring domain comprising less than about 300 amino acids.

In some embodiments, a fusion protein having an anchoring domain comprising at least about 300 amino acids provides greater glycosyl hydrolase activity relative to a fusion protein having an anchoring domain comprising less than about 250 amino acids.

In some embodiments, the fusion protein comprises the anchoring domain of the GPI anchored protein.

In some embodiments, the fusion protein comprises the GPI anchored protein without its native signal peptide or native secretory signal.

In some embodiments, the GPI anchored protein is not native to the engineered host cell.

In some embodiments, the GPI anchored protein is naturally expressed by a S. cerevisiae cell and the engineered host cell is not a S. cerevisiae cell.

In some embodiments, the GPI anchored protein is selected from Tir4, Dan1, or Sed1.

In some embodiments, an anchoring domain of the GPI anchored protein comprises an amino acid sequence that is at least 70% identical, at least 75% identical, at least 80% identical, at least 85% identical, at least 90% identical, or at least 95% identical, to one of SEQ ID NO: 1 to SEQ ID NO: 14.

In some embodiments, the anchoring domain of the GPI anchored protein comprises an amino acid sequence of one of SEQ ID NO: 1 to SEQ ID NO: 14.

In some embodiments, the engineered host cell is a yeast cell.

In some embodiments, the engineered host cell is a Pichia species.

In some embodiments, the Pichia species is Pichia pastoris.

In some embodiments, the engineered host cell comprises a genomic modification that expresses the fusion.

In some embodiments, the fusion protein comprises a portion of the glycosyl hydrolase in addition to its catalytic domain.

In some embodiments, the fusion protein comprises substantially the entire amino acid sequence of the glycosyl hydrolase.

In some embodiments, in the fusion protein, the catalytic domain is N-terminal to the anchoring domain.

In some embodiments, in the fusion protein, the catalytic domain is C-terminal to the anchoring domain.

In some embodiments, the fusion protein comprises a linker between the catalytic domain and the anchoring domain.

In some embodiments, wherein, upon translation, the fusion protein comprises a signal peptide and/or a secretory signal.

In some embodiments, wherein a growth rate of the engineered host cell in a media containing sucrose as a primary carbon source is higher than a growth rate of a control host cell, wherein the control host cell is identical to the engineered host cell, except the control cell does not express the glycosyl hydrolase.

In some embodiments, wherein the engineered eukaryotic cell comprises a genomic modification that overexpresses a secreted recombinant protein and/or comprises an extrachromosomal modification that overexpresses a secreted recombinant protein.

In some embodiments, wherein the secreted recombinant protein is an animal protein.

In some embodiments, wherein the animal protein is an egg protein.

In some embodiments, wherein the egg protein is selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

In some embodiments, wherein the genomic modification and/or the extrachromosomal modification that overexpresses the secreted recombinant protein comprises an inducible promoter.

In some embodiments, wherein the inducible promoter is an AOX1, DAK2, PEX11, FLD1, FGH1, DAS1, DAS2, CAT1, MDH3, HAC1, BiP, RAD30, RVS161-2, MPP10, THP3, TLR, GBP2, PMP20, SHB17, PEX8, PEX4, or TKL3 promoter.

In some embodiments, wherein the genomic modification and/or the extrachromosomal modification that overexpresses a secreted recombinant protein comprises an AOX1, TDH3, MOX, RPS25A, or RPL2A terminator.

T In some embodiments, wherein the genomic modification and/or the extrachromosomal modification that overexpresses a secreted recombinant protein encodes a signal peptide and/or a secretory signal.

In some embodiments, wherein the genomic modification and/or the extrachromosomal modification that overexpresses a secreted recombinant protein comprises codons that are optimized for the species of the engineered eukaryotic cell.

In some embodiments, wherein the secreted recombinant protein is designed to be secreted from the cell and/or is capable of being secreted from the cell.

In some embodiments, wherein the fusion protein comprises an amino acid sequence that is at least 80%, at least 85%, at least 90%, at least 95%, at least 96%, at least 97%, at least 98%, at least 99%, or 100% identical to the amino acid sequence selected from SEQ ID NOs: 315, 332-335, and 342.

In some embodiments, wherein the fusion protein comprises a nucleotide sequence that is at least 80%, at least 85%, at least 90%, at least 95%, at least 96%, at least 97%, at least 98%, at least 99%, or 100% identical to the nucleotide sequence of SEQ ID ON: 314.

Another aspect of the present disclosure is a method of growing/culturing the engineered host cell, wherein the method comprises culturing the engineered host cell with a carbon source that is not naturally utilized by the host cell in the absence of the glycosyl hydrolase.

Another aspect of the present disclosure is a method for growing/culturing a host cell with a carbon source that is not naturally utilized by the host cell, the method comprising: (a) recombinantly producing in the host cell, a fusion protein comprising a catalytic domain of a glycosyl hydrolase capable of digesting sucrose; optionally, wherein the glycosyl hydrolase capable of digesting sucrose is an invertase; (b) recombinantly producing in the host cell a heterologous protein of interest (POI); wherein the host cell does not express the glycosyl hydrolase endogenously; wherein the engineered host cell prior to step (a) does not utilize sucrose as a carbon source as efficiently as glucose, and wherein the glycosyl hydrolase is expressed on the surface of the engineered host cell.

Another aspect of the present disclosure is a method for manufacturing a host cell capable of utilizing a carbon source that is not naturally utilized by the host cell, the method comprising: (a) obtaining a host cell that recombinantly expresses a fusion protein comprising a catalytic domain of a glycosyl hydrolase capable of digesting sucrose; optionally, wherein the glycosyl hydrolase capable of digesting sucrose is an invertase; and (b) genetically modifying the host cell to express a heterologous protein of interest (POI); wherein the host cell does not utilize sucrose as a carbon source as efficiently as glucose in the absence of the glycosyl hydrolase.

Another aspect of the present disclosure is a method for manufacturing a host cell capable of utilizing a carbon source that is not naturally utilized by the host cell, the method comprising: (a) obtaining a host cell that recombinantly expresses a heterologous protein of interest (POI); and (b) genetically modifying the host cell to express a fusion protein comprising a catalytic domain of a glycosyl hydrolase capable of digesting sucrose; optionally, wherein the glycosyl hydrolase capable of digesting sucrose is an invertase; wherein the host cell prior to step (b) does not utilize sucrose as a carbon source as efficiently as glucose.

Any aspect or embodiment described herein can be combined with any other aspect or embodiment as disclosed herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the growth of P. pastoris on minimal nutrient plates containing glucose, fructose and sucrose.

FIG. 2 illustrates an exemplary schematic of a construct to express a surface displayed protein comprising SUC2 and an anchored protein Tir4.

FIG. 3 illustrates the growth of P. pastoris strains using mannose as a sole carbon source.

FIG. 4 illustrates the growth of P. pastoris strains using glucose or sucrose as a sole carbon source. The strains labelled “_D” in FIG. 4 denote that dextrose (glucose) was used as the carbon source in the experimental condition. The strains labelled “_S” in FIG. 4 denote that sucrose was used as the carbon source in the experimental condition.

FIG. 5 is an SDS-PAGE gel comparing protein of interest production in P. pastoris strains using glucose or sucrose as a sole carbon source.

### DETAILED DESCRIPTION

High-yielding recombinant protein expression is a cornerstone of various industries such as therapeutic proteins, food industry, cosmetics, etc. The growth of host cells in readily available media to produce such recombinant proteins is therefore one of the most important factors not only from an economic perspective but also from an environment perspective. Recombinant protein expression using commonly available carbon sources, while maintaining high titers of the recombinant proteins is necessary. The present invention addresses this need. The systems and methods provide high-titer expression of recombinant proteins in large scale production using genetic modifications to the host cell which are capable of utilizing carbon sources not usually utilized by the host cell and are particularly useful for expressing pure heterologous animal derived proteins in a microbial host.

### Host Cell

As used herein, a “host cell” refers to a cell which is capable of protein expression and optionally protein secretion. Such host cell is applied in the methods of the present invention. For that purpose, for the host cell to express a polypeptide, a nucleotide sequence encoding the polypeptide is present or introduced in the cell. Host cells provided by the present invention can be prokaryotes or eukaryotes. As will be appreciated by one of skill in the art, a prokaryotic cell lacks a membrane-bound nucleus, while a eukaryotic cell has a membrane-bound nucleus. Examples of eukaryotic cells include, but are not limited to, vertebrate cells, mammalian cells, human cells, animal cells, invertebrate cells, plant cells, nematodal cells, insect cells, stem cells, fungal cells or yeast cells.

Examples of yeast cells include but are not limited to the Saccharomyces genus (e.g. Saccharomyces cerevisiae, Saccharomyces kluyveri, Saccharomyces uvarum), the Komagataella genus (Komagataella pastoris, Komagataella pseudopastoris or Komagataella phaffii), Kluyveromyces genus (e.g. Kluyveromyces lactis, Kluyveromyces marxianus), the Candida genus (e.g. Candida utilis, Candida cacaoi), the Geotrichum genus (e.g. Geotrichum fermentans), as well as Hansenula polymorpha and Yarrowia lipolytica. A host cell may also be a member of the following species: Arxula spp., Arxula adeninivorans, Kluyveromyces spp., Kluyveromyces lactis, Komagataella phaffii, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Bacillus subtilis, Colletotrichum spp., Colletotrichum gloeosporiodes, Endothia spp., Endothia parasitica, Escherichia coli, Fusarium spp., Fusarium graminearum, Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculo sum, Penicillium purpurogenum, Penicillium roqueforti, Pleurotus spp., Pleurotus ostreatus, Rhizomucor spp., Rhizomucor miehei, Rhizomucor pusillus, Rhizopus spp., Rhizopus arrhizus, Rhizopus oligosporus, Rhizopus oryzae, Trichoderma spp., Trichoderma altroviride, Trichoderma reesei, or Trichoderma vireus.

The genus Pichia is of particular interest. Pichia comprises a number of species, including the species Pichia pastoris, Pichia methanolica, Pichia kluyveri, and Pichia angusta. Most preferred is the species Pichia pastoris.

The former species Pichia pastoris has been divided and renamed to Komagataella pastoris and Komagataella phaffii. Therefore, Pichia pastoris is synonymous for both Komagataella pastoris and Komagataella phaffii.

In some embodiments, the host cell is a Pichia pastoris, Hansenula polymorpha, Trichoderma reesei, Saccharomyces cerevisiae, Kluyveromyces lactis, Yarrowia lipolytica, Pichia methanolica, Candida boidinii, and Komagataella, and Schizosaccharomyces pombe.

### Protein of Interest

The term “protein of interest” (POI) as used herein refers to a protein that is produced by means of recombinant technology in a host cell. More specifically, the protein may either be a polypeptide not naturally occurring in the host cell, i.e. a heterologous protein, or else may be native to the host cell, i.e. a homologous protein to the host cell, but is produced, for example, by transformation with a self-replicating vector containing the nucleic acid sequence encoding the POI, or upon integration by recombinant techniques of one or more copies of the nucleic acid sequence encoding the POI into the genome of the host cell, or by recombinant modification of one or more regulatory sequences controlling the expression of the gene encoding the POI, e.g. of the promoter sequence. In general, the proteins of interest referred to herein may be produced by methods of recombinant expression well known to a person skilled in the art. Exemplary proteins of interest are provided in Table 6. A recombinant POI expressed in a host cell may comprise a sequence with at least 75%, at least 80%, at least 85%, at least 90%, at least 92%, at least 95%, at least 97% or at least 99% sequence identity to any of the sequences in Table 6.

There is no limitation with respect to the protein of interest (POI). The POI may comprise a eukaryotic or prokaryotic polypeptide, variant or derivative thereof. The POI can be any eukaryotic or prokaryotic protein. The protein can be a naturally secreted protein or an intracellular protein, i.e. a protein which is not naturally secreted. The present invention also includes biologically active fragments of proteins. In another embodiment, a POI may be an amino acid chain or present in a complex, such as a dimer, trimer, hetero-dimer, multimer or oligomer.

The protein of interest may be a protein used as nutritional, dietary, digestive, supplements, such as in food products, feed products, or cosmetic products. The food products may be, for example, bouillon, desserts, cereal bars, confectionery, sports drinks, dietary products or other nutrition products. Preferably, the protein of interest is a food additive.

### Glycosyl Hydrolases

In some cases, a heterologous glycosyl hydrolase is produced in a host cell that has been engineered to express or overexpress one or more heterologous recombinant proteins such as the proteins of interest. A glycosyl hydrolase may be a surface-displayed enzyme that hydrolyses a disaccharide which allows a host cell to utilize a carbon source which it previously was unable to utilize or utilize efficiently. In some embodiments, a carbon source which a host cell is previously unable to utilize or utilize efficiently may comprise sucrose, maltose, fructose, high fructose corn syrup, molasses, or some combination thereof. In some embodiments, the carbon source which a host cell is previously unable to utilize or utilize efficiently may be present in a mixture with glucose. In some examples, a glycosyl hydrolase may be an enzyme that hydrolyzes a carbon source, e.g., a disaccharide, to its monomers, e.g., glucose, fructose, and galactose, which can be utilized by the host cell. For example, in some examples, the glycosyl hydrolase may be an invertase such as proteins encoded by the SUC2 or MAL1 genes which cleave a disaccharide sucrose to release glucose and fructose which can be utilized by a yeast such as P. pastoris. In some embodiments, the glycosyl hydrolase may be an invertase such as proteins encoded by the INV1, CINV1, CIN2, INVE, INVA, or SI genes which cleave a disaccharide sucrose to release glucose and fructose which can be utilized by a yeast. Additional non-limiting examples of glycosyl hydrolases include, but are not limited to: invertase, invertase 1, cytosolic invertase 1, Beta-fructofuranosidase, insoluble isoenzyme 2, Alkaline/neutral invertase, Alkaline/neutral invertase A, Alkaline/neutral invertase E, and Sucrase-isomaltase. Exemplary sequences for glycosyl hydrolases are provided in Table 2. A recombinant glycosyl hydrolase expressed in a host cell may comprise a sequence with at least 75%, at least 80%, at least 85%, at least 90%, at least 92%, at least 95%, at least 97% or at least 99% sequence identity to any of the sequences in Table 2.

In certain embodiments, the glycosyl hydrolase is of the family GHS. In certain embodiments, the glycosyl hydrolase is of the family GH7. In certain embodiments, the glycosyl hydrolase is of the family GH9. Such glycosyl hydrolases are found in PCT Application Publication No.: WO2009090381, which is hereby incorporated by reference in its entirety.

An engineered host cell expressing a heterologous glycosyl hydrolase may be cultured with a carbon source that is not naturally utilized by the host cell or not utilized as efficiently as glucose in the absence of the glycosyl hydrolase.

An engineered host cell expressing a heterologous glycosyl hydrolase may have a growth rate in a media containing sucrose as a primary carbon source is higher than a growth rate of a control host cell, wherein the control host cell is identical to the engineered host cell, except the control cell does not express the glycosyl hydrolase.

In some embodiments, an engineered host cell expressing a heterologous glycosyl hydrolase may have a growth rate in a media containing fructose as a primary carbon source is higher than a growth rate of a control host cell, wherein the control host cell is identical to the engineered host cell, except the control cell does not express the glycosyl hydrolase.

In some embodiments, an engineered host cell expressing a heterologous glycosyl hydrolase may have a growth rate in a media containing maltose as a primary carbon source is higher than a growth rate of a control host cell, wherein the control host cell is identical to the engineered host cell, except the control cell does not express the glycosyl hydrolase.

In some embodiments, an engineered host cell expressing a heterologous glycosyl hydrolase may have a growth rate in a media containing high fructose corn syrup as a primary carbon source is higher than a growth rate of a control host cell, wherein the control host cell is identical to the engineered host cell, except the control cell does not express the glycosyl hydrolase.

In some embodiments, an engineered host cell expressing a heterologous glycosyl hydrolase may have a growth rate in a media containing molasses as a primary carbon source is higher than a growth rate of a control host cell, wherein the control host cell is identical to the engineered host cell, except the control cell does not express the glycosyl hydrolase.

In some embodiments, an engineered host cell expressing a heterologous glycosyl hydrolase may have a growth rate in a media containing a disaccharide as a primary carbon source is higher than a growth rate of a control host cell, wherein the control host cell is identical to the engineered host cell, except the control cell does not express the glycosyl hydrolase.

In some embodiments, an engineered host cell expressing a heterologous glycosyl hydrolase may have a growth rate in a media containing a mixture of glucose and a disaccharide as a primary carbon source is higher than a growth rate of a control host cell, wherein the control host cell is identical to the engineered host cell, except the control cell does not express the glycosyl hydrolase.

In some embodiments, an engineered host cell expressing a heterologous glycosyl hydrolase may have a growth rate in a media containing a carbon source that is not glucose as a primary carbon source is higher than a growth rate of a control host cell, wherein the control host cell is identical to the engineered host cell, except the control cell does not express the glycosyl hydrolase.

### Surface Display of Glycosyl Hydrolases

Surface displaying a catalytic domain of an enzyme provides effective and efficient means to project the catalytic domain into the extracellular space, thereby increasing the likelihood that the catalytic domain will encounter and catalyze an enzymatic reaction with its substrate, e.g., protein, lipid, carbohydrate, or another compound. In the present disclosure, a fusion protein is localized to the extracellular surface of a host cell, i.e., is surface displayed. This way, the catalytic domain is unlikely to contact an intracellular, membrane-associated, or cell wall protein, thereby lowering the opportunity for the enzyme to modify, degrade, or the like a substrate needed by the cell. In some embodiments, the fusion protein catalyzes a reaction that cleaves a disaccharide, which would allow the cell to utilize an alternate carbon source that was previously not possible or efficient. By cleaving the disaccharide into monosaccharides, the cell is able to use the monosaccharides even though the culturing medium did not include the monosaccharide. In further embodiments, the fusion protein expresses an enzyme, e.g., a sucrase, that digests an impurity secreted by the cell.

An aspect of the present disclosure is an engineered host cell that expresses a surface-displayed fusion protein. In some embodiments, host cells that can be engineered to express a surface-displayed fusion protein provided by the present invention can be prokaryotes or eukaryotes. As will be appreciated by one of skill in the art, a prokaryotic cell lacks a membrane-bound nucleus, while a eukaryotic cell has a membrane-bound nucleus. Examples of eukaryotic cells include, but are not limited to, vertebrate cells, mammalian cells, human cells, animal cells, invertebrate cells, plant cells, nematodal cells, insect cells, stem cells, fungal cells or yeast cells.

Examples of yeast cells that may be transformed to include one or more expression cassettes include but are not limited to the Saccharomyces genus (e.g. Saccharomyces cerevisiae, Saccharomyces kluyveri, Saccharomyces uvarum), the Komagataella genus (Komagataella pastoris, Komagataella pseudopastoris or Komagataella phaffii), Kluyveromyces genus (e.g. Kluyveromyces lactis, Kluyveromyces mandanus), the Candida genus (e.g. Candida utilis, Candida cacaoi, the Geotrichum genus (e.g. Geotrichum fermentans), as well as Hansenula polymorpha and Yarrowia lipolytica. A host cell may also be a member of the following species: Arxula spp., Arxula adeninivorans, Kluyveromyces spp., Kluyveromyces lactis, Komagataella phaffii, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Bacillus subtilis, Colletotrichum spp., Colletotrichum gloeosporiodes, Endothia spp., Endothia parasitica, Escherichia coli, Fusarium spp., Fusarium graminearum, Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculo sum, Penicillium purpurogenum, Penicillium roqueforti, Pleurotus spp., Pleurotus ostreatus, Rhizomucor spp., Rhizomucor miehei, Rhizomucor pusillus, Rhizopus spp., Rhizopus arrhizus, Rhizopus oligosporus, Rhizopus oryzae, Trichoderma spp., Trichoderma altroviride, Trichoderma reesei, or Trichoderma vireus.

The genus Pichia is of particular interest. Pichia comprises a number of species, including the species Pichia pastoris, Pichia methanolica, Pichia kluyveri, and Pichia angusta. Most preferred is the species Pichia pastoris.

The former species Pichia pastoris has been divided and renamed to Komagataella pastoris and Komagataella phaffii. Therefore, Pichia pastoris is synonymous for both Komagataella pastoris and Komagataella phaffii.

In some embodiments, the host cell is a Pichia pastoris, Hansenula polymorpha, Trichoderma reesei, Saccharomyces cerevisiae, Kluyveromyces lactis, Yarrowia lipolytica, Pichia methanolica, Candida boidinii, and Komagataella, and Schizosaccharomyces pombe.

In some embodiments, the engineered host cell expresses a surface-displayed fusion protein. The fusion protein comprising a catalytic domain of an enzyme and an anchoring domain of a glycosylphosphatidylinositol (GPI)-anchored protein, wherein the anchoring domain comprises at least about 200 amino acids and/or at least about 30% of the residues in the anchoring domain are serines or threonines.

A fusion protein is a protein consisting of at least two domains that are normally encoded by separate genes but have been joined so that they are transcribed and translated as a single unit; thereby, producing a single (fused) polypeptide.

In the present disclosure, a fusion protein comprises at least a catalytic domain of an enzyme such as a glycosyl hydrolase and an anchoring domain of GPI-anchored protein. Typically, a GPI-anchored protein is a cell surface protein, e.g., which is located on the extracellular surface of the cell.

A fusion protein may further comprise linkers that separate the two domains. Linkers can be flexible or rigid; they can be semi-flexible or semi-rigid. Separating the two domains, may promote activity of the catalytic domain in that it reduces steric hindrance upon the catalytic site which may be present if the catalytic site is too closely positioned relative to an anchoring domain. Additionally, a linker may further project the catalytic domain into the extracellular space, thereby increasing the likelihood that the catalytic domain will encounter and catalyze an enzymatic reaction with its substrate, e.g., protein, lipid, carbohydrate, or other compounds.

In embodiments, the anchoring domain comprises at least about 225 amino acids, at least about 250 amino acids, at least about 275 amino acids, at least about 300 amino acids, at least about 325 amino acids, at least about 350 amino acids, at least about 375 amino acids, or at least about 400 amino acids.

In some embodiments, at least about 35% of the residues in the anchoring domain are serines or threonines, at least about 40% of the residues in the anchoring domain are serines or threonines, at least about 45% of the residues in the anchoring domain are serines or threonines, or at least about 50% of the residues in the anchoring domain are serines or threonines.

In various embodiments, the serines or threonines in the anchoring domain are capable of being 0-mannosylated.

In embodiments, a fusion protein having an anchoring domain comprising at least about 325 amino acids provides greater enzymatic activity relative to a fusion protein having an anchoring domain comprising less than about 300 amino acids.

In some embodiments, a fusion protein having an anchoring domain comprising at least about 300 amino acids provides greater enzymatic activity relative to a fusion protein having an anchoring domain comprising less than about 250 amino acids.

In some embodiments, the fusion protein comprises the GPI anchored protein without its native signal peptide. In some embodiments, the fusion protein comprises the GPI anchored protein without a C terminus region having amino acid sequence of GAAKAVIGMGAGALAAVAAML (SEQ ID NO: 336). In some embodiments, the fusion protein comprises the GPI anchored protein with a C terminus region having amino acid sequence of GAAKAVIGMGAGALAAVAAML (SEQ ID NO: 336).

In some embodiments, the GPI anchored protein is not native to the engineered eukaryotic cell.

In various embodiments, the GPI anchored protein is naturally expressed by a S. cerevisiae cell and the engineered eukaryotic cell is not a S. cerevisiae cell.

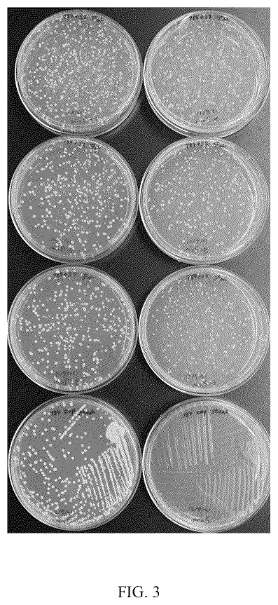

In embodiments, the GPI anchored protein is selected from Tir4, Dan1, Dan4, Sag1, FIG. 2, or Sed1.

In some embodiments, the anchoring domain of the GPI anchored protein comprises an amino acid sequence that is at least 70% identical, at least 75% identical, at least 80% identical, at least 85% identical, at least 90% identical, or at least 95% identical, to one of SEQ ID NO: 1 to SEQ ID NO: 14.

In various embodiments, the anchoring domain of the GPI anchored protein comprises an amino acid sequence of one of SEQ ID NO: 1 to SEQ ID NO: 14.

Sed1p is a major component of the Saccharomyces cerevisiae cell wall. It is required to stabilize the cell wall and for stress resistance in stationary-phase cells. See, e.g., the world wide web (at) uniprot.org/uniprot/Q01589. It is believed that Asn318 (with respect to SEQ ID NO: 13) is the most likely candidate for the GPI attachment site in Sed1p. In some embodiments, a fusion protein comprising a Sed1p anchoring domain has a sequence having at least 95% or more sequence identity with SEQ ID NO:13 or SEQ ID NO: 14. In some cases, the sequence identity may be greater than or about 90%, 95%, 96%, 97%, 98%, 99%, or 100%. In various embodiments, the Sed1p anchoring domain of a fusion protein of the present disclosure comprises a GPI attachment site; thus, the anchoring domain may only require a short fragment of SEQ ID NO: 13 or SEQ ID NO: 14, i.e., a fragment that is 5, 10, 50, 100, 200, or 300 or more amino acids in length, as long as it is capable of projecting the catalytic domain of the fusion protein into the extracellular space. In some embodiments, the anchoring domain comprises, at least, Sed1p's GPI attachment site.

When a linker is present, a fusion protein may have a general structure of: N terminus -(a)-(b)-(c)-C terminus, wherein (a) is comprises a first domain, (b) is one or more linkers, and (c) is a second domain. The first domain may comprise a catalytic domain of an enzyme and the second domain may comprise an anchoring domain of a GPI anchored protein. In some embodiments, in the fusion protein, the catalytic domain is N-terminal to the anchoring domain. The fusion protein may comprise a linker N-terminal to the anchoring domain.

Linkers useful in fusion proteins may comprise one or more sequences of Table 3. In one example, a tandem repeat (of two, three, four, five, six, or more copies) of a linker, e.g., of SEQ ID NO: 33 or SEQ ID NO: 34 is included in a fusion protein.

In embodiments, a fusion protein comprises a Glu-Ala-Glu-Ala (EAEA; SEQ ID NO: 19) spacer dipeptide repeat. The EAEA (SEQ ID NO: 19) is a signal that promotes yields of an expressed protein in certain cell types.

Other linkers are well-known in the art and can be substituted for the linkers of Table 3. For example, in embodiments, the linker may be derived from naturally-occurring multi-domain proteins or are empirical linkers as described, for example, in Chichili et al., (2013), Protein Sci. 22(2):153-167, Chen et al., (2013), Adv Drug Deliv Rev. 65(10):1357-1369, the entire contents of which are hereby incorporated by reference. In embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In embodiments, the linker comprises a polypeptide. In embodiments, the polypeptide is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some cases, the linker is about 59 amino acids long.

The length of a linker may be important to the effectiveness of a surface displayed enzyme's catalytic domain. For example, if a linker is too short, then the catalytic domain of the enzyme may not project far enough away from the cell surface such that it is incapable of interacting with its substrate, e.g., protein, lipid, carbohydrate, or another compound. In this case, the catalytic domain may be buried in the cell wall and/or among other cell surface proteins or sugars. On the other hand, the linker may be too long and/or too rigid to allow adequate contact between a substrate and the catalytic domain of the enzyme.

The secondary structure of a linker may also be important to the effectiveness of a surface displayed enzyme's catalytic domain. More specifically, a linker designed to have a plurality of distinct regions may provide additional flexibility to the fusion protein. As examples, a linker having one or more alpha helices may be superior to a linker having no alpha helices.

The longer linker comprises three subsections: an N-terminal flexible GS linker with higher S content, a rigid linker that forms four turns of an alpha helix, and a flexible GS linker with much higher G content on its C-terminus. Linkers containing only G's and S's in repetitive sequences are commonly used in fusion proteins as flexible spacers that do not introduce secondary structure. In some cases, the ratio of G to S determines the flexibility of the linker. Linkers with higher G content may be more flexible than linkers with higher S content. The structure of the linker of SEQ ID NO: 31 is designed to mimic multi-domain proteins in nature, which often uses alpha helices (sometimes multiple) to separate as well as orient their domains spatially. In fusion proteins of the present disclosure, a complex linker, such as that of SEQ ID NO: 32 can be viewed as a multi-domain protein with the catalytic domain of an enzyme and an anchoring domain of a GPI anchored protein being separate functional domains.

In various embodiments, the fusion protein comprises a linker having an amino acid sequence that is at least 95%, 96%, 97%, 98%, 99%, or 100% identical to SEQ ID NO: 32.

In embodiments, the linker is substantially comprised of glycine and serine residues (e.g. about 30%, or about 40%, or about 50%, or about 60%, or about 70%, or about 80%, or about 90%, or about 95%, or about 96%, or about 97%, or about 98%, or about 99%, or about 100% glycines and serines).

In various embodiments, the engineered eukaryotic cell comprises a genomic modification that expresses the fusion protein and/or comprises an extrachromosomal modification that expresses the fusion protein.

In embodiments, the fusion protein comprises a portion of the enzyme in addition to its catalytic domain.

In some embodiments, the fusion protein comprises substantially the entire amino acid sequence of the enzyme.

In some embodiments, upon translation, the fusion protein comprises a signal peptide and/or a secretory signal. In certain embodiments, the fusion protein comprises a signal peptide and a secretory signal.

In some embodiments, the fusion protein comprises an amino acid sequence having at least at least 75%, at least 80%, at least 85%, at least 90%, at least 95%, at least 96%, at least 97%, at least 98%, at least 99% or 100% amino acid sequence identity to the amino acid sequence selected from SEQ ID NOs: 315, and 332-335. In some embodiments, the fusion protein comprises an amino acid sequence having at least at least 75%, at least 80%, at least 85%, at least 90%, at least 95%, at least 96%, at least 97%, at least 98%, at least 99% or 100% amino acid sequence identity to the amino acid sequence of SEQ ID NO: 315. In some embodiments, the fusion protein comprises an amino acid sequence having at least at least 75%, at least 80%, at least 85%, at least 90%, at least 95%, at least 96%, at least 97%, at least 98%, at least 99% or 100% amino acid sequence identity to the amino acid sequence of SEQ ID NO: 332. In some embodiments, the fusion protein comprises an amino acid sequence having at least at least 75%, at least 80%, at least 85%, at least 90%, at least 95%, at least 96%, at least 97%, at least 98%, at least 99% or 100% amino acid sequence identity to the amino acid sequence of SEQ ID NO: 333. In some embodiments, the fusion protein comprises an amino acid sequence having at least at least 75%, at least 80%, at least 85%, at least 90%, at least 95%, at least 96%, at least 97%, at least 98%, at least 99% or 100% amino acid sequence identity to the amino acid sequence of SEQ ID NO: 334. In some embodiments, the fusion protein comprises an amino acid sequence having at least at least 75%, at least 80%, at least 85%, at least 90%, at least 95%, at least 96%, at least 97%, at least 98%, at least 99% or 100% amino acid sequence identity to the amino acid sequence of SEQ ID NO: 335.

In various embodiments, the engineered eukaryotic cell comprises two or more fusion proteins, three or more fusion proteins, or four fusion proteins.

In some cases, the two or more fusion proteins comprise different enzyme types or the two or more fusion proteins comprise the same enzyme type.

In various cases, the two of the three or more fusion proteins or two of the four or more fusion proteins comprise different enzyme types or two of the three or more fusion proteins or two of the four or more fusion proteins comprise the same enzyme type.

In additional cases, the three of the three or more fusion proteins or three of the four or more fusion proteins comprise different enzyme types or three of the three or more fusion proteins or three of the four or more fusion proteins comprise the same enzyme type.

In various cases, each of the two or more, three or more, or four fusion proteins comprise different enzyme types or each of the two or more, three or more, or four fusion proteins comprise the same enzyme type.

In embodiments, the enzyme types are selected from an enzyme that catalyzes a post-translational modification of a protein secreted by the engineered eukaryotic cell, an enzyme that catalyzes a reaction which allows the engineered eukaryotic cell to rely on alternate carbon sources.

In some embodiments, an engineered host cell expressing a fusion protein may have a growth rate in a media containing fructose as a primary carbon source is higher than a growth rate of a control host cell, wherein the control host cell is identical to the engineered host cell, except the control cell does not express the fusion protein.

In some embodiments, an engineered host cell expressing a fusion protein may have a growth rate in a media containing maltose as a primary carbon source is higher than a growth rate of a control host cell, wherein the control host cell is identical to the engineered host cell, except the control cell does not express the fusion protein.

In some embodiments, an engineered host cell expressing a fusion protein may have a growth rate in a media containing high fructose corn syrup as a primary carbon source is higher than a growth rate of a control host cell, wherein the control host cell is identical to the engineered host cell, except the control cell does not express the fusion protein.

In some embodiments, an engineered host cell expressing a fusion protein may have a growth rate in a media containing molasses as a primary carbon source is higher than a growth rate of a control host cell, wherein the control host cell is identical to the engineered host cell, except the control cell does not express the fusion protein.

In some embodiments, an engineered host cell expressing a fusion protein may have a growth rate in a media containing a disaccharide as a primary carbon source is higher than a growth rate of a control host cell, wherein the control host cell is identical to the engineered host cell, except the control cell does not express the fusion protein.

In some embodiments, an engineered host cell expressing a fusion protein may have a growth rate in a media containing a mixture of glucose and a disaccharide as a primary carbon source is higher than a growth rate of a control host cell, wherein the control host cell is identical to the engineered host cell, except the control cell does not express the fusion protein.

In some embodiments, an engineered host cell expressing a fusion protein may have a growth rate in a media containing a carbon source that is not glucose as a primary carbon source is higher than a growth rate of a control host cell, wherein the control host cell is identical to the engineered host cell, except the control cell does not express the fusion protein.

### Transporter Proteins

In some cases, a heterologous transporter protein is produced in a host cell that has been engineered to express or overexpress one or more heterologous recombinant proteins such as the proteins of interest. A transporter protein may be a protein that allows the host cell to transport a carbon source into the host cell. The host cell then may be able to catalyze a reaction which allows the host cell to utilize a carbon source which it previously was unable to utilize or utilize efficiently. In some embodiments, the transporter protein may be a sucrose permease (such as encoded by the MAL 11 or AGT1 genes) or a maltose permease (such as encoded by the MAL2 gene). Exemplary sequences for glycosyl hydrolases are provided in Table 10. A recombinant glycosyl hydrolase expressed in a host cell may comprise a sequence with at least 75%, at least 80%, at least 85%, at least 90%, at least 92%, at least 95%, at least 97% or at least 99% sequence identity to any of the sequences in Table 10. In certain embodiments, the sucrose permease is a CscB sucrose permease. Exemplary sequences of sucrose permeases can be found in PCT Application Publication No.: WO2022129470, which is hereby incorporated by reference in its entirety.

An engineered host cell expressing a heterologous transporter protein may be cultured with a carbon source that is not naturally utilized by the host cell or not utilized as efficiently as glucose in the absence of the transporter protein.

An engineered host cell expressing a heterologous transporter protein may have a growth rate in a media containing sucrose as a primary carbon source is higher than a growth rate of a control host cell, wherein the control host cell is identical to the engineered host cell, except the control cell does not express the transporter protein.

In some embodiments, an engineered host cell expressing a heterologous transporter protein may have a growth rate in a media containing fructose as a primary carbon source is higher than a growth rate of a control host cell, wherein the control host cell is identical to the engineered host cell, except the control cell does not express the transporter protein.

In some embodiments, an engineered host cell expressing a heterologous transporter protein may have a growth rate in a media containing maltose as a primary carbon source is higher than a growth rate of a control host cell, wherein the control host cell is identical to the engineered host cell, except the control cell does not express the transporter protein.

In some embodiments, an engineered host cell expressing a heterologous transporter protein may have a growth rate in a media containing high fructose corn syrup as a primary carbon source is higher than a growth rate of a control host cell, wherein the control host cell is identical to the engineered host cell, except the control cell does not express the transporter protein.

In some embodiments, an engineered host cell expressing a heterologous transporter protein may have a growth rate in a media containing molasses as a primary carbon source is higher than a growth rate of a control host cell, wherein the control host cell is identical to the engineered host cell, except the control cell does not express the transporter protein.

In some embodiments, an engineered host cell expressing a heterologous transporter protein may have a growth rate in a media containing a disaccharide as a primary carbon source is higher than a growth rate of a control host cell, wherein the control host cell is identical to the engineered host cell, except the control cell does not express the transporter protein.

In some embodiments, an engineered host cell expressing a heterologous transporter protein may have a growth rate in a media containing a mixture of glucose and a disaccharide as a primary carbon source is higher than a growth rate of a control host cell, wherein the control host cell is identical to the engineered host cell, except the control cell does not express the transporter protein.

In some embodiments, an engineered host cell expressing a heterologous transporter protein may have a growth rate in a media containing a carbon source that is not glucose as a primary carbon source is higher than a growth rate of a control host cell, wherein the control host cell is identical to the engineered host cell, except the control cell does not express the transporter protein.

In some cases, the engineered host cell may endogenously express a glycosyl hydrolase which can utilize the alternate carbon source, but it is unable to do so efficiently. In such cases, a transporter protein may increase the uptake of the alternate carbon source and therefore increase the metabolization of the alternate carbon source.

In some cases, the engineered host cell may not express a glycosyl hydrolase which is able to hydrolyze an alternate carbon source. In such examples, the host cell may be engineered to express a heterologous glycosyl hydrolase which is able to hydrolyze the alternate carbon source.

### Expression of Recombinant Proteins

Expression of a recombinant proteins can be provided by an expression vector, a plasmid, a nucleic acid integrated into the host genome or other means. For example, a vector for expression can include: (a) a promoter element, (b) a signal peptide, (c) a heterologous protein sequence, and (d) a terminator element.

Expression vectors that can be used for expression of a recombinant proteins include those containing an expression cassette with elements (a), (b), (c) and (d). In some embodiments, the signal peptide (c) need not be included in the vector. In general, the expression cassette is designed to mediate the transcription of the transgene when integrated into the genome of a cognate host microorganism.

To aid in the amplification of the vector prior to transformation into the host microorganism, a replication origin (e) may be contained in the vector (such as pUC ORIC and pUC (DNA2.0)). To aide in the selection of microorganism stably transformed with the expression vector, the vector may also include a selection marker (f) such as URA3 gene and Zeocin resistance gene (ZeoR). The expression vector may also contain a restriction enzyme site (g) that allows for linearization of the expression vector prior to transformation into the host microorganism to facilitate the expression vectors stable integration into the host genome. In some embodiments the expression vector may contain any subset of the elements (b), (e), (f), and (g), including none of elements (b), (e), (f), and (g). Other expression elements and vector elements known to one of skill in the art can be used in combination or substituted for the elements described herein.

Exemplary promoter elements (a) may include, but are not limited to, a constitutive promoter, inducible promoter, and hybrid promoter. Promoters include, but are not limited to, acu-5, adh1+, alcohol dehydrogenase (ADH1, ADH2, ADH4), AHSB4m, AINV, alcA, α-amylase, alternative oxidase (AOD), alcohol oxidase I (AOX1), alcohol oxidase 2 (AOX2), AXDH, B2, CaMV, cellobiohydrolase I (cbh1), ccg-1, cDNA1, cellular filament polypeptide (cfp), cpc-2, ctr4+, CUP1, dihydroxyacetone synthase (DAS), enolase (ENO, ENO1), formaldehyde dehydrogenase (FLD1), FMD, formate dehydrogenase (FMDH), G1, G6, GAA, GAL1, GAL2, GAL3, GAL4, GAL5, GAL6, GALT, GAL5, GAL5, GAL10, GCW14, gdhA, gla-1, α-glucoamylase (glaA), glyceraldehyde-3-phosphate dehydrogenase (gpdA, GAP, GAPDH), phosphoglycerate mutase (GPM1), glycerol kinase (GUT1), HSP82, inv1+, isocitrate lyase (ICL1), acetohydroxy acid isomeroreductase (ILV5), KAR2, KEX2, O-galactosidase (lac4), LEU2, melO, MET3, methanol oxidase (MOX), nmt1, NSP, pcbC, PETS, peroxin 8 (PEX8), phosphoglycerate kinase (PGK, PGK1), pho1, PHO5, PH089, phosphatidylinositol synthase (PIS1), PYK1, pyruvate kinase (pki1), RPS7, sorbitol dehydrogenase (SDH), 3-phosphoserine aminotransferase (SERI), SSA4, SV40, TEF, translation elongation factor 1 alpha (TEF1), THI11, homoserine kinase (THR1), tpi, TPS1, triose phosphate isomerase (TPI1), XRP2, YPT1, and any combination thereof. Illustrative inducible promoters include methanol-induced promoters, e.g., DAS1 and PEX11. Exemplary promoter sequences are provided in Table 4.

A signal peptide (b), also known as a signal sequence, targeting signal, localization signal, localization sequence, signal peptide, transit peptide, leader sequence, or leader peptide, may support secretion of a protein or polynucleotide. Extracellular secretion of a recombinant or heterologously expressed protein from a host cell may facilitate protein purification. A signal peptide may be derived from a precursor (e.g., prepropeptide, preprotein) of a protein. Signal peptides can be derived from a precursor of a protein other than the signal peptides in native a recombinant protein.

Any nucleic acid sequence that encodes a recombinant protein can be used as (c). Preferably such sequence is codon optimized for the species/genus/kingdom of the host cell.

Exemplary transcriptional terminator elements include, but are not limited to, acu-5, adh1+, alcohol dehydrogenase (ADH1, ADH2, ADH4), AHSB4m, AINV, alcA, α-amylase, alternative oxidase (AOD), alcohol oxidase I (AOX1), alcohol oxidase 2 (AOX2), AXDH, B2, CaMV, cellobiohydrolase I (cbh1), ccg-1, cDNA1, cellular filament polypeptide (cfp), cpc-2, ctr4+, CUP1, dihydroxyacetone synthase (DAS), enolase (ENO, ENO1), formaldehyde dehydrogenase (FLD1), FMD, formate dehydrogenase (FMDH), G1, G6, GAA, GAL1, GAL2, GAL3, GAL4, GAL5, GAL6, GALT, GAL5, GAL5, GAL10, GCW14, gdhA, gla-1, α-glucoamylase (glaA), glyceraldehyde-3-phosphate dehydrogenase (gpdA, GAP, GAPDH), phosphoglycerate mutase (GPM1), glycerol kinase (GUT1), HSP82, inv1+, isocitrate lyase (ICL1), acetohydroxy acid isomeroreductase (ILV5), KAR2, KEX2, (3-galactosidase (lac4), LEU2, melO, MET3, methanol oxidase (MOX), nmt1, NSP, pcbC, PETS, peroxin 8 (PEX8), phosphoglycerate kinase (PGK, PGK1), pho1, PHO5, PH089, phosphatidylinositol synthase (PIS1), PYK1, pyruvate kinase (pki1), RPS7, sorbitol dehydrogenase (SDH), 3-phosphoserine aminotransferase (SERI), SSA4, SV40, TEF, translation elongation factor 1 alpha (TEF1), THI11, homoserine kinase (THR1), tpi, TPS1, triose phosphate isomerase (TPI1), XRP2, YPT1, and any combination thereof. Exemplary promoter sequences are provided in Table 5.

Exemplary selectable markers (f) may include but are not limited to: an antibiotic resistance gene (e.g. zeocin, ampicillin, blasticidin, kanamycin, nurseothricin, chloroamphenicol, tetracycline, triclosan, ganciclovir, and any combination thereof), an auxotrophic marker (e.g. ade1, arg4, his4, ura3, met2, and any combination thereof). Exemplary terminator sequences are provided in Table 8.

In one example, a vector for expression in Pichia sp. can include an AOX1 promoter operably linked to a signal peptide (alpha mating factor) that is fused in frame with a nucleic acid sequence encoding a recombinant protein, and a terminator element (AOX1 terminator) immediately downstream of the nucleic acid sequence encoding a recombinant protein.

In another example, a vector comprising a DAS1 promoter is operably linked to a signal peptide (alpha mating factor) that is fused in frame with a nucleic acid sequence encoding a recombinant protein and a terminator element (AOX1 terminator) immediately downstream of a recombinant protein.

A recombinant protein described herein may be secreted from the one or more host cells. In some embodiments, a recombinant POI is secreted from the host cell. The secreted recombinant POI may be isolated and purified by methods such as centrifugation, fractionation, filtration, affinity purification and other methods for separating protein from cells, liquid and solid media components and other cellular products and byproducts. In some embodiments, a recombinant POI is produced in a Pichia Sp. and secreted from the host cells into the culture media. The secreted recombinant protein such as the POI is then separated from other media components for further use.

In some cases, multiple vectors comprising the gene sequence of a protein may be transfected into one or more host cells. A host cell may comprise more than one copy of the gene encoding the recombinant protein. A single host cell may comprise 2, 3, 4, 5, 6, 7, 8, 9, 10, 11, 12, 13, 14, 15, 16, 17, 18, 19, or 20 copies of the recombinant POI or the fusion protein. A single host cell may comprise one or more vectors for the expression of the POI and/or the fusion protein. A single host cell may comprise 2, 3, 4, 5, 6, 7, 8, 9, or 10 vectors for the POI expression and/or the fusion protein expression. Each vector in the host cell may drive the expression of POI and/or the fusion protein using the same promoter. Alternatively, different promoters may be used in different vectors for POI and/or the fusion protein expression.

A recombinant protein such as the POI or the fusion protein may be recombinantly expressed in one or more host cells. As used herein, a “host” or “host cell” denotes here any protein production host selected or genetically modified to produce a desired product. Exemplary hosts include fungi, such as filamentous fungi, as well as bacteria, yeast, plant, insect, and mammalian cells. A host cell can be an organism that is approved as generally regarded as safe by the U.S. Food and Drug Administration.

In some embodiments, a host cell may be transformed to include one or more expression cassettes. As examples, a host cell may be transformed to express one expression cassette, two expression cassettes, three expression cassettes or more expression cassettes. In one example, a host cell is transformed express a first expression cassette that encodes a first POI and express a second expression cassette that encodes a second POI.

As used herein, a “host cell” refers to a cell which is capable of protein expression and optionally protein secretion. Such host cell is applied in the methods of the present invention. For that purpose, for the host cell to express a polypeptide, a nucleotide sequence encoding the polypeptide is present or introduced in the cell. Host cells provided by the present invention can be prokaryotes or eukaryotes. As will be appreciated by one of skill in the art, a prokaryotic cell lacks a membrane-bound nucleus, while a eukaryotic cell has a membrane-bound nucleus. Examples of eukaryotic cells include, but are not limited to, vertebrate cells, mammalian cells, human cells, animal cells, invertebrate cells, plant cells, nematodal cells, insect cells, stem cells, fungal cells or yeast cells.

Examples of yeast cells that may be transformed to include one or more expression cassettes include but are not limited to the Saccharomyces genus (e.g. Saccharomyces cerevisiae, Saccharomyces kluyveri, Saccharomyces uvarum), the Komagataella genus (Komagataella pastoris, Komagataella pseudopastoris or Komagataella phaffii), Kluyveromyces genus (e.g. Kluyveromyces lactis, Kluyveromyces mandanus), the Candida genus (e.g. Candida utilis, Candida cacaoi, the Geotrichum genus (e.g. Geotrichum fermentans), as well as Hansenula polymorpha and Yarrowia lipolytica. A host cell may also be a member of the following species: Arxula spp., Arxula adeninivorans, Kluyveromyces spp., Kluyveromyces lactis, Komagataella phaffii, Pichia spp., Pichia angusta, Pichia pastoris, Saccharomyces spp., Saccharomyces cerevisiae, Schizosaccharomyces spp., Schizosaccharomyces pombe, Yarrowia spp., Yarrowia lipolytica, Agaricus spp., Agaricus bisporus, Aspergillus spp., Aspergillus awamori, Aspergillus fumigatus, Aspergillus nidulans, Aspergillus niger, Aspergillus oryzae, Bacillus subtilis, Colletotrichum spp., Colletotrichum gloeosporiodes, Endothia spp., Endothia parasitica, Escherichia coli, Fusarium spp., Fusarium graminearum, Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculo sum, Penicillium purpurogenum, Penicillium roqueforti, Pleurotus spp., Pleurotus ostreatus, Rhizomucor spp., Rhizomucor miehei, Rhizomucor pusillus, Rhizopus spp., Rhizopus arrhizus, Rhizopus oligosporus, Rhizopus oryzae, Trichoderma spp., Trichoderma altroviride, Trichoderma reesei, or Trichoderma vireus.

The genus Pichia is of particular interest. Pichia comprises a number of species, including the species Pichia pastoris, Pichia methanolica, Pichia kluyveri, and Pichia angusta. Most preferred is the species Pichia pastoris.

The former species Pichia pastoris has been divided and renamed to Komagataella pastoris and Komagataella phaffii. Therefore, Pichia pastoris is synonymous for both Komagataella pastoris and Komagataella phaffii.

In some embodiments, the host cell is a Pichia pastoris, Hansenula polymorpha, Trichoderma reesei, Saccharomyces cerevisiae, Kluyveromyces lactis, Yarrowia lipolytica, Pichia methanolica, Candida boidinii, and Komagataella, and Schizosaccharomyces pombe.

The term “sequence identity” as used herein in the context of amino acid sequences is defined as the percentage of amino acid residues in a candidate sequence that are identical with the amino acid residues in a selected sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared.

In some embodiments, an engineered host cell expressing a recombinant protein such as the POI or the fusion protein may have a growth rate in a media containing fructose as a primary carbon source is higher than a growth rate of a control host cell, wherein the control host cell is identical to the engineered host cell, except the control cell does not express the recombinant protein such as the POI or the fusion protein.

In some embodiments, an engineered host cell expressing a recombinant protein such as the POI or the fusion protein may have a growth rate in a media containing maltose as a primary carbon source is higher than a growth rate of a control host cell, wherein the control host cell is identical to the engineered host cell, except the control cell does not express the recombinant protein such as the POI or the fusion protein.

In some embodiments, an engineered host cell expressing a recombinant protein such as the POI or the fusion protein may have a growth rate in a media containing high fructose corn syrup as a primary carbon source is higher than a growth rate of a control host cell, wherein the control host cell is identical to the engineered host cell, except the control cell does not express the recombinant protein such as the POI or the fusion protein.

In some embodiments, an engineered host cell expressing a recombinant protein such as the POI or the fusion protein may have a growth rate in a media containing molasses as a primary carbon source is higher than a growth rate of a control host cell, wherein the control host cell is identical to the engineered host cell, except the control cell does not express the recombinant protein such as the POI or the fusion protein.

In some embodiments, an engineered host cell expressing a recombinant protein such as the POI or the fusion protein may have a growth rate in a media containing a disaccharide as a primary carbon source is higher than a growth rate of a control host cell, wherein the control host cell is identical to the engineered host cell, except the control cell does not express the recombinant protein such as the POI or the fusion protein.

In some embodiments, an engineered host cell expressing a recombinant protein such as the POI or the fusion protein may have a growth rate in a media containing a mixture of glucose and a disaccharide as a primary carbon source is higher than a growth rate of a control host cell, wherein the control host cell is identical to the engineered host cell, except the control cell does not express the recombinant protein such as the POI or the fusion protein.

In some embodiments, an engineered host cell expressing a recombinant protein such as the POI or the fusion protein may have a growth rate in a media containing a carbon source that is not glucose as a primary carbon source is higher than a growth rate of a control host cell, wherein the control host cell is identical to the engineered host cell, except the control cell does not express the recombinant protein such as the POI or the fusion protein.

TABLE 1
Anchoring proteins

| Sequence | SEQ ID | |
|---|---|---|
| Info | NO: | Amino acid sequence |
| Tir4 from | SEQ ID NO: | QINELNVVLDDVKTNIADYITLSYTPNSGFSLDQMPAGIMDIAAQLVANPSDDSYTTLYSE |
| Saccharomyces | 1 | VDFSAVEHMLTMVPWYSSRLLPELEAMDASLTTSSSAATSSSEVASSSIASSTSSSVAPSSS |
| cerevisiae | | EVVSSSVASSSSEVASSSVASTSEATSSSAVTSSSAVSSSTESVSSSSVSSSSAVSSSEAVSSS |
| | | PVSSVVSSSAGPASSSVAPYNSTIASSSSTAQTSISTIAPYNSTTTTTPASSASSVIISTRNGTT |
| | | VTETDNTLVTKETTVCDYSSTSAVPASTTGYNNSTKVSTATICSTCKEGTSTATDFSTLKT |
| | | TVTVCDSACQAKKSATVVSVQSKTTGIVEQTENGAAKAVIGMGAGALAAVAAMLL |
| Tir4 from | SEQ ID NO: | QINELNVVLDDVKTNIADYITLSYTPNSGFSLDQMPAGIMDIAAQLVANPSDDSYTTLYSE |
| Saccharomyces | 320 | VDFSAVEHMLTMVPWYSSRLLPELEAMDASLTTSSSAATSSSEVASSSIASSTSSSVAPSSS |
| cerevisiae | | EVVSSSVASSSSEVASSSVASTSEATSSSAVTSSSAVSSSTESVSSSSVSSSSAVSSSEAVSSS |
| | | PVSSVVSSSAGPASSSVAPYNSTIASSSSTAQTSISTIAPYNSTTTTTPASSASSVIISTRNGTT |
| | | VTETDNTLVTKETTVCDYSSTSAVPASTTGYNNSTKVSTATICSTCKEGTSTATDFSTLKT |
| | | TVTVCDSACQAKKSATVVSVQSKTTGIVEQTEN |
| Tir4 from | SEQ ID NO: | MAYSKITLLAALAAIAYAQTQAQINELNVVLDDVKTNIADYITLSYTPNSGFSLDQMPAG |
| Saccharomyces | 2 | IMDIAAQLVANPSDDSYTTLYSEVDFSAVEHMLTMVPWYSSRLLPELEAMDASLTTSSSA |
| cerevisiae | | ATSSSEVASSSIASSTSSSVAPSSSEVVSSSVASSSSEVASSSVASTSEATSSSAVTSSSAVSS |
| (underlined is | | STESVSSSSVSSSSAVSSSEAVSSSPVSSVVSSSAGPASSSVAPYNSTIASSSSTAQTSISTIAP |
| signal peptide, may | | YNSTTTTTPASSASSVIISTRNGTTVTETDNTLVTKETTVCDYSSTSAVPASTTGYNNSTK |
| or may not be | | VSTATICSTCKEGTSTATDFSTLKTTVTVCDSACQAKKSATVVSVQSKTTGIVEQTENGA |
| utilized in design) | | AKAVIGMGAGALAAVAAMLL |
| Tir4 from | SEQ ID NO: | QINELNVVLDDVKTNIADYITLSYTPNSGFSLDQMPAGIMDIAAQLVANPSDDSYTTLYSE |
| Saccharomyces | 320 | VDFSAVEHMLTMVPWYSSRLLPELEAMDASLTTSSSAATSSSEVASSSIASSTSSSVAPSSS |
| cerevisiae | | EVVSSSVASSSSEVASSSVASTSEATSSSAVTSSSAVSSSTESVSSSSVSSSSAVSSSEAVSSS |
| | | PVSSVVSSSAGPASSSVAPYNSTIASSSSTAQTSISTIAPYNSTTTTTPASSASSVIISTRNGTT |
| | | VTETDNTLVTKETTVCDYSSTSAVPASTTGYNNSTKVSTATICSTCKEGTSTATDFSTLKT |
| | | TVTVCDSACQAKKSATVVSVQSKTTGIVEQTEN |
| Tir4 | SEQ ID NO: | QINELNVVLDDVKTNIADYITLSYTPNSGFSLDQMPAGIMDIAAQLVANPSDDSYTTLYSE |
| (NP_014652.1) | 3 | VDFSAVEHMLTMVPWYSSRLLPELEAMDASLTTSSSAATSSSEVASSSIASSTSSSVAPSSS |
| from | | EVVSSSVAPSSSEVVSSSVAPSSSEVVSSSVASSSSEVASSSVAPSSSEVVSSSVASSSSEVA |
| Saccharomyces | | SSSVAPSSSEVVSSSVAPSSSEVVSSSVASSSSEVASSSVAPSSSEVVSSSVASSTSEATSSSA |
| cerevisiae | | VTSSSAVSSSTESVSSSSVSSSSAVSSSEAVSSSPVSSVVSSSAGPASSSVAPYNSTIASSSST |
| | | AQTSISTIAPYNSTTTTTPASSASSVIISTRNGTTVTETDNTLVTKETTVCDYSSTSAVPAST |
| | | TGYNNSTKVSTATICSTCKEGTSTATDFSTLKTTVTVCDSACQAKKSATVVSVQSKTTGI |
| | | VEQTENGAAKAVIGMGAGALAAVAAMLL |
| Tir4 | SEQ ID NO: | MAYSKITLLAALAALAYAQTQAQINELNVVLDDVKTNIADYITLSYTPNSGFSLDQMPAG |
| (NP_014652.1) | 4 | IMDIAAQLVANPSDDSYTTLYSEVDFSAVEHMLTMVPWYSSRLLPELEAMDASLTTSSSA |
| from | | ATSSSEVASSSIASSTSSSVAPSSSEVVSSSVAPSSSEVVSSSVAPSSSEVVSSSVASSSSEVA |
| Saccharomyces | | SSSVAPSSSEVVSSSVASSSSEVASSSVAPSSSEVVSSSVAPSSSEVVSSSVASSSSEVASSSV |
| cerevisiae | | APSSSEVVSSSVASSTSEATSSSAVTSSSAVSSSTESVSSSSVSSSSAVSSSEAVSSSPVSSVV |
| (underlined is | | SSSAGPASSSVAPYNSTIASSSSTAQTSISTIAPYNSTTTTTPASSASSVIISTRNGTTVTETD |
| signal peptide, may | | NTLVTKETTVCDYSSTSAVPASTTGYNNSTKVSTATICSTCKEGTSTATDFSTLKTTVTVC |
| or may not be | | DSACQAKKSATVVSVQSKTTGIVEQTENGAAKAVIGMGAGALAAVAAMLL |
| utilized in design) | | |
| Tir4 | SEQ ID NO: | QINELNVVLDDVKTNIADYITLSYTPNSGFSLDQMPAGIMDIAAQLVANPSDDSYTTLYSE |
| (NP_014652.1) | 321 | VDFSAVEHMLTMVPWYSSRLLPELEAMDASLTTSSSAATSSSEVASSSIASSTSSSVAPSSS |
| from | | EVVSSSVAPSSSEVVSSSVAPSSSEVVSSSVASSSSEVASSSVAPSSSEVVSSSVASSSSEVA |
| Saccharomyces | | SSSVAPSSSEVVSSSVAPSSSEVVSSSVASSSSEVASSSVAPSSSEVVSSSVASSTSEATSSSA |
| cerevisiae | | VTSSSAVSSSTESVSSSSVSSSSAVSSSEAVSSSPVSSVVSSSAGPASSSVAPYNSTIASSSST |
| (without C- | | AQTSISTIAPYNSTTTTTPASSASSVIISTRNGTTVTETDNTLVTKETTVCDYSSTSAVPAST |
| terminus of Tir4 | | TGYNNSTKVSTATICSTCKEGTSTATDFSTLKTTVTVCDSACQAKKSATVVSVQSKTTGI |
| GPI anchor or | | VEQTEN |
| signal peptide or | | |
| signal peptide) | | |
| Dan1 from | SEQ ID NO: | ASVTTTLSPYDERVNLIELAVYVSDIGAHLSEYYAFQALHKTETYPPEIAKAVFAGGDFTT |
| Saccharomyces | 5 | MLTGISGDEVTRMITGVPWYSTRLMGAISEALANEGIATAVPASTTEASSTSTSEASSAAT |
| cerevisiae | | ESSSSSESSAETSSNAASTQATVSSESSSAASTIASSAESSVASSVASSVASSASFANTTAPV |
| | | SSTSSISVTPVVQNGTDSTVTKTQASTVETTITSCSNNVCSTVTKPVSSKAQSTATSVTSSA |
| | | SRVIDVTTNGANKFNNGVFGAAAIAGAAALLL |
| Dan1 from | SEQ ID NO: | MSRISILAVAAALVASATAASVTTTLSPYDERVNLIELAVYVSDIGAHLSEYYAFQALHK |
| Saccharomyces | 6 | TETYPPEIAKAVFAGGDFTTMLTGISGDEVTRMITGVPWYSTRLMGAISEALANEGIATA |
| cerevisiae | | VPASTTEASSTSTSEASSAATESSSSSESSAETSSNAASTQATVSSESSSAASTIASSAESSV |
| (underlined is | | ASSVASSVASSASFANTTAPVSSTSSISVTPVVQNGTDSTVTKTQASTVETTITSCSNNVCS |
| signal peptide, may | | TVTKPVSSKAQSTATSVTSSASRVIDVTTNGANKFNNGVFGAAAIAGAAALLL |
| or may not be | | |
| utilized in design) | | |
| Dan4 from | SEQ ID NO: | ITATTTLSPYDERVNLIELAVYVSDIRAHIFQYYSFRNHHKTETYPSEIAAAVFDYGDFTTR |
| Saccharomyces | 7 | LTGISGDEVTRMITGVPWYSTRLKPAISSALSKDGIYTAIPTSTSTTTTKSSTSTTPTTTITST |
| cerevisiae | | TSTTSTTPTTSTTSTTPTTSTTSTTPTTSTTSTTPTTSTTSTTPTTSTTSTTPTTSTTSTTPTTST |
| | | TSTTPTTSTTSTTPTTSTTPTTSTTSTTSQTSTKSTTPTTSSTSTTPTTSTTPTTSTTSTAPTTS |
| | | TTSTTSTTSTISTAPTTSTTSSTFSTSSASASSVISTTATTSTTFASLTTPATSTASTDHTTSSV |
| | | STTNAFTTSATTTTTSDTYISSSSPSQVTSSAEPTTVSEVTSSVEPTRSSQVTSSAEPTTVSEF |
| | | TSSVEPTRSSQVTSSAEPTTVSEFTSSVEPTRSSQVTSSAEPTTVSEFTSSVEPTRSSQVTSSA |
| | | EPTTVSEFTSSVEPTRSSQVTSSAEPTTVSEFTSSVEPIRSSQVTSSAEPTTVSEVTSSVEPIRS |
| | | SQVTTTEPVSSFGSTFSEITSSAEPLSFSKATTSAESISSNQITISSELIVSSVITSSSEIPSSIEVL |
| | | TSSGISSSVEPTSLVGPSSDESISSTESLSATSTFTSAVVSSSKAADFFTRSTVSAKSDVSGNS |
| | | STQSTTFFATPSTPLAVSSTVVTSSTDSVSPNIPFSEISSSPESSTAITSTSTSFIAERTSSLYLS |
| | | SSNMSSFTLSTFTVSQSIVSSFSMEPTSSVASFASSSPLLVSSRSNCSDARSSNTISSGLFSTIE |
| | | NVRNATSTFTNLSTDEIVITSCKSSCTNEDSVLTKTQVSTVETTITSCSGGICTTLMSPVTTI |
| | | NAKANTLTTTETSTVETTITTCPGGVCSTLTVPVTTITSEATTTATISCEDNEEDITSTETEL |
| | | LTLETTITSCSGGICTTLMSPVTTINAKANTLTTTETSTVETTITTCSGGVCSTLTVPVTTITS |
| | | EATTTATISCEDNEEDVASTKTELLTMETTITSCSGGICTTLMSPVSSFNSKATTSNNAESTI |
| | | PKAIKVSCSAGACTTLTTVDAGISMFTRTGLSITQTTVTNCSGGTCTMLTAPIATATSKVIS |
| | | PIPKASSATSIAHSSASYTVSINTNGAYNFDKDNIFGTAIVAVVALLLL |
| Dan4 from | SEQ ID NO: | MVNISIVAGIVALATSAAAITATTTLSPYDERVNLIELAVYVSDIRAHIFQYYSFRNHHKTE |
| Saccharomyces | 8 | TYPSEIAAAVFDYGDFTTRLTGISGDEVTRMITGVPWYSTRLKPAISSALSKDGIYTAIPTS |
| cerevisiae | | TSTTTTKSSTSTTPTTTITSTTSTTSTTPTTSTTSTTPTTSTTSTTPTTSTTSTTPTTSTTSTTPT |
| (underlined is | | TSTTSTTPTTSTTSTTPTTSTTSTTPTTSTTSTTPTTSTTPTTSTTSTTSQTSTKSTTPTTSSTST |
| signal peptide, may | | TPTTSTTPTTSTTSTAPTTSTTSTTSTTSTISTAPTTSTTSSTFSTSSASASSVISTTATTSTTFA |
| or may not be | | SLTTPATSTASTDHTTSSVSTTNAFTTSATTTTTSDTYISSSSPSQVTSSAEPTTVSEVTSSV |
| utilized in design) | | EPTRSSQVTSSAEPTTVSEFTSSVEPTRSSQVTSSAEPTTVSEFTSSVEPTRSSQVTSSAEPTT |
| | | VSEFTSSVEPTRSSQVTSSAEPTTVSEFTSSVEPTRSSQVTSSAEPTTVSEFTSSVEPIRSSQV |
| | | TSSAEPTTVSEVTSSVEPIRSSQVTTTEPVSSFGSTFSEITSSAEPLSFSKATTSAESISSNQITI |
| | | SSELIVSSVITSSSEIPSSIEVLTSSGISSSVEPTSLVGPSSDESISSTESLSATSTFTSAVVSSSK |
| | | AADFFTRSTVSAKSDVSGNSSTQSTTFFATPSTPLAVSSTVVTSSTDSVSPNIPFSEISSSPES |
| | | STAITSTSTSFIAERTSSLYLSSSNMSSFTLSTFTVSQSIVSSFSMEPTSSVASFASSSPLLVSS |
| | | RSNCSDARSSNTISSGLFSTIENVRNATSTFTNLSTDEIVITSCKSSCTNEDSVLTKTQVSTV |
| | | ETTITSCSGGICTTLMSPVTTINAKANTLTTTETSTVETTITTCPGGVCSTLTVPVTTITSEA |
| | | TTTATISCEDNEEDITSTETELLTLETTITSCSGGICTTLMSPVTTINAKANTLTTTETSTVET |
| | | TITTCSGGVCSTLTVPVTTITSEATTTATISCEDNEEDVASTKTELLTMETTITSCSGGICTT |
| | | LMSPVSSFNSKATTSNNAESTIPKAIKVSCSAGACTTLTTVDAGISMFTRTGLSITQTTVTN |
| | | CSGGTCTMLTAPIATATSKVISPIPKASSATSIAHSSASYTVSINTNGAYNFDKDNIFGTAIV |
| | | AVVALLLL |
| Sag1 from | SEQ ID NO: | ININDITFSNLEITPLTANKQPDQGWTATFDFSIADASSIREGDEFTLSMPHVYRIKLLNSSQ |
| Saccharomyces | 9 | TATISLADGTEAFKCYVSQQAAYLYENTTFTCTAQNDLSSYNTIDGSITFSLNFSDGGSSY |
| cerevisiae | | EYELENAKFFKSGPMLVKLGNQMSDVVNFDPAAFTENVFHSGRSTGYGSFESYHLGMY |
| | | CPNGYFLGGTEKIDYDSSNNNVDLDCSSVQVYSSNDFNDWWFPQSYNDTNADVTCFGS |
| | | NLWITLDEKLYDGEMLWVNALQSLPANVNTIDHALEFQYTCLDTIANTTYATQFSTTREF |
| | | IVYQGRNLGTASAKSSFISTTTTDLTSINTSAYSTGSISTVETGNRTTSEVISHVVTTSTKLS |
| | | PTATTSLTIAQTSIYSTDSNITVGTDIHTTSEVISDVETISRETASTVVAAPTSTTGWTGAMN |
| | | TYISQFTSSSFATINSTPIISSSAVFETSDASIVNVHTENITNTAAVPSEEPTFVNATRNSLNS |
| | | FCSSKQPSSPSSYTSSPLVSSLSVSKTLLSTSFTPSVPTSNTYIKTKNTGYFEHTALTTSSVG |
| | | LNSFSETAVSSQGTKIDTFLVSSLIAYPSSASGSQLSGIQQNFTSTSLMISTYEGKASIFFSAE |
| | | LGSIIFLLLSYLLF |
| Sag1 from | SEQ ID NO: | MFTFLKIILWLFSLALASAININDITFSNLEITPLTANKQPDQGWTATFDFSIADASSIREGD |
| Saccharomyces | 10 | EFTLSMPHVYRIKLLNSSQTATISLADGTEAFKCYVSQQAAYLYENTTFTCTAQNDLSSY |
| cerevisiae | | NTIDGSITFSLNFSDGGSSYEYELENAKFFKSGPMLVKLGNQMSDVVNFDPAAFTENVFH |
| (underlined is | | SGRSTGYGSFESYHLGMYCPNGYFLGGTEKIDYDSSNNNVDLDCSSVQVYSSNDFNDW |
| signal peptide, may | | WFPQSYNDTNADVTCFGSNLWITLDEKLYDGEMLWVNALQSLPANVNTIDHALEFQYT |
| or may not be | | CLDTIANTTYATQFSTTREFIVYQGRNLGTASAKSSFISTTTTDLTSINTSAYSTGSISTVET |
| utilized in design) | | GNRTTSEVISHVVTTSTKLSPTATTSLTIAQTSIYSTDSNITVGTDIHTTSEVISDVETISRET |
| | | ASTVVAAPTSTTGWTGAMNTYISQFTSSSFATINSTPIISSSAVFETSDASIVNVHTENITNT |
| | | AAVPSEEPTFVNATRNSLNSFCSSKQPSSPSSYTSSPLVSSLSVSKTLLSTSFTPSVPTSNTYI |
| | | KTKNTGYFEHTALTTSSVGLNSFSETAVSSQGTKIDTFLVSSLIAYPSSASGSQLSGIQQNF |
| | | TSTSLMISTYEGKASIFFSAELGSIIFLLLSYLLF |
| Fig2 from | SEQ ID NO: | QIVFYQNSSTSLPVPTLVSTSIADFHESSSTGEVQYSSSYSYVQPSIDSFTSSSFLTSFEAPTE |
| Saccharomyces | 11 | TSSSYAVSSSLITSDTFSSYSDIFDEETSSLISTSAASSEKASSTLSSTAQPHRTSHSSSSFELP |
| cerevisiae | | VTAPSSSSLPSSTSLTFTSVNPSQSWTSFNSEKSSALSSTIDFTSSEISGSTSPKSLESFDTTGT |
| | | ITSSYSPSPSSKNSNQTSLLSPLEPLSSSSGDLILSSTIQATTNDQTSKTIPTLVDATSSLPPTL |
| | | RSSSMAPTSGSDSISHNFTSPPSKTSGNYDVLTSNSIDPSLFTTTSEYSSTQLSSLNRASKSE |
| | | TVNFTASIASTPFGTDSATSLIDPISSVGSTASSFVGISTANFSTQGNSNYVPESTASGSSQY |
| | | QDWSSSSLPLSQTTWVVINTTNTQGSVTSTTSPAYVSTATKTVDGVITEYVTWCPLTQTK |
| | | SQAIGVSSSISSVPQASSFSGSSILSSNSSTLAASNNVPESTASGSSQYQDWSSSSLPLSQTT |
| | | WVVINTTNTQGSVTSTTSPAYVSTATKTVDGVITEYVTWCPLTQTKSQAIGISSSTISATQ |
| | | TSKPSSILTLGISTLQLSDATFKGTETINTHLMTESTSITEPTYFSGTSDSFYLCTSEVNLASS |
| | | LSSYPNFSSSEGSTATITNSTVTFGSTSKYPSTSVSNPTEASQHVSSSVNSLTDFTSNSTETI |
| | | AVISNIHKTSSNKDYSLTTTQLKTSGMQTLVLSTVTTTVNGAATEYTTWCPASSIAYTTSI |
| | | SYKTLVLTTEVCSHSECTPTVITSVTATSSTIPLLSTSSSTVLSSTVSEGAKNPAASEVTINT |
| | | QVSATSEATSTSTQVSATSATATASESSTTSQVSTASETISTLGTQNFTTTGSLLFPALSTE |
| | | MINTTVVSRKTLIISTEVCSHSKCVPTVITEVVTSKGTPSNGHSSQTLQTEAVEVTLSSHQT |
| | | VTMSTEVCSNSICTPTVITSVQMRSTPFPYLTSSTSSSSLASTKKSSLEASSEMSTFSVSTQS |
| | | LPLAFTSSEKRSTTSVSQWSNTVLTNTIMSSSSNVISTNEKPSSTTSPYNFSSGYSLPSSSTPS |
| | | QYSLSTATTTINGIKTVYTTWCPLAEKSTVAASSQSSRSVDRFVSSSKPSSSLSQTSIQYTL |
| | | STATTTISGLKTVYTTWCPLTSKSTLGATTQTSSTAKVRITSASSATSTSISLSTSTESESSSG |
| | | YLSKGVCSGTECTQDVPTQSSSPASTLAYSPSVSTSSSSSFSTTTASTLTSTHTSVPLLPSSS |
| | | SISASSPSSTSLLSTSLPSPAFTSSTLPTATAVSSSTFIASSLPLSSKSSLSLSPVSSSILMSQFSS |
| | | SSSSSSSLASLPSLSISPTVDTVSVLQPTTSIATLTCTDSQCQQEVSTICNGSNCDDVTSTAT |
| | | TPPSTVTDTMTCTGSECQKTTSSSCDGYSCKVSETYKSSATISACSGEGCQASATSELNSQ |
| | | YVTMTSVITPSAITTTSVEVHSTESTISITTVKPVTYTSSDTNGELITITSSSQTVIPSVTTIITR |
| | | TKVAITSAPKPTTTTYVEQRLSSSGIATSFVAAASSTWITTPIVSTYAGSASKFLCSKFFMI |
| | | MVMVINFI |
| Fig2 from | SEQ ID NO: | MNSFASLGLIYSVVNLLTRVEAQIVFYQNSSTSLPVPTLVSTSIADFHESSSTGEVQYSSSY |
| Saccharomyces | 12 | SYVQPSIDSFTSSSFLTSFEAPTETSSSYAVSSSLITSDTFSSYSDIFDEETSSLISTSAASSEKA |
| cerevisiae | | SSTLSSTAQPHRTSHSSSSFELPVTAPSSSSLPSSTSLTFTSVNPSQSWTSFNSEKSSALSSTI |
| (underlined is | | DFTSSEISGSTSPKSLESFDTTGTITSSYSPSPSSKNSNQTSLLSPLEPLSSSSGDLILSSTIQAT |
| signal peptide, may | | TNDQTSKTIPTLVDATSSLPPTLRSSSMAPTSGSDSISHNFTSPPSKTSGNYDVLTSNSIDPS |
| or may not be | | LFTTTSEYSSTQLSSLNRASKSETVNFTASIASTPFGTDSATSLIDPISSVGSTASSFVGISTA |
| utilized in design) | | NFSTQGNSNYVPESTASGSSQYQDWSSSSLPLSQTTWVVINTTNTQGSVTSTTSPAYVST |
| | | ATKTVDGVITEYVTWCPLTQTKSQAIGVSSSISSVPQASSFSGSSILSSNSSTLAASNNVPES |
| | | TASGSSQYQDWSSSSLPLSQTTWVVINTTNTQGSVTSTTSPAYVSTATKTVDGVITEYVT |
| | | WCPLTQTKSQAIGISSSTISATQTSKPSSILTLGISTLQLSDATFKGTETINTHLMTESTSITEP |
| | | TYFSGTSDSFYLCTSEVNLASSLSSYPNFSSSEGSTATITNSTVTFGSTSKYPSTSVSNPTEA |
| | | SQHVSSSVNSLTDFTSNSTETIAVISNIHKTSSNKDYSLTTTQLKTSGMQTLVLSTVTTTVN |
| | | GAATEYTTWCPASSIAYTTSISYKTLVLTTEVCSHSECTPTVITSVTATSSTIPLLSTSSSTV |
| | | LSSTVSEGAKNPAASEVTINTQVSATSEATSTSTQVSATSATATASESSTTSQVSTASETIS |
| | | TLGTQNFTTTGSLLFPALSTEMINTTVVSRKTLIISTEVCSHSKCVPTVITEVVTSKGTPSNG |
| | | HSSQTLQTEAVEVTLSSHQTVTMSTEVCSNSICTPTVITSVQMRSTPFPYLTSSTSSSSLAST |
| | | KKSSLEASSEMSTFSVSTQSLPLAFTSSEKRSTTSVSQWSNTVLTNTIMSSSSNVISTNEKPS |
| | | STTSPYNFSSGYSLPSSSTPSQYSLSTATTTINGIKTVYTTWCPLAEKSTVAASSQSSRSVD |
| | | RFVSSSKPSSSLSQTSIQYTLSTATTTISGLKTVYTTWCPLTSKSTLGATTQTSSTAKVRITS |
| | | ASSATSTSISLSTSTESESSSGYLSKGVCSGTECTQDVPTQSSSPASTLAYSPSVSTSSSSSFS |
| | | TTTASTLTSTHTSVPLLPSSSSISASSPSSTSLLSTSLPSPAFTSSTLPTATAVSSSTFIASSLPL |
| | | SSKSSLSLSPVSSSILMSQFSSSSSSSSSLASLPSLSISPTVDTVSVLQPTTSIATLTCTDSQCQ |
| | | QEVSTICNGSNCDDVTSTATTPPSTVTDTMTCTGSECQKTTSSSCDGYSCKVSETYKSSAT |
| | | ISACSGEGCQASATSELNSQYVTMTSVITPSAITTTSVEVHSTESTISITTVKPVTYTSSDTN |
| | | GELITITSSSQTVIPSVTTIITRTKVAITSAPKPTTTTYVEQRLSSSGIATSFVAAASSTWITTP |
| | | IVSTYAGSASKFLCSKFFMIMVMVINFI |
| Sed1 from | SEQ ID NO: | QFSNSTSASSTDVTSSSSISTSSGSVTITSSEAPESDNGTSTAAPTETSTEAPTTAIPTNGTST |
| Saccharomyces | 13 | EAPTTAIPTNGTSTEAPTDTTTEAPTTALPTNGTSTEAPTDTTTEAPTTGLPTNGTTSAFPPT |
| cerevisiae | | TSLPPSNTTTTPPYNPSTDYTTDYTVVTEYTTYCPEPTTFTTNGKTYTVTEPTTLTITDCPC |
| | | TIEKPTTTSTTEYTVVTEYTTYCPEPTTFTTNGKTYTVTEPTTLTITDCPCTIEKSEAPESSV |
| | | PVTESKGTTTKETGVTTKQTTANPSLTVSTVVPVSSSASSHSVVINSNGANVVVPGALGL |
| | | AGVAMLFL |
| Sed1 from | SEQ ID NO: | MKLSTVLLSAGLASTTLAQFSNSTSASSTDVTSSSSISTSSGSVTITSSEAPESDNGTSTAAP |
| Saccharomyces | 14 | TETSTEAPTTAIPTNGTSTEAPTTAIPTNGTSTEAPTDTTTEAPTTALPTNGTSTEAPTDTTT |
| cerevisiae | | EAPTTGLPTNGTTSAFPPTTSLPPSNTTTTPPYNPSTDYTTDYTVVTEYTTYCPEPTTFTTN |
| (underlined is | | GKTYTVTEPTTLTITDCPCTIEKPTTTSTTEYTVVTEYTTYCPEPTTFTTNGKTYTVTEPTT |
| signal peptide, may | | LTITDCPCTIEKSEAPESSVPVTESKGTTTKETGVTTKQTTANPSLTVSTVVPVSSSASSHS |
| or may not be | | VVINSNGANVVVPGALGLAGVAMLFL |
| utilized in design) | | |

TABLE 2
Carbon utilization proteins

| Sequence | SEQ ID | |
|---|---|---|
| Info | NO: | Amino acid sequences |
| Saccharomyces | SEQ ID NO: 15 | SMTNETSDRPLVHFTPNKGWMNDPNGLWYDEKDAKWHLYFQYNPNDTVWGTPLFWGHATSD |
| cerevisiae | | DLTNWEDQPIAIAPKRNDSGAFSGSMVVDYNNTSGFFNDTIDPRQRCVAIWTYNTPESEEQYISY |
| SUC2 | | SLDGGYTFTEYQKNPVLAANSTQFRDPKVFWYEPSQKWIMTAAKSQDYKIEIYSSDDLKSWKLE |
| (without | | SAFANEGFLGYQYECPGLIEVPTEQDPSKSYWVMFISINPGAPAGGSFNQYFVGSFNGTHFEAFD |
| peptides | | NQSRVVDFGKDYYALQTFFNTDPTYGSALGIAWASNWEYSAFVPTNPWRSSMSLVRKFSLNTE |
| that are | | YQANPETELINLKAEPILNISNAGPWSRFATNTTLTKANSYNVDLSNSTGTLEFELVYAVNTTQTI |
| cleaved | | SKSVFADLSLWFKGLEDPEEYLRMGFEVSASSFFLDRGNSKVKFVKENPYFTNRMSVNNQPFKS |
| off post- | | ENDLSYYKVYGLLDQNILELYFNDGDVVSTNTYFMTTGNALGSVNMTTGVDNLFYIDKFQVRE |
| translationally) | | VK |
| Saccharomyces | SEQ ID NO: 16 | MLLQAFLFLLAGFAAKISASMTNETSDRPLVHFTPNKGWMNDPNGLWYDEKDAKWHLYFQYN |
| cerevisiae | | PNDTVWGTPLFWGHATSDDLTNWEDQPIAIAPKRNDSGAFSGSMVVDYNNTSGFFNDTIDPRQR |
| SUC2 | | CVAIWTYNTPESEEQYISYSLDGGYTFTEYQKNPVLAANSTQFRDPKVFWYEPSQKWIMTAAKS |
| (including | | QDYKIEIYSSDDLKSWKLESAFANEGFLGYQYECPGLIEVPTEQDPSKSYWVMFISINPGAPAGGS |
| peptides | | FNQYFVGSFNGTHFEAFDNQSRVVDFGKDYYALQTFFNTDPTYGSALGIAWASNWEYSAFVPT |
| that are | | NPWRSSMSLVRKFSLNTEYQANPETELINLKAEPILNISNAGPWSRFATNTTLTKANSYNVDLSN |
| cleaved | | STGTLEFELVYAVNTTQTISKSVFADLSLWFKGLEDPEEYLRMGFEVSASSFFLDRGNSKVKFVK |
| off post- | | ENPYFTNRMSVNNQPFKSENDLSYYKVYGLLDQNILELYFNDGDVVSTNTYFMTTGNALGSVN |
| translationally) | | MTTGVDNLFYIDKFQVREVK |
| UniProt | | |
| KB - | | |
| P00724 | | |
| (INV2_ | | |
| YEAST) | | |
| Pichia | SEQ ID NO: 17 | MTIESQEPWWKSAVVYQVWPASFKDSNGDGIGDLNGITSELDHIKSLGTDVIWLSPHYASPLDD |
| angusta | | MGYDISDYNAINPQFGTMEDMDRLLAEIKKRDMRLILDLVINHTSSEHAWFKESRSSRDNPKRD |
| MAL1 | | WYIWKDNANNWLSFFSGSAWSYDEKTKQYYLRLFAETQPDLNWENPKTREAIYKSALEFWYE |
| (including | | KGVSGFRIDTAGLYSKVQTFEDAPVTFPGEKYQPAGPLINSGPRIHEFHKEMYEKVTSRYDAMTV |
| peptides | | GEVGHCSKADALKYVSAKEKEMNMMFLFDTVDVGSDKSDRFRYKGFTLTDFKDAIINQSNFIFD |
| that are | | DETGELNDAWSTVFIENHDQPRCVTRFGNTSNKLFWSRSAKMLALLQTTLTGTLFVYQGQEIGM |
| cleaved | | TNVSPKWDISEYLDINTINYWNAFNETEHSDEEKAELLKIINLLARDNARTPVQWDSSENGGFGG |
| off post- | | KPWMRINDNYKDINVASQKEDPDSVLNFYRNAIKTRKHYSETLIFGRFEVQDYDNQEIFYYTKTS |
| translationally) | | NKGQKKMAVVLNFTDREVEYPIPQGKLLLSNIANNITGKLQPYEGRLIEVN |
| UniProt | | |
| KB - | | |
| Q9P8G8 | | |
| (Q9P8G8_ | | |
| PICAN) | | |
| Saccharomyces | SEQ ID NO: 322 | MLLQAFLFLLAGFAAKISASMTNETSDRPLVHFTPNKGWMNDPNGLWYDAKEGKWHLYFQYN |
| cerevisiae | | PNDTVWGLPLFWGHATSDDLTHWQDEPVAIAPKRKDSGAYSGSMVIDYNNTSGFFNDTIDPRQ |
| SUC1 | | RCVAIWTYNTPESEEQYISYSLDGGYTFTEYQKNPVLAANSTQFRDPKVFWYEPSKKWIMTAAK |
| (invertase 1) | | SQDYKIEIYSSDDLKSWKLESAFANEGFLGYQYECPGLIEVPSEQDPSKSHWVMFISINPGAPAGG |
| Unitprot | | SFNQYFVGSFNGHHFEAFDNQSRVVDFGKDYYALQTFFNTDPTYGSALGIAWASNWEYSAFVPS |
| Accession: | | NPWRSSMSLVRPFSLNTEYQANPETELINLKAEPILNISSAGPWSRFATNTTLTKANSYNVDLSNS |
| P10594 | | TGTLEFELVYAVNTTQTISKSVFADLSLWFKGLEDPEEYLRMGFEVSASSFFLDRGNSKVKFVKE |
| | | NPYFTNRMSVNNQPFKSENDLSYYKVYGLLDQNILELYFNDGDVVSTNTYFMTTGNALGSVNM |
| | | TTGVDNLFYIDKFQVREVK |
| Kluyveromyces | SEQ ID NO: 323 | MLKLLSLMVPLASAAVIHRRDANISAIASEWNSTSNSSSSLSLNRPAVHYSPEEGWMNDPNGLW |
| lactis | | YDAKEEDWHIYYQYYPDAPHWGLPLTWGHAVSKDLTVWDEQGVAFGPEFETAGAFSGSMVID |
| INV1 | | YNNTSGFFNSSTDPRQRVVAIWTLDYSGSETQQLSYSHDGGYTFTEYSDNPVLDIDSDAFRDPKV |
| (invertase) | | FWYQGEDSESEGNWVMTVAEADRFSVLIYSSPDLKNWTLESNFSREGYLGYNYECPGLVKVPY |
| Unitprot | | VKNTTYASAPGSNITSSGPLHPNSTVSFSNSSSIAWNASSVPLNITLSNSTLVDETSQLEEVGYAW |
| Accession: | | VMIVSFNPGSILGGSGTEYFIGDFNGTHFEPLDKQTRFLDLGKDYYALQTFFNTPNEVDVLGIAW |
| Q9Y746 | | ASNWQYANQVPTDPWRSSMSLVRNFTITEYNINSNTTALVLNSQPVLDFTSLRKNGTSYTLENLT |
| | | LNSSSHEVLEFEDPTGVFEFSLEYSVNFTGIHNWVFTDLSLYFQGDKDSDEYLRLGYEANSKQFF |
| | | LDRGHSNIPFVQENPFFTQRLSVSNPPSSNSSTFDVYGIVDRNIIELYFNNGTVTSTNTFFFSTGNNI |
| | | GSIIVKSGVDDVYEIESLKVNQFYVD |
| Cyberlindnera | SEQ ID NO: 324 | MSLTKDASEDQEDIKSLTMNTSLVDSSIYRPLVHLTPPVGWMNDPNGLFYDSSESTYHVYYQYN |
| jadinii | | PNDTIWGLPLYWGHATSDDLLTWDHHAPAIGPENDDEGIYSGSIVIDYDNTSGFFDDSTRPEQRI |
| INV1 | | VAIYTNNLPDVETQDIAYSTDGGYTFEKYENNPVIDVNSTQFRDPKVIWYEETEQWVMTVAKSQ |
| (invertase) | | EYKIQIYTSDNLKDWSLASNFSTKGYVGYQYECPGLFEATIENPKSGDPEKKWVMVLAINPGSPL |
| Unitprot | | GGSINEYFVGDFNGTEFIPDDDATRFMDTGKDFYAFQAFFNAPENRSIGVAWSSNWQYSNQVPD |
| Accession: | | PDGYRSSMSSIREYTLRYVSTNPESEQLILCQKPFFVNETDLKVVEEYKVSNSSLTVDHTFGSSFA |
| O94224 | | NSNTTGLLDFNMTFTVNGTTDVTQKDSVTFELRIKSNQSDEAIALGYDYNNEQFYINRATESYFQ |
| | | RTNQFFQERWSTYVQPLTITESGDKQYQLYGLVDNNILELYFNDGAFTSTNTFFLEKGKPSNVDI |
| | | VASSSKEAYHRGPAD |
| Oryza | SEQ ID NO: 325 | MELAVGAGGMRRSASHTSLSESDDFDLSRLLNKPRINVERQRSFDDRSLSDVSYSGGGHGGTRG |
| sativa | | GFDGMYSPGGGLRSLVGTPASSALHSFEPHPIVGDAWEALRRSLVFFRGQPLGTIAAFDHASEEV |
| japonica | | LNYDQVFVRDFVPSALAFLMNGEPEIVRHFLLKTLLLQGWEKKVDRFKLGEGAMPASFKVLHD |
| (rice) | | SKKGVDTLHADFGESAIGRVAPVDSGFWWIILLRAYTKSTGDLTLAETPECQKGMRLILSLCLSE |
| CINV1 | | GFDTFPTLLCADGCCMIDRRMGVYGYPIEIQALFFMALRCALQLLKHDNEGKEFVERIATRLHAL |
| (invertase) | | SYHMRSYYWLDFQQLNDIYRYKTEEYSHTAVNKFNVIPDSIPDWLFDFMPCQGGFFIGNVSPAR |
| Unitprot | | MDFRWFALGNMIAILSSLATPEQSTAIMDLIEERWEELIGEMPLKICYPAIENHEWRIVTGCDPKN |
| Accession: | | TRWSYHNGGSWPVLLWLLTAACIKTGRPQIARRAIDLAERRLLKDGWPEYYDGKLGRYVGKQA |
| Q69T31 | | RKFQTWSIAGYLVAKMMLEDPSHLGMISLEEDKAMKPVLKRSASWTN |
| Arabidopsis | SEQ ID NO: 326 | MEGVGLRAVGSHCSLSEMDDLDLTRALDKPRLKIERKRSFDERSMSELSTGYSRHDGIHDSPRG |
| thaliana | | RSVLDTPLSSARNSFEPHPMMAEAWEALRRSMVFFRGQPVGTLAAVDNTTDEVLNYDQVFVRD |
| Alkaline/ | | FVPSALAFLMNGEPDIVKHFLLKTLQLQGWEKRVDRFKLGEGVMPASFKVLHDPIRETDNIVAD |
| neutral | | FGESAIGRVAPVDSGFWWIILLRAYTKSTGDLTLSETPECQKGMKLILSLCLAEGFDTFPTLLCAD |
| invertase | | GCSMIDRRMGVYGYPIEIQALFFMALRSALSMLKPDGDGREVIERIVKRLHALSFHMRNYFWLD |
| CINV1 | | HQNLNDIYRFKTEEYSHTAVNKFNVMPDSIPEWVFDFMPLRGGYFVGNVGPAHMDFRWFALGN |
| INVA | | CVSILSSLATPDQSMAIMDLLEHRWAELVGEMPLKICYPCLEGHEWRIVTGCDPKNTRWSYHNG |
| UnitProt | | GSWPVLLWQLTAACIKTGRPQIARRAVDLIESRLHRDCWPEYYDGKLGRYVGKQARKYQTWSI |
| Accession | | AGYLVAKMLLEDPSHIGMISLEEDKLMKPVIKRSASWPQL |
| No.: | | |
| Q9LQF2 | | |
| Arabidopsis | SEQ ID NO: 327 | MSAIYLLRKISTKTPSRFHRSLFFSTFSKDSPPDLSRTTSIRHLSSSQRFVSSSIYCFPQSKILPNRFSE |
| thaliana | | KTTGISVRQFSTSVETNLSDKSFERIHVQSDAILERIHKNEEEVETVSIGSEKVVREESEAEKEAWR |
| Alkaline/ | | ILENAVVRYCGSPVGTVAANDPGDKMPLNYDQVFIRDFVPSALAFLLKGEGDIVRNFLLHTLQL |
| neutral | | QSWEKTVDCYSPGQGLMPASFKVRTVALDENTTEEVLDPDFGESAIGRVAPVDSGLWWIILLRA |
| invertase | | YGKITGDFSLQERIDVQTGIKLIMNLCLADGFDMFPTLLVTDGSCMIDRRMGIHGHPLEIQSLFYS |
| A, | | ALRCSREMLSVNDSSKDLVRAINNRLSALSFHIREYYWVDIKKINEIYRYKTEEYSTDATNKFNIY |
| mitochondrial | | PEQIPPWLMDWIPEQGGYLLGNLQPAHMDFRFFTLGNFWSIVSSLATPKQNEAILNLIEAKWDDII |
| INVE | | GNMPLKICYPALEYDDWRIITGSDPKNTPWSYHNSGSWPTLLWQFTLACMKMGRPELAEKALA |
| UnitProt | | VAEKRLLADRWPEYYDTRSGKFIGKQSRLYQTWTVAGFLTSKLLLANPEMASLLFWEEDYELL |
| Accession | | DICACGLRKSDRKKCSRVAAKTQILVR |
| No.: | | |
| UnitProt | | |
| Accession | | |
| No.: | | |
| Q9FXA8 | | |
| Arabidopsis | SEQ ID NO: 328 | MAASETVLRVPLGSVSQSCYLASFFVNSTPNLSFKPVSRNRKTVRCTNSHEVSSVPKHSFHSSNS |
| thaliana | | VLKGKKFVSTICKCQKHDVEESIRSTLLPSDGLSSELKSDLDEMPLPVNGSVSSNGNAQSVGTKSI |
| Alkaline/ | | EDEAWDLLRQSVVFYCGSPIGTIAANDPNSTSVLNYDQVFIRDFIPSGIAFLLKGEYDIVRNFILYT |
| neutral | | LQLQSWEKTMDCHSPGQGLMPCSFKVKTVPLDGDDSMTEEVLDPDFGEAAIGRVAPVDSGLW |
| invertase | | WIILLRAYGKCTGDLSVQERVDVQTGIKMILKLCLADGFDMFPTLLVTDGSCMIDRRMGIHGHP |
| E, | | LEIQALFYSALVCAREMLTPEDGSADLIRALNNRLVALNFHIREYYWLDLKKINEIYRYQTEEYS |
| chloroplastic | | YDAVNKFNIYPDQIPSWLVDFMPNRGGYLIGNLQPAHMDFRFFTLGNLWSIVSSLASNDQSHAIL |
| INVE | | DFIEAKWAELVADMPLKICYPAMEGEEWRIITGSDPKNTPWSYHNGGAWPTLLWQLTVASIKM |
| UnitProt | | GRPELAEKAVELAERRISLDKWPEYYDTKRARFIGKQARLYQTWSIAGYLVAKLLLANPAAAKF |
| Accession | | LTSEEDSDLRNAFSCMLSANPRRTRGPKKAQQPFIV |
| No.: | | |
| Q9FK88 | | |
| Oryza | SEQ ID NO: 329 | MGVLGSRVAWAWLVQLLLLQQLAGASHVVYDDLELQAAATTADGVPPSIVDSELRTGYHFQPP |
| sativa | | KNWINDPNAPMYYKGWYHLFYQYNPKGAVWGNIVWAHSVSRDLINWVALKPAIEPSIRADKY |
| japonica | | GCWSGSATMMADGTPVIMYTGVNRPDVNYQVQNVALPRNGSDPLLREWVKPGHNPVIVPEGGI |
| (rice) | | NATQFRDPTTAWRGADGHWRLLVGSLAGQSRGVAYVYRSRDFRRWTRAAQPLHSAPTGMWE |
| Beta- | | CPDFYPVTADGRREGVDTSSAVVDAAASARVKYVLKNSLDLRRYDYYTVGTYDRKAERYVPD |
| fructo- | | DPAGDEHHIRYDYGNFYASKTFYDPAKRRRILWGWANESDTAADDVAKGWAGIQAIPRKVWL |
| furanosidase, | | DPSGKQLLQWPIEEVERLRGKWPVILKDRVVKPGEHVEVTGLQTAQADVEVSFEVGSLEAAERL |
| insoluble | | DPAMAYDAQRLCSARGADARGGVGPFGLWVLASAGLEEKTAVFFRVFRPAARGGGAGKPVVL |
| isoenzyme 2 | | MCTDPTKSSRNPNMYQPTFAGFVDTDITNGKISLRSLIDRSVVESFGAGGKACILSRVYPSLAIGK |
| CIN2 | | NARLYVFNNGKAEIKVSQLTAWEMKKPVMMNGA |
| Unit | | |
| Prot | | |
| Accession | | |
| No.: | | |
| Q0JDC5 | | |
| Rattus | SEQ ID NO: 330 | MAKKKFSALEISLIVLFIIVTAIAIALVTVLATKVPAVEEIKSPTPTSNSTPTSTPTSTSTPTSTSTPSP |
| norvegicus | | GKCPPEQGEPINERINCIPEQHPTKAICEERGCCWRPWNNTVIPWCFFADNHGYNAESITNENAGL |
| (rat) | | KATLNRIPSPTLFGEDIKSVILTTQTQTGNRFRFKITDPNNKRYEVPHQFVKEETGIPAADTLYDVQ |
| Sucrase- | | VSENPFSIKVIRKSNNKVLCDTSVGPLLYSNQYLQISTRLPSEYIYGFGGHIHKRFRHDLYWKTWP |
| isomaltase, | | IFTRDEIPGDNNHNLYGHQTFFMGIGDTSGKSYGVFLMNSNAMEVFIQPTPIITYRVTGGILDFYIF |
| intestinal | | LGDTPEQVVQQYQEVHWRPAMPAYWNLGFQLSRWNYGSLDTVSEVVRRNREAGIPYDAQVTD |
| Si Gene | | IDYMEDHKEFTYDRVKFNGLPEFAQDLHNHGKYIIILDPAISINKRANGAEYQTYVRGNEKNVW |
| UnitProt | | VNESDGTTPLIGEVWPGLTVYPDFTNPQTIEWWANECNLFHQQVEYDGLWIDMNEVSSFIQGSL |
| Accession | | NLKGVLLIVLNYPPFTPGILDKVMYSKTLCMDAVQHWGKQYDVHSLYGYSMAIATEQAVERVF |
| No.: | | PNKRSFILTRSTFGGSGRHANHWLGDNTASWEQMEWSITGMLEFGIFGMPLVGATSCGFLADTT |
| P23739 | | EELCRRWMQLGAFYPFSRNHNAEGYMEQDPAYFGQDSSRHYLTIRYTLLPFLYTLFYRAHMFGE |
| | | TVARPFLYEFYDDTNSWIEDTQFLWGPALLITPVLRPGVENVSAYIPNATWYDYETGIKRPWRKE |
| | | RINMYLPGDKIGLHLRGGYIIPTQEPDVTTTASRKNPLGLIVALDDNQAAKGELFWDDGESKDSI |
| | | EKKMYILYTFSVSNNELVLNCTHSSYAEGTSLAFKTIKVLGLREDVRSITVGENDQQMATHTNFT |
| | | FDSANKILSITALNFNLAGSFIVRWCRTFSDNEKFTCYPDVGTATEGTCTQRGCLWQPVSGLSNV |
| | | PPYYFPPENNPYTLTSIQPLPTGITAELQLNPPNARIKLPSNPISTLRVGVKYHPNDMLQFKIYDAQ |
| | | HKRYEVPVPLNIPDTPTSSNERLYDVEIKENPFGIQVRRRSSGKLIWDSRLPGFGFNDQFIQISTRLP |
| | | SNYLYGFGEVEHTAFKRDLNWHTWGMFTRDQPPGYKLNSYGFHPYYMALENEGNAHGVLLLN |
| | | SNGMDVTFQPTPALTYRTIGGILDFYMFLGPTPEIATRQYHEVIGFPVMPPYWALGFQLCRYGYR |
| | | NTSEIEQLYNDMVAANIPYDVQYTDINYMERQLDFTIGERFKTLPEFVDRIRKDGMKYIVILAPAI |
| | | SGNETQPYPAFERGIQKDVFVKWPNTNDICWPKVWPDLPNVTIDETITEDEAVNASRAHVAFPDF |
| | | FRNSTLEWWAREIYDFYNEKMKFDGLWIDMNEPSSFGIQMGGKVLNECRRMMTLNYPPVFSPE |
| | | LRVKEGEGASISEAMCMETEHILIDGSSVLQYDVHNLYGWSQVKPTLDALQNTTGLRGIVISRST |
| | | YPTTGRWGGHWLGDNYTTWDNLEKSLIGMLELNLFGIPYIGADICGVFHDSGYPSLYFVGIQVG |
| | | AFYPYPRESPTINFTRSQDPVSWMKLLLQMSKKVLEIRYTLLPYFYTQMHEAHAHGGTVIRPLM |
| | | HEFFDDKETWEIYKQFLWGPAFMVTPVVEPFRTSVTGYVPKARWFDYHTGADIKLKGILHTFSA |
| | | PFDTINLHVRGGYILPCQEPARNTHLSRQNYMKLIVAADDNQMAQGTLFGDDGESIDTYERGQY |
| | | TSIQFNLNQTTLTSTVLANGYKNKQEMRLGSIHIWGKGTLRISNANLVYGGRKHQPPFTQEEAKE |
| | | TLIFDLKNMNVTLDEPIQITWS |
| Oryctolagus | SEQ ID NO: 331 | MAKRKFSGLEITLIVLFVIVFILAIALIAVLATKTPAVEEVNPSSSTPTTTSTTTSTSGSVSCPSELNE |
| cuniculus | | VVNERINCIPEQSPTQAICAQRNCCWRPWNNSDIPWCFFVDNHGYNVEGMTTTSTGLEARLNRK |
| (Rabbit) | | STPTLFGNDINNVLLTTESQTANRLRFKLTDPNNKRYEVPHQFVTEFAGPAATETLYDVQVTENP |
| Sucrase- | | FSIKVIRKSNNRILFDSSIGPLVYSDQYLQISTRLPSEYMYGFGEHVHKRFRHDLYWKTWPIFTRD |
| isomaltase, | | QHTDDNNNNLYGHQTFFMCIEDTTGKSFGVFLMNSNAMEIFIQPTPIVTYRVIGGILDFYIFLGDT |
| intestinal | | PEQVVQQYQELIGRPAMPAYWSLGFQLSRWNYNSLDVVKEVVRRNREALIPFDTQVSDIDYME |
| Si Gene | | DKKDFTYDRVAYNGLPDFVQDLHDHGQKYVIILDPAISINRRASGEAYESYDRGNAQNVWVNE |
| UnitProt | | SDGTTPIVGEVWPGDTVYPDFTSPNCIEWWANECNIFHQEVNYDGLWIDMNEVSSFVQGSNKGC |
| Accession | | NDNTLNYPPYIPDIVDKLMYSKTLCMDSVQYWGKQYDVHSLYGYSMAIATERAVERVFPNKRS |
| No.: | | FILTRSTFAGSGRHAAHWLGDNTATWEQMEWSITGMLEFGLFGMPLVGADICGFLAETTEELCR |
| P07768 | | RWMQLGAFYPFSRNHNADGFEHQDPAFFGQDSLLVKSSRHYLNIRYTLLPFLYTLFYKAHAFGE |
| | | TVARPVLHEFYEDTNSWVEDREFLWGPALLITPVLTQGAETVSAYIPDAVWYDYETGAKRPWR |
| | | KQRVEMSLPADKIGLHLRGGYIIPIQQPAVTTTASRMNPLGLIIALNDDNTAVGDFFWDDGETKD |
| | | TVQNDNYILYTFAVSNNNLNITCTHELYSEGTTLAFQTIKILGVTETVTQVTVAENNQSMSTHSN |
| | | FTYDPSNQVLLIENLNFNLGRNFRVQWDQTFLESEKITCYPDADIATQEKCTQRGCIWDTNTVNP |
| | | RAPECYFPKTDNPYSVSSTQYSPTGITADLQLNPTRTRITLPSEPITNLRVEVKYHKNDMVQFKIF |
| | | DPQNKRYEVPVPLDIPATPTSTQENRLYDVEIKENPFGIQIRRRSTGKVIWDSCLPGFAFNDQFIQI |
| | | STRLPSEYIYGFGEAEHTAFKRDLNWHTWGMFTRDQPPGYKLNSYGFHPYYMALEDEGNAHGV |
| | | LLLNSNAMDVTFMPTPALTYRVIGGILDFYMFLGPTPEVATQQYHEVIGHPVMPPYWSLGFQLC |
| | | RYGYRNTSEIIELYEGMVAADIPYDVQYTDIDYMERQLDFTIDENFRELPQFVDRIRGEGMRYIIIL |
| | | DPAISGNETRPYPAFDRGEAKDVFVKWPNTSDICWAKVWPDLPNITIDESLTEDEAVNASRAHA |
| | | AFPDFFRNSTAEWWTREILDFYNNYMKFDGLWIDMNEPSSFVNGTTTNVCRNTELNYPPYFPEL |
| | | TKRTDGLHFRTMCMETEHILSDGSSVLHYDVHNLYGWSQAKPTYDALQKTTGKRGIVISRSTYP |
| | | TAGRWAGHWLGDNYARWDNMDKSIIGMMEFSLFGISYTGADICGFFNDSEYHLCTRWTQLGAF |
| | | YPFARNHNIQFTRRQDPVSWNQTFVEMTRNVLNIRYTLLPYFYTQLHEIHAHGGTVIRPLMHEFF |
| | | DDRTTWDIFLQFLWGPAFMVTPVLEPYTTVVRGYVPNARWFDYHTGEDIGIRGQVQDLTLLMN |
| | | AINLHVRGGHILPCQEPARTTFLSRQKYMKLIVAADDNHMAQGSLFWDDGDTIDTYERDLYLSV |
| | | QFNLNKTTLTSTLLKTGYINKTEIRLGYVHVWGIGNTLINEVNLMYNEINYPLIFNQTQAQEILNI |
| | | DLTAHEVTLDDPIEISWS |
| Homo | SEQ ID NO: 343 | MARKKFSGLEISLIVLFVIVTIIALALIVVLATKTPAVDEISDSTSTPATTRVTTNPSDSGKCPNVLN |
| sapiens | | DPVNVRINCIPEQFPTEGICAQRGCCWRPWNDSLIPWCFFVDNHGYNVQDMTTTSIGVEAKLNRI |
| Sucrase- | | PSPTLFGNDINSVLFTTQNQTPNRFRFKITDPNNRRYEVPHQYVKEFTGPTVSDTLYDVKVAQNP |
| isomaltase, | | FSIQVIRKSNGKTLFDTSIGPLVYSDQYLQISTRLPSDYIYGIGEQVHKRFRHDLSWKTWPIFTRDQ |
| intestinal | | LPGDNNNNLYGHQTFFMCIEDTSGKSFGVFLMNSNAMEIFIQPTPIVTYRVTGGILDFYILLGDTP |
| Si Gene | | EQVVQQYQQLVGLPAMPAYWNLGFQLSRWNYKSLDVVKEVVRRNREAGIPFDTQVTDIDYME |
| UnitProt | | DKKDFTYDQVAFNGLPQFVQDLHDHGQKYVIILDPAISIGRRANGTTYATYERGNTQHVWINES |
| Accession | | DGSTPIIGEVWPGLTVYPDFTNPNCIDWWANECSIFHQEVQYDGLWIDMNEVSSFIQGSTKGCNV |
| No.: | | NKLNYPPFTPDILDKLMYSKTICMDAVQNWGKQYDVHSLYGYSMAIATEQAVQKVFPNKRSFIL |
| P14410 | | TRSTFAGSGRHAAHWLGDNTASWEQMEWSITGMLEFSLFGIPLVGADICGFVAETTEELCRRW |
| | | MQLGAFYPFSRNHNSDGYEHQDPAFFGQNSLLVKSSRQYLTIRYTLLPFLYTLFYKAHVFGETVA |
| | | RPVLHEFYEDTNSWIEDTEFLWGPALLITPVLKQGADTVSAYIPDAIWYDYESGAKRPWRKQRV |
| | | DMYLPADKIGLHLRGGYIIPIQEPDVTTTASRKNPLGLIVALGENNTAKGDFFWDDGETKDTIQN |
| | | GNYILYTFSVSNNTLDIVCTHSSYQEGTTLAFQTVKILGLTDSVTEVRVAENNQPMNAHSNFTYD |
| | | ASNQVLLIADLKLNLGRNFSVQWNQIFSENERFNCYPDADLATEQKCTQRGCVWRTGSSLSKAP |
| | | ECYFPRQDNSYSVNSARYSSMGITADLQLNTANARIKLPSDPISTLRVEVKYHKNDMLQFKIYDP |
| | | QKKRYEVPVPLNIPTTPISTYEDRLYDVEIKENPFGIQIRRRSSGRVIWDSWLPGFAFNDQFIQISTR |
| | | LPSEYIYGFGEVEHTAFKRDLNWNTWGMFTRDQPPGYKLNSYGFHPYYMALEEEGNAHGVFLL |
| | | NSNAMDVTFQPTPALTYRTVGGILDFYMFLGPTPEVATKQYHEVIGHPVMPAYWALGFQLCRY |
| | | GYANTSEVRELYDAMVAANIPYDVQYTDIDYMERQLDFTIGEAFQDLPQFVDKIRGEGMRYIIIL |
| | | DPAISGNETKTYPAFERGQQNDVFVKWPNTNDICWAKVWPDLPNITIDKTLTEDEAVNASRAHV |
| | | AFPDFFRTSTAEWWAREIVDFYNEKMKFDGLWIDMNEPSSFVNGTTTNQCRNDELNYPPYFPEL |
| | | TKRTDGLHFRTICMEAEQILSDGTSVLHYDVHNLYGWSQMKPTHDALQKTTGKRGIVISRSTYP |
| | | TSGRWGGHWLGDNYARWDNMDKSIIGMMEFSLFGMSYTGADICGFFNNSEYHLCTRWMQLG |
| | | AFYPYSRNHNIANTRRQDPASWNETFAEMSRNILNIRYTLLPYFYTQMHEIHANGGTVIRPLLHE |
| | | FFDEKPTWDIFKQFLWGPAFMVTPVLEPYVQTVNAYVPNARWFDYHTGKDIGVRGQFQTFNAS |
| | | YDTINLHVRGGHILPCQEPAQNTFYSRQKHMKLIVAADDNQMAQGSLFWDDGESIDTYERDLYL |
| | | SVQFNLNQTTLTSTILKRGYINKSETRLGSLHVWGKGTTPVNAVTLTYNGNKNSLPFNEDTTNMI |
| | | LRIDLTTHNVTLEEPIEINWS |

TABLE 3
Linkers

| Sequence | SEQ ID | |
|---|---|---|
| Info | NO: | Amino Acid sequence |
| N-terminal | SEQ ID | EAEA |
| addition | NO: 19 | |
| EAEA | | |
| GGGS | SEQ ID | GGGGS |
| linker | NO: 20 | |
| GSS linker | | GSS |
| A rigid | SEQ ID | EAAAREAAAREAAAREAAAR |
| linker that | NO: 22 | |
| forms 4 | | |
| turns of an | | |
| alpha helix | | |
| Full linker | SEQ ID | GSSGSSGSSGSSGSSGSSGSSGSSEAAAREA |
| | NO: 23 | AAREAAAREAAARGGGGSGGGGSGGGGS |
| A flexible | SEQ ID | GSSGSSGSSGSSGSSGSSGSSGSS |
| GS linker | NO: 24 | |
| with higher | | |
| S content | | |
| A flexible | SEQ ID | GGGGSGGGGSGGGGS |
| GS linker | NO: 25 | |
| with much | | |
| higher G | | |
| content | | |
| (flex | SEQ ID | Nucleotide sequence |
| linkers) | NO: 339 | GGTTCATCAGGGTCCTCAGGATCATCCGGTA |
| | | GTAGTGGTTCATCCGGTTCATCCGGATCAAG |
| | | TGGCTCCTCTGAAGCTGCAGCAAGGGAGGCT |
| | | GCAGCCCGTGAGGCAGCCGCTAGAGAAGCCG |
| | | CCGCTAGGGGTGGTGGCGGCTCTGGCGGAGG |
| | | CGGTTCCGGTGGCGGAGGCTCT |

TABLE 4
Promoters

| Sequence | SEQ ID | |
|---|---|---|
| Info | NO: | Amino Acid sequence |
| AOX1 | SEQ ID NO: 26 | GATCTAACATCCAAAGACGAAAGGTTGAATGAAACCTTTTTGCCATCCGACATCCACA |
| promoter | | GGTCCATTCTCACACATAAGTGCCAAACGCAACAGGAGGGGATACACTAGCAGCAGA |
| | | CCGTTGCAAACGCAGGACCTCCACTCCTCTTCTCCTCAACACCCACTTTTGCCATCGAA |
| | | AAACCAGCCCAGTTATTGGGCTTGATTGGAGCTCGCTCATTCCAATTCCTTCTATTAGG |
| | | CTACTAACACCATGACTTTATTAGCCTGTCTATCCTGGCCCCCCTGGCGAGGTTCATGT |
| | | TTGTTTATTTCCGAATGCAACAAGCTCCGCATTACACCCGAACATCACTCCAGATGAG |
| | | GGCTTTCTGAGTGTGGGGTCAAATAGTTTCATGTTCCCCAAATGGCCCAAAACTGACA |
| | | GTTTAAACGCTGTCTTGGAACCTAATATGACAAAAGCGTGATCTCATCCAAGATGAAC |
| | | TAAGTTTGGTTCGTTGAAATGCTAACGGCCAGTTGGTCAAAAAGAAACTTCCAAAAGT |
| | | CGGCATACCGTTTGTCTTGTTTGGTATTGATTGACGAATGCTCAAAAATAATCTCATTA |
| | | ATGCTTAGCGCAGTCTCTCTATCGCTTCTGAACCCCGGTGCACCTGTGCCGAAACGCA |
| | | AATGGGGAAACACCCGCTTTTTGGATGATTATGCATTGTCTCCACATTGTATGCTTCCA |
| | | AGATTCTGGTGGGAATACTGCTGATAGCCTAACGTTCATGATCAAAATTTAACTGTTC |
| | | TAACCCCTACTTGACAGCAATATATAAACAGAAGGAAGCTGCCCTGTCTTAAACCTTT |
| | | TTTTTTATCATCATTATTAGCTTACTTTCATAATTGCGACTGGTTCCAATTGACAAGCT |
| | | TTTGATTTTAACGACTTTTAACGACAACTTGAGAAGATCAAAAAACAACTAATTATTG |
| | | GATCCCGA |
| DAK2 | SEQ ID NO: 27 | AAATAAGCATGTTTGTTTCAGATCAAAGATTAGCGTTTCAAAGTTGTGGAAAAGTGAC |
| promoter | | CATGCAACAATATGCAACACATTCGGATTATCTGATAAGTTTCAAAGCTACTAAGTAA |
| | | GCCCGTTTCAAGTCTCCAGACCGACATCTGCCATCCAGTGATTTTCTTAGTCCTGAAA |
| | | AATACGATGTGTAAACATAAACCACAAAGATCGGCCTCCGAGGTTGAACCCTTACGA |
| | | AAGAGACATCTGGTAGCGCCAATGCCAAAAAAAAATCACACCAGAAGGACAATTCCC |
| | | TTCCCCCCCAGCCCATTAAAGCTTACCATTTCCTATTCCAATACGTTCCATAGAGGGCA |
| | | TCGCTCGGCTCATTTTCGCGTGGGTCATACTAGAGCGGCTAGCTAGTCGGCTGTTTGA |
| | | GCTCTCTAATCGAGGGGTAAGGATGTCTAATATGTCATAATGGCTCACTATATAAAGA |
| | | ACCCGCTTGCTCAACCTTCGACTCCTTTCCCGATCCTTTGCTTGTTGCTTCTTCTTTTAT |
| | | AACAGGAAACAAAGGAATTTATACACTTTAAGAATT |
| PEX11 | SEQ ID NO: 28 | CTTCCCCATTTCACTGACAGTTTGTAGAAATAGGGCAACAATTGATGCAAATCGATTT |
| promoter | | TCAACGCATTGGTTTTGATAGCATTGATGATCTTGGAGCTGTAAAAGTCCGGCTGGAT |
| | | AAGCTCAATGAAATAGGTTGGTTGATCTGGATCTTCTTTTGGGTCATTTTGTTCGCTCT |
| | | GTATTTCACAAATTGCCAGAATCTCTGCCAACCACAGTGGTAGGTCCAACTTGGTGTT |
| | | CTGAATCACAGGCTTCCCCGGGTTGTTCTCTAAATAACCGAGGCCCGGCACAGAAATC |
| | | GTAAACCGACACGGTATCTTTTGTCCGTCCGCCAGTATCTCATCAAGGTCGTAGTAGC |
| | | CCATGATGAGTATCAAAGGGGATTTGGTTATGCGATGCAACGAGAGATTGTTTATCCC |
| | | AGATGCTGATGTAAAAACCTTAACCAGCGTGACAGTAGAAATAAGACACGTTAAAAT |
| | | TACCCGCGCTTCCCTAACAATTGGCTCTGCCTTTCGGCAAGTTTCTAACTGCCCTCCCC |
| | | TCTCACATGCACCACGAACTTACCGTTCGCTCCTAGCAGAACCACCCCAAAGTTTAAT |
| | | CAGGACCGCATTTTAGCCTATTGCTGTAGAACCCCACAACATAACCTGGTCCAGAGCC |
| | | AGCCCTTTATATATGGTAAATCCCGTTTGAACTTCGAAGTGGAATCGGAATTTTTACA |
| | | TCAAAGAAACTGATACTGAAACTTTTGGCTTCGACTTGGACTTTCTCTTAATC |
| FLD1 | SEQ ID NO: 29 | AAATCAGCCATTAATCTCACCTCAGTTTTTGAATCAGTAGAATTTTCAATGAAACAAA |
| promoter | | CGGTTGGTATATTATTTGATAGGGTAGCCAAATTTCCAAAAATGAACTTTTCATCAGG |
| | | TAATATCTTGAATACCGTAATGTAGTGACTATTGGAAGAAACTGCTATCAAATTATAT |
| | | TTCGGATAGAAATCCAAACCCCAGACTGATCTCTTGAGTCTCAACTCTAAGTCAGCCG |
| | | CGACTCTAATTATCTGTGGATTAGGAGTTAGTGTGGACAAAGCATCAGTATAGTATAA |
| | | CTTTACGGTTCCATTATCAGACGCTATTGCAAGAACTTCCTTTCCATTGATCTCTCCAA |
| | | TTCGACAGTAATTGATATCATAAGGTAGGTCTGGAAACACACTGGCGCTTGTATCCCA |
| | | TTCTGCAGGAATTTCTGGAACGGTGGTAATGGTAGTTATCCAACGGAGTTGGGGTAGT |
| | | TGGTATATCTGGATATGCCGCCTATAGGATAAAAACAGGAGAGAGTGAACCTTGCTT |
| | | ACGGCTACTAGATTGTTCTTGTACTCGGAATTGTCGTTATCGGAAACTAGACTAATCT |
| | | CATCTGTGTGTTGCAGTACTATTGAGTCGTTGTAGTATCTACCAGGAGGGCATTCCAT |
| | | GAACTAGTGAGACAAATGAGTTGGATTTTCTCAATAGACATATGCAAGAATGCTACA |
| | | CAACGGATGTCGCACTCTTTTTCTTAGTTGATAATATCATCCAATCAGAAGACACGGG |
| | | CTAGAAGGACTTGCTCCCGAAGGATAATCCACTGCTACTATCTCCCTTCCTCACATAT |
| | | AGTCTTGCAGGGCTCATGCCCCTTTCTCCTTCGAACTGCCCGATGAGGAAGTCTTTAG |
| | | CCTATCAAGGAATTCGGGACCATCATCAATTTTTAGAGCCTTACCTGATCGCAATCAG |
| | | GATTTCACTACTCATATAAATACATCACTCAAACTCCAACTTTGCTTGTTCATACAATT |
| | | CTTGATATTCACAGGATC |
| FGH1 | SEQ ID NO: 30 | GTGAATTTGTCACGGAATTGACCAAGAGGTCAGACGATCCTGTATCCCATTGAGCCGT |
| promoter | | TATGCTTTGTGGGGGAAACCCTATTTCTATCGTACTAAGAAAACCAATGGTGAACTCA |
| | | TATTCGGTATCAATGGCGACGATTCCAGCATAGCCTGTAGACAGTAACAACACTAGG |
| | | GCAACAGCAACTAACATATCTTCATTGATGAAACGTTGTGATCGGTGTGACTTTTATA |
| | | GTAAAAGCTACAACTGTTTGAAATACCAAGATATCATTGTGAATGGCTCAAAAGGGT |
| | | AATACATCTGAAAAACCTGAAGTGTGGAAAATTCCGATGGAGCCAACTCATGATAAC |
| | | GCAGAAGTCCCATTTTGCCATCTTCTCTTGGTATGAAACGGTAGAAAATGATCCGAGT |
| | | ATGCCAATTGATACTCTTGATTCATGCCCTATAGTTTGCGTAGGGTTTAATTGATCTCC |
| | | TGGTCTATCGATCTGGGACGCAATGTAGACCCCATTAGTGGAAACACTGAAAGGGAT |
| | | CCAACACTCTAGGCGGACCCGCTCACAGTCATTTCAGGACAATCACCACAGGAATCA |
| | | ACTACTTCTCCCAGTCTTCCTTGCGTGAAGCTTCAAGCCTACAACATAACACTTCTTAC |
| | | TTAATCTTTGATTCTCGAATTGTTTACCCAATCTTGACAACTTAGCCTAAGCAATACTC |
| | | TGGGGTTATATATAGCAATTGCTCTTCCTCGCTGTAGCGTTCATTCCATCTTTCTAGAA |
| | | TTCGT |
| DAS2 | SEQ ID NO: 31 | CCTGTTGATAAGACGCATTCTAGAGTTGTTTCATGAAAGGGTTACGGGTGTTGATTGG |
| promoter | | TTTGAGATATGCCAGAGGACAGATCAATCTGTGGTTTGCTAAACTGGAAGTCTGGTAA |
| | | GGACTCTAGCAAGTCCGTTACTCAAAAAGTCATACCAAGTAAGATTACGTAACACCTG |
| | | GGCATGACTTTCTAAGTTAGCAAGTCACCAAGAGGGTCCTATTTAACGTTTGGCGGTA |
| | | TCTGAAACACAAGACTTGCCTATCCCATAGTACATCATATTACCTGTCAAGCTATGCT |
| | | ACCCCACAGAAATACCCCAAAAGTTGAAGTGAAAAAATGAAAATTACTGGTAACTTC |
| | | ACCCCATAACAAACTTAATAATTTCTGTAGCCAATGAAAGTAAACCCCATTCAATGTT |
| | | CCGAGATTTAGTATACTTGCCCCTATAAGAAACGAAGGATTTCAGCTTCCTTACCCCA |
| | | TGAACAGAAATCTTCCATTTACCCCCCACTGGAGAGATCCGCCCAAACGAACAGATA |
| | | ATAGAAAAAAGAAATTCGGACAAATAGAACACTTTCTCAGCCAATTAAAGTCATTCC |
| | | ATGCACTCCCTTTAGCTGCCGTTCCATCCCTTTGTTGAGCAACACCATCGTTAGCCAGT |
| | | ACGAAAGAGGAAACTTAACCGATACCTTGGAGAAATCTAAGGCGCGAATGAGTTTAG |
| | | CCTAGATATCCTTAGTGAAGGGTTGTTCCGATACTTCTCCACATTCAGTCATAGATGG |
| | | GCAGCTTTGTTATCATGAAGAGACGGAAACGGGCATTAAGGGTTAACCGCCAAATTA |
| | | TATAAAGACAACATGTCCCCAGTTTAAAGTTTTTCTTTCCTATTCTTGTATCCTGAGTG |
| | | ACCGTTGTGTTTAATATAACAAGTTCGTTTTAACTTAAGACCAAAACCAGTTACAACA |
| | | AATTATAACCCCTCTAAACACTAAAGTTCACTCTTATCAAACTATCAAACATCAAAAG |
| | | AATTCGCG |
| CAT1 | SEQ ID NO: 32 | TAATCGAACTCCGAATGCGGTTCTCCTGTAACCTTAATTGTAGCATAGATCACTTAAA |
| promoter | | TAAACTCATGGCCTGACATCTGTACACGTTCTTATTGGTCTTTTAGCAATCTTGAAGTC |
| | | TTTCTATTGTTCCGGTCGGCATTACCTAATAAATTCGAATCGAGATTGCTAGTACCTGA |
| | | TATCATATGAAGTAATCATCACATGCAAGTTCCATGATACCCTCTACTAATGGAATTG |
| | | AACAAAGTTTAAGCTTCTCGCACGAGACCGAATCCATACTATGCACCCCTCAAAGTTG |
| | | GGATTAGTCAGGAAAGCTGAGCAATTAACTTCCCTCGATTGGCCTGGACTTTTCGCTT |
| | | AGCCTGCCGCAATCGGTAAGTTTCATTATCCCAGCGGGGTGATAGCCTCTGTTGCTCA |
| | | TCAGGCCAAAATCATATATAAGCTGTAGACCCAGCACTTCAATTACTTGAAATTCACC |
| | | ATAACACTTGCTCTAGTCAAGACTTACAATTAAA |
| MDH3 | SEQ ID NO: 33 | TAGCTTGGGTAGGACTTGACAAGTACGGCTTCCGTGGTCATACCAAACGCCTTTGTTA |
| promoter | | CCGTTGGCTATACCTAATGACCAAGGCATTTGTGGATTATAACGGTATCGTAGTTGAA |
| | | AAATATGACGTAACCACTGGTACTAGCCCCCACAAGGTTGATGCTGAATACGGGAAT |
| | | CAAGGTGCCGATTTTAAAGGAGTAGCCACTGAAGGGTTTGGCTGGGTCAATGCCTCTT |
| | | TTATTTTGGGATTAACCTACTTAGATGTCCAAGGCATCCGTGCGATAGGCGCCGTTAC |
| | | GTCCCCTGATGTATTTTTCAGGAAGCTCAAACCTTGGGAACGCGCAAGTTATGGCCTA |
| | | AGGCCATGTAACGAGATAGTCAAGTCAAACTAGAAGTATACGGTTTCCCCGCAGAAA |
| | | TAGCAGAAATAGGCGACAAATACATACAACATTTTCATTGTGATAGGGGGCGGCGGT |
| | | TCCTAGGAGGGACAACCCCCAGAAACCTTGTAGACTACGTTTTCACGACGATGGGTTA |
| | | TTACTGTAAAGGAAGAATATACTACCCACCAGTTGAATGTTTGAACGGATCAAAGGTC |
| | | GAAGGGAGTACACGGCCCAACCAACGTAGCTACCGGAGAAAGCAAGACTTTCCCAAA |
| | | CCAAATAGCTCCGGGTTTCTTCTCCGGCAACCCGTCAGTTTTTGTGTGGCCGGACAAA |
| | | AATTCGCACCCTCAGTCTAATTGAAAGGTCGGGCTCCGAGCTCTAGGCGTTTGCGCAT |
| | | GTAATATTGCATCCCCTCCCATAGATAATACTGCGCGAACACAGGGTGCAAATTATGA |
| | | TGACCACACATGCCAGTGACCAAAACAGTTTTTTAGTCTTTAAAAACCCTCGGAACTT |
| | | CTGAGTATATAAAGGCTTCTCATTTCCTACAAGCAAACAAAGAAGAAACTTCCACTTT |
| | | CTAACTTTTTATCTATAGACTTTAGAGTTACAACCAACGAACAATAACAAA |
| HAC1 | SEQ ID NO: 34 | TGAAGCTTATCTGCTGAGCAAGTTGTTTGACCAAACTTGAGTCAACAGTGGTTAACTA |
| promoter | | TATCCTCTATTATTTTAGATGGGAGCACATCAAGTGTACGGGAACAATGCAATCGACA |
| | | ACCTGTAGCCTGACATACATAGCCATCTTGAATTGACAAAACTTAGAATGTCTTGAAT |
| | | GTGATAGATATGAGTTCCCAAAAATCTCTTTTACGATTTCCCAGTTGCGGTGTACTATT |
| | | ACACAGAGGATATCATAGCAGACTTACAATCCTCAGGCATAAAACGAGCTTTCTTATC |
| | | AAAGTGTATTCAAATGGACCATTTGATTGCACCAAGGCATTAGCCCCAAACCATACCA |
| | | CACAGTAACTTGATATTCTCAGCATGCATGGAAATTCCACTCATAACGCGCTATTCAC |
| | | CGCGAATACTTATCTATGAAACTGGGTTCTTTAGTATTCTTTGCCAAATTTCACCGATT |
| | | AGAAATTATTAGGTAATATAATTTCTTTGGGGAACCCCTTCCCGTTACGCCCGCTGCG |
| | | GCTTTGTGGTTCTTTTCCAGTCTTGAGCAAATTACATCTGGTCTAGACAGTTCTTCCGT |
| | | GCCCCAGTATGCGAGCGCAAACTTTCAATCAAACCTCGTAGCAAATTGGTACTTGAAC |
| | | TTCGTATTTAACCGCTATTAAATGTACTGACTCTTACATTATGAAAAATTTTGATAAAG |
| | | ATTTTATATTTCATCTCAGTTAATCTCCTAATAATAATAGTCTGCATAACTCAAACGGT |
| | | ACTTCCTTTTCGGAACGCGAAGAGTAGTCTCTATGTCATTCTCACACTATCCGCAGCG |
| | | CAATAGAGAACGAGCATGTTACCCGACTCATCCCTTGTCGATTCGGAAACGATTTATA |
| | | AATACAATTAGATCGCCACCGATCTTCTTTTGTCAATATTATAAAAATAGTACAGATT |
| | | TTCCTTAGTCGAATCAGATCGCAGAAA |
| BiP | SEQ ID NO: 35 | AGATCTGAGGGTGTATACGATGTATCGTGCCGAACACATGCACTTGACGGCACAGCA |
| promoter | | AATGGTATTCAAGAAGACCACTTTAGAATGGGAGTTAATAGGGATGGTTTCATGGAG |
| | | GTTAAAACACTTCAAGGAGGCATCTGAAGCATTCAAGTATGCACTAGGTCTGAGGTTT |
| | | TCGGTCAAGGCATGCAAGAAATTAATTGTATTCTATCTGAACGAACGCTCCAGAATGA |
| | | ACCAGCCAGAAACCTCAATTGCCCTCAACAACTTAAATCAATCCACATTATCCATCCA |
| | | AGAGATTCTCAAGTATCGTTCGTTCCTCGATATCAACCTAATTTCAAACTTGGTCAAA |
| | | CTAGGAGTTTGGAATCACCGCTGGTATGCTGAGTTTTCTCCAAAACTCATAGAAAGCC |
| | | TTGCGGTTGTTGTGGAGAACGGAGGGCTTATCAAGGTAGAAAACGAGGTTAAGGCTA |
| | | CCTATTTCGATTCACAAGATGGAGTTTACGACTTGATGAACGAGGTATTCAAGTTCAT |
| | | GAAGCATTACGATTATCCTGGGACTGACAACTAAGAGCTCCTAGTGAAGACTTGAGA |
| | | TGGACATGATAAACAATTATAGTGAAAATAGAAACCATAATACAATATTCTAATAGA |
| | | GGAACCGTTTACCTGTGGTTCCTATTGTGGCCTACTGTTACTAGCTAGTGTAATACACC |
| | | CTTGCCTCAGCTTTGCAAGTTGACAACTCAGCCAAATGATCTTTGAATGCGCGAAACC |
| | | TCAAGGTCCATCGAATTTTCTCGAATTTTCAGTGTTTTCATACAGCGTGTCATCTTCTT |
| | | TCGCGTACTTATTAAAATCGTACCCAGATCCCTTCTTCTTCCTTAATTTCAATTCCAAC |
| | | ACTCAAGA |
| RAD30 | SEQ ID NO: 36 | AGATCTTGCAAAATACCTTTCCAGCTTTCCAGCTTCCTAGCACTCATCTTGAAGATATC |
| promoter | | AAATATTCTCCATTCAAACCAACATCAAAAAATAGAATAATTATAATCAGTTTGAAGA |
| | | GCAAGAGTAATTTTAAAGGAAACACATTCATGGTCAGCTAGAAGGTTGACTGAAGAG |
| | | TCGCAAGATATCTGAGAATAAAAAAGAGCATAGCTAACAAGATGAGTAAACACGGCA |
| | | AACAGATTTAGGAACAGGTGAAGGGTTTCTGGCTCTTCAATGTATATCCTGCTAGCCA |
| | | CCCATTCAGAAATAACACAAAGTAGGACCCTACTGAAAAATAAATTTAATACATCTTC |
| | | ATCCTCTCATTAAACCACCGACCACTCAAACCATACCAGCCTTGTCCAATTCCATGCA |
| | | TCGTGCTATCCGTCAGAATTTTCAGTGTTAATCGAATCGGTCATTATAGCTCCGTCTGG |
| | | GGCGACAACTTGTCATCACAGAATAGCACAATTATGCGTTGGAATCGTCAAAAAATC |
| | | ACCTCCAGGTCTGTATACATACAGAACTGGTTGTAACGACAACCTTGTTTGATTGAGG |
| | | TGACTGGAAGGTGGAAAGAAAGGGAGGAAATAAATATTGCAAGGAAAGAAAAAAAA |
| | | ATTGTTCACAGTCACCTCTTCACCTTCGCGATTTCATGTTTCTTTCATGTGCTAACTGAT |
| | | CCCAGGGCTTCTCCAGCGCCCTTATCTGTTAG |
| RVS161-2 | SEQ ID NO: 37 | CTGCCCATCTATGACTGAATGTGGAGAAGTATCGGAACAACCCTTCACTAAGGATATC |
| promoter | | TAGGCTAAACTCATTCGCGCCTTAGATTTCTCCAAGGTATCGGTTAAGTTTCCTCTTTC |
| | | GTACTGGCTAACGATGGTGTTGCTCAACAAAGGGATGGAACGGCAGCTAAAGGGAGT |
| | | GCATGGAATGACTTTAATTGGCTGAGAAAGTGTTCTATTTGTCCGAATTTCTTTTTTCT |
| | | ATTATCTGTTCGTTTGGGCGGATCTCTCCAGTGGGGGGTAAATGGAAGATTTCTGTTC |
| | | ATGGGGTAAGGAAGCTGAAATCCTTCGTTTCTTATAGGGGCAAGTATACTAAATCTCG |
| | | GAACATTGAATGGGGTTTACTTTCATTGGCTACAGAAATTATTAAGTTTGTTATGGGG |
| | | TGAAGTTACCAGTAATTTTCATTTTTTCACTTCAACTTTTGGGGTATTTCTGTGGGGTA |
| | | GCATAGCTTGACAGGTAATATGATGTACTATGGGATAGGCAAGTCTTGTGTTTCAGAT |
| | | ACCGCCAAACGTTAAATAGGACCCTCTTGGTGACTTGCTAACTTAGAAAGTCATGCCC |
| | | AGGTGTTACGTAATCTTACTTGGTATGACTTTTTGAGTAACGGACTTGCTAGAGTCCTT |
| | | ACCAGACTTCCAGTTTAGCAAACCACAGATTGATCTGTCCTCTGGCATATCTCAAACC |
| | | AATCAACACCCGTAACCCTTTCATGAAACAACTCTAGAATGCGTCTTATCAACAGGAT |
| | | TGCCCAAAACAGTAATTGGGGCGGTGGAATCTACATGGGAGTTCCATCGTTGTCTCGG |
| | | TTTTTCTCCCTATAAGCTACTCTGGAGACGAAGTAACTAACACCCTCAAATATCATT |
| MPP10 | SEQ ID NO: 38 | TCTGAATCCGACCTCCTCTAATCTACCACTGAAGAGAAGCAGTGTATTGTTCGTCTAC |
| promoter | | GTAAATTTGAATGTGTAAATGGCAAACATGGCTTCGGGGATGATTTGGCATATATATT |
| | | ATTGTAGCATCGTCTGTGGCTCTATGAGTTGTGTGGCGGATGATGAAAAGTTTCGTGC |
| | | TGATCCCACAATGCGGCATTTACCAAATGGGGAAAGACCAGATTTCTTCGCTGCGCCA |
| | | GCTAGGGACAGCATAATGTTCCAAGAAGAAGCGATTACAGGTGGATTACAAAGCGTT |
| | | CGTCTGCAGTTGATGTTCTACGTGATGGGTATGAGTTGTAGTGCTACGCTCCATGAAT |
| | | ACTTCTAATTTGTCGTTGACAATCCATGAATAATTTAAGTTTGCTTCCCAAGAGTCTAT |
| | | TGCGAAGGGTGAGCCGAATCTCTTGGCGTATGCACCCGACTCGTCGGCTTTTGTGCGT |
| | | TCCTTGCAAAGCTCGGTAGCAATCCGTTGGTGGGAGAAATTTGTCTCACGAATTTCAG |
| | | TTGGGAGTAGCTGTTCCTGGTAGCAAGTTCGAGGGGATCTGTGCTCATAAAACGTGCT |
| | | CACGCCAAAAATATTCTTACAAAATCTTCGCGGGGTGTTTGTCTTACATAATCGATTG |
| | | GATATTTTCTTCAAATTTTTTTTTCTTACTGAAGTCCCCTATAGAG |
| THP3 | SEQ ID NO: 39 | TCTTGCCAGTTGTCTCCTAAGATGTCATCGGAGTAGGCTCGGCTAAAGAGTAGTAATG |
| promoter | | CATCAAGACCAACCAAAACACCTTCCACGAGTTCAGATGAACCTTTTAATAACTTCAG |
| | | GTCACTTTGATGCCGGCACAACTGGGCGAGTTTCGTATAGTTAACTCTGATCTTGCAC |
| | | TCCAGAACGGGAATAGGATTGACTTTTTGCTTCCGAGAAACGATTTGCTCTCTCTTCGT |
| | | CTGGCTTTTCACTTTATATCGCACGGAATCAATGGATGGAACTCCTAAAGCTCCTAAC |
| | | TTCGATGATTTGCTAGCCATGACTCTGTGGGACATTTTCTTGCATCTCGTTTGTAACCT |
| | | GTCTGTTCCTACACTAAGTTTATGAGAGGCTACTTTGGATTCTAGCCTCGGTGGTAAA |
| | | GTGGGAGATAACAACGGCATAAGGCAAGAACCAGAAGTACCATAACGGTCTGGTAA |
| | | AGTTGGTGATAACTTAATTGGAAGAGTGTAAGTAAGACGTGGCTTGTAATAAGGCTTT |
| | | CCATCAAAAAGGTTCTCCGGGTTGGAGTTTGTGAGGCTCACATCTTTGATCAGTCTTTC |
| | | AATATAAATTGGTAACGTTGATGACAATGCCGGAGGTAATTTCTGTAGTTGTTGATAT |
| | | ACGCAGATAACAGATTCAAATCTCCATTGGTTTTCATCATTGTGGCTTAAATTAGATC |
| | | AGAACATGGTAGTATTTAAAAATGGATCTCTTTGCAGATTTACTCAATATAGCGAAAA |
| | | AAGGAGACATTCGTTACAAAATATGAAGATAATTCGCCTCATAACTCGATTAATCAAA |
| | | ACAGACGGTCCAGTTCTTCTTTTGGTAGT |
| GBP2 | SEQ ID NO: 40 | ATCTGTACTGGTACTGACAAAGGTTATCCAGAATCCGAGACATTTCAACAACAGAGAT |
| promoter | | TCCAGGCTTCAAAACATCCATTTTATCACCAATATCTAGTAATGCTTGCAACAATTCTG |
| | | GATACTTCTTCTGTGTAACCAAATCTCTTATAAACTGAACAGCTTTCTGTACGTTGTCG |
| | | TCAGTAGTTGGATCAACCTCAGTGGTGACCTGGCCTATCGGTTTTCCAAAAGACTTGT |
| | | TTATCACGTCCGAAAGCTCCCATTTTTGCAGATGCGCAACTTTAAAAGGCCTGGCTTG |
| | | AACATTTGCATCTCTTGTTGTGTGTTCTTTGAGAAAATATTCATCGATCTGGGTGCTTC |
| | | CAACGACAGAAGATACTCTTCTGAGACCAGAAAGTCCCCAGCCATGCTTCCTAATTAC |
| | | AAAATATTTGTAGGAAGATCCCTGATTAGGACAAAGTTGTCTTCTCATGAGTTCAACT |
| | | GAAACTGGGGCTCAAACGGATTATGAAAGGGGTGATTAAAGGTTTTCCTAGCCTTACT |
| | | TTCCAAATGTCGACCGAGACGAACATTTAAAATCCTAACATCAGAAATTTCTATCCTT |
| | | AATCTCATTGATGGTTAGTACACTTCGCAGAGTCTCCACATTTGCAGACCCTCCTGGA |
| | | TAACCAAAGCTTATCTAACAGCGGCATTGGACCTTTGAAAAGACCCTC |
| DAS1 | SEQ ID NO: 41 | AAATCTGAACACGATGAAACCTCCCCGTAGATTCCACCGCCCCGTTACTTTTTTGGGC |
| promoter | | AATCCCGTTGATAAGATCCATTTTAGAGTTGTTTCTGAAAGGATTACAGGCGTTGAAG |
| | | GGTCAGAGAGATGCCAGAGAACAGACCAATTGGTAGTTTGCTAAAGTGGACGTCTGG |
| | | CAGGTGCTCTATCGTGTTCTTTATTTAGGGCGTTACACTTAGTAGGATTACGTAACAAT |
| | | TTGGCTTAACCTTCTAAGTTAGAAAGAAACCAAGAGGGGTCCTCTTTAACGTTCAGCA |
| | | GTATCTAAAACACAAAACCTGCCCTCATAATACATCATTCTATCTGTCAAGCTGTGCT |
| | | ACCCCACAGAAATACCCCCAAGAGTTAAAGTGAAAAGAAAAGCTAAATCTGTTAGAC |
| | | TTCACCCCATAACAAACTTGATAGTTCCTGTAGCCAATGAAAGTTAACCCCATTCAAT |
| | | GTTCCGAGATCTAGTATGCTTGCTCCTATAAGGAACGAAGGGTTCCAGCTTCCTTACC |
| | | CCATCAATGGAAATCTCCTATTTACCCCCCACTGGAAAGATCCGTCCGAACGAACGGA |
| | | TAATAGAAAAAAGAAATTCGGACAAAATAGAACACTTATTTAGCCAATGAAATCCAT |
| | | TTCCAGCATCTCCTTCAACTGCCGTTCCATCCCCTTTGTTGAGCTACACCATCGTCAGC |
| | | CAGTACCGAATAGGAAACTTAACCGATATCTTGGAGAATTCTAATGCGCGAATGAGTT |
| | | TAGCCTAGATATCCTTAGTGAAGGGTTGTTCCGATACTTCTCCACATTCAGTCATTTCA |
| | | GATGGGCAGCATTGTTATCATGAAGAAACGGAAACGGGCAGTAAGGGTTAACCGCCA |
| | | AATTATATAAAGACAACATGTCCCCAGTTTAAAGTTTTTCTTTCCTATTCTTGTATCCT |
| | | GAGTGACCGTTGTGTTTAAAATAACAAGTTCGTTTTAACTTAAGACCAAAACCAGTTA |
| | | CAACAAATTATTCCCCAACTAAACACTAAAGTTCACTCTTATCAAACTATCAAACATC |
| | | AAAG |
| Methanol | SEQ ID NO: 42 | CTTCCCCATTTCACTGACAGTTTGTAGAAATAGGGCAACAATTGATGCAAATCGATTT |
| inducible | | TCAACGCATTGGTTTTGATAGCATTGATGATCTTGGAGCTGTAAAAGTCCGGCTGGAT |
| promoter | | AAGCTCAATGAAATAGGTTGGTTGATCTGGATCTTCTTTTGGGTCATTTTGTTCGCTCT |
| | | GTATTTCACAAATTGCCAGAATCTCTGCCAACCACAGTGGTAGGTCCAACTTGGTGTT |
| | | CTGAATCACAGGCTTCCCCGGGTTGTTCTCTAAATAACCGAGGCCCGGCACAGAAATC |
| | | GTAAACCGACACGGTATCTTTTGTCCGTCCGCCAGTATCTCATCAAGGTCGTAGTAGC |
| | | CCATGATGAGTATCAAAGGGGATTTGGTTATGCGATGCAACGAGAGATTGTTTATCCC |
| | | AGATGCTGATGTAAAAACCTTAACCAGCGTGACAGTAGAAATAAGACACGTTAAAAT |
| | | TACCCGCGCTTCCCTAACAATTGGCTCTGCCTTTCGGCAAGTTTCTAACTGCCCTCCCC |
| | | TCTCACATGCACCACGAACTTACCGTTCGCTCCTAGCAGAACCACCCCAAAGTTTAAT |
| | | CAGGACCGCATTTTAGCCTATTGCTGTAGAACCCCACAACATAACCTGGTCCAGAGCC |
| | | AGCCCTTTATATATGGTAAATCCCGTTTGAACTTCGAAGTGGAATCGGAATTTTTACA |
| | | TCAAAGAAACTGATACTGAAACTTTTGGCTTCGACTTGGACTTTCTCTTAATCGAATTC |
| | | GT |
| GCW14 | SEQ ID NO: 43 | CAGGTGAACCCACCTAACTATTTTTAACTGGCATCCAGTGAGCTCGCTGGGTGAAAGC |
| promoter | | CAACCATCTTTTGTTTCGGGGAACCGTGCTCGCCCCGTAAAGTTAATTTTTTTTTCCCG |
| | | CGCAGCTTTAATCTTTCGGCAGAGAAGGCGTTTTCATCGTAGCGTGGGAACAGAATAA |
| | | TCAGTTCATGTGCTATACAGGCACATGGCAGCAGTCACTATTTTGCTTTTTAACCTTAA |
| | | AGTCGTTCATCAATCATTAACTGACCAATCAGATTTTTTGCATTTGCCACTTATCTAAA |
| | | AATACTTTTGTATCTCGCAGATACGTTCAGTGGTTTCCAGGACAACACCCAAAAAAAG |
| | | GTATCAATGCCACTAGGCAGTCGGTTTTATTTTTGGTCACCCACGCAAAGAAGCACCC |
| | | ACCTCTTTTAGGTTTTAAGTTGTGGGAACAGTAACACCGCCTAGAGCTTCAGGAAAAA |
| | | CCAGTACCTGTGACCGCAATTCACCATGATGCAGAATGTTAATTTAAACGAGTGCCAA |
| | | ATCAAGATTTCAACAGACAAATCAATCGATCCATAGTTACCCATTCCAGCCTTTTCGT |
| | | CGTCGAGCCTGCTTCATTCCTGCCTCAGGTGCATAACTTTGCATGAAAAGTCCAGATT |
| | | AGGGCAGATTTTGAGTTTAAAATAGGAAATATAAACAAATATACCGCGAAAAAGGTT |
| | | TGTTTATAGCTTTTCGCCTGGTGCCGTACGGTATAAATACATACTCTCCTCCCCCCCCT |
| | | GGTTCTCTTTTTCTTTTGTTACTTACATTTTACCGTTCCGT |
| FDH1 | SEQ ID NO: 44 | AAATAAATGGCAGAAGGATCAGCCTGGACGAAGCAACCAGTTCCAACTGCTAAGTAA |
| promoter | | AGAAGATGCTAGACGAAGGAGACTTCAGAGGTGAAAAGTTTGCAAGAAGAGAGCTG |
| | | CGGGAAATAAATTTTCAATTTAAGGACTTGAGTGCGTCCATATTCGTGTACGTGTCCA |
| | | ACTGTTTTCCATTACCTAAGAAAAACATAAAGATTAAAAAGATAAACCCAATCGGGA |
| | | AACTTTAGCGTGCCGTTTCGGATTCCGAAAAACTTTTGGAGCGCCAGATGACTATGGA |
| | | AAGAGGAGTGTACCAAAATGGCAAGTCGGGGGCTACTCACCGGATAGCCAATACATT |
| | | CTCTAGGAACCAGGGATGAATCCAGGTTTTTGTTGTCACGGTAGGTCAAGCATTCACT |
| | | TCTTAGGAATATCTCGTTGAAAGCTACTTGAAATCCCATTGGGTGCGGAACCAGCTTC |
| | | TAATTAAATAGTTCGATGATGTTCTCTAAGTGGGACTCTACGGCTCAAACTTCTACAC |
| | | AGCATCATCTTAGTAGTCCCTTCCCAAAACACCATTCTAGGTTTCGGAACGTAACGAA |
| | | ACAATGTTCCTCTCTTCACATTGGGCCGTTACTCTAGCCTTCCGAAGAACCAATAAAA |
| | | GGGACCGGCTGAAACGGGTGTGGAAACTCCTGTCCAGTTTATGGCAAAGGCTACAGA |
| | | AATCCCAATCTTGTCGGGATGTTGCTCCTCCCAAACGCCATATTGTACTGCAGTTGGT |
| | | GCGCATTTTAGGGAAAATTTACCCCAGATGTCCTGATTTTCGAGGGCTACCCCCAACT |
| | | CCCTGTGCTTATACTTAGTCTAATTCTATTCAGTGTGCTGACCTACACGTAATGATGTC |
| | | GTAACCCAGTTAAATGGCCGAAAAACTATTTAAGTAAGTTTATTTCTCCTCCAGATGA |
| | | GACTCTCCTTCTTTTCTCCGCTAGTTATCAAACTATAAACCTATTTTACCTCAAATACC |
| | | TCCAACATCACCCACTTAAACAGAATT |
| FBA1 | SEQ ID NO: 45 | TGCTTAAGTAATTGAAAACAGTGTTGTGATTATATAAGCATGGTATTTGAATAGAACT |
| promoter | | ACTGGGGTTAACTTATCTAGTAGGATGGAAGTTGAGGGAGATCAAGATGCTTAAAGA |
| | | AAAGGATTGGCCAATATGAAAGCCATAATTAGCAATACTTATTTAATCAGATAATTGT |
| | | GGGGCATTGTGACTTGACTTTTACCAGGACTTCAAACCTCAACCATTTAAACAGTTAT |
| | | AGAAGACGTACCGTCACTTTTGCTTTTAATGTGATCTAAATGTGATCACATGAACTCA |
| | | AACTAAAATGATATCTTTTACTGGACAAAAATGTTATCCTGCAAACAGAAAGCTTTCT |
| | | TCTATTCTAAGAAGAACATTTACATTGGTGGGAAACCTGAAAACAGAAAATAAATAC |
| | | TCCCCAGTGACCCTATGAGCAGGATTTTTGCATCCCTATTGTAGGCCTTTCAAACTCAC |
| | | ACCTAATATTTCCCGCCACTCACACTATCAATGATCACTTCCCAGTTCTCTTCTTCCCC |
| | | TATTCGTACCATGCAACCCTTACACGCCTTTTCCATTTCGGTTCGGATGCGACTTCCAG |
| | | TCTGTGGGGTACGTAGCCTATTCTCTTAGCCGGTATTTAAACATACAAATTCACCCAA |
| | | ATTCTACCTTGATAAGGTAATTGATTAATTTCATAAATGAATTCGCG |
| GAP | SEQ ID NO: 46 | TTTTTGTAGAAATGTCTTGGTGTCCTCGTCCAATCAGGTAGCCATCTCTGAAATATCTG |
| promoter | | GCTCCGTTGCAACTCCGAACGACCTGCTGGCAACGTAAAATTCTCCGGGGTAAAACTT |
| | | AAATGTGGAGTAATGGAACCAGAAACGTCTCTTCCCTTCTCTCTCCTTCCACCGCCCG |
| | | TTACCGTCCCTAGGAAATTTTACTCTGCTGGAGAGCTTCTTCTACGGCCCCCTTGCAGC |
| | | AATGCTCTTCCCAGCATTACGTTGCGGGTAAAACGGAGGTCGTGTACCCGACCTAGCA |
| | | GCCCAGGGATGGAAAAGTCCCGGCCGTCGCTGGCAATAATAGCGGGCGGACGCATGT |
| | | CATGAGATTATTGGAAACCACCAGAATCGAATATAAAAGGCGAACACCTTTCCCAAT |
| | | TTTGGTTTCTCCTGACCCAAAGACTTTAAATTTAATTTATTTGTCCCTATTTCAATCAAT |
| | | TGAACAACTAT |
| PGK | SEQ ID NO: 47 | AAATAGCAGTTTGCGGTTTCTTGATTTCATGGGGGGAACAAACAATAGTGTTGCCTTA |
| promoter | | ATTCTAATTGGCATTGTTGCTTGGAATCGAAATTGGGGGATAACGTCATATCTGAAAA |
| | | GTAAACAACTTCGGGAAATCAGGCTGTTTGAATGGCTTGGAAGCGAGATAGAAAGGG |
| | | GATAGCGAGATAGAGGGGGCGGAGTAGACGAAGGGTGTTAAACTGCTGAAATCTCTC |
| | | AATCTGGAAGAAACGGAATAAATTAACTCCTTGCGATAATAAAATCCGAGTCCGTTAT |
| | | GACCCCACACCGTGTTGACCACGGCATACCCCATGGAATCTGGTACAAAGCGTCAGTC |
| | | TTGAAGACACCATCACGTGTAGGAGACTGATTGTCTGACCGTCCAGCAAAAAGGGCA |
| | | TTATAAATCTTGCTGTTAAAGGGGTGAGGGGAGATGCAGGTTGTTCTTTTATTCGCCTT |
| | | GAACTTTTTAATTTTCCCGGGGTTGCGGAGCGTGAACAGTTAGCCCGATCTGATAGCT |
| | | TGCAAGATTCAACAGTTTATCCACTACAGGTCAGAGAGATCGCCGCAGAAGAAATGC |
| | | TCGTCTCGTGTTCCAGCACACATACTGGTGAAGTCGTTATTTTGCCGAAGGGGGGGTA |
| | | ATAAGGTTATGCACCCCCTCTCCACACCCCAGAATCATTTTTTAGCTGGGTTCAAGGC |
| | | ATTAGACTTTGCACATTTTTCCCTTAAACACCCTTGAAACGCGGATAAACAGTTGCAT |
| | | GTGCATCCTAAAACTAGGTGAGATGCGTACTCCGTGCTCCGATAATAACAGTGGTGTT |
| | | GGGGTTGCTGCTAGCTCACGCACTCCGTTCTTTTTTTTCAACCAGCAAAATTCGATGGG |
| | | GAGAAACTTGGGGTACTTTGCCGACTCCTCCACCATGCTGGTATATAAATAATACTCG |
| | | CCCACTTTTCGTTTGCTGCTTTTATATTTCATAGACTGAAAAAGACTCTTCTTCTACTTT |
| | | TTCATAATATATCTCAGATATCACTACTATAG |
| TEFg_ | SEQ ID NO: 48 | GCGATTTAAATTCGCGAAAGAACAGCCTAATAAACTCCGAAGCATGATGGCCTCTATC |
| promoter | | CGGAAAACGTTAAGAGATGTGGCAACAGGAGGGCACATAGAATTTTTAAAGACGCTG |
| | | AAGAATGCTATCATAGTCCGTAAAAATGTGATAGTACTTTGTTTAGTGCGTACGCCAC |
| | | TTATTCGGGGCCAATAGCTAAACCCAGGTTTGCTGGCAGCAAATTCAACTGTAGATTG |
| | | AATCTCTCTAACAATAATGGTGTTCAATCCCCTGGCTGGTCACGGGGAGGACTATCTT |
| | | GCGTGATCCGCTTGGAAAATGTTGTGTATCCCTTTCTCAATTGCGGAAAGCATCTGCT |
| | | ACTTCCCATAGGCACCAGTTACCCAATTGATATTTCCAAAAAAGATTACCATATGTTC |
| | | ATCTAGAAGTATAAATACAAGTGGACATTCAATGAATATTTCATTCAATTAGTCATTG |
| | | ACACTTTCATCAACTTACTACGTCTTATTCAACAATGAATTCGCG |
| PMP20 | SEQ ID NO: 49 | ACACAGTTATTATTCATTTAAATGTCAAAACAGTAGTGATAAAAGGCTATGAAGGAG |
| promoter | | GTTGTCTAGGGGCTCGCGGAGGAAAGTGATTCAAACAGACCTGCCAAAAAGAGAAAA |
| | | AAGAGGGAATCCCTGTTCTTTCCAATGGAAATGACGTAACTTTAACTTGAAAAATACC |
| | | CCAACCAGAAGGGTTCAAACTCAACAAGGATTGCGTAATTCCTACAAGTAGCTTAGA |
| | | GCTGGGGGAGAGACAACTGAAGGCAGCTTAACGATAACGCGGGGGGATTGGTGCAC |
| | | GACTCGAAAGGAGGTATCTTAGTCTTGTAACCTCTTTTTTCCAGAGGCTATTCAAGATT |
| | | CATAGGCGATATCGATGTGGAGAAGGGTGAACAATATAAAAGGCTGGAGAGATGTCA |
| | | ATGAAGCAGCTGGATAGATTTCAAATTTTCTAGATTTCAGAGTAATCGCACAAAACGA |
| | | AGGAATCCCACCAAGACAAAAAAAAAAATTCTAAGGAATTCCGAAACG |
| SHB17 | SEQ ID NO: 50 | AAATTCTTTTTACGTGGTGCGCATACTGGACAGAGGCAGAGTCTCAATTTCTTCTTTTG |
| promoter | | AGACAGGCTACTACAGCCTGTGATTCCTCTTGGTACTTGGATTTGCTTTTATCTGGCTC |
| | | CGTTGGGAACTGTGCCTGGGTTTTGAAGTATCTTGTGGATGTGTTTCTAACACTTTTTC |
| | | AATCTTCTTGGAGTGAGAATGCAGGACTTTGAACATCGTCTAGCTCGTTGGTAGGTGA |
| | | ACCGTTTTACCTTGCATGTGGTTAGGAGTTTTCTGGAGTAACCAAGACCGTCTTATCAT |
| | | CGCCGTAAAATCGCTCTTACTGTCGCTAATAATCCCGCTGGAAGAGAAGTTCGAACAG |
| | | AAGTAGCACGCAAAGCTCTTGTCAAATGAGAATTGTTAATCGTTTGACAGGTCACACT |
| | | CGTGGGCTATGTACGATCAACTTGCCGGCTGTTGCTGGAGAGATGACACCAGTTGTGG |
| | | CATGGCCAATTGGTATTCAGCCGTACCACTGTATGGAAAATGAGATTATCTTGTTCTT |
| | | GATCTAGTTTCTTGCCATTTTAGAGTTGCCACATTCGTAGGTTTCAGTACCAATAATGG |
| | | TAACTTCCAAACTTCCAACGCAGATACCAGAGATCTGCCGATCCTTCCCCAACAATAG |
| | | GAGCTTACTACGCCATACATATAGCCTATCTATTTTCACTTTCGCGTGGGTGCTTCTAT |
| | | ATAAACGGTTCCCCATCTTCCGTTTCATACTACTTGAATTTTAAGCACTAAAGAATT |
| PEX8 | SEQ ID NO: 51 | AAATTAACCAGTGTTTTCTTATCTATTTGTCTTTTTACACTAAAGTGAAGTACGAATCC |
| promoter | | ATGCGATTGATTCCTCCTCAGATATCAGCTGAATTCTTGCTTATGTAATACTTGCGCGA |
| | | ACTACATGTGAACTTAGGATTCGATAAGGCTGGGGGGTCAACCAACCCCACTTCAAA |
| | | GAGCCGACCCGTATAAATAGCCTCTGCGTCCTCAGATCAACAAGACGAAGCAATTTTT |
| | | TTTTACCTATCTTCAGGTGCCTGTTAG |
| PEX4 | SEQ ID NO: 52 | AGGGAGGCAATTAGTTGTCCTTGTGGAATCAAAAGAGCACAAGAAACCTGTGATTGA |
| promoter | | AAGTCTGGGCTGTCTGGGGTTGGCAAGAAAATCATAAAGTTTATATAGTACATTTGTT |
| | | AGTTGCTTCTTTGAATGACACCTTGATCTACATGTTGTTCTTCCCAGTTCCCACCGCGA |
| | | AGTTTCTCTAACTCTCAATCTCTCTTTCCCCACTTGATAATCCAAAGAA |
| TKL3 | SEQ ID NO: 53 | gtcgaggaaagggtcgtttcggggagttaaatatttttggctatgtagcagacatgtttcgacgctggcgtcgc |
| Promoter | | gtcgatcggaaaatattaccccaggaacaagcacttgcttgggttagccaccaccctgcgcaagcctttttgcc |
| | | ggctctacacagggccaatgaaatctgggcggaatctgaaaccgatgaaacggacgacactggcaacaagctca |
| | | ctgcactattttttttttctagtgaaatagcctatcctcgtctcgctcccctcatacctgtaaaggggtgcaat |
| | | ttagcctcgttccagccattcacgggccactcaacaacacgtcggctaccatggggtgcttgggcaccaaaagg |
| | | cctataaataggcccccatccgtctgctacacagtcatctctgtcttttcttccc |

TABLE 5
Signal Peptides

| Sequence | SEQ ID | |
|---|---|---|
| Info | NO: | Amino Acid sequence |
| Signal Peptide | SEQ ID NO: 56 | MFTPVRRRVRTAALALSAAAALVLGSTAASGASATPSPAPAP |
| Signal Peptide | SEQ ID NO: 57 | MKLSTVLLSAGLASTTLA |
| Signal Peptide | SEQ ID NO: 58 | MRFPSIFTAVLFAASSALA |
| Signal Peptide | SEQ ID NO: 59 | MVSLRSIFTSSILAAGLTRAHG |
| Signal Peptide | SEQ ID NO: 60 | MKFPVPLLFLLQLFFIIATQG |
| Signal Peptide | SEQ ID NO: 61 | MQVKSIVNLLLACSLAVA |
| Signal Peptide | SEQ ID NO: 62 | MQFNWNIKTVASILSALTLAQA |
| Signal Peptide | SEQ ID NO: 63 | MYRNLIIATALTCGAYSAYVPSEPWSTLTPDASLESALKDYSQTFGIAIKSLDADKIKR |
| Signal Peptide | SEQ ID NO: 64 | MNLYLITLLFASLCSAITLPKR |
| Signal Peptide | SEQ ID NO: 65 | MFEKSKFVVSFLLLLQLFCVLGVHG |
| Signal Peptide | SEQ ID NO: 66 | MQFNSVVISQLLLTLASVSMG |
| Signal Peptide | SEQ ID NO: 67 | MKSQLIFMALASLVASAPLEHQQQHHKHEKR |
| Signal Peptide | SEQ ID NO: 68 | MKFAISTLLIILQAAAVFA |
| Signal Peptide | SEQ ID NO: 69 | MKLLNFLLSFVTLFGLLSGSVFA |
| Signal Peptide | SEQ ID NO: 70 | MIFNLKTLAAVAISISQVSA |
| Signal Peptide | SEQ ID NO: 71 | MKISALTACAVTLAGLAIAAPAPKPEDCTTTVQKRHQHKR |
| Signal Peptide | SEQ ID NO: 72 | MSYLKISALLSVLSVALA |
| Signal Peptide | SEQ ID NO: 73 | MLSTILNIFILLLFIQASLQ |
| Signal Peptide | SEQ ID NO: 74 | MKLSTNLILAIAAASAVVSAAPVAPAEEAANHLHKR |
| Signal Peptide | SEQ ID NO: 75 | MFKSLCMLIGSCLLSSVLA |
| Signal Peptide | SEQ ID NO: 76 | MKLAALSTIALTILPVALA |
| Signal Peptide | SEQ ID NO: 77 | MSFSSNVPQLFLLLVLLTNIVSG |
| Signal Peptide | SEQ ID NO: 78 | MQLQYLAVLCALLLNVQSKNVVDFSRFGDAKISPDDTDLESRERKR |
| Signal Peptide | SEQ ID NO: 79 | MKIHSLLLWNLFFIPSILG |
| Signal Peptide | SEQ ID NO: 80 | MSTLTLLAVLLSLQNSALA |
| Signal Peptide | SEQ ID NO: 81 | MINLNSFLILTVTLLSPALALPKNVLEEQQAKDDLAKR |
| Signal Peptide | SEQ ID NO: 82 | MFSLAVGALLLTQAFG |
| Signal Peptide | SEQ ID NO: 83 | MKILSALLLLFTLAFA |
| Signal Peptide | SEQ ID NO: 84 | MKVSTTKFLAVFLLVRLVCA |
| Signal Peptide | SEQ ID NO: 85 | MQFGKVLFAISALAVTALG |
| Signal Peptide | SEQ ID NO: 86 | MWSLFISGLLIFYPLVLG |
| Signal Peptide | SEQ ID NO: 87 | MRNHLNDLVVLFLLLTVAAQA |
| Signal Peptide | SEQ ID NO: 88 | MFLKSLLSFASILTLCKA |
| Signal Peptide | SEQ ID NO: 89 | MFVFEPVLLAVLVASTCVTA |
| Signal Peptide | SEQ ID NO: 90 | MFSPILSLEIILALATLQSVFA |
| Signal Peptide | SEQ ID NO: 91 | MIINHLVLTALSIALA |
| Signal Peptide | SEQ ID NO: 92 | MLALVRISTLLLLALTASA |
| Signal Peptide | SEQ ID NO: 93 | MRPVLSLLLLLASSVLA |
| Signal Peptide | SEQ ID NO: 94 | MVLIQNFLPLFAYTLFFNQRAALA |
| Signal Peptide | SEQ ID NO: 95 | MVSLTRLLITGIATALQVNA |
| Signal Peptide | SEQ ID NO: 96 | MIFDGTTMSIAIGLLSTLGIGAEA |
| Signal Peptide | SEQ ID NO: 97 | MVLVGLLTRLVPLVLLAGTVLLLVFVVLSGG |
| Signal Peptide | SEQ ID NO: 98 | MLSILSALTLLGLSCA |
| Signal Peptide | SEQ ID NO: 99 | MRLLHISLLSIISVLTKANA |
| Signal Peptide | SEQ ID NO: 100 | MRFPSIFTAVLFAASSALAAPVNTTTEDETAQIPAEAVIGYLDLEGDFDVAVLPFSNSTNN |
| | | GLLFINTTIASIAAKEEGVSLDKREAEA |
| Signal Peptide | SED ID NO: 344 | MRFPSIFTAVLFAASSALAAPVQTTTEDELEGDFDVAVLPFSASIAAKEEGVSLEKREAEA |
| Signal Peptide | SEQ ID NO: 101 | MFKSVVYSILAASLANA |
| Signal Peptide | SEQ ID NO: 102 | MLLQAFLFLLAGFAAKISA |
| Signal Peptide | SEQ ID NO: 103 | MASSNLLSLALFLVLLTHANS |
| Signal Peptide | SEQ ID NO: 104 | MNIFYIFLFLLSFVQGLEHTHRRGSLVKR |
| Signal Peptide | SEQ ID NO: 105 | MLIIVLLFLATLANSLDCSGDVFFGYTRGDKTDVHKSQALTAVKNIKR |
| Signal Peptide | SEQ ID NO: 106 | MESVSSLFNIFSTIMVNYKSLVLALLSVSNLKYARGMPTSERQQGLEER |
| Signal Peptide | SEQ ID NO: 107 | MFAFYFLTACISLKGVFG |
| Signal Peptide | SEQ ID NO: 108 | MRFSTTLATAATALFFTASQVSA |
| Signal Peptide | SEQ ID NO: 109 | MKFAYSLLLPLAGVSASVINYKR |
| Signal Peptide | SEQ ID NO: 110 | MKFFAIAALFAAAAVAQPLEDR |
| Signal Peptide | SEQ ID NO: 111 | MQFFAVALFATSALA |
| Signal Peptide | SEQ ID NO: 112 | MKWVTFISLLFLFSSAYSRGVFRR |
| Signal Peptide | SEQ ID NO: 113 | MRSLLILVLCFLPLAALG |
| Signal Peptide | SEQ ID NO: 114 | MKVLILACLVALALA |
| Signal Peptide | SEQ ID NO: 115 | MFNLKTILISTLASIAVA |
| Signal Peptide | SEQ ID NO: 116 | MYRKLAVISAFLATARAQSA |
| WT | SEQ ID NO: 117 | MRFPSIFTAVLFAASSALAAPVNTTTEDETAQIPAEAVIGYLDLEGDFDVAVLPFSNSTNN |
| | | GLLFINTTIASIAAKEEGVQLDKR |
| App3 | SEQ ID NO: 118 | MRFPPIFTAALFAASSALAAPANTTTEDETAQIPAEAVIGYLDSEGDSDVAVLPFSNSTNN |
| | | GLSFINTTIASIAAKEEGVQLDKR |
| App8 | SEQ ID NO: 119 | MRFPSIFTAVLFAASSALAAPANTTTEDETAQIPAEAVISYSDLEGDFDAAALPLSNSTNN |
| | | GLSSTNTTIASIAAKEEGVQLDKR |
| App9 | SEQ ID NO: 120 | MRPPSIFTAVLFAASSALAAPANTTTEDETTQIPAEAVATYLDLEGDVDVAVLPFSSSTN |
| | | NGLSFINTTIASIAAKEEGVQLDKR |
| App10 | SEQ ID NO: 121 | MRFPSIFTAALFAASSALAAPANTTTEGETAQTPAEAVIGYRDLEGDFDVAVLPFPNSTN |
| | | NGLLFTNTTTASIAAKEEGVQLDKR |
| appS1 | SEQ ID NO: 122 | MRFPSIFTAVLLAAPSALAAPANATTEDEAAQIPAEAVIGYLDLEGDFDAAVLPFSNSTN |
| | | NGLLSINTTIASIAAKEEGVQLDKR |
| appS4 | SEQ ID NO: 123 | MRFPSIFTAVVFAASSALAAPANTTAEDETAQIPAEAVIGYLGLEGDSDVAALPLSDSTN |
| | | NGSLSTNTTIASIAAKEEGVQLDKR |
| appS6 | SEQ ID NO: 124 | MRLPSIFTAAVFAASSALAAPANTTTEDETAQIPAEAAIGYLDLEGDSDVAVLPLSNSTN |
| | | NGLLFINTTIASIAAKEEGVQLDKR |
| appS8 | SEQ ID NO: 125 | MRFPSIFTAVLFAASSALAAPANTTTEDETAQIPAEAVIGYLDLEGDFDVAVLPFSNSTND |
| | | GLSFINTTTASIAAKEEGVQLDKR |
| a-Factor | SEQ ID NO: 126 | MRFPSIFTAVLFAASSALAAPVNTTTEDETAQIPA |
| PpScw11p | SEQ ID NO: 127 | MLSTILNIFILLLFIQASLQAPIPVVTKYVTEGIAVV |
| PpDse4p | SEQ ID NO: 128 | MSFSSNVPQLFLLLVLLTNIVSGAVISVWSTSKVTK |
| PpExglp | SEQ ID NO: 129 | MNLYLITLLFASLCSAITLPKRDIIWDYSSEKIMG |
| a-EGFP | SEQ ID NO: 130 | MRFPSIFTAVLFAASSALAAPVNTTTEDETAQIPA |
| S-EGFP | SEQ ID NO: 131 | MLSTILNIFILLLFIQASLQEFDYKDDDDKMVSKG |
| D-EGFP | SEQ ID NO: 132 | MSFSSNVPQLFLLLVLLTNIVSGEFDYKDDDDKMV |
| E-EGFP | SEQ ID NO: 133 | MNLYLITLLFASLCSAEFDYKDDDDKMVSKGEELF |
| a-CALB | SEQ ID NO: 134 | MRFPSIFTAVLFAASSALAAPVNTTTEDETAQIPA |
| S-CALB | SEQ ID NO: 135 | MLSTILNIFILLLFIQASLQEFLPSGSDPAFSQPK |
| D-CALB | SEQ ID NO: 136 | MSFSSNVPQLFLLLVLLTNIVSGEFLPSGSDPAFS |
| E-CALB | SEQ ID NO: 137 | MNLYLITLLFASLCSAEFLPSGSDPAFSQPKSVLD |
| Amylase (AA) | SEQ ID NO: 138 | MVAWWSLFLYGLQVAAPALAAEVDCSRFPNATDKEGKDVLVCNKDLRPICGTDGVTY |
| | | TNDCLLCAYSIEFGTNISKEHDGECKETVPMNCSSYANTTSEDGKVMVLCNRAFNPVCG |
| | | TDGVTYDNECLLCAHKVEQGASVDKRHDGGCRKELAAVSVDCSEYPKPDCTAEDRPLC |
| | | GSDNKTYGNKCNFCNAVVESNGTLTLSHFGKC |
| Alpha K (AK) | SEQ ID NO: 139 | MRFPSIFTAVLFAASSALAAPVNTTTEDELEGDFDVAVLPFSASIAAKEEGVSLEKRAEV |
| | | DCSRFPNATDKEGKDVLVCNKDLRPICGTDGVTYTNDCLLCAYSIEFGTNISKEHDGECK |
| | | ETVPMNCSSYANTTSEDGKVMVLCNRAFNPVCGTDGVTYDNECLLCAHKVEQGASVD |
| | | KRHDGGCRKELAAVSVDCSEYPKPDCTAEDRPLCGSDNKTYGNKCNFCNAVVESNGTL |
| | | TLSHFGKC |
| Alpha T (AT) | SEQ ID NO: 140 | MRFPSIFTAVLFAASSALAAEVDCSRFPNATDKEGKDVLVCNKDLRPICGTDGVTYTND |
| | | CLLCAYSIEFGTNISKEHDGECKETVPMNCSSYANTTSEDGKVMVLCNRAFNPVCGTDG |
| | | VTYDNECLLCAHKVEQGASVDKRHDGGCRKELAAVSVDCSEYPKPDCTAEDRPLCGSD |
| | | NKTYGNKCNFCNAVVESNGTLTLSHFGKC |
| Glucoamyl | SEQ ID NO: 141 | MSFRSLLALSGLVCSGLAAEVDCSRFPNATDKEGKDVLVCNKDLRPICGTDGVTYTNDC |
| (GA) | | LLCAYSIEFGTNISKEHDGECKETVPMNCSSYANTTSEDGKVMVLCNRAFNPVCGTDGV |
| | | TYDNECLLCAHKVEQGASVDKRHDGGCRKELAAVSVDCSEYPKPDCTAEDRPLCGSDN |
| | | KTYGNKCNFCNAVVESNGTLTLSHFGKC |
| Ovomucoid | SEQ ID NO: 144 | MAMAGVFVLFSFVLCGFLPDAAFG |
| signal peptide | | |
| Lysozyme | SEQ ID NO: 145 | MRSLLILVLCFLPLAALG |
| signal peptide | | |
| Ovalbumin | SEQ ID NO: 146 | MRFPSIFTAVLFAASSALAAPVNTTTEDETAQIPAEAVIGYSDLEGDFDVAVLPFSNSTNN |
| Signal Peptide | | GLLFINTTIASIAAKEEGVSLDKREAEA |
| Ovotransferrin | SEQ ID NO: 147 | MKLILCTVLSLGIAAVCFA |
| Signal Peptide | | |
| Bovine | SEQ ID NO: 148 | MKLFVPALLSLGALGLCLA |
| Lactoferrin | | |
| Signal Peptide | | |
| Porcine | SEQ ID NO: 149 | MKLFIPALLFLGTLGLCLA |
| Lactoferrin | | |
| Signal Peptide | | |
| Kid Lipase | SEQ ID NO: 150 | MESKALLLLALSVWLQSLTVSHG |
| Signal Peptide | | |
| Porcine | SEQ ID NO: 151 | MLLIWTLSLLLGAVLG |
| Lipase Signal | | |
| Peptide | | |

TABLE 6
Proteins of Interest

| SEQ ID | Sequence | |
|---|---|---|
| NO. | Info | Sequence |
| Ovomucoid | SEQ ID NO: | AEVDCSRFPNATDKEGKDVLVCNKDLRPICGTDGVTYTNDCLLCAYSIEFGTNISKEHDGECK |
| (canonical) | 152 | ETVPMNCSSYANTTSEDGKVMVLCNRAFNPVCGTDGVTYDNECLLCAHKVEQGASVDKRH |
| | | DGGCRKELAAVSVDCSEYPKPDCTAEDRPLCGSDNKTYGNKCNFCNAVVESNGTLTLSHFGK |
| | | C* |
| Ovomucoid | SEQ ID NO: | AEVDCSRFPNATDMEGKDVLVCNKDLRPICGTDGVTYTNDCLLCAYSVEFGTNISKEHDGEC |
| | 153 | KETVPMNCSSYANTTSEDGKVMVLCNRAFNPVCGTDGVTYDNECLLCAHKVEQGASVDKR |
| | | HDGGCRKELAAVSVDCSEYPKPDCTAEDRPLCGSDNKTYGNKCNFCNAVVESNGTLTLSHFG |
| | | KC* |
| Ovomucoid | SEQ ID NO: | AEVDCSRFPNATDMEGKDVLVCNKDLRPICGTDGVTYTNDCLLCAYSVEFGTNISKEHDGEC |
| G162M | 154 | KETVPMNCSSYANTTSEDGKVMVLCNRAFNPVCGTDGVTYDNECLLCAHKVEQGASVDKR |
| F167A | | HDGGCRKELAAVSVDCSEYPKPDCTAEDRPLCGSDNKTYMNKCNACNAVVESNGTLTLSHF |
| | | GKC* |
| Ovomucoid | SEQ ID NO: | MAMAGVFVLFSFVLCGFLPDAAFGAEVDCSRFPNATDKEGKDVLVCNKDLRPICGTDGVTYT |
| isoform 1 | 155 | NDCLLCAYSIEFGTNISKEHDGECKETVPMNCSSYANTTSEDGKVMVLCNRAFNPVCGTDGV |
| precursor full | | TYDNECLLCAHKVEQGASVDKRHDGGCRKELAAVSVDCSEYPKPDCTAEDRPLCGSDNKTY |
| length | | GNKCNFCNAVVESNGTLTLSHFGKC |
| Ovomucoid | SEQ ID NO: | MAMAGVFVLFSFVLCGFLPDAVFGAEVDCSRFPNATDMEGKDVLVCNKDLRPICGTDGVTY |
| [Gallus gallus] | 156 | TNDCLLCAYSVEFGTNISKEHDGECKETVPMNCSSYANTTSEDGKVMVLCNRAFNPVCGTDG |
| | | VTYDNECLLCAHKVEQGASVDKRHDGGCRKELAAVSVDCSEYPKPDCTAEDRPLCGSDNKT |
| | | YGNKCNFCNAVVESNGTLTLSHFGKC |
| Ovomucoid | SEQ ID NO: | MAMAGVFVLFSFVLCGFLPDAAFGAEVDCSRFPNATDKEGKDVLVCNKDLRPICGTDGVTYT |
| isoform 2 | 157 | NDCLLCAYSIEFGTNISKEHDGECKETVPMNCSSYANTTSEDGKVMVLCNRAFNPVCGTDGV |
| precursor | | TYDNECLLCAHKVEQGASVDKRHDGGCRKELAAVDCSEYPKPDCTAEDRPLCGSDNKTYGN |
| [Gallus gallus] | | KCNFCNAVVESNGTLTLSHFGKC |
| Ovomucoid | SEQ ID NO: | AEVDCSRFPNATDKEGKDVLVCNKDLRPICGTDGVTYNNECLLCAYSIEFGTNISKEHDGECK |
| [Gallus gallus] | 158 | ETVPMNCSSYANTTSEDGKVMVLCNRAFNPVCGTDGVTYDNECLLCAHKVEQGASVDKRH |
| | | DGECRKELAAVSVDCSEYPKPDCTAEDRPLCGSDNKTYGNKCNFCNAVVESNGTLTLSHFGK |
| | | C |
| Ovomucoid | SEQ ID NO: | MAMAGVFVLFSFALCGFLPDAAFGVEVDCSRFPNATNEEGKDVLVCTEDLRPICGTDGVTYS |
| [Numida | 159 | NDCLLCAYNIEYGTNISKEHDGECREAVPVDCSRYPNMTSEEGKVLILCNKAFNPVCGTDGVT |
| meleagris] | | YDNECLLCAHNVEQGTSVGKKHDGECRKELAAVDCSEYPKPACTMEYRPLCGSDNKTYDNK |
| | | CNFCNAVVESNGTLTLSHFGKC |
| PREDICTED: | SEQ ID NO: | MQTITWRQPQGDHLRSRAPAATCRAGQYLTMAMAGIFVLFSFALCGFLPDAAFGVEVDCSRF |
| Ovomucoid | 160 | PNTTNEEGKDVLVCTEDLRPICGTDGVTHSECLLCAYNIEYGTNISKEHDGECREAVPMDCSR |
| isoform X1 | | YPNTTNEEGKVMILCNKALNPVCGTDGVTYDNECVLCAHNLEQGTSVGKKHDGGCRKELAA |
| [Meleagris | | VSVDCSEYPKPACTLEYRPLCGSDNKTYGNKCNFCNAVVESNGTLTLSHFGKC |
| gallopavo] | | |
| Ovomucoid | SEQ ID NO: | VEVDCSRFPNTTNEEGKDVLVCTEDLRPICGTDGVTHSECLLCAYNIEYGTNISKEHDGECRE |
| [Meleagris | 161 | AVPMDCSRYPNTTSEEGKVMILCNKALNPVCGTDGVTYDNECVLCAHNLEQGTSVGKKHDG |
| gallopavo] | | ECRKELAAVSVDCSEYPKPACTLEYRPLCGSDNKTYGNKCNFCNAVVESNGTLTLSHFGKC |
| PREDICTED: | SEQ ID NO: | MQTITWRQPQGDHLRSRAPAATCRAGQYLTMAMAGIFVLFSFALCGFLPDAAFGVEVDCSRF |
| Ovomucoid | 162 | PNTTNEEGKDVLVCTEDLRPICGTDGVTHSECLLCAYNIEYGTNISKEHDGECREAVPMDCSR |
| isoform X2 | | YPNTTNEEGKVMILCNKALNPVCGTDGVTYDNECVLCAHNLEQGTSVGKKHDGGCRKELAA |
| [Meleagris | | VDCSEYPKPACTLEYRPLCGSDNKTYGNKCNFCNAVVESNGTLTLSHFGKC |
| gallopavo] | | |
| Ovomucoid | SEQ ID NO: | EYGTNISIKHNGECKETVPMDCSRYANMTNEEGKVMMPCDRTYNPVCGTDGVTYDNECQLC |
| [Bambusicola | 163 | AHNVEQGTSVDKKHDGVCGKELAAVSVDCSEYPKPECTAEERPICGSDNKTYGNKCNFCNA |
| thoracicus] | | VVYVQP |
| Ovomucoid | SEQ ID NO: | VDCSRFPNTTNEEGKDVLACTKELHPICGTDGVTYSNECLLCYYNIEYGTNISKEHDGECTEA |
| [Callipepla | 164 | VPVDCSRYPNTTSEEGKVLIPCNRDFNPVCGSDGVTYENECLLCAHNVEQGTSVGKKHDGGC |
| squamata] | | RKEFAAVSVDCSEYPKPDCTLEYRPLCGSDNKTYASKCNFCNAVVIWEQEKNTRHHASHSVF |
| | | FISARLVC |
| Ovomucoid | SEQ ID NO: | MLPLGLREYGTNTSKEHDGECTEAVPVDCSRYPNTTSEEGKVRILCKKDINPVCGTDGVTYD |
| [Colinus | 165 | NECLLCSHSVGQGASIDKKHDGGCRKEFAAVSVDCSEYPKPACMSEYRPLCGSDNKTYVNKC |
| virginianus] | | NFCNAVVYVQPWLHSRCRLPPTGTSFLGSEGRETSLLTSRATDLQVAGCTAISAMEATRAAAL |
| | | LGLVLLSSFCELSHLCFSQASCDVYRLSGSRNLACPRIFQPVCGTDNVTYPNECSLCRQMLRSR |
| | | AVYKKHDGRCVKVDCTGYMRATGGLGTACSQQYSPLYATNGVIYSNKCTFCSAVANGEDID |
| | | LLAVKYPEEESWISVSPTPWRMLSAGA |
| Ovomucoid- | SEQ ID NO: | MSWWGIKPALERPSQEQSTSGQPVDSGSTSTTTMAGIFVLLSLVLCCFPDAAFGVEVDCSRFP |
| like isoform | 166 | NTTNEEGKEVLLCTKDLSPICGTDGVTYSNECLLCAYNIEYGTNISKDHDGECKEAVPVDCST |
| X2 | | YPNMTNEEGKVMLVCNKMFSPVCGTDGVTYDNECMLCAHNVEQGTSVGKKYDGKCKKEV |
| [Ansercygnoides | | ATVDCSDYPKPACTVEYMPLCGSDNKTYDNKCNFCNAVVDSNGTLTLSHFGKC |
| domesticus] | | |
| Ovomucoid- | SEQ ID NO: | MSSQNQLHRRRRPLPGGQDLNKYYWPHCTSDRFSWLLHVTAEQFRHCVCIYLQPALERPSQE |
| like isoform | 167 | QSTSGQPVDSGSTSTTTMAGIFVLLSLVLCCFPDAAFGVEVDCSRFPNTTNEEGKEVLLCTKDL |
| X1 | | SPICGTDGVTYSNECLLCAYNIEYGTNISKDHDGECKEAVPVDCSTYPNMTNEEGKVMLVCN |
| [Ansercygnoides | | KMFSPVCGTDGVTYDNECMLCAHNVEQGTSVGKKYDGKCKKEVATVDCSDYPKPACTVEY |
| domesticus] | | MPLCGSDNKTYDNKCNFCNAVVDSNGTLTLSHFGKC |
| Ovomucoid | SEQ ID NO: | VEVDCSRFPNTTNEEGKDEVVCPDELRLICGTDGVTYNHECMLCFYNKEYGTNISKEQDGEC |
| [Coturnix | 168 | GETVPMDCSRYPNTTSEDGKVTILCTKDFSFVCGTDGVTYDNECMLCAHNVVQGTSVGKKH |
| japonica] | | DGECRKELAAVSVDCSEYPKPACPKDYRPVCGSDNKTYSNKCNFCNAVVESNGTLTLNHFGK |
| | | C |
| Ovomucoid | SEQ ID NO: | MAMAGVFLLFSFALCGFLPDAAFGVEVDCSRFPNTTNEEGKDEVVCPDELRLICGTDGVTYN |
| [Coturnix | 169 | HECMLCFYNKEYGTNISKEQDGECGETVPMDCSRYPNTTSEDGKVTILCTKDFSFVCGTDGVT |
| japonica] | | YDNECMLCAHNIVQGTSVGKKHDGECRKELAAVSVDCSEYPKPACPKDYRPVCGSDNKTYS |
| | | NKCNFCNAVVESNGTLTLNHFGKC |
| Ovomucoid | SEQ ID NO: | MAGVFVLLSLVLCCFPDAAFGVEVDCSRFPNTTNEEGKDVLLCTKELSPVCGTDGVTYSNEC |
| [Anas | 170 | LLCAYNIEYGTNISKDHDGECKEAVPADCSMYPNMTNEEGKMTLLCNKMFSPVCGTDGVTY |
| platyrhynchos] | | DNECMLCAHNVEQGTSVGKKYDGKCKKEVATVDCSGYPKPACTMEYMPLCGSDNKTYGNK |
| | | CNFCNAVVDSNGTLTLSHFGEC |
| Ovomucoid, | SEQ ID NO: | QVDCSRFPNTTNEEGKEVLLCTKELSPVCGTDGVTYSNECLLCAYNIEYGTNISKDHDGECKE |
| partial [Anas | 171 | AVPADCSMYPNMTNEEGKMTLLCNKMFSPVCGTDGVTYDNECMLCAHNVEQGTSVGKKYD |
| platyrhynchos] | | GKCKKEVATVSVDCSGYPKPACTMEYMPLCGSDNKTYGNKCNFCNAVV |
| Ovomucoid- | SEQ ID NO: | MTMPGAFVVLSFVLCCFPDATFGVEVDCSTYPNTTNEEGKEVLVCSKILSPICGTDGVTYSNE |
| like [Tyto | 172 | CLLCANNIEYGTNISKYHDGECKEFVPVNCSRYPNTTNEEGKVMLICNKDLSPVCGTDGVTYD |
| alba] | | NECLLCAHNLEPGTSVGKKYDGECKKEIATVDCSDYPKPVCSLESMPLCGSDNKTYSNKCNF |
| | | CNAVVDSNETLTLSHFGKC |
| Ovomucoid | SEQ ID NO: | MTMAGVFVLLSFALCCFPDAAFGVEVDCSTYPNTTNEEGKEVLVCTKILSPICGTDGVTYSNE |
| [Balearica | 173 | CLLCAYNIEYGTNVSKDHDGECKEVVPVDCSRYPNSTNEEGKVVMLCSKDLNPVCGTDGVT |
| regulorum | | YDNECVLCAHNVESGTSVGKKYDGECKKETATVDCSDYPKPACTLEYMPFCGSDSKTYSNK |
| gibbericeps] | | CNFCNAVVDSNGTLTLSHFGKC |
| Turkey | SEQ ID NO: | MTTAGVFVLLSFALCSFPDAAFGVEVDCSTYPNTTNEEGKEVLVCTKILSPICGTDGVTYSNEC |
| vulture | 174 | LLCAYNIEYGTNVSKDHDGECKEFVPVDCSRYPNTTNEDGKVVLLCNKDLSPICGTDGVTYD |
| [Cathartes | | NECLLCARNLEPGTSVGKKYDGECKKEIATVDCSDYPKPVCSLEYMPLCGSDSKTYSNKCNF |
| aura] OVD | | CNAVVDSNGTLTLSHFGKC |
| (native | | |
| sequence) | | |
| Ovomucoid- | SEQ ID NO: | MTTAGVFVLLSFTLCSFPDAAFGVEVDCSPYPNTTNEEGKEVLVCNKILSPICGTDGVTYSNEC |
| like [Cuculus | 175 | LLCAYNLEYGTNISKDYDGECKEVAPVDCSRHPNTTNEEGKVELLCNKDLNPICGTNGVTYD |
| canorus] | | NECLLCARNLESGTSIGKKYDGECKKEIATVDCSDYPKPVCTLEEMPLCGSDNKTYGNKCNFC |
| | | NAVVDSNGTLTLSHFGKC |
| Ovomucoid | SEQ ID NO: | MTTAVVFVLLSFALCCFPDAAFGVEVDCSTYPNSTNEEGKDVLVCPKILGPICGTDGVTYSNE |
| [Antrostomus | 176 | CLLCAYNIQYGTNVSKDHDGECKEIVPVDCSRYPNTTNEEGKVVFLCNKNFDPVCGTDGDTY |
| carolinensis] | | DNECMLCARSLEPGTTVGKKHDGECKREIATVDCSDYPKPTCSAEDMPLCGSDSKTYSNKCN |
| | | FCNAVVDSNGTLTLSRFGKC |
| Ovomucoid | SEQ ID NO: | MTMTGVFVLLSFAICCFPDAAFGVEVDCSTYPNTTNEEGKEVLVCTKILSPICGTDGVTYSNEC |
| [Cariama | 177 | LLCAYNIEYGTNVSKDHDGECKEVVPVDCSKYPNTTNEEGKVVLLCSKDLSPVCGTDGVTYD |
| cristata] | | NECLLCARNLEPGSSVGKKYDGECKKEIATIDCSDYPKPVCSLEYMPLCGSDSKTYDNKCNFC |
| | | NAVVDSNGTLTLSHFGKC |
| Ovomucoid- | SEQ ID NO: | MTTAGVFVLLSFVLCCFPDAVFGVEVDCSTYPNTTNEEGKEVLVCTKILSPICGTDGVTYSNE |
| like isoform | 178 | CLLCAYNIEYGTNVSKDHDGECKEVVPVNCSRYPNTTNEEGKVVLRCSKDLSPVCGTDGVTY |
| X2 | | DNECLMCARNLEPGAVVGKNYDGECKKEIATVDCSDYPKPVCSLEYMPLCGSDSKTYSNKC |
| [Pygoscelis | | NFCNAVVDSNGTLTLSHFGKC |
| adeliae] | | |
| Ovomucoid- | SEQ ID NO: | MTTAGVFVLLSIALCCFPDAAFGVEVDCSAYSNTTSEEGKEVLSCTKILSPICGTDGVTYSNEC |
| like [Nipponia | 179 | LLCAYNIEYGTNISKDHDGECKEVVSVDCSRYPNTTNEEGKAVLLCNKDLSPVCGTDGVTYD |
| nippon] | | NECLLCAHNLEPGTSVGKKYDGACKKEIATVDCSDYPKPVCTLEYLPLCGSDSKTYSNKCDF |
| | | CNAVVDSNGTLTLSHFGKC |
| Ovomucoid- | SEQ ID NO: | MTTAGVFVLLSFALCCFPDAAFGVEVDCSTYPNTTNEEGKEVLVCTKILSPICGTDGTTYSNEC |
| like [Phaethon | 180 | LLCAYNIEYGTNVSKDHDGECKVVPVDCSKYPNTTNEDGKVVLLCNKALSPICGTDRVTYDN |
| lepturus] | | ECLMCAHNLEPGTSVGKKHDGECQKEVATVDCSDYPKPVCSLEYMPLCGSDGKTYSNKCNF |
| | | CNAVVNSNGTLTLSHFEKC |
| Ovomucoid- | SEQ ID NO: | MTTAGVFVLLSFVLCCFFPDAAFGVEVDCSTYPNTTNEEGKEVLVCAKILSPVCGTDGVTYSN |
| like isoform | 181 | ECLLCAHNIENGTNVGKDHDGKCKEAVPVDCSRYPNTTDEEGKVVLLCNKDVSPVCGTDGV |
| X1 | | TYDNECLLCAHNLEAGTSVDKKNDSECKTEDTTLAAVSVDCSDYPKPVCTLEYLPLCGSDNK |
| [Melopsittacus | | TYSNKCRFCNAVVDSNGTLTLSRFGKC |
| undulatus] | | |
| Ovomucoid | SEQ ID NO: | MTTAGVFVLLSFALCCSPDAAFGVEVDCSTYPNTTNEEGKEVLACTKILSPICGTDGVTYSNE |
| [Podiceps | 182 | CLLCAYNMEYGTNVSKDHDGKCKEVVPVDCSRYPNTTNEEGKVVLLCNKDLSPVCGTDGVT |
| cristatus] | | YDNECLLCARNLEPGASVGKKYDGECKKEIATVDCSDYPKPVCSLEHMPLCGSDSKTYSNKC |
| | | TFCNAVVDSNGTLTLSHFGKC |
| Ovomucoid- | SEQ ID NO: | MTTAGVFVLLSFALCCFPDAAFGVEVDCSTYPNTTNEEGREVLVCTKILSPICGTDGVTYSNEC |
| like [Fulmarus | 183 | LLCAYNIEYGTNVSKDHDGECKEVAPVGCSRYPNTTNEEGKVVLLCNKDLSPVCGTDGVTYD |
| glacialis] | | NECLLCARHLEPGTSVGKKYDGECKKEIATVDCSDYPKPVCSLEYMPLCGSDSKTYSNKCNF |
| | | CNAVLDSNGTLTLSHFGKC |
| Ovomucoid | SEQ ID NO: | MTTAGVFVLLSFALCCFPDAVFGVEVDCSTYPNTTNEEGKEVLVCTKILSPICGTDGVTYSNE |
| [Aptenodytes | 184 | CLLCAYNIEYGTNVSKDHDGECKEVVPVDCSRYPNTTNEEGKVVLRCNKDLSPVCGTDGVTY |
| forsteri] | | DNECLMCARNLEPGAIVGKKYDGECKKEIATVDCSDYPKPVCSLEYMPLCGSDSKTYSNKCN |
| | | FCNAVVDSNGTLILSHFGKC |
| Ovomucoid- | SEQ ID NO: | MTTAGVFVLLSFVLCCFPDAVFGVEVDCSTYPNTTNEEGKEVLVCTKILSPICGTDGVTYSNE |
| like isoform | 185 | CLLCAYNIEYGTNVSKDHDGECKEVVPVDCSRYPNTTNEEGKVVLRCSKDLSPVCGTDGVTY |
| X1 | | DNECLMCARNLEPGAVVGKNYDGECKKEIATVDCSDYPKPVCSLEYMPLCGSDSKTYSNKC |
| [Pygoscelis | | NFCNAVVDSNGTLTLSHFGKC |
| adeliae] | | |
| Ovomucoid | SEQ ID NO: | MSSQNQLPSRCRPLPGSQDLNKYYQPHCTGDRFCWLFYVTVEQFRHCICIYLQLALERPSHEQ |
| isoform X1 | 186 | SGQPADSRNTSTMTTAGVFVLLSFALCCFPDAVFGVEVDCSTYPNTTNEEGKEVLVCTKILSPI |
| [Aptenodytes | | CGTDGVTYSNECLLCAYNIEYGTNVSKDHDGECKEVVPVDCSRYPNTTNEEGKVVLRCNKD |
| forsteri] | | LSPVCGTDGVTYDNECLMCARNLEPGAIVGKKYDGECKKEIATVDCSDYPKPVCSLEYMPLC |
| | | GSDSKTYSNKCNFCNAVVDSNGTLILSHFGKC |
| Ovomucoid, | SEQ ID NO: | MTTAVVFVLLSFALCCFPDAAFGVEVDCSTYPNSTNEEGKDVLVCPKILGPICGTDGVTYSNE |
| partial | 187 | CLLCAYNIQYGTNVSKDHDGECKEIVPVDCSRYPNTTNEEGKVVFLCNKNFDPVCGTDGDTY |
| [Antrostomus | | DNECMLCARSLEPGTTVGKKHDGECKREIATVDCSDYPKPTCSAEDMPLCGSDSKTYSNKCN |
| carolinensis] | | FCNAVV |
| rOVD as | SEQ ID NO: | EAEAAEVDCSRFPNATDKEGKDVLVCNKDLRPICGTDGVTYTNDCLLCAYSIEFGTNISKEHD |
| expressed in | 188 | GECKETVPMNCSSYANTTSEDGKVMVLCNRAFNPVCGTDGVTYDNECLLCAHKVEQGASVD |
| pichia | | KRHDGGCRKELAAVSVDCSEYPKPDCTAEDRPLCGSDNKTYGNKCNFCNAVVESNGTLTLS |
| secreted form | | HFGKC |
| 1 | | |
| rOVD as | SEQ ID NO: | EEGVSLEKREAEAAEVDCSRFPNATDKEGKDVLVCNKDLRPICGTDGVTYTNDCLLCAYSIEF |
| expressed in | 189 | GTNISKEHDGECKETVPMNCSSYANTTSEDGKVMVLCNRAFNPVCGTDGVTYDNECLLCAH |
| pichia | | KVEQGASVDKRHDGGCRKELAAVSVDCSEYPKPDCTAEDRPLCGSDNKTYGNKCNFCNAVV |
| secreted form | | ESNGTLTLSHFGKC |
| 2 | | |
| rOVD [gallus] | SEQ ID NO: | MRFPSIFTAVLFAASSALAAPVNTTTEDETAQIPAEAVIGYSDLEGDFDVAVLPFSNSTNNGLL |
| coding | 190 | FINTTIASIAAKEEGVSLEKREAEAAEVDCSRFPNATDKEGKDVLVCNKDLRPICGTDGVTYTN |
| sequence | | DCLLCAYSIEFGTNISKEHDGECKETVPMNCSSYANTTSEDGKVMVLCNRAFNPVCGTDGVT |
| containing an | | YDNECLLCAHKVEQGASVDKRHDGGCRKELAAVSVDCSEYPKPDCTAEDRPLCGSDNKTYG |
| alpha mating | | NKCNFCNAVVESNGTLTLSHFGKC |
| factor signal | | |
| sequence | | |
| (bolded) as | | |
| expressed in | | |
| pichia | | |
| Turkey | SEQ ID NO: | MRFPSIFTAVLFAASSALAAPVNTTTEDETAQIPAEAVIGYSDLEGDFDVAVLPFSNSTNNGLL |
| vulture OVD | 191 | FINTTIASIAAKEEGVSLEKREAEAVEVDCSTYPNTTNEEGKEVLVCTKILSPICGTDGVTYSNE |
| coding | | CLLCAYNIEYGTNVSKDHDGECKEFVPVDCSRYPNTTNEDGKVVLLCNKDLSPICGTDGVTY |
| sequence | | DNECLLCARNLEPGTSVGKKYDGECKKEIATVDCSDYPKPVCSLEYMPLCGSDSKTYSNKCN |
| containing | | FCNAVVDSNGTLTLSHFGKC |
| secretion | | |
| signals as | | |
| expressed in | | |
| pichia bolded | | |
| is an alpha | | |
| mating factor | | |
| signal | | |
| sequence | | |
| Turkey | SEQ ID NO: | EAEAVEVDCSTYPNTTNEEGKEVLVCTKILSPICGTDGVTYSNECLLCAYNIEYGTNVSKDHD |
| vulture OVD | 192 | GECKEFVPVDCSRYPNTTNEDGKVVLLCNKDLSPICGTDGVTYDNECLLCARNLEPGTSVGK |
| in secreted | | KYDGECKKEIATVDCSDYPKPVCSLEYMPLCGSDSKTYSNKCNFCNAVVDSNGTLTLSHFGK |
| form | | C |
| expressed in | | |
| Pichia | | |
| Humming bird | SEQ ID NO: | MTMAGVFVLLSFILCCFPDTAFGVEVDCSIYPNTTSEEGKEVLVCTETLSPICGSDGVTYNNEC |
| OVD (native | 193 | QLCAYNVEYGTNVSKDHDGECKEIVPVDCSRYPNTTEEGRVVMLCNKALSPVCGTDGVTYD |
| sequence) | | NECLLCARNLESGTSVGKKFDGECKKEIATVDCTDYPKPVCSLDYMPLCGSDSKTYSNKCNF |
| | | CNAVMDSNGTLTLNHFGKC |
| Humming bird | SEQ ID NO: | MRFPSIFTAVLFAASSALAAPVNTTTEDETAQIPAEAVIGYSDLEGDFDVAVLPFSNSTNNGLL |
| OVD coding | 194 | FINTTIASIAAKEEGVSLDKREAEAVEVDCSIYPNTTSEEGKEVLVCTETLSPICGSDGVTYNNE |
| sequence as | | CQLCAYNVEYGTNVSKDHDGECKEIVPVDCSRYPNTTEEGRVVMLCNKALSPVCGTDGVTY |
| expressed in | | DNECLLCARNLESGTSVGKKFDGECKKEIATVDCTDYPKPVCSLDYMPLCGSDSKTYSNKCN |
| Pichia | | FCNAVMDSNGTLTLNHFGKC |
| Humming bird | SEQ ID NO: | EAEAVEVDCSIYPNTTSEEGKEVLVCTETLSPICGSDGVTYNNECQLCAYNVEYGTNVSKDHD |
| OVD in | 195 | GECKEIVPVDCSRYPNTTEEGRVVMLCNKALSPVCGTDGVTYDNECLLCARNLESGTSVGKK |
| secreted form | | FDGECKKEIATVDCTDYPKPVCSLDYMPLCGSDSKTYSNKCNFCNAVMDSNGTLTLNHFGKC |
| from Pichia | | |
| Ovalbumin | SEQ ID NO: | MFFYNTDFRMGSISAANAEFCFDVFNELKVQHTNENILYSPLSIIVALAMVYMGARGNTEYQ |
| related protein | 196 | MEKALHFDSIAGLGGSTQTKVQKPKCGKSVNIHLLFKELLSDITASKANYSLRIANRLYAEKSR |
| X | | PILPIYLKCVKKLYRAGLETVNFKTASDQARQLINSWVEKQTEGQIKDLLVSSSTDLDTTLVLV |
| | | NAIYFKGMWKTAFNAEDTREMPFHVTKEESKPVQMMCMNNSFNVATLPAEKMKILELPFAS |
| | | GDLSMLVLLPDEVSGLERIEKTINFEKLTEWTNPNTMEKRRVKVYLPQMKIEEKYNLTSVLM |
| | | ALGMTDLFIPSANLTGISSAESLKISQAVHGAFMELSEDGIEMAGSTGVIEDIKHSPELEQFRAD |
| | | HPFLFLIKHNPTNTIVYFGRYWSP* |
| Ovalbumin | SEQ ID NO: | MDSISVTNAKFCFDVFNEMKVHHVNENILYCPLSILTALAMVYLGARGNTESQMKKVLHFDS |
| related protein | 197 | ITGAGSTTDSQCGSSEYVHNLFKELLSEITRPNATYSLEIADKLYVDKTFSVLPEYLSCARKFYT |
| Y | | GGVEEVNFKTAAEEARQLINSWVEKETNGQIKDLLVSSSIDFGTTMVFINTIYFKGIWKIAFNT |
| | | EDTREMPFSMTKEESKPVQMMCMNNSFNVATLPAEKMKILELPYASGDLSMLVLLPDEVSGL |
| | | ERIEKTINFDKLREWTSTNAMAKKSMKVYLPRMKIEEKYNLTSILMALGMTDLFSRSANLTGI |
| | | SSVDNLMISDAVHGVFMEVNEEGTEATGSTGAIGNIKHSLELEEFRADHPFLFFIRYNPTNAILF |
| | | FGRYWSP* |
| Ovalbumin | SEQ ID NO: | MGSIGAASMEFCFDVFKELKVHHANENIFYCPIAIMSALAMVYLGAKDSTRTQINKVVRFDKL |
| | 198 | PGFGDSIEAQCGTSVNVHSSLRDILNQITKPNDVYSFSLASRLYAEERYPILPEYLQCVKELYRG |
| | | GLEPINFQTAADQARELINSWVESQINGIIRNVLQPSSVDSQTAMVLVNAIVFKGLWEKAFKD |
| | | EDTQAMPFRVTEQESKPVQMMYQIGLFRVASMASEKMKILELPFASGTMSMLVLLPDEVSGL |
| | | EQLESIINFEKLTEWTSSNVMEERKIKVYLPRMKMEEKYNLTSVLMAMGITDVFSSSANLSGIS |
| | | SAESLKISQAVHAAHAEINEAGREVVGSAEAGVDAASVSEEFRADHPFLFCIKHIATNAVLFFG |
| | | RCVSP* |
| Chicken | SEQ ID NO: | MRFPSIFTAVLFAASSALAAPVNTTTEDETAQIPAEAVIGYSDLEGDFDVAVLPFSNSTNNGLL |
| Ovalbumin | 199 | FINTTIASIAAKEEGVSLDKREAEAGSIGAASMEFCFDVFKELKVHHANENIFYCPIAIMSALAM |
| with bolded | | VYLGAKDSTRTQINKVVRFDKLPGFGDSIEAQCGTSVNVHSSLRDILNQITKPNDVYSFSLASR |
| signal | | LYAEERYPILPEYLQCVKELYRGGLEPINFQTAADQARELINSWVESQINGIIRNVLQPSSVDS |
| sequence | | QTAMVLVNAIVFKGLWEKAFKDEDTQAMPFRVTEQESKPVQMMYQIGLFRVASMASEKMKI |
| | | LELPFASGTMSMLVLLPDEVSGLEQLESIINFEKLTEWTSSNVMEERKIKVYLPRMKMEEKYN |
| | | LTSVLMAMGITDVFSSSANLSGISSAESLKISQAVHAAHAEINEAGREVVGSAEAGVDAASVS |
| | | EEFRADHPFLFCIKHIATNAVLFFGRCVSP |
| Chicken OVA | SEQ ID NO: | EAEAGSIGAASMEFCFDVFKELKVHHANENIFYCPIAIMSALAMVYLGAKDSTRTQINKVVRF |
| sequence as | 200 | DKLPGFGDSIEAQCGTSVNVHSSLRDILNQITKPNDVYSFSLASRLYAEERYPILPEYLQCVKEL |
| secreted from | | YRGGLEPINFQTAADQARELINSWVESQTNGIIRNVLQPSSVDSQTAMVLVNAIVFKGLWEKA |
| pichia | | FKDEDTQAMPFRVTEQESKPVQMMYQIGLFRVASMASEKMKILELPFASGTMSMLVLLPDEV |
| | | SGLEQLESIINFEKLTEWTSSNVMEERKIKVYLPRMKMEEKYNLTSVLMAMGITDVFSSSANL |
| | | SGISSAESLKISQAVHAAHAEINEAGREVVGSAEAGVDAASVSEEFRADHPFLFCIKHIATNAV |
| | | LFFGRCVSP |
| Predicted | SEQ ID NO: | MRVPAQLLGLLLLWLPGARCGSIGAASMEFCFDVFKELKVHHANENIFYCPIAIMSALAMVY |
| Ovalbumin | 201 | LGAKDSTRTQINKVVRFDKLPGFGDSIEAQCGTSVNVHSSLRDILNQITKPNDVYSFSLASRLY |
| [Achromobacter | | AEERYPILPEYLQCVKELYRGGLEPINFQTAADQARELINSWVESQTNGIIRNVLQPSSVDSQT |
| denitrificans] | | AMVLVNAIVFKGLWEKAFKDEDTQAMPFRVTEQESKPVQMMYQIGLFRVASMASEKMKILE |
| | | LPFASGTMSMLVLLPDEVSGLEQLESIINFEKLTEWTSSNVMEERKIKVYLPRMKMEEKYNLT |
| | | SVLMAMGITDVFSSSANLSGISSAESLKISQAVHAAHAEINEAGREVVGSAEAGVDAASVSEEF |
| | | RADHPFLFCIKHIATNAVLFFGRCVSPLEIKRAAAHHHHHH |
| OLLAS | SEQ ID NO: | MTSGFANELGPRLMGKLTMGSIGAASMEFCFDVFKELKVHHANENIFYCPIAIMSALAMVYL |
| epitope- | 202 | GAKDSTRTQINKVVRFDKLPGFGDSIEAQCGTSVNVHSSLRDILNQITKPNDVYSFSLASRLYA |
| tagged | | EERYPILPEYLQCVKELYRGGLEPINFQTAADQARELINSWVESQTNGIIRNVLQPSSVDSQTA |
| ovalbumin | | MVLVNAIVFKGLWEKTFKDEDTQAMPFRVTEQESKPVQMMYQIGLFRVASMASEKMKILEL |
| | | PFASGTMSMLVLLPDEVSGLEQLESIINFEKLTEWTSSNVMEERKIKVYLPRMKMEEKYNLTS |
| | | VLMAMGITDVFSSSANLSGISSAESLKISQAVHAAHAEINEAGREVVGSAEAGVDAASVSEEF |
| | | RADHPFLFCIKHIATNAVLFFGRCVSPSR |
| Serpin family | SEQ ID NO: | MGGRRVRWEVYISRAGYVNRQIAWRRHHRSLTMRVPAQLLGLLLLWLPGARCGSIGAASME |
| protein | 203 | FCFDVFKELKVHHANENIFYCPIAIMSALAMVYLGAKDSTRTQINKVVRFDKLPGFGDSIEAQ |
| [Achromobacter | | CGTSVNVHSSLRDILNQITKPNDVYSFSLASRLYAEERYPILPEYLQCVKELYRGGLEPINFQTA |
| denitrificans] | | ADQARELINSWVESQTNGIIRNVLQPSSVDSQTAMVLVNAIVFKGLWEKAFKDEDTQAMPFR |
| | | VTEQESKPVQMMYQIGLFRVASMASEKMKILELPFASGTMSMLVLLPDEVSGLEQLESIINFE |
| | | KLTEWTSSNVMEERKIKVYLPRMKMEEKYNLTSVLMAMGITDVFSSSANLSGISSAESLKISQ |
| | | AVHAAHAEINEAGREVVGSAEAGVDAASVSEEFRADHPFLFCIKHIATNAVLFFGRCVSPLEIK |
| | | RAAAHHHHHH |
| PREDICTED: | SEQ ID NO: | MGSIGAVSMEFCFDVFKELKVHHANENIFYSPFTIISALAMVYLGAKDSTRTQINKVVRFDKLP |
| ovalbumin | 204 | GFGDSVEAQCGTSVNVHSSLRDILNQITKPNDVYSFSLASRLYAEETYPILPEYLQCVKELYRG |
| isoform X1 | | GLESINFQTAADQARGLINSWVESQTNGMIKNVLQPSSVDSQTAMVLVNAIVFKGLWEKAFK |
| [Meleagris | | DEDTQAIPFRVTEQESKPVQMMYQIGLFKVASMASEKMKILELPFASGTMSMWVLLPDEVSG |
| gallopavo] | | LEQLETTISFEKMTEWISSNIMEERRIKVYLPRMKMEEKYNLTSVLMAMGITDLFSSSANLSGI |
| | | SSAGSLKISQAVHAAYAEIYEAGREVIGSAEAGADATSVSEEFRVDHPFLYCIKHNLTNSILFFG |
| | | RCISP |
| Ovalbumin | SEQ ID NO: | MGSIGAVSMEFCFDVFKELKVHHANENIFYSPFTIISALAMVYLGAKDSTRTQINKVVRFDKLP |
| precursor | 205 | GFGDSVEAQCGTSVNVHSSLRDILNQITKPNDVYSFSLASRLYAEETYPILPEYLQCVKELYRG |
| [Meleagris | | GLESINFQTAADQARGLINSWVESQTNGMIKNVLQPSSVDSQTAMVLVNAIVFKGLWEKAFK |
| gallopavo] | | DEDTQAIPFRVTEQESKPVQMMYQIGLFKVASMASEKMKILELPFASGTMSMWVLLPDEVSG |
| | | LEQLETTISFEKMTEWISSNIMEERRIKVYLPRMKMEEKYNLTSVLMAMGITDLFSSSANLSGI |
| | | SSAGSLKISQAAHAAYAEIYEAGREVIGSAEAGADATSVSEEFRVDHPFLYCIKHNLTNSILFFG |
| | | RCISP |
| Hypothetical | SEQ ID NO: | YYRVPCMVLCTAFHPYIFIVLLFALDNSEFTMGSIGAVSMEFCFDVFKELRVHHPNENIFFCPF |
| protein | 206 | AIMSAMAMVYLGAKDSTRTQINKVIRFDKLPGFGDSTEAQCGKSANVHSSLKDILNQITKPND |
| [Bambusicola | | VYSFSLASRLYADETYSIQSEYLQCVNELYRGGLESINFQTAADQARELINSWVESQINGIIRN |
| thoracicus] | | VLQPSSVDSQTAMVLVNAIVFRGLWEKAFKDEDTQTMPFRVTEQESKPVQMMYQIGSFKVAS |
| | | MASEKMKILELPLASGTMSMLVLLPDEVSGLEQLETTISFEKLTEWTSSNVMEERKIKVYLPR |
| | | MKMEEKYNLTSVLMAMGITDLFRSSANLSGISLAGNLKISQAVHAAHAEINEAGRKAVSSAE |
| | | AGVDATSVSEEFRADRPFLFCIKHIATKVVFFFGRYTSP |
| Egg albumin | SEQ ID NO: | MGSIGAASMEFCFDVFKELKVHHANDNMLYSPFAILSTLAMVFLGAKDSTRTQINKVVHFDK |
| | 207 | LPGFGDSIEAQCGTSVNVHSSLRDILNQITKQNDAYSFSLASRLYAQETYTVVPEYLQCVKELY |
| | | RGGLESVNFQTAADQARGLINAWVESQINGIIRNILQPSSVDSQTAMVLVNAIAFKGLWEKAF |
| | | KAEDTQTIPFRVTEQESKPVQMMYQIGSFKVASMASEKMKILELPFASGTMSMLVLLPDDVSG |
| | | LEQLESIISFEKLTEWTSSSIMEERKVKVYLPRMKMEEKYNLTSLLMAMGITDLFSSSANLSGIS |
| | | SVGSLKISQAVHAAHAEINEAGRDVVGSAEAGVDATEEFRADHPFLFCVKHIETNAILLFGRC |
| | | VSP |
| Ovalbumin | SEQ ID NO: | MASIGAVSTEFCVDVYKELRVHHANENIFYSPFTIISTLAMVYLGAKDSTRTQINKVVRFDKLP |
| isoform X2 | 208 | GFGDSIEAQCGTSVNVHSSLRDILNQITKPNDVYSFSLASRLYAEETYPILPEYLQCVKELYRG |
| [Numida | | GLESINFQTAADQARELINSWVESQTSGIIKNVLQPSSVNSQTAMVLVNAIYFKGLWERAFKD |
| meleagris] | | EDTQAIPFRVTEQESKPVQMMSQIGSFKVASVASEKVKILELPFVSGTMSMLVLLPDEVSGLEQ |
| | | LESTISTEKLTEWTSSSIMEERKIKVFLPRMRMEEKYNLTSVLMAMGMTDLFSSSANLSGISSA |
| | | ESLKISQAVHAAYAEIYEAGREVVSSAEAGVDATSVSEEFRVDHPFLLCIKHNPTNSILFFGRCI |
| | | SP |
| Ovalbumin | SEQ ID NO: | MALCKAFHPYIFIVLLFDVDNSAFTMASIGAVSTEFCVDVYKELRVHHANENIFYSPFTIISTLA |
| isoform X1 | 209 | MVYLGAKDSTRTQINKVVRFDKLPGFGDSIEAQCGTSVNVHSSLRDILNQITKPNDVYSFSLAS |
| [Numida | | RLYAEETYPILPEYLQCVKELYRGGLESINFQTAADQARELINSWVESQTSGIIKNVLQPSSVNS |
| meleagris] | | QTAMVLVNAIYFKGLWERAFKDEDTQAIPFRVTEQESKPVQMMSQIGSFKVASVASEKVKILE |
| | | LPFVSGTMSMLVLLPDEVSGLEQLESTISTEKLTEWTSSSIMEERKIKVFLPRMRMEEKYNLTS |
| | | VLMAMGMTDLFSSSANLSGISSAESLKISQAVHAAYAEIYEAGREVVSSAEAGVDATSVSEEF |
| | | RVDHPFLLCIKHNPTNSILFFGRCISP |
| PREDICTED: | SEQ ID NO: | MGSIGAASMEFCFDVFKELKVHHANDNMLYSPFAILSTLAMVFLGAKDSTRTQINKVVHFDK |
| Ovalbumin | 210 | LPGFGDSIEAQCGTSANVHSSLRDILNQITKQNDAYSFSLASRLYAQETYTVVPEYLQCVKELY |
| isoform X2 | | RGGLESVNFQTAADQARGLINAWVESQINGIIRNILQPSSVDSQTAMVLVNAIAFKGLWEKAF |
| [Coturnix | | KAEDTQTIPFRVTEQESKPVQMMHQIGSFKVASMASEKMKILELPFASGTMSMLVLLPDDVSG |
| japonica] | | LEQLESTISFEKLTEWTSSSIMEERKVKVYLPRMKMEEKYNLTSLLMAMGITDLFSSSANLSGI |
| | | SSVGSLKISQAVHAAYAEINEAGRDVVGSAEAGVDATEEFRADHPFLFCVKHIETNAILLFGRC |
| | | VSP |
| PREDICTED: | SEQ ID NO: | MGLCTAFHPYIFIVLLFALDNSEFTMGSIGAASMEFCFDVFKELKVHHANDNMLYSPFAILSTL |
| ovalbumin | 211 | AMVFLGAKDSTRTQINKVVHFDKLPGFGDSIEAQCGTSANVHSSLRDILNQITKQNDAYSFSL |
| isoform X1 | | ASRLYAQETYTVVPEYLQCVKELYRGGLESVNFQTAADQARGLINAWVESQINGIIRNILQPS |
| [Coturnix | | SVDSQTAMVLVNAIAFKGLWEKAFKAEDTQTIPFRVTEQESKPVQMMHQIGSFKVASMASEK |
| japonica] | | MKILELPFASGTMSMLVLLPDDVSGLEQLESTISFEKLTEWTSSSIMEERKVKVYLPRMKMEE |
| | | KYNLTSLLMAMGITDLFSSSANLSGISSVGSLKISQAVHAAYAEINEAGRDVVGSAEAGVDAT |
| | | EEFRADHPFLFCVKHIETNAILLFGRCVSP |
| Egg albumin | SEQ ID NO: | MGSIGAASMEFCFDVFKELKVHHANDNMLYSPFAILSTLAMVFLGAKDSTRTQINKVVHFDK |
| | 212 | LPGFGDSIEAQCGTSANVHSSLRDILNQITKQNDAYSFSLASRLYAQETYTVVPEYLQCVKELY |
| | | RGGLESVNFQTAADQARGLINAWVESQINGIIRNILQPSSVDSQTAMVLVNAIAFKGLWEKAF |
| | | KAEDTQTIPFRVTEQESKPVQMMHQIGSFKVASMASEKMKILELPFASGTMSMLVLLPDDVSG |
| | | LEQLESTISFEKLTEWTSSSIMEERKVKVYLPRMKMEEKYNLTSLLMAMGITDLFSSSANLSGI |
| | | SSVGSLKIPQAVHAAYAEINEAGRDVVGSAEAGVDATEEFRADHPFLFCVKHIETNAILLFGRC |
| | | VSP |
| ovalbumin | SEQ ID NO: | MGSIGAASTEFCFDVFRELRVQHVNENIFYSPFSIISALAMVYLGARDNTRTQIDKVVHFDKLP |
| [Anas | 213 | GFGESMEAQCGTSVSVHSSLRDILTQITKPSDNFSLSFASRLYAEETYAILPEYLQCVKELYKG |
| platyrhynchos] | | GLESISFQTAADQARELINSWVESQINGIIKNILQPSSVDSQTTMVLVNAIYFKGMWEKAFKDE |
| | | DTQAMPFRMTEQESKPVQMMYQVGSFKVAMVTSEKMKILELPFASGMMSMFVLLPDEVSGL |
| | | EQLESTISFEKLTEWTSSTMMEERRMKVYLPRMKMEEKYNLTSVFMALGMTDLFSSSANMSG |
| | | ISSTVSLKMSEAVHAACVEIFEAGRDVVGSAEAGMDVTSVSEEFRADHPFLFFIKHNPTNSILFF |
| | | GRWMSP |
| PREDICTED: | SEQ ID NO: | MGSIGAASTEFCFDVFRELKVQHVNENIFYSPLSIISALAMVYLGARDNTRTQIDQVVHFDKIP |
| ovalbumin- | 214 | GFGESMEAQCGTSVSVHSSLRDILTEITKPSDNFSLSFASRLYAEETYTILPEYLQCVKELYKGG |
| like | | LESISFQTAADQARELINSWVESQINGIIKNILQPSSVDSQTTMVLVNAIYFKGMWEKAFKDED |
| [Ansercygnoides | | TQTMPFRMTEQESKPVQMMYQVGSFKLATVTSEKVKILELPFASGMMSMCVLLPDEVSGLEQ |
| domesticus] | | LETTISFEKLTEWTSSTMMEERRMKVYLPRMKMEEKYNLTSVFMALGMTDLFSSSANMSGIS |
| | | STVSLKMSEAVHAACVEIFEAGRDVVGSAEAGMDVTSVSEEFRADHPFLFFIKHNPSNSILFFG |
| | | RWISP |
| PREDICTED: | SEQ ID NO: | MGSIGAASTEFCFDVFKELKVQHVNENIFYSPLTIISALSMVYLGARENTRAQIDKVLHFDKMP |
| Ovalbumin- | 215 | GFGDTIESQCGTSVSIHTSLKDMFTQITKPSDNYSLSFASRLYAEETYPILPEYLQCVKELYKGG |
| like [Aquila | | LETISFQTAAEQARELINSWVESQTNGMIKNILQPSSVDPQTKMVLVNAIYFKGVWEKAFKDE |
| chrysaetos | | DTQEVPFRVTEQESKPVQMMYQIGSFKVAVMASEKMKILELPYASGQLSMLVLLPDDVSGLE |
| canadensis] | | QLESAITFEKLMAWTSSTTMEERKMKVYLPRMKIEEKYNLTSVLMALGVTDLFSSSANLSGIS |
| | | SAESLKISKAVHEAFVEIYEAGSEVVGSTEAGMEVTSVSEEFRADHPFLFLIKHNPTNSILFFGR |
| | | CFSP |
| PREDICTED: | SEQ ID NO: | MGSIGAASTEFCFDVFKELKVQHVNENIFYSPLTIISALSMVYLGARENTRTQIDKVLHFDKMT |
| Ovalbumin- | 216 | GFGDTVESQCGTSVSIHTSLKDIFTQITKPSDNYSLSLASRLYAEETYPILPEYLQCVKELYKGG |
| like | | LETVSFQTAAEQARELINSWVESQTNGMIKNILQPSSVDPQTKMVLVNAIYFKGVWEKAFKD |
| [Haliaeetus | | EDTQEVPFRVTEQESKPVQMMYQIGSFKVAVMASEKMKILELPYASGQLSMLVLLPDDVSGL |
| albicilla] | | EQLESAITSEKLMEWTSSTTMEERKMKVYLPRMKIEEKYNLTSVLMALGVTDLFSSSADLSGI |
| | | SSAESLKISKAVHEAFVEIYEAGSEVVGSTEGGMEVTSVSEEFRADHPFLFLIKHKPTNSILFFG |
| | | RCFSP |
| PREDICTED: | SEQ ID NO: | MGSIGAASTEFCFDVFKELKVQHVNENIFYSPLTIISALSMVYLGARENTRTQIDKVLHFDKMT |
| Ovalbumin- | 217 | GFGDTVESQCGTSVSIHTSLKDIFTQITKPSDNYSLSLASRLYAEETYPILPEYLQCVKELYKGG |
| like | | LETVSFQTAAEQARELINSWVESQTNGMIKNILQPSSVDPQTKMVLVNAIYFKGVWEKAFKD |
| [Haliaeetus | | EDTQEVPFRVTEQESKPVQMMYQIGSFKVAVMASEKMKILELPYASGQLSMLVLLPDDVSGL |
| leucocephalus] | | EQLESAITSEKLMEWTSSTTMEERKMKVYLPRMKIEEKYNLTSVLMALGVTDLFSSSADLSGI |
| | | SSAESLKISKAVHEAFVEIYEAGSEVVGSTEGGMEVTSFSEEFRADHPFLFLIKHKPTNSILFFGR |
| | | CFSP |
| PREDICTED: | SEQ ID NO: | MGSIGAASTEFCFDVFKELKVQHVNENIFYSPLSIISALSMVYLGARENTRAQIDKVVHFDKIT |
| Ovalbumin | 218 | GFGETIESQCGTSVSVHTSLKDMFTQITKPSDNYSLSFASRLYAEETYPILPEYLQCVKELYKG |
| [Fulmarus | | GLETTSFQTAADQARELINSWVESQTNGMIKNILQPGSVDPQTEMVLVNAIYFKGMWEKAFK |
| glacialis] | | DEDTQAVPFRMTEQESKTVQMMYQIGSFKVAVMASEKMKILELPYASGELSMLVMLPDDVS |
| | | GLEQLETAITFEKLMEWTSSNMMEERKMKVYLPRMKMEEKYNLTSVLMALGVTDLFSSSAN |
| | | LSGISSAESLKMSEAVHEAFVEIYEAGSEVVGSTGAGMEVTSVSEEFRADHPFLFLIKHNPTNSI |
| | | LFFGRCFSP |
| PREDICTED: | SEQ ID NO: | MGSIGAASTEFCFDVFKELRVQHVNENVCYSPLIIISALSLVYLGARENTRAQIDKVVHFDKIT |
| Ovalbumin- | 219 | GFGESIESQCGTSVSVHTSLKDMFNQITKPSDNYSLSVASRLYAEERYPILPEYLQCVKELYKG |
| like | | GLESISFQTAADQAREAINSWVESQTNGMIKNILQPSSVDPQTEMVLVNAIYFKGMWQKAFK |
| [Chlamydotis | | DEDTQAVPFRISEQESKPVQMMYQIGSFKVAVMAAEKMKILELPYASGELSMLVLLPDEVSG |
| macqueenii] | | LEQLENAITVEKLMEWTSSSPMEERIMKVYLPRMKIEEKYNLTSVLMALGITDLFSSSANLSGI |
| | | SAEESLKMSEAVHQAFAEISEAGSEVVGSSEAGIDATSVSEEFRADHPFLFLIKHNATNSILFFG |
| | | RCFSP |
| PREDICTED: | SEQ ID NO: | MGSISAASTEFCFDVFKELKVQHVNENIFYSPLSIISALSMVYLGARENTRAQIEKVVHFDKITG |
| Ovalbumin | 220 | FGESIESQCSTSVSVHTSLKDMFTQITKPSDNYSLSFASRFYAEETYPILPEYLQCVKELYKGGL |
| like [Nipponia | | ETINFRTAADQARELINSWVESQTNGMIKNILQPGSVDPQTDMVLVNAIYFKGMWEKAFKDE |
| nippon] | | DTQALPFRVTEQESKPVQMMYQIGSFKVAVLASEKVKILELPYASGQLSMLVLLPDDVSGLEQ |
| | | LETAITVEKLMEWTSSNNMEERKIKVYLPRIKIEEKYNLTSVLMALGITDLFSSSANLSGISSAE |
| | | SLKVSEAIHEAFVEIYEAGSEVAGSTEAGIEVTSVSEEFRADHPFLFLIKHNATNSILFFGRCFSP |
| PREDICTED: | SEQ ID NO: | MVSIGAASTEFCFDVFKELKVQHVNENIFYSPLSIISALSMVYLGARENTRAQIDKVVHFDKIT |
| Ovalbumin- | 221 | GFEETIESQCSTSVSVHTSLKDMFTQITKPSDNYSLSFASRLYAEETYPILPEYLQCVKELYKGG |
| like isoform | | LETISFQTAADQARELINSWVESQTDGMIKNILQPGSVDPQTEMVLVNAIYFKGMWEKAFKDE |
| X2 [Gavia | | DTQAVPFRMTEQESKPVQMMYQIGSFKVAVMASEKMKILELPYASGGMSMLVMLPDDVSGL |
| stellata] | | EQLETAITFEKLMEWTSSNMMEERKMKVYLPRMKMEEKYNLTSVLMALGMTDLFSSSANLS |
| | | GISSAESLKMSEAVHEAFVEIYEAGSEAVGSTGAGMEVTSVSEEFRADHPFLFLIKHNPTNSILF |
| | | FGRCFSP |
| PREDICTED: | SEQ ID NO: | MGSIGAASTEFCFDVFKELKVQHVNENIFYSPLSIISALSMVYLGARENTRAQIDKVVHFDKIT |
| Ovalbumin | 222 | GFGEPIESQCGISVSVHTSLKDMITQITKPSDNYSLSFASRLYAEETYPILPEYLQCVKELYKGG |
| [Pelecanus | | LETISFQTAADQARELINSWVENQTNGMIKNILQPGSVDPQTEMVLVNAVYFKGMWEKAFKD |
| crispus] | | EDTQAVPFRMTEQESKPVQMMYQIGSFKVAVMASEKIKILELPYASGELSMLVLLPDDVSGLE |
| | | QLETAITLDKLTEWTSSNAMEERKMKVYLPRMKIEKKYNLTSVLIALGMTDLFSSSANLSGISS |
| | | AESLKMSEAIHEAFLEIYEAGSEVVGSTEAGMEVTSVSEEFRADHPFLFLIKHNPTNSILFFGRC |
| | | LSP |
| PREDICTED: | SEQ ID NO: | MGSIGAASTEFCFDVFKELKVQHVNENIFYSPLTIISALSMVYLGARENTRAQIDKVVHFDKIP |
| Ovalbumin- | 223 | GFGDTTESQCGTSVSVHTSLKDMFTQITKPSDNYSVSFASRLYAEETYPILPEFLECVKELYKG |
| like | | GLESISFQTAADQARELINSWVESQTNGMIKNILQPGSVDSQTEMVLVNAIYFKGMWEKAFK |
| [Charadrius | | DEDTQTVPFRMTEQETKPVQMMYQIGTFKVAVMPSEKMKILELPYASGELCMLVMLPDDVS |
| vociferus] | | GLEELESSITVEKLMEWTSSNMMEERKMKVFLPRMKIEEKYNLTSVLMALGMTDLFSSSANL |
| | | SGISSAEPLKMSEAVHEAFIEIYEAGSEVVGSTGAGMEITSVSEEFRADHPFLFLIKHNPTNSILF |
| | | FGRCVSP |
| PREDICTED: | SEQ ID NO: | MGSIGAVSTEFCFDVFKELKVQHVNENIFYSPLSIISALSMVYLGARENTRAQIDKVVHFDKIT |
| Ovalbumin- | 224 | GSGETIEAQCGTSVSVHTSLKDMFTQITKPSENYSVGFASRLYADETYPIIPEYLQCVKELYKG |
| like | | GLEMISFQTAADQARELINSWVESQTNGMIKNILQPGSVDPQTEMILVNAIYFKGVWEKAFKD |
| [Eurypyga | | EDTQAVPFRMTEQESKPVQMMYQFGSFKVAAMAAEKMKILELPYASGALSMLVLLPDDVSG |
| helias] | | LEQLESAITFEKLMEWTSSNMMEEKKIKVYLPRMKMEEKYNFTSVLMALGMTDLFSSSANLS |
| | | GISSADSLKMSEVVHEAFVEIYEAGSEVVGSTGSGMEAASVSEEFRADHPFLFLIKHNPTNSILF |
| | | FGRCFSP |
| PREDICTED: | SEQ ID NO: | MVSIGAASTEFCFDVFKELKVQHVNENIFYSPLSIISALSMVYLGARENTRAQIDKVVHFDKIT |
| Ovalbumin- | 225 | GFEETIESQVQKKQCSTSVSVHTSLKDMFTQITKPSDNYSLSFASRLYAEETYPILPEYLQCVKE |
| like isoform | | LYKGGLETISFQTAADQARELINSWVESQTDGMIKNILQPGSVDPQTEMVLVNAIYFKGMWE |
| X1 [Gavia | | KAFKDEDTQAVPFRMTEQESKPVQMMYQIGSFKVAVMASEKMKILELPYASGGMSMLVMLP |
| stellata] | | DDVSGLEQLETAITFEKLMEWTSSNMMEERKMKVYLPRMKMEEKYNLTSVLMALGMTDLF |
| | | SSSANLSGISSAESLKMSEAVHEAFVEIYEAGSEAVGSTGAGMEVTSVSEEFRADHPFLFLIKH |
| | | NPTNSILFFGRCFSP |
| PREDICTED: | SEQ ID NO: | MGSIGAASGEFCFDVFKELKVQHVNENIFYSPLSIISALSMVYLGARENTRAQIDKVVHFDKIIG |
| Ovalbumin - | 226 | FGESIESQCGTSVSVHTSLKDMFAQITKPSDNYSLSFASRLYAEETFPILPEYLQCVKELYKGGL |
| like [Egretta | | ETLSFQTAADQARELINSWVESQTNGMIKDILQPGSVDPQTEMVLVNAIYFKGVWEKAFKDE |
| garzetta] | | DTQTVPFRMTEQESKPVQMMYQIGSFKVAVVAAEKIKILELPYASGALSMLVLLPDDVSSLEQ |
| | | LETAITFEKLTEWTSSNIMEERKIKVYLPRMKIEEKYNLTSVLMDLGITDLFSSSANLSGISSAES |
| | | LKVSEAIHEAIVDIYEAGSEVVGSSGAGLEGTSVSEEFRADHPFLFLIKHNPTSSILFFGRCFSP |
| PREDICTED: | SEQ ID NO: | MGSIGAASTEFCFDVFKELKVQHVNENIFYSPLSIISALSMVYLGARENTRAQIDKVVHFDKIT |
| Ovalbumin- | 227 | GSGEAIESQCGTSVSVHISLKDMFTQITKPSDNYSLSFASRLYAEETYPILPEYLQCVKELYKEG |
| like [Balearica | | LATISFQTAADQAREFINSWVESQTNGMIKNILQPGSVDPQTQMVLVNAIYFKGVWEKAFKDE |
| regulorum | | DTQAVPFRMTKQESKPVQMMYQIGSFKVAVMASEKMKILELPYASGQLSMLVMLPDDVSGL |
| gibbericeps] | | EQIENAITFEKLMEWTNPNMMEERKMKVYLPRMKMEEKYNLTSVLMALGMTDLFSSSANLS |
| | | GISSAESLKMSEAVHEAFVEIYEAGSEVVGSTGAGIEVTSVSEEFRADHPFLFLIKHNPTNSILFF |
| | | GRCFSP |
| PREDICTED: | SEQ ID NO: | MGSIGEASTEFCIDVFRELKVQHVNENIFYSPLSIISALSMVYLGARENTRAQIDQVVHFDKITG |
| Ovalbumin- | 228 | FGDTVESQCGSSLSVHSSLKDIFAQITQPKDNYSLNFASRLYAEETYPILPEYLQCVKELYKGG |
| like [Nestor | | LETISFQTAADQARELINSWVESQTNGMIKNILQPSSVDPQTEMVLVNAIYFKGVWEKAFKDE |
| notabilis] | | ETQAVPFRITEQENRPVQIMYQFGSFKVAVVASEKIKILELPYASGQLSMLVLLPDEVSGLEQL |
| | | ENAITFEKLTEWTSSDIMEEKKIKVFLPRMKIEEKYNLTSVLVALGIADLFSSSANLSGISSAESL |
| | | KMSEAVHEAFVEIYEAGSEVVGSSGAGIEAASDSEEFRADHPFLFLIKHKPTNSILFFGRCFSP |
| PREDICTED: | SEQ ID NO: | MGSIGAASTEFCFDIFNELKVQHVNENIFYSPLSIISALSMVYLGARENTKAQIDKVVHFDKITG |
| Ovalbumin- | 229 | FGESIESQCSTSASVHTSFKDMFTQITKPSDNYSLSFASRLYAEETYPILPEYSQCVKELYKGGL |
| like | | ESISFQTAADQARELINSWVESQTNGMIKNILQPGSVDPQTELVLVNAIYFKGTWEKAFKDKD |
| [Pygoscelis | | TQAVPFRVTEQESKPVQMMYQIGSYKVAVIASEKMKILELPYASGELSMLVLLPDDVSGLEQL |
| adeliae] | | ETAITFEKLMEWTSSNMMEERKVKVYLPRMKIEEKYNLTSVLMALGMTDLFSPSANLSGISSA |
| | | ESLKMSEAIHEAFVEIYEAGSEVVGSTEAGMEVTSVSEEFRADHPFLFLIKCNLTNSILFFGRCF |
| | | SP |
| Ovalbumin- | SEQ ID NO: | MGSISTASTEFCFDVFKELKVQHVNENIFYSPLSIISALSMVYLGARENTRAQIEKVVHFDKITG |
| like [Athene | 230 | FGESIESQCGTSVSVHTSLKDMLIQISKPSDNYSLSFASKLYAEETYPILPEYLQCVKELYKGGL |
| cunicularia] | | ESINFQTAADQARQLINSWVESQTNGMIKDILQPSSVDPQTEMVLVNAIYFKGIWEKAFKDED |
| | | TQEVPFRITEQESKPVQMMYQIGSFKVAVIASEKIKILELPYASGELSMLIVLPDDVSGLEQLET |
| | | AITFEKLIEWTSPSIMEERKTKVYLPRMKIEEKYNLTSVLMALGMTDLFSPSANLSGISSAESLK |
| | | MSEAIHEAFVEIYEAGSEVVGSAEAGMEATSVSEFRVDHPFLFLIKHNPANIILFFGRCVSP |
| PREDICTED: | SEQ ID NO: | MGSIGAASTEFCFDVFKELKVQHVNENIFYSPLTIISALSLVYLGARENTRAQIDKVFHFDKISG |
| Ovalbumin- | 231 | FGETTESQCGTSVSVHTSLKEMFTQITKPSDNYSVSFASRLYAEDTYPILPEYLQCVKELYKGG |
| like [Calidris | | LETISFQTAADQAREVINSWVESQTNGMIKNILQPGSVDSQTEMVLVNAIYFKGMWEKAFKD |
| pugnax] | | EDTQTMPFRITEQERKPVQMMYQAGSFKVAVMASEKMKILELPYASGEFCMLIMLPDDVSGL |
| | | EQLENSFSFEKLMEWTTSNMMEERKMKVYIPRMKMEEKYNLTSVLMALGMTDLFSSSANLS |
| | | GISSAETLKMSEAVHEAFMEIYEAGSEVVGSTGSGAEVTGVYEEFRADHPFLFLVKHKPTNSIL |
| | | FFGRCVSP |
| PREDICTED: | SEQ ID NO: | MGSIGAASTEFCFDIFNELKVQHVNENIFYSPLSIISALSMVYLGARENTKAQIDKVVHFDKITG |
| Ovalbumin | 232 | FGETIESQCSTSVSVHTSLKDTFTQITKPSDNYSLSFASRLYAEETYPILPEYSQCVKELYKGGL |
| [Aptenodytes | | ETISFQTAADQARELINSWVESQTNGMIKNILQPGSVDPQTELVLVNAIYFKGTWEKAFKDKD |
| forsteri] | | TQAVPFRVTEQESKPVQMMYQIGSYKVAVIASEKMKILELPYASRELSMLVLLPDDVSGLEQL |
| | | ETAITFEKLMEWTSSNMMEERKVKVYLPRMKIEEKYNLTSVLMALGMTDLFSPSANLSGISSA |
| | | ESLKMSEAVHEAFVEIYEAGSEVVGSTGAGMEVTSVSEEFRADHPFLFLIKCNPTNSILFFGRC |
| | | FSP |
| PREDICTED: | SEQ ID NO: | MGSISAASAEFCLDVFKELKVQHVNENIFYSPLSIISALSMVYLGARENTRAQIDKVVHFDKIT |
| Ovalbumin- | 233 | GSGETIEFQCGTSANIHPSLKDMFTQITRLSDNYSLSFASRLYAEERYPILPEYLQCVKELYKGG |
| like [Pterocles | | LETISFQTAADQARELINSWVESQTNGMIKNILQPGSVNPQTEMVLVNAIYFKGLWEKAFKDE |
| gutturalis] | | DTQTVPFRMTEQESKPVQMMYQVGSFKVAVMASDKIKILELPYASGELSMLVLLPDDVTGLE |
| | | QLETSITFEKLMEWTSSNVMEERTMKVYLPHMRMEEKYNLTSVLMALGVTDLFSSSANLSGI |
| | | SSAESLKMSEAVHEAFVEIYESGSQVVGSTGAGTEVTSVSEEFRVDHPFLFLIKHNPTNSILFFG |
| | | RCFSP |
| Ovalbumin- | SEQ ID NO: | MGSIGAASVEFCFDVFKELKVQHVNENIFYSPLSIISALSMVYLGARENTKAQIDKVVHFDKIA |
| like [Falco | 234 | GFGEAIESQCVTSASIHSLKDMFTQITKPSDNYSLSFASRLYAEEAYSILPEYLQCVKELYKGGL |
| peregrinus] | | ETISFQTAADQARDLINSWVESQTNGMIKNILQPGAVDLETEMVLVNAIYFKGMWEKAFKDE |
| | | DTQTVPFRMTEQESKPVQMMYQVGSFKVAVMASDKIKILELPYASGQLSMVVVLPDDVSGL |
| | | EQLEASITSEKLMEWTSSSIMEEKKIKVYFPHMKIEEKYNLTSVLMALGMTDLFSSSANLSGIS |
| | | SAEKLKVSEAVHEAFVEISEAGSEVVGSTEAGTEVTSVSEEFKADHPFLFLIKHNPTNSILFFGR |
| | | CFSP |
| PREDICTED: | SEQ ID NO: | MGSIGAASSEFCFDIFKELKVQHVNENIFYSPLSIISALSMVYLGARENTRAQIDKVVPFDKITA |
| Ovalbumin - | 235 | SGESIESQCSTSVSVHTSLKDIFTQITKSSDNHSLSFASRLYAEETYPILPEYLQCVKELYEGGLE |
| like isoform | | TISFQTAADQARELINSWIESQTNGRIKNILQPGSVDPQTEMVLVNAIYFKGMWEKAFKDEDT |
| X2 | | QAVPFRMTEQESKPVQVMHQIGSFKVAVLASEKIKILELPYASGELSMLVLLPDDVSGLEQLE |
| [Phalacrocorax | | TAITFEKLMEWTSPNIMEERKIKVFLPRMKIEEKYNLTSVLMALGITDLFSPLANLSGISSAESL |
| carbo] | | KMSEAIHEAFVEISEAGSEVIGSTEAEVEVINDPEEFRADHPFLFLIKHNPTNSILFFGRCFSP |
| PREDICTED: | SEQ ID NO: | MGSIGAASTEFCFDVFKELKAQYVNENIFYSPMTIITALSMVYLGSKENTRAQIAKVAHFDKIT |
| Ovalbumin- | 236 | GFGESIESQCGASASIQFSLKDLFTQITKPSGNHSLSVASRIYAEETYPILPEYLECMKELYKGGL |
| like [Merops | | ETINFQTAANQARELINSWVERQTSGMIKNILQPSSVDSQTEMVLVNAIYFRGLWEKAFKVED |
| nubicus] | | TQATPFRITEQESKPVQMMHQIGSFKVAVVASEKIKILELPYASGRLTMLVVLPDDVSGLKQL |
| | | ETTITFEKLMEWTTSNIMEERKIKVYLPRMKIEEKYNLTSVLMALGLTDLFSSSANLSGISSAES |
| | | LKMSEAVHEAFVEIYEAGSEVVASAEAGMDATSVSEEFRADHPFLFLIKDNTSNSILFFGRCFS |
| | | P |
| PREDICTED: | SEQ ID NO: | MGSIGAASTEFCFDVFKELKGQHVNENIFFCPLSIVSALSMVYLGARENTRAQIVKVAHFDKIA |
| Ovalbumin- | 237 | GFAESIESQCGTSVSIHTSLKDMFTQITKPSDNYSLNFASRLYAEETYPIIPEYLQCVKELYKGG |
| like [Tauraco | | LETISFQTAADQAREIINSWVESQTNGMIKNILRPSSVHPQTELVLVNAVYFKGTWEKAFKDE |
| erythrolophus] | | DTQAVPFRITEQESKPVQMMYQIGSFKVAAVTSEKMKILEVPYASGELSMLVLLPDDVSGLEQ |
| | | LETAITAEKLIEWTSSTVMEERKLKVYLPRMKIEEKYNLTTVLTALGVTDLFSSSANLSGISSA |
| | | QGLKMSNAVHEAFVEIYEAGSEVVGSKGEGTEVSSVSDEFKADHPFLFLIKHNPTNSIVFFGRC |
| | | FSP |
| PREDICTED: | SEQ ID NO: | MGSIGAASTEFCFDVFKELKVHHVNENILYSPLAIISALSMVYLGAKENTRDQIDKVVHFDKIT |
| Ovalbumin - | 238 | GIGESIESQCSTAVSVHTSLKDVFDQITRPSDNYSLAFASRLYAEKTYPILPEYLQCVKELYKGG |
| like [Cuculus | | LETIDFQTAADQARQLINSWVEDETNGMIKNILRPSSVNPQTKIILVNAIYFKGMWEKAFKDED |
| canorus] | | TQEVPFRITEQETKSVQMMYQIGSFKVAEVVSDKMKILELPYASGKLSMLVLLPDDVYGLEQL |
| | | ETVITVEKLKEWTSSIVMEERITKVYLPRMKIMEKYNLTSVLTAFGITDLFSPSANLSGISSTESL |
| | | KVSEAVHEAFVEIHEAGSEVVGSAGAGIEATSVSEEFKADHPFLFLIKHNPTNSILFFGRCFSP |
| Ovalbumin | SEQ ID NO: | MGSIGAASTEFCLDVFKELKVQHVNENIFYSPLSIISALSMVYLGARENTRAQIDKVVHFDKIT |
| [Antrostomus | 239 | GFEDSIESQCGTSVSVHTSLKDMFTQITKPSDNYSVGFASRLYAAETYQILPEYSQCVKELYKG |
| carolinensis] | | GLETINFQKAADQATELINSWVESQTNGMIKNILQPSSVDPQTQIFLVNAIYFKGMWQRAFKE |
| | | EDTQAVPFRISEKESKPVQMMYQIGSFKVAVIPSEKIKILELPYASGLLSMLVILPDDVSGLEQL |
| | | ENAITLEKLMQWTSSNMMEERKIKVYLPRMRMEEKYNLTSVFMALGITDLFSSSANLSGISSA |
| | | ESLKMSDAVHEASVEIHEAGSEVVGSTGSGTEASSVSEEFRADHPYLFLIKHNPTDSIVFFGRCF |
| | | SP |
| PREDICTED: | SEQ ID NO: | MGSIGAASTEFCFDVFKELKFQHVDENIFYSPLTIISALSMVYLGARENTRAQIDKVVHFDKIA |
| Ovalbumin- | 240 | GFEETVESQCGTSVSVHTSLKDMFAQITKPSDNYSLSFASRLYAEETYPILPEYLQCVKELYKG |
| like | | GLETISFQTAADQARDLINSWVESQTNGMIKNILQPSSVGPQTELILVNAIYFKGMWQKAFKD |
| [Opisthocomus | | EDTQEVPFRMTEQQSKPVQMMYQTGSFKVAVVASEKMKILALPYASGQLSLLVMLPDDVSG |
| hoazin] | | LKQLESAITSEKLIEWTSPSMMEERKIKVYLPRMKIEEKYNLTSVLMALGITDLFSPSANLSGIS |
| | | SAESLKMSQAVHEAFVEIYEAGSEVVGSTGAGMEDSSDSEEFRVDHPFLFFIKHNPTNSILFFG |
| | | RCFSP |
| PREDICTED: | SEQ ID NO: | MGSIGPLSVEFCCDVFKELRIQHPRENIFYSPVTIISALSMVYLGARDNTKAQIEKAVHFDKIPG |
| Ovalbumin- | 241 | FGESIESQCGTSLSIHTSLKDIFTQITKPSDNYTVGIASRLYAEEKYPILPEYLQCIKELYKGGLEP |
| like | | INFQTAAEQARELINSWVESQTNGMIKNILQPSSVNPETDMVLVNAIYFKGLWEKAFKDEDIQ |
| [Lepidothrix | | TVPFRITEQESKPVQMMFQIGSFRVAEITSEKIRILELPYASGQLSLWVLLPDDISGLEQLETAIT |
| coronata] | | FENLKEWTSSTKMEERKIKVYLPRMKIEEKYNLTSVLTSLGITDLFSSSANLSGISSAESLKVSS |
| | | AFHEASVEIYEAGSKVVGSTGAEVEDTSVSEEFRADHPFLFLIKHNPSNSIFFFGRCFSP |
| PREDICTED: | SEQ ID NO: | MGSIGTASAEFCFDVFKELKVHHVNENIFYSPLSIISALSMVYLGARENTKTQMEKVIHFDKIT |
| Ovalbumin | 242 | GLGESMESQCGTGVSIHTALKDMLSEITKPSDNYSLSLASRLYAEQTYAILPEYLQCIKELYKE |
| [Struthio | | SLETVSFQTAADQARELINSWIESQTNGVIKNFLQPGSVDSQTELVLVNAIYFKGMWEKAFKD |
| camelus | | EDTQEVPFRITEQESRPVQMMYQAGSFKVATVAAEKIKILELPYASGELSMLVLLPDDISGLEQ |
| australis] | | LETTISFEKLTEWTSSNMMEDRNMKVYLPRMKIEEKYNLTSVLIALGMTDLFSPAANLSGISA |
| | | AESLKMSEAIHAAYVEIYEADSEIVSSAGVQVEVTSDSEEFRVDHPFLFLIKHNPTNSVLFFGRC |
| | | ISP |
| PREDICTED: | SEQ ID NO: | MGSIGAVSTEFSCDVFKELRIHHVQENIFYSPVTIISALSMIYLGARDSTKAQIEKAVHFDKIPGF |
| Ovalbumin- | 243 | GESIESQCGTSLSIHTSIKDMFTKITKASDNYSIGIASRLYAEEKYPILPEYLQCVKELYKGGLESI |
| like | | SFQTAAEQAREIINSWVESQTNGMIKNILQPSSVDPQTDIVLVNAIYFKGLWEKAFRDEDTQTV |
| [Acanthisitta | | PFKITEQESKPVQMMYQIGSFKVAEITSEKIKILEVPYASGQLSLWVLLPDDISGLEKLETAITFE |
| chloris] | | NLKEWTSSTKMEERKIKVYLPRMKIEEKYNLTSVLTALGITDLFSSSANLSGISSAESLKVSEAF |
| | | HEAIVEISEAGSKVVGSVGAGVDDTSVSEEFRADHPFLFLIKHNPTSSIFFFGRCFSP |
| PREDICTED: | SEQ ID NO: | MGSIGAASTEFCFDVFKELKVQHVNENIFYSPLSIISALSMVYLGARENTRAQIDKVVHFDKIA |
| Ovalbumin- | 244 | GFGESTESQCGTSVSAHTSLKDMSNQITKLSDNYSLSFASRLYAEETYPILPEYSQCVKELYKG |
| like [Tyto | | GLESISFQTAAYQARELINAWVESQTNGMIKDILQPGSVDSQTKMVLVNAIYFKGIWEKAFKD |
| alba] | | EDTQEVPFRMTEQETKPVQMMYQIGSFKVAVIAAEKIKILELPYASGQLSMLVILPDDVSGLE |
| | | QLETAITFEKLTEWTSASVMEERKIKVYLPRMSIEEKYNLTSVLIALGVTDLFSSSANLSGISSA |
| | | ESLRMSEAIHEAFVETYEAGSTESGTEVTSASEEFRVDHPFLFLIKHKPTNSILFFGRCFSP |
| PREDICTED: | SEQ ID NO: | MGSIGAASSEFCFDIFKELKVQHVNENIFYSPLSIISALSMVYLGARENTRAQIDKVVPFDKITA |
| Ovalbumin - | 245 | SGESIESQVQKIQCSTSVSVHTSLKDIFTQITKSSDNHSLSFASRLYAEETYPILPEYLQCVKELY |
| like isoform | | EGGLETISFQTAADQARELINSWIESQTNGRIKNILQPGSVDPQTEMVLVNAIYFKGMWEKAF |
| X1 | | KDEDTQAVPFRMTEQESKPVQVMHQIGSFKVAVLASEKIKILELPYASGELSMLVLLPDDVSG |
| [Phalacrocorax | | LEQLETAITFEKLMEWTSPNIMEERKIKVFLPRMKIEEKYNLTSVLMALGITDLFSPLANLSGIS |
| carbo] | | SAESLKMSEAIHEAFVEISEAGSEVIGSTEAEVEVTNDPEEFRADHPFLFLIKHNPTNSILFFGRC |
| | | FSP |
| Ovalbumin- | SEQ ID NO: | MGSIGPLSVEFCCDVFKELRIQHARENIFYSPVTIISALSMVYLGARDNTKAQIEKAVHFDKIPG |
| like [Pipra | 246 | FGESIESQCGTSLSIHTSLKDIFTQITKPSDNYTVGIASRLYAEEKYPILPEYLQCIKELYKGGLEP |
| filicauda] | | ISFQTAAEQARELINSWVESQINGIIKNILQPSSVNPETDMVLVNAIYFKGLWEKAFKDEGTQT |
| | | VPFRITEQESKPVQMMFQIGSFRVAEIASEKIRILELPYASGQLSLWVLLPDDISGLEQLETAITF |
| | | ENLKEWTSSTKMEERKIKVYLPRMKIEEKYNLTSVLTSLGITDLFSSSANLSGISSAERLKVSSA |
| | | FHEASMEINEAGSKVVGAGVDDTSVSEEFRVDRPFLFLIKHNPSNSIFFFGRCFSP |
| Ovalbumin | SEQ ID NO: | MGSIGAASTEFCFDMFKELKVHHVNENIIYSPLSIISILSMVFLGARENTKTQMEKVIHFDKITG |
| [Dromaius | 247 | FGESLESQCGTSVSVHASLKDILSEITKPSDNYSLSLASKLYAEETYPVLPEYLQCIKELYKGSL |
| novaehollandiae] | | ETVSFQTAADQARELINSWVETQTNGVIKNFLQPGSVDPQTEMVLVDAIYFKGTWEKAFKDE |
| | | DTQEVPFRITEQESKPVQMMYQAGSFKVATVAAEKMKILELPYASGELSMFVLLPDDISGLEQ |
| | | LETTISIEKLSEWTSSNMMEDRKMKVYLPHMKIEEKYNLTSVLVALGMTDLFSPSANLSGIST |
| | | AQTLKMSEAIHGAYVEIYEAGSEMATSTGVLVEAASVSEEFRVDHPFLFLIKHNPSNSILFFGR |
| | | CIFP |
| Chain A, | SEQ ID NO: | MGSIGAASTEFCFDMFKELKVHHVNENIIYSPLSIISILSMVFLGARENTKTQMEKVIHFDKITG |
| Ovalbumin | 248 | FGESLESQCGTSVSVHASLKDILSEITKPSDNYSLSLASKLYAEETYPVLPEYLQCIKELYKGSL |
| | | ETVSFQTAADQARELINSWVETQTNGVIKNFLQPGSVDPQTEMVLVDAIYFKGTWEKAFKDE |
| | | DTQEVPFRITEQESKPVQMMYQAGSFKVATVAAEKMKILELPYASGELSMFVLLPDDISGLEQ |
| | | LETTISIEKLSEWTSSNMMEDRKMKVYLPHMKIEEKYNLTSVLVALGMTDLFSPSANLSGIST |
| | | AQTLKMSEAIHGAYVEIYEAGSEMATSTGVLVEAASVSEEFRVDHPFLFLIKHNPSNSILFFGR |
| | | CIFPHHHHHH |
| Ovalbumin- | SEQ ID NO: | MGSIGPLSVEFCCDVFKELRIQHARENIFYSPVTIISALSMVYLGARDNTKAQIEKAVHFDKIPG |
| like [Corapipo | 249 | FGESIESQCGTSLSIHTSLKDIFTQITKPSDNYTVGIASRLYAEEKYPILPEYLQCIKELYKGGLEP |
| altera] | | ISFQTAAEQARELINSWVESQTNGMIKNILQPSAVNPETDMVLVNAIYFKGLWEKAFKDEGTQ |
| | | TVPFRITEQESKPVQMMFQIGSFRVAEITSEKIRILELPYASGQLSLWVLLPDDISGLEQLETAIT |
| | | FENLKEWTSSTKMEERKIKVYLPRMKIEEKYNLTSVLTSLGITDLFSSSANLSGISSAERLKVSS |
| | | AFHEASMEIYEAGSKVVGSTGAGVDDTSVSEEFRVDRPFLFLIKHNPSNSIFFFGRCFSP |
| Ovalbumin- | SEQ ID NO: | MEDQRGNTGFTMGSIGAASTEFCIDVFRELRVQHVNENIFYSPLTIISALSMVYLGARENTRAQ |
| like protein | 250 | IDQVVHFDKIAGFGDTVESQCGSSPSVHNSLKTVXAQITQPRDNYSLNLASRLYAEESYPILPE |
| [Amazona | | YLQCVKELYNGGLETVSFQTAADQARELINSWVESQINGIIKNILQPSSVDPQTEMVLVNAIYF |
| aestiva] | | KGLWEKAFKDEETQAVPFRITEQENRPVQMMYQFGSFKVAXVASEKIKILELPYASGQLSML |
| | | VLLPDEVSGLEQNAITFEKLTEWTSSDLMEERKIKVFFPRVKIEEKYNLTAVLVSLGITDLFSSS |
| | | ANLSGISSAENLKMSEAVHEAXVEIYEAGSEVAGSSGAGIEVASDSEEFRVDHPFLFLIXHNPT |
| | | NSILFFGRCFSP |
| PREDICTED: | SEQ ID NO: | MGSIGAASTEFCIDVFRELRVQHVNENIFYSPLSIISALSMVYLGARENTRAQIDEVFHFDKIAG |
| Ovalbumin- | 251 | FGDTVDPQCGASLSVHKSLQNVFAQITQPKDNYSLNLASRLYAEESYPILPEYLQCVKELYNE |
| like | | GLETVSFQTGADQARELINSWVENQTNGVIKNILQPSSVDPQTEMVLVNAIYFKGLWQKAFK |
| [Melopsittacus | | DEETQAVPFRITEQENRPVQMMYQFGSFKVAVVASEKVKILELPYASGQLSMWVLLPDEVSG |
| undulatus] | | LEQLENAITFEKLTEWTSSDLTEERKIKVFLPRVKIEEKYNLTAVLMALGVTDLFSSSANFSGIS |
| | | AAENLKMSEAVHEAFVEIYEAGSEVVGSSGAGIEAPSDSEEFRADHPFLFLIKHNPTNSILFFGR |
| | | CFSP |
| Ovalbumin- | SEQ ID NO: | MGSIGPLSVEFCCDVFKELRIQHARDNIFYSPVTIISALSMVYLGARDNTKAQIEKAVHFDKIPG |
| like | 252 | FGESIESQCGTSLSVHTSLKDIFTQITKPRENYTVGIASRLYAEEKYPILPEYLQCIKELYKGGLE |
| [Neopelma | | PISFQTAAEQARELINSWVESQTNGMIKNILQPSSVNPETDMVLVNAIYFKGLWKKAFKDEGT |
| chrysocephalum] | | QTVPFRITEQESKPVQMMFQIGSFRVAEITSEKIRILELPYASGQLSLWVLLPDDISGLEQLESAI |
| | | TFENLKEWTSSTKMEERKIKVYLPRMKIEEKYNLTSVLTSLGITDLFSSSANLSGISSAEKLKVS |
| | | SAFHEASMEIYEAGNKVVGSTGAGVDDTSVSEEFRVDRPFLFLIKHNPSNSIFFFGRCFSP |
| PREDICTED: | SEQ ID NO: | MGSIGAASAEFCVDVFKELKDQHVNNIVFSPLMIISALSMVNIGAREDTRAQIDKVVHFDKITG |
| Ovalbumin- | 253 | YGESIESQCGTSIGIYFSLKDAFTQITKPSDNYSLSFASKLYAEETYPILPEYLKCVKELYKGGLE |
| like [Buceros | | TISFQTAADQARELINSWVESQTNGMIKNILQPSSVDPQTEMVLVNAIYFKGLWEKAFKDEDT |
| rhinoceros | | QAVPFRITEQESKPVQMMYQIGSFKVAVIASEKIKILELPYASGQLSLLVLLPDDVSGLEQLESA |
| silvestris] | | ITSEKLLEWTNPNIMEERKTKVYLPRMKIEEKYNLTSVLVALGITDLFSSSANLSGISSAEGLKL |
| | | SDAVHEAFVEIYEAGREVVGSSEAGVEDSSVSEEFKADRPFIFLIKHNPTNGILYFGRYISP |
| PREDICTED: | SEQ ID NO: | MGSIGAANTDFCFDVFKELKVHHANENIFYSPLSIVSALAMVYLGARENTRAQIDKALHFDKI |
| Ovalbumin- | 254 | LGFGETVESQCDTSVSVHTSLKDMLIQITKPSDNYSFSFASKIYTEETYPILPEYLQCVKELYKG |
| like [Cariama | | GVETISFQTAADQAREVINSWVESHTNGMIKNILQPGSVDPQTKMVLVNAVYFKGIWEKAFK |
| cristata] | | EEDTQEMPFRINEQESKPVQMMYQIGSFKLTVAASENLKILEFPYASGQLSMMVILPDEVSGL |
| | | KQLETSITSEKLIKWTSSNTMEERKIRVYLPRMKIEEKYNLKSVLMALGITDLFSSSANLSGISS |
| | | AESLKMSEAVHEAFVEIYEAGSEVTSSTGTEMEAENVSEEFKADHPFLFLIKHNPTDSIVFFGR |
| | | CMSP |
| Ovalbumin | SEQ ID NO: | MGSIGPLSVEFCCDVFKELRIQHARENIFYSPVTIISALSMVYLGARDNTKAQIEKAVHFDKIPG |
| [Manacus | 255 | FGESIESQCGTSLSIHTSLKDIFTQITKPSDNYTVGIASRLYAEEKYPILPEYLQCIKELYKGGLEP |
| vitellinus] | | ISFQTAAEQARELINSWVESQTNGMIKNILQPSSVNPETDMVLVNAIYFKGLWEKAFKDESTQ |
| | | TVPFRITEQESKPVQMMFQIGSFRVAEIASEKIRILELPYASGQLSLWVLLPDDISGLEQLETAIT |
| | | FENLKEWTSSTKMEERKIKVYLPRMKIEEKYNLTSVLTSLGITDLFSSSANLSGISSAERLKVSS |
| | | AFHEASMEIYEAGSRVVEAGVDDTSVSEEFRVDRPFLFLIKHNPSNSIFFFGRCFSP |
| Ovalbumin- | SEQ ID NO: | MGSIGPVSTEFCCDIFKELRIQHARENIIYSPVTIISALSMVYLGARDNTKAQIEKAVHFDKIPGF |
| like | 256 | GESIESQCGTSLSIHTSLKDILTQITKPSDNYTVGIASRLYAEEKYPILSEYLQCIKELYKGGLEPI |
| [Empidonax | | SFQTAAEQARELINSWVESQTNGMIKNILQPSSVNPETDMVLVNAIYFKGLWEKAFKDEGTQT |
| traillii] | | VPFRITEQESKPVQMMFQIGSFKVAEITSEKIRILELPYASGKLSLWVLLPDDISGLEQLETAITF |
| | | ENLKEWTSSTRMEERKIKVYLPRMKIEEKYNLTSVLTSLGITDLFSSSANLSGISSAERLKVSSA |
| | | FHEVFVEIYEAGSKVEGSTGAGVDDTSVSEEFRADHPFLFLVKHNPSNSIIFFGRCYLP |
| PREDICTED: | SEQ ID NO: | MGSTGAASMEFCFALFRELKVQHVNENIFFSPVTIISALSMVYLGARENTRAQLDKVAPFDKIT |
| Ovalbumin- | 257 | GFGETIGSQCSTSASSHTSLKDVFTQITKASDNYSLSFASRLYAEETYPILPEYLQCVKELYKGG |
| like | | LESISFQTAADQARELINSWVESQTNGMIKDILRPSSVDPQTKIILITAIYFKGMWEKAFKEEDT |
| [Leptosomus | | QAVPFRMTEQESKPVQMMYQIGSFKVAVIPSEKLKILELPYASGQLSMLVILPDDVSGLEQLET |
| discolor] | | AITTEKLKEWTSPSMMKERKMKVYFPRMRIEEKYNLTSVLMALGITDLFSPSANLSGISSAESL |
| | | KVSEAVHEASVDIDEAGSEVIGSTGVGTEVTSVSEEIRADHPFLFLIKHKPTNSILFFGRCFSP |
| Hypothetical | SEQ ID NO: | MEHAQLTQLVNSNMTSNTCHEADEFENIDFRMDSISVTNTKFCFDVFNEMKVHHVNENILYS |
| protein | 258 | PLSILTALAMVYLGARGNTESQMKKALHFDSITGAGSTTDSQCGSSEYIHNLFKEFLTEITRTN |
| H355_008077 | | ATYSLEIADKLYVDKTFTVLPEYINCARKFYTGGVEEVNFKTAAEEARQLINSWVEKETNGQI |
| [Colinus | | KDLLVPSSVDFGTMMVFINTIYFKGIWKTAFNTEDTREMPFSMTKQESKPVQMMCLNDTFNM |
| virginianus] | | ATLPAEKMRILELPYASGELSMLVLLPDEVSGLEQIEKAINFEKLREWTSTNAMEKKSMKVYL |
| | | PRMKIEEKYNLTSTLMALGMTDLFSRSANLTGISSVENLMISDAVHGAFMEVNEEGTEAAGST |
| | | GAIGNIKHSVEFEEFRADHPFLFLIRYNPTNVILFFDNSEFTMGSIGAVSTEFCFDVFKELRVHH |
| | | ANENIFYSPFTVISALAMVYLGAKDSTRTQINKVVRFDKLPGFGDSIEAQCGTSANVHSSLRDI |
| | | LNQITKPNDIYSFSLASRLYADETYTILPEYLQCVKELYRGGLESINFQTAADQARELINSWVES |
| | | QTSGIIRNVLQPSSVDSQTAMVLVNAIYFKGLWEKGFKDEDTQAMPFRVTEQENKSVQMMY |
| | | QIGTFKVASVASEKMKILELPFASGTMSMWVLLPDEVSGLEQLETTISIEKLTEWTSSSVMEER |
| | | KIKVFLPRMKMEEKYNLTSVLMAMGMTDLFSSSANLSGISSTLQKKGFRSQELGDKYAKPML |
| | | ESPALTPQVTAWDNSWIVAHPAAIEPDLCYQIMEQKWKPFDWPDFRLPMRVSCRFRTMEALN |
| | | KANTSFALDFFKHECQEDDDENILFSPFSISSALATVYLGAKGNTADQMAKTEIGKSGNIHAGF |
| | | KALDLEINQPTKNYLLNSVNQLYGEKSLPFSKEYLQLAKKYYSAEPQSVDFLGKANEIRREINS |
| | | RVEHQTEGKIKNLLPPGSIDSLTRLVLVNALYFKGNWATKFEAEDTRHRPFRINMHTTKQVPM |
| | | MYLRDKFNWTYVESVQTDVLELPYVNNDLSMFILLPRDITGLQKLINELTFEKLSAWTSPELM |
| | | EKMKMEVYLPRFTVEKKYDMKSTLSKMGIEDAFTKVDSCGVTNVDEITTHIVSSKCLELKHIQ |
| | | INKKLKCNKAVAMEQVSASIGNFTIDLFNKLNETSRDKNIFFSPWSVSSALALTSLAAKGNTAR |
| | | EMAEDPENEQAENIHSGFKELMTALNKPRNTYSLKSANRIYVEKNYPLLPTYIQLSKKYYKAE |
| | | PYKVNFKTAPEQSRKEINNWVEKQTERKIKNFLSSDDVKNSTKSILVNAIYFKAEWEEKFQAG |
| | | NTDMQPFRMSKNKSKLVKMMYMRHTFPVLIMEKLNFKMIELPYVKRELSMFILLPDDIKDST |
| | | TGLEQLERELTYEKLSEWADSKKMSVTLVDLHLPKFSMEDRYDLKDALKSMGMASAFNSNA |
| | | DFSGMTGFQAVPMESLSASTNSFTLDLYKKLDETSKGQNIFFASWSIATALAMVHLGAKGDT |
| | | ATQVAKGPEYEETENIHSGFKELLSAINKPRNTYLMKSANRLFGDKTYPLLPKFLELVARYYQ |
| | | AKPQAVNFKTDAEQARAQINSWVENETESKIQNLLPAGSIDSHTVLVLVNAIYFKGNWEKRFL |
| | | EKDTSKMPFRLSKTETKPVQMMFLKDTFLIHHERTMKFKIIELPYVGNELSAFVLLPDDISDNT |
| | | TGLELVERELTYEKLAEWSNSASMMKAKVELYLPKLKMEENYDLKSVLSDMGIRSAFDPAQ |
| | | ADFTRMSEKKDLFISKVIHKAFVEVNEEDRIVQLASGRLTGRCRTLANKELSEKNRTKNLFFSP |
| | | FSISSALSMILLGSKGNTEAQIAKVLSLSKAEDAHNGYQSLLSEINNPDTKYILRTANRLYGEKT |
| | | FEFLSSFIDSSQKFYHAGLEQTDFKNASEDSRKQINGWVEEKTEGKIQKLLSEGIINSMTKLVLV |
| | | NAIYFKGNWQEKFDKETTKEMPFKINKNETKPVQMMFRKGKYNMTYIGDLETTVLEIPYVDN |
| | | ELSMIILLPDSIQDESTGLEKLERELTYEKLMDWINPNMMDSTEVRVSLPRFKLEENYELKPTL |
| | | STMGMPDAFDLRTADFSGISSGNELVLSEVVHKSFVEVNEEGTEAAAATAGIMLLRCAMIVA |
| | | NFTADHPFLFFIRHNKTNSILFCGRFCSP |
| PREDICTED: | SEQ ID NO: | MGSIGTASTEFCFDMFKEMKVQHANQNIIFSPLTIISALSMVYLGARDNTKAQMEKVIHFDKIT |
| Ovalbumin | 259 | GFGESVESQCGTSVSIHTSLKDMLSEITKPSDNYSLSLASRLYAEETYPILPEYLQCMKELYKG |
| isoform X2 | | GLETVSFQTAADQARELINSWVESQTNGVIKNFLQPGSVDPQTEMVLVNAIYFKGMWEKAFK |
| [Apteryx | | DEDTQEVPFRITEQESKPVQMMYQVGSFKVATVAAEKMKILEIPYTHRELSMFVLLPDDISGL |
| australis | | EQLETTISFEKLTEWTSSNMMEERKVKVYLPHMKIEEKYNLTSVLMALGMTDLFSPSANLSGI |
| mantelli] | | STAQTLMMSEAIHGAYVEIYEAGREMASSTGVQVEVTSVLEEVRADKPFLFFIRHNPTNSMVV |
| | | FGRYMSP |
| Hypothetical | SEQ ID NO: | MTSNTCHEADEFENIDFRMDSISVTNTKFCFDVFNEMKVHHVNENILYSPLSILTALAMVYLG |
| protein | 260 | ARGNTESQMKKALHFDSITGGGSTTDSQCGSSEYIHNLFKEFLTEITRTNATYSLEIADKLYVD |
| ASZ78_006007 | | KTFTVLPEYINCARKFYTGGVEEVNFKTAAEEARQLMNSWVEKETNGQIKDLLVPSSVDFGT |
| [Callipepla | | MMVFINTIYFKGIWKTAFNTEDTREMPFSMTKQESKPVQMMCLNDTFNMVTLPAEKMRILEL |
| squamata] | | PYASGELSMLVLLPDEVSGLERIEKAINFEKLREWTSTNAMEKKSMKVYLPRMKIEEKYNLTS |
| | | TLMALGMTDLFSRSANLTGISSVDNLMISDAVHGAFMEVNEEGTEAAGSTGAIGNIKHSVEFE |
| | | EFRADHPFLFLIRYNPTNVILFFDNSEFTMGSIGAVSTEFCFDVFKELRVHHANENIFYSPFTIISA |
| | | LAMVYLGAKDSTRTQINKVVRFDKLPGFGDSIEAQCGTSANVHSSLRDILNQITKPNDIYSFSL |
| | | ASRLYADETYTILPEYLQCVKELYRGGLESINFQTAADQARELINSWVESQTSGIIRNVLQPSSV |
| | | DSQTAMVLVNAIYFKGLWEKGFKDEDTQAIPFRVTEQENKSVQMMYQIGTFKVASVASEKM |
| | | KILELPFASGTMSMWVLLPDEVSGLEQLETTISIEKLTEWTSSSVMEERKIKVFLPRMKMEEKY |
| | | NLTSVLMAMGMTDLFSSSANLSGISSTLQKKGFRSQELGDKYAKPMLESPALTPQATAWDNS |
| | | WIVAHPPAIEPDLYYQIMEQKWKPFDWPDFRLPMRVSCRFRTMEALNKANTSFALDFFKHEC |
| | | QEDDSENILFSPFSISSALATVYLGAKGNTADQMAKVLHFNEAEGARNVTTTIRMQVYSRTDQ |
| | | QRLNRRACFQKTEIGKSGNIHAGFKGLNLEINQPTKNYLLNSVNQLYGEKSLPFSKEYLQLAK |
| | | KYYSAEPQSVDFVGTANEIRREINSRVEHQTEGKIKNLLPPGSIDSLTRLVLVNALYFKGNWAT |
| | | KFEAEDTRHRPFRINTHTTKQVPMMYLSDKFNWTYVESVQTDVLELPYVNNDLSMFILLPRDI |
| | | TGLQKLINELTFEKLSAWTSPELMEKMKMEVYLPRFTVEKKYDMKSTLSKMGIEDAFTKVDN |
| | | CGVTNVDEITIHVVPSKCLELKHIQINKELKCNKAVAMEQVSASIGNFTIDLFNKLNETSRDKN |
| | | IFFSPWSVSSALALTSLAAKGNTAREMAEDPENEQAENIHSGFNELLTALNKPRNTYSLKSAN |
| | | RIYVEKNYPLLPTYIQLSKKYYKAEPHKVNFKTAPEQSRKEINNWVEKQTERKIKNFLSSDDV |
| | | KNSTKLILVNAIYFKAEWEEKFQAGNTDMQPFRMSKNKSKLVKMMYMRHTFPVLIMEKLNF |
| | | KMIELPYVKRELSMFILLPDDIKDSTTGLEQLERELTYEKLSEWADSKKMSVTLVDLHLPKFS |
| | | MEDRYDLKDALRSMGMASAFNSNADFSGMTGERDLVISKVCHQSFVAVDEKGTEAAAATA |
| | | VIAEAVPMESLSASTNSFTLDLYKKLDETSKGQNIFFASWSIATALTMVHLGAKGDTATQVAK |
| | | GPEYEETENIHSGFKELLSALNKPRNTYSMKSANRLFGDKTYPLLPTKTKPVQMMFLKDTFLI |
| | | HHERTMKFKIIELPYMGNELSAFVLLPDDISDNTTGLELVERELTYEKLAEWSNSASMMKVKV |
| | | ELYLPKLKMEENYDLKSALSDMGIRSAFDPAQADFTRMSEKKDLFISKVIHKAFVEVNEEDRI |
| | | VQLASGRLTGNTEAQIAKVLSLSKAEDAHNGYQSLLSEINNPDTKYILRTANRLYGEKTFEFLS |
| | | SFIDSSQKFYHAGLEQTDFKNASEDSRKQINGWVEEKTEGKIQKLLSEGIINSMTKLVLVNAIY |
| | | FKGNWQEKFDKETTKEMPFKINKNETKPVQMMFRKGKYNMTYIGDLETTVLEIPYVDNELS |
| | | MIILLPDSIQDESTGLEKLERELTYEKLMDWINPNMMDSTEVRVSLPRFKLEENYELKPTLSTM |
| | | GMPDAFDLRTADFSGISSGNELVLSEVVHKSFVEVNEEGTEAAAATAGIMLLRCAMIVANFTA |
| | | DHPFLFFIRHNKTNSILFCGRFCSP |
| PREDICTED: | SEQ ID NO: | MASIGAASTEFCFDVFKELKTQHVKENIFYSPMAIISALSMVYIGARENTRAEIDKVVHFDKIT |
| Ovalbumin- | 261 | GFGNAVESQCGPSVSVHSSLKDLITQISKRSDNYSLSYASRIYAEETYPILPEYLQCVKEVYKG |
| like | | GLESISFQTAADQARENINAWVESQTNGMIKNILQPSSVNPQTEMVLVNAIYLKGMWEKAFK |
| [Mesitornis | | DEDTQTMPFRVTQQESKPVQMMYQIGSFKVAVIASEKMKILELPYTSGQLSMLVLLPDDVSG |
| unicolor] | | LEQVESAITAEKLMEWTSPSIMEERTMKVYLPRMKMVEKYNLTSVLMALGMTDLFTSVANL |
| | | SGISSAQGLKMSQAIHEAFVEIYEAGSEAVGSTGVGMEITSVSEEFKADLSFLFLIRHNPTNSIIF |
| | | FGRCISP |
| Ovalbumin, | SEQ ID NO: | MGSIGAASTEFCFDVFRELRVQHVNENIFYSPFSIISALAMVYLGARDNTRTQIDKISQFQALSD |
| partial [Anas | 262 | EHLVLCIQQLGEFFVCTNRERREVTRYSEQTEDKTQDQNTGQIHKIVDTCMLRQDILTQITKPS |
| platyrhynchos] | | DNFSLSFASRLYAEETYAILPEYLQCVKELYKGGLESISFQTAADQARELINSWVESQINGIIKN |
| | | ILQPSSVDSQTTMVLVNAIYFKGMWEKAFKDEDTQAMPFRMTEQESKPVQMMYQVGSFKVA |
| | | MVTSEKMKILELPFASGMMSMFVLLPDEVSGLEQLESTISFEKLTEWTSSTMMEERRMKVYLP |
| | | RMKMEEKYNLTSVFMALGMTDLFSSSANMSGISSTVSLKMSEAVHAACVEIFEAGRDVVGSA |
| | | EAGMDVTSVSEEFRADHPFLFFIKHNPTNSILFFGRWMSP |
| PREDICTED: | SEQ ID NO: | MGSIGAASAEFCLDIFKELKVQHVNENIIFSPMTIISALSLVYLGAKEDTRAQIEKVVPFDKIPGF |
| Ovalbumin- | 263 | GEIVESQCPKSASVHSSIQDIFNQIIKRSDNYSLSLASRLYAEESYPIRPEYLQCVKELDKEGLETI |
| like [Chaetura | | SFQTAADQARQLINSWVESQTNGMIKNILQPSSVNSQTEMVLVNAIYFRGLWQKAFKDEDTQ |
| pelagica] | | AVPFRITEQESKPVQMMQQIGSFKVAEIASEKMKILELPYASGQLSMLVLLPDDVSGLEKLESS |
| | | ITVEKLIEWTSSNLTEERNVKVYLPRLKIEEKYNLTSVLAALGITDLFSSSANLSGISTAESLKLS |
| | | RAVHESFVEIQEAGHEVEGPKEAGIEVTSALDEFRVDRPFLFVTKHNPTNSILFLGRCLSP |
| PREDICTED: | SEQ ID NO: | MGSISAASGEFCLDIFKELKVQHVNENIFYSPMVIVSALSLVYLGARENTRAQIDKVIPFDKITG |
| Ovalbumin- | 264 | SSEAVESQCGTPVGAHISLKDVFAQIAKRSDNYSLSFVNRLYAEETYPILPEYLQCVKELYKGG |
| like | | LETISFQTAADQAREIINSWVESQTDGKIKNILQPSSVDPQTKMVLVSAIYFKGLWEKSFKDED |
| [Apaloderma | | TQAVPFRVTEQESKPVQMMYQIGSFKVAALAAEKIKILELPYASEQLSMLVLLPDDVSGLEQLE |
| vittatum] | | KKISYEKLTEWTSSSVMEEKKIKVYLPRMKIEEKYNLTSILMSLGITDLFSSSANLSGISSTKSLK |
| | | MSEAVHEASVEIYEAGSEASGITGDGMEATSVFGEFKVDHPFLFMIKHKPTNSILFFGRCISP |
| Ovalbumin- | SEQ ID NO: | MGSIGPVSTEVCCDIFRELRSQSVQENVCYSPLLIISTLSMVYIGAKDNTKAQIEKAIHFDKIPGF |
| like [Corvus | 265 | GESTESQCGTSVSIHTSLKDIFTQITKPSDNYSISIARRLYAEEKYPILPEYIQCVKELYKGGLESI |
| cornix cornix] | | SFQTAAEKSRELINSWVESQTNGTIKNILQPSSVSSQTDMVLVSAIYFKGLWEKAFKEEDTQTI |
| | | PFRITEQESKPVQMMSQIGTFKVAEIPSEKCRILELPYASGRLSLWVLLPDDISGLEQLETAITFE |
| | | NLKEWTSSSKMEERKIRVYLPRMKIEEKYNLTSVLKSLGITDLFSSSANLSGISSAESLKVSAAF |
| | | HEASVEIYEAGSKGVGSSEAGVDGTSVSEEIRADHPFLFLIKHNPSDSILFFGRCFSP |
| PREDICTED: | SEQ ID NO: | MGSIGAASTEFCFDVFKELKVQHVNENIIISPLSIISALSMVYLGAREDTRAQIDKVVHFDKITG |
| Ovalbumin- | 266 | FGEAIESQCPTSESVHASLKETFSQLTKPSDNYSLAFASRLYAEETYPILPEYLQCVKELYKGGL |
| like [Calypte | | ETINFQTAAEQARQVINSWVESQTDGMIKSLLQPSSVDPQTEMILVNAIYFRGLWERAFKDED |
| anna] | | TQELPFRITEQESKPVQMMSQIGSFKVAVVASEKVKILELPYASGQLSMLVLLPDDVSGLEQLE |
| | | SSITVEKLIEWISSNTKEERNIKVYLPRMKIEEKYNLTSVLVALGITDLFSSSANLSGISSAESLKI |
| | | SEAVHEAFVEIQEAGSEVVGSPGPEVEVTSVSEEWKADRPFLFLIKHNPTNSILFFGRYISP |
| PREDICTED: | SEQ ID NO: | MGSIGPVSTEVCCDIFRELRSQSVQENVCYSPLLIISTLSMVYIGAKDNTKAQIEKAIHFDKIPGF |
| Ovalbumin | 267 | GESTESQCGTSVSIHTSLKDIFTQITKPSDNYSISIARRLYAEEKYPILQEYIQCVKELYKGGLESI |
| [Corvus | | SFQTAAEKSRELINSWVESQTNGTIKNILQPSSVSSQTDMVLVSAIYFKGLWEKAFKEEDTQTI |
| brachyrhynchos] | | PFRITEQESKPVQMMSQIGTFKVAEIPSEKCRILELPYASGRLSLWVLLPDDISGLEQLETSITFE |
| | | NLKEWTSSSKMEERKIRVYLPRMKIEEKYNLTSVLKSLGITDLFSSSANLSGISSAESLKVSAVF |
| | | HEASVEIYEAGSKGVGSSEAGVDGTSVSEEIRADHPFLFLIKHNPSDSILFFGRCFSP |
| Hypothetical | SEQ ID NO: | MLNLMHPKQFCCTMGSIGPVSTEVCCDIFRELRSQSVQENVCYSPLLIISTLSMVYIGAKDNTK |
| protein | 268 | AQIEKAIHFDKIPGFGESTESQCGTSVSIHTSLKDIFTQITKPSDNYSISIASRLYAEEKYPILPEYI |
| DUI87_08270 | | QCVKELYKGGLESISFQTAAEKSRELINSWVESQTNGTIKNILQPSSVSSQTDMVLVSAIYFKG |
| [Hirundo | | LWEKAFKEEDTQTVPFRITEQESKPVQMMSQIGTFKVAEIPSEKCRILELPYASGRLSLWVLLP |
| rustica rustica] | | DDISGLEQLETAITSENLKEWTSSSKMEERKIKVYLPRMKIEEKYNLTSVLKSLGITDLFSSSAN |
| | | LSGISSAESLKVSGAFHEAFVEIYEAGSKAVGSSGAGVEDTSVSEEIRADHPFLFFIKHNPSDSIL |
| | | FFGRCFSP |
| Ostrich OVA | SEQ ID NO: | EAEAGSIGTASAEFCFDVFKELKVHHVNENIFYSPLSIISALSMVYLGARENTKTQMEKVIHFD |
| sequence as | 269 | KITGLGESMESQCGTGVSIHTALKDMLSEITKPSDNYSLSLASRLYAEQTYAILPEYLQCIKELY |
| secreted from | | KESLETVSFQTAADQARELINSWIESQTNGVIKNFLQPGSVDSQTELVLVNAIYFKGMWEKAF |
| pichia | | KDEDTQEVPFRITEQESRPVQMMYQAGSFKVATVAAEKIKILELPYASGELSMLVLLPDDISGL |
| | | EQLETTISFEKLTEWTSSNMMEDRNMKVYLPRMKIEEKYNLTSVLIALGMTDLFSPAANLSGI |
| | | SAAESLKMSEAIHAAYVEIYEADSEIVSSAGVQVEVTSDSEEFRVDHPFLFLIKHNPTNSVLFFG |
| | | RCISP |
| Ostrich | SEQ ID NO: | MRFPSIFTAVLFAASSALAAPVNTTTEDETAQIPAEAVIGYSDLEGDFDVAVLPFSNSTNNGLL |
| construct | 270 | FINTTIASIAAKEEGVSLEKREAEAGSIGTASAEFCFDVFKELKVHHVNENIFYSPLSIISALSMV |
| (secretion | | YLGARENTKTQMEKVIHFDKITGLGESMESQCGTGVSIHTALKDMLSEITKPSDNYSLSLASRL |
| signal + | | YAEQTYAILPEYLQCIKELYKESLETVSFQTAADQARELINSWIESQTNGVIKNFLQPGSVDSQ |
| mature | | TELVLVNAIYFKGMWEKAFKDEDTQEVPFRITEQESRPVQMMYQAGSFKVATVAAEKIKILE |
| protein) | | LPYASGELSMLVLLPDDISGLEQLETTISFEKLTEWTSSNMMEDRNMKVYLPRMKIEEKYNLT |
| | | SVLIALGMTDLFSPAANLSGISAAESLKMSEAIHAAYVEIYEADSEIVSSAGVQVEVTSDSEEFR |
| | | VDHPFLFLIKHNPTNSVLFFGRCISP |
| Duck OVA | SEQ ID NO: | EAEAGSIGAASTEFCFDVFRELRVQHVNENIFYSPFSIISALAMVYLGARDNTRTQIDKVVHFD |
| sequence as | 271 | KLPGFGESMEAQCGTSVSVHSSLRDILTQITKPSDNFSLSFASRLYAEETYAILPEYLQCVKELY |
| secreted from | | KGGLESISFQTAADQARELINSWVESQINGIIKNILQPSSVDSQTTMVLVNAIYFKGMWEKAF |
| pichia | | KDEDTQAMPFRMTEQESKPVQMMYQVGSFKVAMVTSEKMKILELPFASGMMSMFVLLPDE |
| | | VSGLEQLESTISFEKLTEWTSSTMMEERRMKVYLPRMKMEEKYNLTSVFMALGMTDLFSSSA |
| | | NMSGISSTVSLKMSEAVHAACVEIFEAGRDVVGSAEAGMDVTSVSEEFRADHPFLFFIKHNPT |
| | | NSILFFGRWMSP |
| Duck | SEQ ID NO: | MRFPSIFTAVLFAASSALAAPVNTTTEDETAQIPAEAVIGYSDLEGDFDVAVLPFSNSTNNGLL |
| construct | 272 | FINTTIASIAAKEEGVSLEKREAEAGSIGAASTEFCFDVFRELRVQHVNENIFYSPFSIISALAMV |
| (secretion | | YLGARDNTRTQIDKVVHFDKLPGFGESMEAQCGTSVSVHSSLRDILTQITKPSDNFSLSFASRL |
| signal + | | YAEETYAILPEYLQCVKELYKGGLESISFQTAADQARELINSWVESQINGIIKNILQPSSVDSQT |
| mature | | TMVLVNAIYFKGMWEKAFKDEDTQAMPFRMTEQESKPVQMMYQVGSFKVAMVTSEKMKIL |
| protein) | | ELPFASGMMSMFVLLPDEVSGLEQLESTISFEKLTEWTSSTMMEERRMKVYLPRMKMEEKYN |
| | | LTSVFMALGMTDLFSSSANMSGISSTVSLKMSEAVHAACVEIFEAGRDVVGSAEAGMDVTSV |
| | | SEEFRADHPFLFFIKHNPTNSILFFGRWMSP |
| Ovoglobulin | SEQ ID NO: | TRAPDCGGILTPLGLSYLAEVSKPHAEVVLRQDLMAQRASDLFLGSMEPSRNRITSVKVADL |
| G2 | 273 | WLSVIPEAGLRLGIEVELRIAPLHAVPMPVRISIRADLHVDMGPDGNLQLLTSACRPTVQAQST |
| | | REAESKSSRSILDKVVDVDKLCLDVSKLLLFPNEQLMSLTALFPVTPNCQLQYLPLAAPVFSKQ |
| | | GIALSLQTTFQVAGAVVPVPVSPVPFSMPELASTSTSHLILALSEHFYTSLYFTLERAGAFNMTI |
| | | PSMLTTATLAQKITQVGSLYHEDLPITLSAALRSSPRVVLEEGRAALKLFLTVHIGAGSPDFQSF |
| | | LSVSADVTAGLQLSVSDTRMMISTAVIEDAELSLAASNVGLVRAALLEELFLAPVCQQVPAW |
| | | MDDVLREGVHLPHLSHFTYTDVNVVVHKDYVLVPCKLKLRSTMA* |
| Ovoglobulin | SEQ ID NO: | MDSISVTNAKFCFDVFNEMKVHHVNENILYCPLSILTALAMVYLGARGNTESQMKKVLHFDS |
| G3 | 274 | ITGAGSTTDSQCGSSEYVHNLFKELLSEITRPNATYSLEIADKLYVDKTFSVLPEYLSCARKFYT |
| | | GGVEEVNFKTAAEEARQLINSWVEKETNGQIKDLLVSSSIDFGTTMVFINTIYFKGIWKIAFNT |
| | | EDTREMPFSMTKEESKPVQMMCMNNSFNVATLPAEKMKILELPYASGDLSMLVLLPDEVSGL |
| | | ERIEKTINFDKLREWTSTNAMAKKSMKVYLPRMKIEEKYNLTSILMALGMTDLFSRSANLTGI |
| | | SSVDNLMISDAVHGVFMEVNEEGTEATGSTGAIGNIKHSLELEEFRADHPFLFFIRYNPTNAILF |
| | | FGRYWSP* |
| β-ovomucin | SEQ ID NO: | CSTWGGGHFSTFDKYQYDFTGTCNYIFATVCDESSPDFNIQFRRGLDKKIARIIIELGPSVIIVEK |
| | 275 | DSISVRSVGVIKLPYASNGIQIAPYGRSVRLVAKLMEMELVVMWNNEDYLMVLTEKKYMGK |
| | | TCGMCGNYDGYELNDFVSEGKLLDTYKFAALQKMDDPSEICLSEEISIPAIPHKKYAVICSQLL |
| | | NLVSPTCSVPKDGFVTRCQLDMQDCSEPGQKNCTCSTLSEYSRQCAMSHQVVFNWRTENFCS |
| | | VGKCSANQIYEECGSPCIKTCSNPEYSCSSHCTYGCFCPEGTVLDDISKNRTCVHLEQCPCTLN |
| | | GETYAPGDTMKAACRTCKCTMGQWNCKELPCPGRCSLEGGSFVTTFDSRSYRFHGVCTYILM |
| | | KSSSLPHNGTLMAIYEKSGYSHSETSLSAIIYLSTKDKIVISQNELLTDDDELKRLPYKSGDITIF |
| | | KQSSMFIQMHTEFGLELVVQTSPVFQAYVKVSAQFQGRTLGLCGNYNGDTTDDFMTSMDITE |
| | | GTASLFVDSWRAGNCLPAMERETDPCALSQLNKISAETHCSILTKKGTVFETCHAVVNPTPFY |
| | | KRCVYQACNYEETFPYICSALGSYARTCSSMGLILENWRNSMDNCTITCTGNQTFSYNTQACE |
| | | RTCLSLSNPTLECHPTDIPIEGCNCPKGMYLNHKNECVRKSHCPCYLEDRKYILPDQSTMTGGI |
| | | TCYCVNGRLSCTGKLQNPAESCKAPKKYISCSDSLENKYGATCAPTCQMLATGIECIPTKCES |
| | | GCVCADGLYENLDGRCVPPEECPCEYGGLSYGKGEQIQTECEICTCRKGKWKCVQKSRCSST |
| | | CNLYGEGHITTFDGQRFVFDGNCEYILAMDGCNVNRPLSSFKIVTENVICGKSGVTCSRSISIYL |
| | | GNLTIILRDETYSISGKNLQVKYNVKKNALHLMFDIIIPGKYNMTLIWNKHMNFFIKISRETQET |
| | | ICGLCGNYNGNMKDDFETRSKYVASNELEFVNSWKENPLCGDVYFVVDPCSKNPYRKAWAE |
| | | KTCSIINSQVFSACHNKVNRMPYYEACVRDSCGCDIGGDCECMCDAIAVYAMACLDKGICID |
| | | WRTPEFCPVYCEYYNSHRKTGSGGAYSYGSSVNCTWHYRPCNCPNQYYKYVNIEGCYNCSH |
| | | DEYFDYEKEKCMPCAMQPTSVTLPTATQPTSPSTSSASTVLTETTNPPV* |
| Lysozyme | SEQ ID NO: | KVFGRCELAAAMKRHGLDNYRGYSLGNWVCAAKFESNFNTQATNRNTDGSTDYGILQINSR |
| | 276 | WWCNDGRTPGSRNLCNIPCSALLSSDITASVNCAKKIVSDGNGMNAWVAWRNRCKGTDVQ |
| | | AWIRGCRL* |
| Lysozyme | SEQ ID NO: | KVFGRCELAAAMKRHGLDNYRGYSLGNWVCVAKFESNFNTQATNRNTDGSTDYGILQINSR |
| | 277 | WWCNDGRTPGSRNLCNIPCSALLSSDITASVNCAKKIVSDGNGMSAWVAWRNRCKGTDVQA |
| | | WIRGCRL* |
| Lysozyme C | SEQ ID NO: | KVFERCELARTLKRLGMDGYRGISLANWMCLAKWESGYNTRATNYNAGDRSTDYGIFQINS |
| (Human) | 278 | RYWCNDGKTPGAVNACHLSCSALLQDNIADAVACAKRVVRDPQGIRAWVAWRNRCQNRD |
| | | VRQYVQGCGV* |
| Lysozyme C | SEQ ID NO: | KVFERCELARTLKKLGLDGYKGVSLANWLCLTKWESSYNTKATNYNPSSESTDYGIFQINSK |
| (Bos taurus) | 279 | WWCNDGKTPNAVDGCHVSCRELMENDIAKAVACAKHIVSEQGITAWVAWKSHCRDHDVSS |
| | | YVEGCTL* |
| Ovoinhibitor | SEQ ID NO: | IEVNCSLYASGIGKDGTSWVACPRNLKPVCGTDGSTYSNECGICLYNREHGANVEKEYDGEC |
| | 280 | RPKHVMIDCSPYLQVVRDGNTMVACPRILKPVCGSDSFTYDNECGICAYNAEHHTNISKLHD |
| | | GECKLEIGSVDCSKYPSTVSKDGRTLVACPRILSPVCGTDGFTYDNECGICAHNAEQRTHVSK |
| | | KHDGKCRQEIPEIDCDQYPTRKTTGGKLLVRCPRILLPVCGTDGFTYDNECGICAHNAQHGTE |
| | | VKKSHDGRCKERSTPLDCTQYLSNTQNGEAITACPFILQEVCGTDGVTYSNDCSLCAHNIELG |
| | | TSVAKKHDGRCREEVPELDCSKYKTSTLKDGRQVVACTMIYDPVCATNGVTYASECTLCAH |
| | | NLEQRTNLGKRKNGRCEEDITKEHCREFQKVSPICTMEYVPHCGSDGVTYSNRCFFCNAYVQ |
| | | SNRTLNLVSMAAC* |
| Cystatin | SEQ ID NO: | MAGARGCVVLLAAALMLVGAVLGSEDRSRLLGAPVPVDENDEGLQRALQFAMAEYNRASN |
| | 281 | DKYSSRVVRVISAKRQLVSGIKYILQVEIGRTTCPKSSGDLQSCEFHDEPEMAKYTTCTFVVYS |
| | | IPWLNQIKLLESKCQ* |
| Porcine | SEQ ID NO: | SEVCFPRLGCFSDDAPWAGIVQRPLKILPWSPKDVDTRFLLYTNQNQNNYQELVADPSTITNS |
| Lipase | 282 | NFRMDRKTRFIIHGFIDKGEEDWLSNICKNLFKVESVNCICVDWKGGSRTGYTQASQNIRIVG |
| | | AEVAYFVEVLKSSLGYSPSNVHVIGHSLGSHAAGEAGRRTNGTIERITGLDPAEPCFQGTPELV |
| | | RLDPSDAKFVDVIHTDAAPIIPNLGFGMSQTVGHLDFFPNGGKQMPGCQKNILSQIVDIDGIWE |
| | | GTRDFVACNHLRSYKYYADSILNPDGFAGFPCDSYNVFTANKCFPCPSEGCPQMGHYADRFP |
| | | GKTNGVSQVFYLNTGDASNFARWRYKVSVTLSGKKVTGHILVSLFGNEGNSRQYEIYKGTLQ |
| | | PDNTHSDEFDSDVEVGDLQKVKFIWYNNNVINPTLPRVGASKITVERNDGKVYDFCSQETVR |
| | | EEVLLTLNPC* |
| Kid Lipase | SEQ ID NO: | GLVAADRITGGKDFRDIESKFALRTPEDTAEDTCHLIPGVTESVANCHFNHSSKTFVVIHGWTV |
| | 283 | TGMYESWVPKLVAALYKREPDSNVIVVDWLSRAQQHYPVSAGYTKLVGQDVAKFMNWMA |
| | | DEFNYPLGNVHLLGYSLGAHAAGIAGSLTSKKVNRITGLDPAGPNFEYAEAPSRLSPDDADFV |
| | | DVLHTFTRGSPGRSIGIQKPVGHVDIYPNGGTFQPGCNIGEALRVIAERGLGDVDQLVKCSHER |
| | | SVHLFIDSLLNEENPSKAYRCNSKEAFEKGLCLSCRKNRCNNMGYEINKVRAKRSSKMYLKT |
| | | RSQMPYKVFHYQVKIHFSGTESNTYTNQAFEISLYGTVAESENIPFTLPEVSTNKTYSFLLYTEV |
| | | DIGELLMLKLKWISDSYFSWSNWWSSPGFDIGKIRVKAGETQKKVIFCSREKMSYLQKGKSPV |
| | | IFVKCHDKSLNRKSG* |
| Porcine | SEQ ID NO: | APKKGVRWCVISTAEYSKCRQWQSKIRRTNPMFCIRRASPTDCIRAIAAKRADAVTLDGGLVF |
| Lactoferrin | 284 | EADQYKLRPVAAEIYGTEENPQTYYYAVAVVKKGFNFQLNQLQGRKSCHTGLGRSAGWNIPI |
| | | GLLRRFLDWAGPPEPLQKAVAKFFSQSCVPCADGNAYPNLCQLCIGKGKDKCACSSQEPYFG |
| | | YSGAFNCLHKGIGDVAFVKESTVFENLPQKADRDKYELLCPDNTRKPVEAFRECHLARVPSH |
| | | AVVARSVNGKENSIWELLYQSQKKFGKSNPQEFQLFGSPGQQKDLLFRDATIGFLKIPSKIDSK |
| | | LYLGLPYLTAIQGLRETAAEVEARQAKVVWCAVGPEELRKCRQWSSQSSQNLNCSLASTTED |
| | | CIVQVLKGEADAMSLDGGFIYTAGKCGLVPVLAENQKSRQSSSSDCVHRPTQGYFAVAVVRK |
| | | ANGGITWNSVRGTKSCHTAVDRTAGWNIPMGLLVNQTGSCKFDEFFSQSCAPGSQPGSNLCA |
| | | LCVGNDQGVDKCVPNSNERYYGYTGAFRCLAENAGDVAFVKDVTVLDNINGQNTEEWARE |
| | | LRSDDFELLCLDGTRKPVTEAQNCHLAVAPSHAVVSRKEKAAQVEQVLLTEQAQFGRYGKD |
| | | CPDKFCLFRSETKNLLFNDNTEVLAQLQGKTTYEKYLGSEYVTAIANLKQCSVSPLLEACAFM |
| | | MR* |
| Bovine | SEQ ID NO: | APRKNVRWCTISQPEWFKCRRWQWRMKKLGAPSITCVRRAFALECIRAIAEKKADAVTLDG |
| Lactoferrin | 285 | GMVFEAGRDPYKLRPVAAEIYGTKESPQTHYYAVAVVKKGSNFQLDQLQGRKSCHTGLGRS |
| | | AGWIIPMGILRPYLSWTESLEPLQGAVAKFFSASCVPCIDRQAYPNLCQLCKGEGENQCACSSR |
| | | EPYFGYSGAFKCLQDGAGDVAFVKETTVFENLPEKADRDQYELLCLNNSRAPVDAFKECHLA |
| | | QVPSHAVVARSVDGKEDLIWKLLSKAQEKFGKNKSRSFQLFGSPPGQRDLLFKDSALGFLRIP |
| | | SKVDSALYLGSRYLTTLKNLRETAEEVKARYTRVVWCAVGPEEQKKCQQWSQQSGQNVTC |
| | | ATASTTDDCIVLVLKGEADALNLDGGYIYTAGKCGLVPVLAENRKSSKHSSLDCVLRPTEGYL |
| | | AVAVVKKANEGLTWNSLKDKKSCHTAVDRTAGWNIPMGLIVNQTGSCAFDEFFSQSCAPGA |
| | | DPKSRLCALCAGDDQGLDKCVPNSKEKYYGYTGAFRCLAEDVGDVAFVKNDTVWENTNGE |
| | | STADWAKNLNREDFRLLCLDGTRKPVTEAQSCHLAVAPNHAVVSRSDRAAHVKQVLLHQQA |
| | | LFGKNGKNCPDKFCLFKSETKNLLFNDNTECLAKLGGRPTYEEYLGTEYVTALANLKKCSTSP |
| | | LLEACAFLTR* |
| Lysozyme | SEQ ID NO: | KVFGRCELAAAMKRHGLDNYRGYSLGNWVCAAKFESNFNTQATNRNTDGSTDYGILQINSR |
| | 276 | WWCNDGRTPGSRNLCNIPCSALLSSDITASVNCAKKIVSDGNGMNAWVAWRNRCKGTDVQ |
| | | AWIRGCRL* |
| Lysozyme | SEQ ID NO: | KVFGRCELAAAMKRHGLDNYRGYSLGNWVCVAKFESNFNTQATNRNTDGSTDYGILQINSR |
| | 277 | WWCNDGRTPGSRNLCNIPCSALLSSDITASVNCAKKIVSDGNGMSAWVAWRNRCKGTDVQA |
| | | WIRGCRL* |
| Lysozyme C | SEQ ID NO: | KVFERCELARTLKRLGMDGYRGISLANWMCLAKWESGYNTRATNYNAGDRSTDYGIFQINS |
| (Human) | 278 | RYWCNDGKTPGAVNACHLSCSALLQDNIADAVACAKRVVRDPQGIRAWVAWRNRCQNRD |
| | | VRQYVQGCGV* |
| Lysozyme C | SEQ ID NO: | KVFERCELARTLKKLGLDGYKGVSLANWLCLTKWESSYNTKATNYNPSSESTDYGIFQINSK |
| (Bos taurus) | 279 | WWCNDGKTPNAVDGCHVSCRELMENDIAKAVACAKHIVSEQGITAWVAWKSHCRDHDVSS |
| | | YVEGCTL* |
| Ovoinhibitor | SEQ ID NO: | IEVNCSLYASGIGKDGTSWVACPRNLKPVCGTDGSTYSNECGICLYNREHGANVEKEYDGEC |
| | 280 | RPKHVMIDCSPYLQVVRDGNTMVACPRILKPVCGSDSFTYDNECGICAYNAEHHTNISKLHD |
| | | GECKLEIGSVDCSKYPSTVSKDGRTLVACPRILSPVCGTDGFTYDNECGICAHNAEQRTHVSK |
| | | KHDGKCRQEIPEIDCDQYPTRKTTGGKLLVRCPRILLPVCGTDGFTYDNECGICAHNAQHGTE |
| | | VKKSHDGRCKERSTPLDCTQYLSNTQNGEAITACPFILQEVCGTDGVTYSNDCSLCAHNIELG |
| | | TSVAKKHDGRCREEVPELDCSKYKTSTLKDGRQVVACTMIYDPVCATNGVTYASECTLCAH |
| | | NLEQRTNLGKRKNGRCEEDITKEHCREFQKVSPICTMEYVPHCGSDGVTYSNRCFFCNAYVQ |
| | | SNRTLNLVSMAAC* |
| Cystatin | SEQ ID NO: | MAGARGCVVLLAAALMLVGAVLGSEDRSRLLGAPVPVDENDEGLQRALQFAMAEYNRASN |
| | 281 | DKYSSRVVRVISAKRQLVSGIKYILQVEIGRTTCPKSSGDLQSCEFHDEPEMAKYTTCTFVVYS |
| | | IPWLNQIKLLESKCQ* |
| Porcine | SEQ ID NO: | SEVCFPRLGCFSDDAPWAGIVQRPLKILPWSPKDVDTRFLLYTNQNQNNYQELVADPSTITNS |
| Lipase | 282 | NFRMDRKTRFIIHGFIDKGEEDWLSNICKNLFKVESVNCICVDWKGGSRTGYTQASQNIRIVG |
| | | AEVAYFVEVLKSSLGYSPSNVHVIGHSLGSHAAGEAGRRTNGTIERITGLDPAEPCFQGTPELV |
| | | RLDPSDAKFVDVIHTDAAPIIPNLGFGMSQTVGHLDFFPNGGKQMPGCQKNILSQIVDIDGIWE |
| | | GTRDFVACNHLRSYKYYADSILNPDGFAGFPCDSYNVFTANKCFPCPSEGCPQMGHYADRFP |
| | | GKTNGVSQVFYLNTGDASNFARWRYKVSVTLSGKKVTGHILVSLFGNEGNSRQYEIYKGTLQ |
| | | PDNTHSDEFDSDVEVGDLQKVKFIWYNNNVINPTLPRVGASKITVERNDGKVYDFCSQETVR |
| | | EEVLLTLNPC* |
| Kid Lipase | SEQ ID NO: | GLVAADRITGGKDFRDIESKFALRTPEDTAEDTCHLIPGVTESVANCHFNHSSKTFVVIHGWTV |
| | 283 | TGMYESWVPKLVAALYKREPDSNVIVVDWLSRAQQHYPVSAGYTKLVGQDVAKFMNWMA |
| | | DEFNYPLGNVHLLGYSLGAHAAGIAGSLTSKKVNRITGLDPAGPNFEYAEAPSRLSPDDADFV |
| | | DVLHTFTRGSPGRSIGIQKPVGHVDIYPNGGTFQPGCNIGEALRVIAERGLGDVDQLVKCSHER |
| | | SVHLFIDSLLNEENPSKAYRCNSKEAFEKGLCLSCRKNRCNNMGYEINKVRAKRSSKMYLKT |
| | | RSQMPYKVFHYQVKIHFSGTESNTYTNQAFEISLYGTVAESENIPFTLPEVSTNKTYSFLLYTEV |
| | | DIGELLMLKLKWISDSYFSWSNWWSSPGFDIGKIRVKAGETQKKVIFCSREKMSYLQKGKSPV |
| | | IFVKCHDKSLNRKSG* |
| Porcine | SEQ ID NO: | APKKGVRWCVISTAEYSKCRQWQSKIRRTNPMFCIRRASPTDCIRAIAAKRADAVTLDGGLVF |
| Lactoferrin | 284 | EADQYKLRPVAAEIYGTEENPQTYYYAVAVVKKGFNFQLNQLQGRKSCHTGLGRSAGWNIPI |
| | | GLLRRFLDWAGPPEPLQKAVAKFFSQSCVPCADGNAYPNLCQLCIGKGKDKCACSSQEPYFG |
| | | YSGAFNCLHKGIGDVAFVKESTVFENLPQKADRDKYELLCPDNTRKPVEAFRECHLARVPSH |
| | | AVVARSVNGKENSIWELLYQSQKKFGKSNPQEFQLFGSPGQQKDLLFRDATIGFLKIPSKIDSK |
| | | LYLGLPYLTAIQGLRETAAEVEARQAKVVWCAVGPEELRKCRQWSSQSSQNLNCSLASTTED |
| | | CIVQVLKGEADAMSLDGGFIYTAGKCGLVPVLAENQKSRQSSSSDCVHRPTQGYFAVAVVRK |
| | | ANGGITWNSVRGTKSCHTAVDRTAGWNIPMGLLVNQTGSCKFDEFFSQSCAPGSQPGSNLCA |
| | | LCVGNDQGVDKCVPNSNERYYGYTGAFRCLAENAGDVAFVKDVTVLDNINGQNTEEWARE |
| | | LRSDDFELLCLDGTRKPVTEAQNCHLAVAPSHAVVSRKEKAAQVEQVLLTEQAQFGRYGKD |
| | | CPDKFCLFRSETKNLLFNDNTEVLAQLQGKTTYEKYLGSEYVTAIANLKQCSVSPLLEACAFM |
| | | MR* |
| Bovine | SEQ ID NO: | APRKNVRWCTISQPEWFKCRRWQWRMKKLGAPSITCVRRAFALECIRAIAEKKADAVTLDG |
| Lactoferrin | 285 | GMVFEAGRDPYKLRPVAAEIYGTKESPQTHYYAVAVVKKGSNFQLDQLQGRKSCHTGLGRS |
| | | AGWIIPMGILRPYLSWTESLEPLQGAVAKFFSASCVPCIDRQAYPNLCQLCKGEGENQCACSSR |
| | | EPYFGYSGAFKCLQDGAGDVAFVKETTVFENLPEKADRDQYELLCLNNSRAPVDAFKECHLA |
| | | QVPSHAVVARSVDGKEDLIWKLLSKAQEKFGKNKSRSFQLFGSPPGQRDLLFKDSALGFLRIP |
| | | SKVDSALYLGSRYLTTLKNLRETAEEVKARYTRVVWCAVGPEEQKKCQQWSQQSGQNVTC |
| | | ATASTTDDCIVLVLKGEADALNLDGGYIYTAGKCGLVPVLAENRKSSKHSSLDCVLRPTEGYL |
| | | AVAVVKKANEGLTWNSLKDKKSCHTAVDRTAGWNIPMGLIVNQTGSCAFDEFFSQSCAPGA |
| | | DPKSRLCALCAGDDQGLDKCVPNSKEKYYGYTGAFRCLAEDVGDVAFVKNDTVWENTNGE |
| | | STADWAKNLNREDFRLLCLDGTRKPVTEAQSCHLAVAPNHAVVSRSDRAAHVKQVLLHQQA |
| | | LFGKNGKNCPDKFCLFKSETKNLLFNDNTECLAKLGGRPTYEEYLGTEYVTALANLKKCSTSP |
| | | LLEACAFLTR* |

TABLE 7
Miscellaneous

| | SEQ ID | |
|---|---|---|
| Sequence Info | NO: | Amino acid sequence |
| CCW12 homolog | SEQ ID NO: | MFEKSKFVVSFLLLLQLFCVLGVHGQESGNGTTSDTAYACDIGATPFDGFNATIYQYQAS |
| GQ68_01574 | 286 | DDNSIQDPVFMSTGYLQRNQLHSTTGVTNPGFNIFTAGVATTTLYGIPNVNYQNMLLELK |
| (chr1) | | GYFRADASGNYGLSLRNIDDSAILFFGRETAFECCNENLIPLDEAPTDYSLFTIKEGEASTN |
| | | PDSYTYTQYLEAGRYYPVRTFFANIRTRAVFNFTMTLPDGSELTDFQNYIFQFGALNQQQ |
| | | CQAEIVTRENYTTTTEPWTGTFEATTTVIPSGTEPGTVIVQTPYSTIDSTSTWTGTFTTFTTD |
| | | ADGSTIAVVPSSTIDDHFASTETVLTDTAISTTVITVTSCGTSKCTKTTALTGVTQRTLTIDD |
| | | RTTVVTTYCPLPTDVATIKTASVSGSEVVQTIYTAKHSQAVSYVHPSTVTITREVCDAQTC |
| | | TQATIVTGEILQTTVVDSGSTTVVPKYVPVETHEPTFELSTL |
| CCW14 homolog | SEQ ID NO: | MQFTFASTSVVVSLIAALAKPAVATPPACLLACAAEVVKESSDCDALNNIQCICENEGSAI |
| GQ68_01658 | 287 | HACLESTCPDGLSSTALQSFEDVCESVGTEANLDESSSSQSSSSSSSSESSSSSVSSSSSSASS |
| (PAS_chr1-4_ | | SSETSSSVTSSSVTSSSTAVSSSTESSSSVEPSTSHSSSHSSSEVSSTVAPTTSVAPTTSSITT |
| 0510) | | SSTSLTSATTSSVTISIEPTSDAADKVIIPGLAGLVGALAVGLI |
| CCW22 homologs | SEQ ID NO: | MQYRSLFLGSALLAAANAAVYNTTVTDVVSELETTVLTITSCAEDKCITSKSTGLITTSTL |
| GQ68_02511 | 288 | TKHGVVTVVTTVCDLPSTTKSYVPPAKTTTIPPPEKTTTTVPPPAKTTTTVPPPAKTTSTVP |
| (chr1) | | PPAKTSSHHESTITVTVPSSTSTKKIETESTTYHFVTQTTTARNITPPAITTQSHGAAGMNA |
| | | ANFVGLGAAAVAAAALVL |
| CCW22 homolog | SEQ ID NO: | MSLLLFLVLGAFLLSSVKAADIGAFRLRVYTPGRFTNGALNFNNWGYQYLDASSSNGQL |
| GQ68_03003 | 289 | FAGYATVTSVTTFLAPDDEGFVWGSSLGGYPGFLGIGAGATAFHLTGIPGDALSWYIEDN |
| (chr3) | | ILKTSSPTYVCSRNDGDVVVGIEANTRWLAMHDTSQLPPNYYCFQADYEIVALWYIPDTT |
| | | STWTGTETSTTTDDDGSVIELVPTPLPDTTSTWTGTFTTFTTDDDGSVIELVPTPLPDSTST |
| | | WTGTYTTFTTDEDGSTIAVVPSSTIDSTSTWTGTYTTFTTDEDGSTIAVVPSSTIDSTSTWT |
| | | GTYTTFTTDEDGSTIAVYHHLLSTPHPPGLVLTPRSLPMRMEVLLLWYHHLLSTLHPPGL |
| | | VLTPRSLPMRMEVLLLWYHRLLSTPHPGLVLTPRSLPMRMEVLLLYHHLLSTPHPPGLVL |
| | | TPRSLPMRMEVLLLWY |
| FLO5 homolog | SEQ ID NO: | MKLQLQSFVFFLLSAVNVLADDSYGCSIATSPRSTGFVANLYEFPNMAISNAELKTYVRY |
| GQ68_04296 | 290 | RYKEGRLYDTISNIISPYFYYQGQGANSAYGTLYGRPNVYLYNFSMELKGYFRPPITGQY |
| (chr4) | | TIDFNGANVDDAAMVFFGKAGAFDCCNSDYILPEQSAEYSLYSVYPHTATDQILSATIYL |
| | | EAGKYYPLRVTYTNIGNIGSLDLRVVLPSGASITSLGAFVYQFPNNLSPGTCTPDVEYFTT |
| | | TTQAWTGTYETTYTVPPSGTQPGTVIIETPESYVTTTQPWTGTYETTYTVPPTGTEPGTVII |
| | | ETPESYVTTTQPWTGTYETTYTVPPSGTEPGTVIIETPESYVTTTQPWTGTYETTYTVPPSG |
| | | TEPGTVIIETPESYVTTTQPWTGTYETTYTVPPSGTEPGIVIIETPESYVTTTQPWTGTYETT |
| | | YTVPPSGTEPGTVVIETPEITDCEAVCCGAVPTSDPLRRRDVCDCETFCCPGDTNCETYVT |
| | | TTQPWTGTYETTYTVPPSGTEPGTVIIETPESYVTTTQPWTGTYETTYTVPPSGTEPGIVIIE |
| | | TPESYVTTTQPWTGTYETTYTVPPTGTEPGTVIIETPESYVTTTQPWTGTYETTYTVPPSGT |
| | | EPGIVIIETPESYVTTTQPWTGTYETTYTVPPSGTEPGTVIIETPESYVTTTQPWTGTYETTY |
| | | TVPPTGTEPGTVIIETPESYVTTTQPWTGTYETTYTVPPSGTEPGIVIIETPESYVTTTQPWT |
| | | GTYETTYTVPPTGTEPGTVIIETPESYVTTTQPWTGTYETTYTVPPTGTEPGTVIIETPESYV |
| | | TTTQPWTGTYETTYTVPPSGTEPGTVIIETPESYVTTTQPWTGTYETTYTVPPSGTEPGTV |
| | | VIETPEITDCEAVCCGAVPTSDPLRRRDVCDCETFCCPGDTNCETYVTTTQPWTGTYETT |
| | | YTVPPSGTEPGTVIIETPESYVTTTQPWTGTYETTYTVPPTGTEPGTVIIETPESYVTTTQPW |
| | | TGTYETTYTVPPSGTQPGTVIIETPESYVTTTQPWTGTYETTYTVPPTGTEPGTVIIETPESY |
| | | VTTTQPWTGTYETTYTVPPSGTEPGTVIIETPESYVTTTQPWTGTYETTYTVPPSGTQPGT |
| | | VIIETPESYVTTTQPWTGTYETTYTVPPTGTEPGTVIIETPESYVTTTQPWTGTYETTYTVP |
| | | PSGTEPGIVIIETPESYVTTTQPWTGTYETTYTVPPTGTEPGTVIIETPESYVTTTQPWTGTY |
| | | ETTYTVPPTGTEPGTVIIETPESYVTTTQPWTGTYETTYTVPPSGTEPGTVIIETPESYVTTT |
| | | QPWTGTYETTYTVPPSGTEPGTVVIETPEITDCEAVCCGAVPTSDPLRRRDVCDCETFCCP |
| | | GDTNCETYVTTTQPWTGTYETTYTVPPSGTEPGTVIIETPESYVTTTQPWTGTYETTYTVP |
| | | PTGTEPGTVIIETPESYVTTTQPWTGTYETTYTVPPSGTQPGTVIIETPESYVTTTQPWTGT |
| | | YETTYTVPPTGTEPGTVIIETPESYVTTTQPWTGTYETTYTVPPSGTEPGTVIIETPESYVTT |
| | | TQPWTGTYETTYTVPPSGTQPGTVIIETPESYVTTTQPWTGTYETTYTVPPTGTEPGTVIIE |
| | | TPESYVTTTQPWTGTYETTYTVPPSGTEPGIVIIETPESYVTTTQPWTGTYETTYTVPPTGT |
| | | EPGTVIIETPESYVTTTQPWTGTYETTYTVPPSGTEPGTVIIETPESYVTTTQPWTGTYETT |
| | | YTVPPSGTQPGTVIIETPESYVTTTQPWTGTYETTYTVPPSGTEPGTVIVETPDVPGSYVTT |
| | | TQPWTGTYETTHTVPPTGTEPGTVVVETPDVPGSYVTTTQPWTGTYETTHTVPPTGTEPG |
| | | TVVVETPDVPGSYVTTTQPWTGTYETTYTVPPSGTEPGTVIVETPDVPGSYVTTTQPWTG |
| | | TYETTHTVPPTGTEPGTVVVETPDVPGSYVTTTQPWTGVYKTTYTVPPSGTIPGTVIIETPF |
| | | GYFNTSSISTKTDKRTITSVVPCSQCSESKTQYITPTGPGDVTVIISQPPSKITLSSPEDKTKT |
| | | DFITSTGSIGGGSPPSHPNDKPGIITTPTQPIGGGNPSDIPSAISSVSSGGNSRASVPSFSTSS |
| | | AISVQVSSLYDENSGSTFEVSLLFSVVSGFFLTLMV |
| FLO5 homolog | SEQ ID NO: | MKFPVPLLFLLQLFFIIATQGDESGNGDESDTAYGCDITSNAFDGFDATIYEYNANDLKLI |
| GQ68_03011 | 291 | RDPVFMSTGYLGRNVLNKISGVTVPGFNIWNPRSRTATVYGVQNVNYYNMVLELKGYF |
| (PAS_chr3_1145) | | KAAVSGDYKLTLSNIDDSSMLFFGKNTAFQCCDTGSIPVDQAPTDYSLFTIKPSNQVNSEV |
| | | ISSTQYLEAGKYYPVRIVFVNALERALFNFKLTIPSGTVLDDFQDYIYQFGALDENSCYET |
| | | TVSKITEWTTYTTPWTGTFETTRTITPTGTEGTVVIETPESYVTTTQPWTGTYETTYTVPPT |
| | | GTEPGTVIIETPEIIDCEAVCCGPFLTAFSFRKREECQCENICCPGDTNCETYVTTTQPWTG |
| | | TYETTYTVPPTGTEPGTVIIETPESYVTTTQPWTGTYETTYTVPPTGTEPGTVIIETPESYVT |
| | | TTQPWTGTYETTYTVPPSGTEPGTVVIETPEIVDCEAYCCASVAIKKRELCQCENFCCSW |
| | | DQSCQTYVTTTQPWTGTYETTYTVPPTGTEPGTVIIETPESYVTTTQPWTGTYETTYTVPP |
| | | TGTEPGTVIIETPESYVTTTQPWTGTYETTYTVPPTGTEPGTVIIETPEIIDCEAVCCGPFLT |
| | | AFSFRKREECQCENICCPGDTNCETYVTTTQPWTGTYETTYTVPPTGTEPGTVIIETPESYV |
| | | TTTQPWTGTYETTYTVPPTGTEPGTVIIETPESYVTTTQPWTGTYETTYTVPPTGTEPGTVI |
| | | IETPEIINCEAVCCGPFLTAFSFRKREECQCENICCPGDTNCETYVTTTQPWTGTYETTYTV |
| | | PPTGTEPGTVIIETPESYVTTTQPWTGTYETTYTVPSTGTEPGTVIIETPESYVTTTQPWTGT |
| | | YETTFTVPPTGTEPGTVVIETPESYVTTTQPWTGTYETTYSVPPSGTEPGTVVIETPEASTA |
| | | RTKFTTVTSSWTGVFTTTKTLPASGTEPATIVIQTPTGYFNTSSLVSTRTKTNVDTVTRVIP |
| | | CPICTAPKTITVVPEEPNESVSVIISQPQSSSTDTTLSKPDSVRVISQPETASQMDTSLSKTDS |
| | | AVISTETAGNNIIPLAGSHSYNTIVTTVTDSPQVAQSTTATSSSNVHLTISTQTTTPSLVYSS |
| | | SLSTVHQVSPSNGGFRSSITVHPLLSVIGAIFGALFM |
| FLO5 homolog | SEQ ID NO: | MTKFTILLLVLLKFYSILAIEVDGSANGQPLAHPIVVEVHEATKWITHTSPWTGTPEAIRT |
| GQ68_03079 | 292 | VTGETPYEQKIARYDEFNPRLANREIIDCVAFCCGDATSSPSITEPESTATELPESYVTINRP |
| (chr3) | | WSLSWIPDVPPGSPYWSTSTIPPSGTEPGTVIIYFYLYDDARKRREINFGSTQPYHGRPKLL |
| | | GSIEKRELCQCDAVCCLGDLSCEVYVTTTQPWTGTYETTYTITPTGSEPGTVIIETPELYVT |
| | | TTQPWTGTYETTYTITPTGSEPGTVIIETPESYVTTTQPWTGTYETTYTITPTGSEPGTVIIET |
| | | PESYVTTTQPWTGTYETTYTITPTGSEPGTVIIETPESYVTTTQPWTGTYETTYTVPPSGTE |
| | | PGAVIIETPELYVTTTQPWTGTYETTYTITPTGSEPGTVIIETPESYVTTTQPWTGTYETTYT |
| | | VPPSGTEPGTVIIETPELYVTTTQPWTGTYETTYTITPTGSEPGTVIVEIPVSYVNSTQISTST |
| | | YDTTDTVLSSGVEPGTIAIETPIVYLNTSVSAFSRPWTKIDTVTQFSSCAVCSKPETITVTPE |
| | | NPIDTVTIIISQPQSTSQSNTPTSFKANSTSAFSRFDEDSIPVFGSYSYEITVNIDVNTEDDTT |
| | | TNLNADTTIIIGSLSAIRTVAGSSSNYHASNISPTINSQKTASSVVVHSDSSATVYQFSPSNG |
| | | APWLSVQISTLLSVVGTLLAAVLL |
| FLO5 homolog | SEQ ID NO: | MNFRYLLILPIYASIVLGQVGDFQLLLNAKEPIRNSPSLLSSNYGNLTLPAMANGALESHF |
| GQ68_04277 | 293 | DYGNAYVGDDQITVVYHLPDEHGQINAYRQDTDEYIGYLGLVTDDYGEYTYLSVIMPG |
| (chr4) | | VQYDQTTSVNWYIENEELKSTSINVQPLLGCYYKNPPQYSWYWASIDEPGNIASSNFVCE |
| | | PCKVYVDFVPTPSADITSMWTGSETSWTTDADGTVIELVPTPSADATSVWTGDHTTWTT |
| | | DDDGNVIEQIPTPSADITSMWTGSETSWTTDADGTVIELVPTPSADITSMWTGSETSWTTD |
| | | ADGTVIELVPTPSADATSVWTGDHTTWTTDDDGNVIEQIPTPSADITSMWTGSETSWTTD |
| | | ADGTVIELVPTPSADATSVWTGDHTTWTTDDDGNVIEQIPTPSADITSMWTGSETSWTTD |
| | | ADGTVIELVPTPSADITSMWTGSETSWTTDADGTVIELVPTPSADATSVWTGDHTTWTTD |
| | | DDGNVIEQIPTPSADITSMWTGSETSWTTDADGTVIELVPTPSADITSMWTGSETSWTTDA |
| | | DGTVIELVPTPSADITSMWTGSETSWTTDADGTVIELVPTPSADATSVWTGDHTTWTTDD |
| | | DGNVIEQIPTPSADITSMWTGSETSWTTDADGTVIELVPTPSADITSMWTGSETSWTTDAD |
| | | GTVIELVPTPSADITSMWTGSETSWTTDADGTVIELVPTPSADITSMWTGSETSWTTDAD |
| | | GTVIELVPTPSADITSMWTGSETSWTTDADGTVIELVPTPSADITSMWTGSETSWTTDAD |
| | | GTVIELVPTPSADATSVWTGDHTTWTTDDDGNVIEQIPTPSADITSMWTGSETSWTTDAD |
| | | GTVIELVPTPSADITSMWTGSETSWTTDADGTVIELVPTPSADATSVWTGDHTTWTTDDD |
| | | GNVIEQIPTPSADITSMWTGSETSWTTDADGTVIELVPTPSADATSVWTGDHTTWTTDDD |
| | | GNVIEQIPTPSADITSMWTGSETSWTTDADGTVIELVPTPSADITSMWTGSETSWTTDADG |
| | | TVIELVPTPSADITSMWTGSETSWTTDADGTVIELVPTPSADATSVWTGDHTTWTTDDDG |
| | | NVIEQIPTPSADITSMWTGSETSWTTDADGTVIELVPTPSADITSMWTGSETSWTTDADGT |
| | | VIELVPTPSADATSVWTGDHTTWTTDDDGNVIEQIPTPSADITSMWTGSETSWTTDADGT |
| | | VIELVPTPSADITSMWTGSETSWTTDADGTVIELVPTPSADITSMWTGSETSWTTDADGT |
| | | VIELVPTPSADATSVWTGDHTTWTTDDDGNVIEQIPTPSADITSMWTGSETSWTTDADGT |
| | | VIELVPTPSADITSMWTGSETSWTTDADGTVIELVPTPSADITSMWTGSETSWTTDADGT |
| | | VIELVPTPSADATSVWTGDHTTWTTDDDGNVIEQIPTPSADITSMWTGSETSWTTDADGT |
| | | VIELVPTPSADITSMWTGSETSWTTDADGTVIELVPTPSADATSVWTGDHTTWTTDDDG |
| | | NVIEQIPTPSADITSMWTGSETSWTTDADGTVIELVPTPSADITSMWTGSETSWTTDADGT |
| | | VIELVPTPSADITSMWTGSETSWTTDADGTVIELVPTPSADATSVWTGDHTTWTTDDDG |
| | | NVIEQIPTPSADITSMWTGSETSWTTDADGTVIELVPTPSADITSMWTGSETSWTTDADGT |
| | | VIELVPTPSADITSMWTGSETSWTTDADGTVIELVPTPSADATSVWTGDHTTWTTDDDG |
| | | NVIEQIPTPSADITSMWTGSETSWTTDADGTVIELVPTPSADITSMWTGSETSWTTDADGT |
| | | VIELVPTPSADITSMWTGSETSWTTDADGTVIELVPTPSADTTSVWTGSYTTWTTDEDGT |
| | | VIEQVPTPSADTPSADTTSVWTGSYTTWTTDEDGTVIEQVPTPSADTTSVWTGSYTTWTT |
| | | DEDGTVIEQVPTPSADTPSADTTSVWTGSYTTWTTEVGDGGSSTVVELVPTESSTSTNVM |
| | | QTPVPSSGVSDGVSVFNGFNVEVFHYPADNYELANEISFLSYGYENLGLVTTVTGVSDIN |
| | | FDTDSNWPYYIDRDALGNTGSYVNATIEYEGFFRAPVDGEYVFSFSSTDYNSILFVGSPAA |
| | | ADQALQKREVQFLKPETSPDYVLLFNNTRDLGKTVSTTQYLLADQYYPLRVVIAAISQH |
| | | ALLDFQIKLPNGASLTQYQGYVYNFALEGSESTTVIGDKTSTWTGSYTTWTTDSDGSTIV |
| | | VVPPATITADKTSTWTGSYTTWTTDSDGSTVVICPSITSDHNDKPSESTLTDSSISTTVVTV |
| | | TSCDIEKCTKTTALTGVRETTLTTGGTTTVVTTYCPLPTDIVTVKTTSIDGSEVLQTIYTAK |
| | | PNHVVPDVQTSTVTITREVCDAFTCTHATIVTGEILKTTTLADTHYTTVVPVYVPLETYQP |
| | | AVELSTLETVLKSSDLASGPVVTAGSVQPSYQSGGVAESSLTVSEFEAHSTSDTVSQPSTIS |
| | | LQTGEANALKWSSFFGAALVPLVNVFFV |
| FLO5 homolog | SEQ ID NO: | MQNTNDKLIIRTFYSISTIHGLLSINIFSDTRVYKFAIYSTDAVSLEPRTKNNMSLVTVLACF |
| GQ68_01371 | 294 | IIFAAHAFGQDTFYMLKVRTLTPNGYPLADSLSNPMQYWDLYYVPGGPRRLESSFVNWQ |
| (chr1) | | PTTAAPINQFYCRLGTDGHMTGYNRVTGSVIGKLSFGTNAATALAFGSYDGDPSYPPQAF |
| | | SISSSVSGTMTYLNVHYVNARSITWYSTTTATGETNVYINVASTGYTGDRTTYQAELWV |
| | | EPFVPNIPVDTTTSIWTGSQTSYTTEVGENGGSTVIELIPTPPADATSTWTGTYTTRTTDAD |
| | | GSVIEQIPTPSADTTSVWTGTYTTWTTDADGSVIEQIPTPSADTTSVWTGTYTTWTTDAD |
| | | GSVIEQIPTPSADTTATWTGTETSYTTDVGEDGSSTVIELVPTPSADTTATWTGTETSYTT |
| | | DVGEDGSSTVVELVPTPSADTTATWTGTETSYTTDVGEDGSSTVIELVPTPSADTTATWT |
| | | GTETSYTTDVGEDGSSTVIELVPTPSADTTATWTGTETSYTTDVGEDGSSTVVELVPTPSA |
| | | DTTATWTGTETSYTTDVGEDGSSTVIELVPTPSADTTATWTGTETSYTTDVGEDGSSTVIE |
| | | LVPTPSADTTATWTGTETSYTTDVGEDGSSTVIELVPTPSADTTATWTGTETSYTTDVGE |
| | | DGSSTVIELVPTPSADTTATWTGTETSYTTDVGEDGSSTVIELVPTPSADTTATWTGTETS |
| | | YTTDVGEDGSSTVIELVPTPSADTTATWTGTETSYTTDVGEDGSSTVIELVPTPSADTTAT |
| | | WTGTETSYTTDVGEDGSSTVIELVPTPSADTTATWTGTETSYTTDVGEDGSSTVIELVPTP |
| | | SADTTATWTGTETSYTTDVGEDGSSTVIELVPTPSADTTATWTGTETSYTTDVGEDGSST |
| | | VIELVPTPSADTTATWTGTETSYTTDVGEDGSSTVIELVPTPTPSADTTATWTGTETSYTT |
| | | DVGEDGSSTVIELVPTPSADTTATWTGTETSYTTDVGEDGSSTVIELVPTPSADTTATWTG |
| | | TETSYTTDVGEDGSSTVIELVPTPSADTTATWTGTETSYTTDVGEDGSSTVIELVPTPTPSA |
| | | DTTATWTGTETSYTTDVGEDGSSTVIELVPTPSADTTATWTGTETSYTTDVGEDGSSTVIE |
| | | LVPTPSADTTATWTGTETSYTTDVGEDGSSTVVELVPTPTPSADTTATWTGTETSYTTDV |
| | | GEDGSSTVVELVPTPSADTTATWTGTETSYTTDVGEDGSSTVIELVPTPSADTTATWTGT |
| | | ETSYTTDVGEDGSSTVVELVPTPSADTTATWTGTETSYTTDVGEDGSSTVVELVPTPTAD |
| | | TTATWTGTETSYTTDVGEDGSSTVIELVPTPSADTTATWTGTETSYTTDVGEDGSSTVVE |
| | | LVPTPSADTTATWTGTETSYTTDVGEDGSSTVVELVPTPSADTTATWTGTETSYTTDVGE |
| | | DGSSTVIELVPTPSADTTATWTGTETSYTTDVGEDGSSTVVELVPTPSADTTATWTGTETS |
| | | YTTDVGEDGSSTVVELVPTPTADTTATWTGTETSYTTDVGEDGSSTVIELVPTPSADTTA |
| | | TWTGTETSYTTDVGEDGSSTVVELVPTPSADTTATWTGTETSYTTDVGEDGSSTVIELVP |
| | | TPSADTTATWTGTETSYTTDVGEDGSSTVIELVPTPSADTTATWTGTETSYTTDVGEDGS |
| | | STVIELVPTPTPSADTTATWTGTETSYTTDVGEDGSSTVIELVPTPSADTTATWTGTETSY |
| | | TTDVGEDGSSTVIELVPTPTPSADTTATWTGTETSYTTDVGEDGSSTVVELVPTPSADTTA |
| | | TWTGTETSYTTDVGEDGSSTVIELVPTPSADTTATWTGTETSYTTDVGEDGSSTVVELVP |
| | | TPSADTTATWTGTETSYTTDVGEDGSSTVIELVPTPSADTTATWTGTETSYTTDVGEDGS |
| | | STVIELVPTPSADTTATWTGTETSYTTDVGEDGSSTVIELVPTPSADTTATWTGTETSYTT |
| | | DVGEDGSSTVIELVPTPSADTTATWTGTETSYTTDVGEDGSSTVIELVPTPSADTTATWTG |
| | | TETSYTTDVGEDGSSTVIELVPTPSADTTATWTGTETSYTTDVGEDGSSTVIELVPTPSAD |
| | | TTATWTGTETSYTTDVGEDGSSTVIELVPTPSADTTATWTGTETSYTTDVGEDGSSTVIEL |
| | | VPTPSADTTATWTGTETSYTTDVGEDGSSTVIELVPTPSADTTATWTGTETSYTTDVGED |
| | | GSSTVVELVPTPTPSADTTATWTGTETSYTTDVGEDGSSTVIELVPSDTETATNIVETPVPS |
| | | SGVSDGVSVFDGFNVEVFHYPADNYELANEIGFLSYGYENLGLVTNATGVSDINFDTDSN |
| | | WPYYIDRDALGNTGSYVNATIEYEGFFRAPVDGEYVFSFSNTDYNSILFVGSPAAAGQAL |
| | | QKRRVQFLKPETSPDHVLLFNNTRDLGQTISTTQYLLADQYYPLRVVIAAISQHALLDFQI |
| | | KLPNGALLTQYQGYVYNFALEGSESTTVIGDKTSTWTGSYTTWTTDSDGSTVVVVPSATI |
| | | TADKTSTWTGSYTTWTTDSDGSTIVICPSITSDHNDKPSESTLTDGSISTTVVTVTSCDIEK |
| | | CTKTTALTGVTETTLTTGGTTTVVTTYCPLPTDIVTVKTTSISGSEVLQTIYTAKPSHVVPN |
| | | VHTLTVTITREVCDAFTCTQATIVTGEILKTTTLADTHSTTVVPVYVPLESYQSAVELSTL |
| | | ETVLKSSDFASGSAVTAGSAQPSYQSGGVAESSLTGSELEAHSTSDTVSQPSTISPQTGEA |
| | | NALRWSSFFGAALVPLVNVFFV |
| FLO5 homolog | SEQ ID NO: | MTKLTILLSVLLQLFSVLAEVPKKTEWSSHTTYWTSTLEALRTVTPTGTERAVIGEAPYE |
| GQ68_04678 | 295 | YKLIGNDQFDPGLNAKREIIDCEAVCCGAVPTSDPLKRRDVCECENVCCPGDDCETYVTT |
| (PAS_chr4_0363) | | TQPWTGTYETTYTVPPSGTEPGTVVIETPEITDCEAVCCGAVPTSDPLRRRDVCECENVCC |
| | | PGDDCETYVTTTQPWTGTYETTYTVPPSGTEPGTVVIETPEITDCEAVCCGAVPTSDPLRR |
| | | RDVCECENVCCPGDDCETYVTTTQPWTGTYETTYTVPPTGTEPGTVVIETPVTYVTTTQP |
| | | WTGTYETTYTVPPTGTEPGTVVIETPEITDCEAVCCGAVPTSDPLRRRDVCECENVCCPG |
| | | DDCETYVTTTQPWTGTYETTYTVPPTGTEPGTVVIETPVTYVTTTQPWTGTYETTYTVPP |
| | | TGTEPGTVVIETPVTYVTTTQPWTGTYETTYTVPPTGTEPGTVVIETPVTYVTTTQPWTGT |
| | | YETTYTIPPTGTEPGTVVIETPEITDCEAVCCGAVPTSDPLRRRDVCECENVCCPGDDCET |
| | | YVTTTQPWTGTYETTYTVPPTGTEPGTVVIETPVTYVTTTQPWTGTYETTYTVPPTGTEP |
| | | GTVVIETPVTYVTTTQPWTGTYETTYTVPPTGTEPGTVVIETPVTYVTTTKPWTGTYETT |
| | | HTVPASGTEPGTVIIETPIKYLNTSISASTSTWTKINTVTQFISCPVCTIPKTITVTPKISNE |
| | | TVTIIISQPHGTSSRTTTVVKTDGASVSSHSYKTALTTDVKPEEKTSTKLGTVTTVSGSHSAID |
| | | TVTGSLSDYHASSIPHTVKSEEKASSTVTHTISSSTVYQVSPSNGASWLSVRLNTALSIIGT |
| | | LFAAVFI |
| FLO5 homolog | SEQ ID NO: | MSKTKNGGSEFVHIAYVFHIEASTPSDYINMIQIVLFPHQAQITKRMNLVTLLVCNLLCVS |
| GQ68_04282 | 296 | LTLGQGVYRLKFPALVVTGRESVGTTVVNYDFLVGNTGQYGDLGEFFYDGEPYYCWNS |
| (chr4) | | TDSQPLSCSSSSSLLISTQNVTISHPDEDGTVYAYAERDGGLLGRFTVGSVSADWPQWAVI |
| | | VYSTSSSAHPSSWYVDDNKLKLTSGLGPNNSTTLQACYFTQSSGRDRYAISLEGSPAYTG |
| | | QVSCQATEFDLEFIPPSADTTSIWDGSYTTWTTDSNGIVVEQIPTPSADTTSIWTGSETSWT |
| | | TDSDGTVIELVPTPSADATSIWTGDHTTWTTDSEGNVIEQIPTPSADTTSIWTGSETSWTT |
| | | DSDGTVIELVPTPSADATSIWTGDHTTWTTDSEGNVIEQIPTPSADATSIWTGDHTTWTTD |
| | | REGNVIEQIPTPSADTTSIWTGSETSWTTDSDGTVIELVPTPSADATSIWTGDHTTWTTDSE |
| | | GNVIEQIPTPSADATSIWTGSETSWTTDSDGTVIELVPTPSADATSIWTGDHTTWTTDSEG |
| | | NVIEQIPTPSADATSIWTGDHTTWTTDSEGNVIEQIPTPSADTTSIWTGSETSWTTDSDGTV |
| | | IELVPTPSADATSIWTGDHTTWTTDSEGNVIEQIPTPSADATSIWTGDHTTWTTDSEGNVI |
| | | EQIPTPSADTTSIWTGSETSWTTDSDGTVIELVPTPSADATSIWTGDHTTWTTDSEGNVIE |
| | | QIPTPSADATSIWTGDHTTWTTDSEGNVIEQIPTPSADTTSIWTGSETSWTTDSDGTVIELV |
| | | PTPSADATSIWTGDHTTWTTDSEGNVIEQIPTPSADATSIWTGSETSWTTDSDGTVIELVPT |
| | | PSADATSIWTGDHTTWTTDSEGNVIEQIPTPSADATSIWTGDHTTWTTDSEGNVIEQIPTPS |
| | | ADTTSIWTGDHTTWTTEVGGDGSSIVVELVPSETGTATNVVQTPVPSSGISDGVSALDGF |
| | | NVEVFHYPADNYELANEISFLSYGYENLGLVTTATGVSDINFDTDSNWPSYIDRNALGNT |
| | | GSYVNATIKYEGFFRAPVDGDYEFSFSNIDYNSILFVGSAAADQALRKREAQFLKPETSPN |
| | | HILFFNNSRDVGQTISTTQYLSADSYYPLRVVIAAVSQHALLDFQIKLPNGVSLTQFQGYV |
| | | YNFALEGAESTTVIGDKTSTWTGTYTTWTTDSEGSTIVLCPSIISDHNGKPADTTLTDGSIS |
| | | TTVVTVTSCDIKKCTKTTALTGVTQKTLTVKGTTTVVTAYCPLPTDVATVKTISVGGSEV |
| | | LQTVYTAKPSHIVPDVQTLTVTITREVCDALTCIPATIVTGEILKTTTLADTHSTTVIPVYV |
| | | PLETHQPALDLITLETVLKSSDFANGPAITSVSVESLSHQSGVVVSEFDSDSTSGAVSQPSS |
| | | AVSLQTGKASALKWSPFLGAAVISLFNVFFV |
| FLO5 homolog | SEQ ID NO: | MNLFTILAWGFLYVPLVLGEGYYSLNFDARVPIALGILGSSYQKYTIMADRSLLGGSNIDL |
| GQ68_03013 | 297 | DVTFSGIIELLTNRVHIVVSLPDADGRVSVYDMYSGTSLGYLSFVCSLTTCEVHAVSSSSG |
| (PAS_chr3_0015) | | ATTWTLDGNQLIPTSPSTVYACYRSLVGLLAQYTLNDRTSITAQCEQTNLYVELAIPAFPE |
| | | TTAVWTGTYTTWTTDESGSVIEQMPTPSADTTTTWTGTYTTWTTDADGSVIEQIPTPPAD |
| | | TTSVWTGTYTTRTTDADGSVIEQIPTPSADTTSIWTGTYTTWTTDADGSVIEQIPTPSADTT |
| | | SVWTGTYTTWTTDADGSVIEQIPTPSADTTSVWTGTYTTWTTDADGSVIEQIPTPSADTTS |
| | | VWTGTYTTWTTDADGSVIEQIPTPSTDTTLAPSADTTSIWTGTYTTWTTDADGSVIEQIPT |
| | | PSADTTSIWTGTYTTWTTDADGSVIEQIPTPSADTTSVWTGTYTTWTTDADGSVIEQIPTP |
| | | STDTTLAPSADTTSIWTGTYTTWTTDADGSVIEQIPTPSADTTSVWTGTYTTWTTDADGS |
| | | VIEQIPTPSADTTSVWTGTYTTWTTDADGSVIEQIPTPSADTTSVWTGTYTTWTTDADGS |
| | | VIEQIPTPSADTTSVWTGTYTTWTTDADGSVIEQIPTPSADTTSVWTGTYTTWTTDADGS |
| | | VIEQIPTPSADTTSIWTGTYTTWTTDADGSVIEQIPTPSADTTSVWTGTYTTWTTDADGSV |
| | | IEQIPTPSADTTLAPSADTTSIWTGTYTTWTTDADGSVIEQIPTPSADTTSIWTGTYTTWTT |
| | | DADGSVIEQIPTPSADTTSVWTGTYTTWTTDADGSVIEQIPTPSADTTSVWTGTYTTWTT |
| | | DADGSVIEQIPTPSADTTSVWTGTYTTWTTDADGSVIEQIPTPSADTTSVWTGTYTTWTT |
| | | DADGSVIEQIPTPSADTTSVWTGTYTTWTTDADGSVIEQIPTPSADTTSVWTGTYTTWTT |
| | | DADGSVIEQIPTPSTDTTLAPSADTTSIWTGTYTTWTTDADGSVIEQIPTPSADTTSVWTGT |
| | | YTTWTTDADGSVIEQIPTPSADTTSVWTGTYTTWTTDADGSVIEQIPTPSADTTSVWTGT |
| | | YTTWTTDADGSVIEQIPTPSTDTTLAPSADTTSIWTGTYTTWTTDADGSVIEQIPTPSADTT |
| | | SIWTGTYTTWTTDADGSVIEQIPTPSADTTSVWTGTYTTWTTDADGSVIEQIPTPSADTTS |
| | | VWTGTYTTWTTDADGSVIEQIPTPSADTTSVWTGTYTTWTTDADGSVIEQIPTPSADTTS |
| | | VWTGTYTTWTTDADGSVIEQSPTPSAYTTSVWTGTYTTWTTDADGSVIEQIPTPSADTTS |
| | | VWTGTYTTWTTDADGSVIEQIPTPSADTTSVWTGTYTTWTTDADGSVIEQIPTPSADTTS |
| | | VWTGTYTTWTTDADGSVIEQIPTPSADTTSVWTGTYTTWTTDADGSVIEQIPTPSADTTS |
| | | VWTGTYTTWTTDADGSVIEQIPTPSADTTSVWTGTYTTWTTDADGSVIEQSPTPSAYTTS |
| | | VWTGTYTTWTTDADGSVIEQIPTPSADTTLAPSADTTSIWTGTYTTWTTDADGSVIEQIPT |
| | | PSADTTSIWTGTYTTWTTDADGSVIEQIPTPSADTTSVWTGTYTTWTTDADGSVIEQIPTP |
| | | SADTTSVWTGTYTTWTTDADGSVIEQIPTPSADTTSVWTGTYTTWTTDADGSVIEQIPTPS |
| | | TDTTLAPSADTTSIWTGTYTTWTTDADGSVIEQIPTPSADTTSVWTGTYTTWTTDADGSV |
| | | IEQIPTPSTDTTLAPSADTTSIWTGTYTTWTTDADGSVIEQIPTPSADTTSVWTGTYTTWTT |
| | | DADGSVIEQIPTPSADTTSVWTGTYTTWTTDADGSVIEQIPTPSADTTSVWTGTYTTWTT |
| | | DADGSVIEQIPTPSADTTLAPSADTTSIWTGTYTTWTTDADGSVIEQIPTPSADTTSVWTG |
| | | TYTTWTTDAAGTVIEVIPSGTSISSDVIPTPLPTSGVDIDTIPYDAFNVAVYHYPADNYELA |
| | | NNLGFLTSGYEGLGQVTTATSVGNINFDTSSGWPYYIESNALGNTGSYVNATIEYVGFFQ |
| | | APANGNYELSFSNIDYNAILFLGSPATDSSLAKREVQFLKPETSSEYVLFFDHGKDAGQTV |
| | | STTQYLSAGLYYPLRIVLAAVSERAQLDFQITLPDGRVLDQYQGYVYNFAHEGIESATSS |
| | | AHETSWSRFTNSTIYSHSSTIGIITSSTDAPHSVINPTAIETTSTDTSISTVAVTTSICDTKD |
| | | CVKTTVITPNSPLPTQTVSLTTTTIDRSEVVQTAHSAVPSQFAPDAHPSAVTITREQCDAYSCS |
| | | QATIVSGKVLQTTTVSDSTTVVPLDTPQLSVEASTLETRLKSTQSSRAPTVTVQTSQSSRH |
| | | SEDVTESSVHVSEFDAQSTSATSASALQAPSSISLQTGGANTLRLSAFLGTALLPMLNVLFI |
| SED1 homolog | SEQ ID NO: | MQFSIVATLALAGSALAAYSNVTYTYETTITDVVTELTTYCPEPTTFVHKNKTITVTAPTT |
| (GQ68_01572) | 298 | LTITDCPCTISKTTKITTDVPPTTHSTPHTTTTHVPSTSTPAPTHSVSTISHGGAAKAGVAGL |
| | | AGVAAAAAYFL |
| Erp1 | SEQ ID NO: | MLLTSLLQVFACCLVLPAQVTAFYYYTSGAERKCFHKELSKGTLFQATYKAQIYDDQLQ |
| | 299 | NYRDAGAQDFGVLIDIEETFDDNHLVVHQKGSASGDLTFLASDSGEHKICIQPEAGGWLI |
| | | KAKTKIDVEFQVGSDEKLDSKGKATIDILHAKVNVLNSKIGEIRREQKLMRDREATFRDA |
| | | SEAVNSRAMWWIVIQLIVLAVTCGWQMKHLGKFFVKQKIL |
| Erp2 | SEQ ID NO: | MIKSTIALPSFFIVLILALVNSVAASSSYAPVAISLPAFSKECLYYDMVTEDDSLAVGYQVL |
| | 300 | TGGNFEIDFDITAPDGSVITSEKQKKYSDFLLKSFGVGKYTFCFSNNYGTALKKVEITLEK |
| | | EKTLTDEHEADVNNDDIIANNAVEEIDRNLNKITKTLNYLRAREWRNMSTVNSTESRLT |
| | | WLSILIIIIIAVISIAQVLLIQFLFTGRQKNYV |
| Emp24 | SEQ ID NO: | MASFATKFVIACFLFFSASAHNVLLPAYGRRCFFEDLSKGDELSISFQFGDRNPQSSSQLT |
| | 301 | GDFIIYGPERHEVLKTVRDTSHGEITLSAPYKGHFQYCFLNENTGIETKDVTFNIHGVVYV |
| | | DLDDPNTNTLDSAVRKLSKLTREVKDEQSYIVIRERTHRNTAESTNDRVKWWSIFQLGV |
| | | VIANSLFQIYYLRRFFEVTSLV |
| Erv25 | SEQ ID NO: | MQVLQLWLTTLISLVVAVQGLHFDIAASTDPEQVCIRDFVTEGQLVVADIHSDGSVGDG |
| | 302 | QKLNLFVRDSVGNEYRRKRDFAGDVRVAFTAPSSTAFDVCFENQAQYRGRSLSRAIELDI |
| | | ESGAEARDWNKISANEKLKPIEVELRRVEEITDEIVDELTYLKNREERLRDTNESTNRRVR |
| | | NFSILVIIVLSSLGVWQVNYLKNYFKTKHII |
| Erp3 | SEQ ID NO: | MSNLCVLFFQFFFLAQFFAEASPLTFELNKGRKECLYTLTPEIDCTISYYFAVQQGESNDF |
| | 303 | DVNYEIFAPDDKNKPIIERSGERQGEWSFIGQHKGEYAICFYGGKAHDKIVDLDFKYNCE |
| | | RQDDIRNERRKARKAQRNLRDSKTDPLQDSVENSIDTIERQLHVLERNIQYYKSRNTRNH |
| | | HTVCSTEHRIVMFSIYGILLIIGMSCAQIAILEFIFRESRKHNV* |
| Erp5 | SEQ ID NO: | MKYNIVHGICLLFAITQAVGAVHFYAKSGETKCFYEHLSRGNLLIGDLDLYVEKDGLFEE |
| | 304 | DPESSLTITVDETFDNDHRVLNQKNSHTGDVTFTALDTGEHRFCFTPFYSKKSATLRVFIE |
| | | LEIGNVEALDSKKKEDMNSLKGRVGQLTQRLSSIRKEQDAIREKEAEFRNQSESANSKIM |
| | | TWSVFQLLILLGTCAFQLRYLKNFFVKQKVV |
| Flo5-2 from | SEQ ID NO: | DESGNGDESDTAYGCDITSNAFDGFDATIYEYNANDLKLIRDPVFMSTGYLGRNVLNKIS |
| Komagataella | 305 | GVTVPGFNIWNPRSRTATVYGVQNVNYYNMVLELKGYFKAAVSGDYKLTLSNIDDSSM |
| phaffii | | LFFGKNTAFQCCDTGSIPVDQAPTDYSLFTIKPSNQVNSEVISSTQYLEAGKYYPVRIVFV |
| | | NALERALFNFKLTIPSGTVLDDFQDYIYQFGALDENSCYETTVSKITEWTTYTTPWTGTFE |
| | | TTRTITPTGTEGTVVIETPESYVTTTQPWTGTYETTYTVPPTGTEPGTVIIETPEIIDCEAVC |
| | | CGPFLTAFSFRKREECQCENICCPGDTNCETYVTTTQPWTGTYETTYTVPPTGTEPGTVIIE |
| | | TPESYVTTTQPWTGTYETTYTVPPTGTEPGTVIIETPESYVTTTQPWTGTYETTYTVPPSGT |
| | | EPGTVVIETPEIVDCEAYCCASVAIKKRELCQCENFCCSWDQSCQTYVTTTQPWTGTYET |
| | | TYTVPPTGTEPGTVIIETPESYVTTTQPWTGTYETTYTVPPTGTEPGTVIIETPESYVTTTQP |
| | | WTGTYETTYTVPPTGTEPGTVIIETPEIIDCEAVCCGPFLTAFSFRKREECQCENICCPGDT |
| | | NCETYVTTTQPWTGTYETTYTVPPTGTEPGTVIIETPESYVTTTQPWTGTYETTYTVPPTG |
| | | TEPGTVIIETPESYVTTTQPWTGTYETTYTVPPTGTEPGTVIIETPEIINCEAVCCGPFLTAFS |
| | | FRKREECQCENICCPGDTNCETYVTTTQPWTGTYETTYTVPPTGTEPGTVIIETPESYVTTT |
| | | QPWTGTYETTYTVPSTGTEPGTVIIETPESYVTTTQPWTGTYETTFTVPPTGTEPGTVVIET |
| | | PESYVTTTQPWTGTYETTYSVPPSGTEPGTVVIETPESYVTTTQPWTGTYETTYSVPPSGT |
| | | EPGTVVIETPEASTARTKFTTVTSSWTGVFTTTKTLPASGTEPATIVIQTPTGYFNTSSLVST |
| | | RTKTNVDTVTRVIPCPICTAPKTITVVPEEPNESVSVIISQPQSSSTDTTLSKPDSVRVISQPE |
| | | TASQMDTSLSKTDSAVISTETAGNNIIPLAGSHSYNTIVTTVTDSPQVAQSTTATSSSNVHL |
| | | TISTQTTTPSLVYSSSLSTVHQVSPSNGGFRSSITVHPLLSVIGAIFGALFM |
| Flo5-2 from | SEQ ID NO: | MKFPVPLLFLLQLFFIIATQGDESGNGDESDTAYGCDITSNAFDGFDATIYEYNANDLKLI |
| Komagataella | 306 | RDPVFMSTGYLGRNVLNKISGVTVPGFNIWNPRSRTATVYGVQNVNYYNMVLELKGYF |
| phaffii | | KAAVSGDYKLTLSNIDDSSMLFFGKNTAFQCCDTGSIPVDQAPTDYSLFTIKPSNQVNSEV |
| (underlined is | | ISSTQYLEAGKYYPVRIVFVNALERALFNFKLTIPSGTVLDDFQDYIYQFGALDENSCYET |
| signal peptide, | | TVSKITEWTTYTTPWTGTFETTRTITPTGTEGTVVIETPESYVTTTQPWTGTYETTYTVPPT |
| used in some | | GTEPGTVIIETPEIIDCEAVCCGPFLTAFSFRKREECQCENICCPGDTNCETYVTTTQPWTG |
| versions and not | | TYETTYTVPPTGTEPGTVIIETPESYVTTTQPWTGTYETTYTVPPTGTEPGTVIIETPESYVT |
| others) | | TTQPWTGTYETTYTVPPSGTEPGTVVIETPEIVDCEAYCCASVAIKKRELCQCENFCCSW |
| | | DQSCQTYVTTTQPWTGTYETTYTVPPTGTEPGTVIIETPESYVTTTQPWTGTYETTYTVPP |
| | | TGTEPGTVIIETPESYVTTTQPWTGTYETTYTVPPTGTEPGTVIIETPEIIDCEAVCCGPFLT |
| | | AFSFRKREECQCENICCPGDTNCETYVTTTQPWTGTYETTYTVPPTGTEPGTVIIETPESYV |
| | | TTTQPWTGTYETTYTVPPTGTEPGTVIIETPESYVTTTQPWTGTYETTYTVPPTGTEPGTVI |
| | | IETPEIINCEAVCCGPFLTAFSFRKREECQCENICCPGDTNCETYVTTTQPWTGTYETTYTV |
| | | PPTGTEPGTVIIETPESYVTTTQPWTGTYETTYTVPSTGTEPGTVIIETPESYVTTTQPWTGT |
| | | YETTFTVPPTGTEPGTVVIETPESYVTTTQPWTGTYETTYSVPPSGTEPGTVVIETPESYVT |
| | | TTQPWTGTYETTYSVPPSGTEPGTVVIETPEASTARTKFTTVTSSWTGVFTTTKTLPASGT |
| | | EPATIVIQTPTGYFNTSSLVSTRTKTNVDTVTRVIPCPICTAPKTITVVPEEPNESVSVIISQP |
| | | QSSSTDTTLSKPDSVRVISQPETASQMDTSLSKTDSAVISTETAGNNIIPLAGSHSYNTIVTT |
| | | VTDSPQVAQSTTATSSSNVHLTISTQTTTPSLVYSSSLSTVHQVSPSNGGFRSSITVHPLLS |
| | | VIGAIFGALFM |
| Flo11 from | SEQ ID NO: | SSGKTCPTSEVSPACYANQWETTFPPSDIKITGATWVQDNIYDVTLSYEAESLELENLTEL |
| Komagataella | 307 | KIIGLNSPTGGTKLVWSLNSKVYDIDNPAKWTTTLRVYTKSSADDCYVEMYPFQIQVDW |
| phaffii | | CEAGASTDGCSAWKWPKSYDYDIGCDNMQDGVSRKHHPVYKWPKKCSSNCGVEPTTS |
| | | DEPEEPTTSEEPEEPTTSEEPEEPTSSDEEPTTSEEPEEPTTSDEPEEPTTSEEPEEPTTSEEP |
| | | EEPTTSEEPTTSEEPEEPTSSDEEPTTSDEPEEPTTSDEPEEPTTSEEPTTSEEPEEPTTSSEE |
| | | PTPSEEPEGPTCPTSEVSPACYADQWETTFPPSDIKITGATWVEDNIYDVTLSYEAESLELENL |
| | | TELKIIGLNSPTGGTKVVWSLNSGIYDIDNPAKWTTTLRVYTKSSADDCYVEMYPFQIQVD |
| | | WCEAGASTDGCSAWKWPKSYDYDIGCDNMQDGVSRKHHPVYKWPKKCSSDCGVEPTT |
| | | SDEPEEPTTSEEPVEPTSSDEEPTTSEEPTTSEEPEEPTTSDEPEEPTTSEEPEEPTTSEEPEE |
| | | PTTSEEPTTSEEPEEPTSSDEEPTTSDEPEEPTTSEEPEEPTTSEEPEEPTTSEEPEEPTTSDE |
| | | PEEPTTSEEPEEPTTSEEPEEPTSSDEEPTTSEEPEEPTTSEEPEEPTTSEEPEEPTTSEEPEE |
| | | PTSSDEEPTTSEEPEEPTTSDEPEEPTTSEEPEEPTTSEEPEEPTSSDEEPTTSEEPEEPTTSD |
| | | EPEEPTTSEEPEEPTTSEEPEEPTTSEEPEEPTTSEEPEEPTSSDEEPTTSEEPEEPTTSDEPE |
| | | EPTTSEEPEEPTTSEEPEEPTTSEEPEEPTTSDEEPGTTEEPLVPTTKTETDVSTTLLTVTDCG |
| | | TKTCTKSLVITGVTKETVTTHGKTTVITTYCPLPTETVTPTPVTVTSTIYADESVTKTTVYTTG |
| | | AVEKTVTVGGSSTVVVVHTPLTTAVVQSQSTDEIKTVVTARPSTTTIVRDVCYNSVCSVATIV |
| | | TGVTEKTITFSTGSITVVPTYVPLVESEEHQRTASTSETRATSVVVPTVVGQSSSASATSSIF |
| | | PSVTIHEGVANTVKNSMISGAVALLFNALFL |
| Flo11 from | SEQ ID NO: | MVSLRSIFTSSILAAGLTRAHGSSGKTCPTSEVSPACYANQWETTFPPSDIKITGATWVQD |
| Komagataella | 308 | NIYDVTLSYEAESLELENLTELKIIGLNSPTGGTKLVWSLNSKVYDIDNPAKWTTTLRVYT |
| phaffii | | KSSADDCYVEMYPFQIQVDWCEAGASTDGCSAWKWPKSYDYDIGCDNMQDGVSRKHH |
| | | PVYKWPKKCSSNCGVEPTTSDEPEEPTTSEEPEEPTTSEEPEEPTSSDEEPTTSEEPEEPTTS |
| | | DEPEEPTTSEEPEEPTTSEEPEEPTTSEEPTTSEEPEEPTSSDEEPTTSDEPEEPTTSDEPEEP |
| | | TTSEEPTTSEEPEEPTTSSEEPTPSEEPEGPTCPTSEVSPACYADQWETTFPPSDIKITGATWV |
| | | EDNIYDVTLSYEAESLELENLTELKIIGLNSPTGGTKVVWSLNSGIYDIDNPAKWTTTLRV |
| | | YTKSSADDCYVEMYPFQIQVDWCEAGASTDGCSAWKWPKSYDYDIGCDNMQDGVSRK |
| | | HHPVYKWPKKCSSDCGVEPTTSDEPEEPTTSEEPVEPTSSDEEPTTSEEPTTSEEPEEPTTS |
| | | DEPEEPTTSEEPEEPTTSEEPEEPTTSEEPTTSEEPEEPTSSDEEPTTSDEPEEPTTSEEPEEP |
| | | TTSEEPEEPTTSEEPEEPTTSDEPEEPTTSEEPEEPTTSEEPEEPTSSDEEPTTSEEPEEPTTS |
| | | EEPEEPTTSEEPEEPTTSEEPEEPTSSDEEPTTSEEPEEPTTSDEPEEPTTSEEPEEPTTSEEP |
| | | EEPTSSDEEPTTSEEPEEPTTSDEPEEPTTSEEPEEPTTSEEPEEPTTSEEPEEPTTSEEPEEP |
| | | TSSDEEPTTSEEPEEPTTSDEPEEPTTSEEPEEPTTSEEPEEPTTSEEPEEPTTSDEEPGTTEE |
| | | PLVPTTKTETDVSTTLLTVTDCGTKTCTKSLVITGVTKETVTTHGKTTVITTYCPLPTETVTPT |
| | | PVTVTSTIYADESVTKTTVYTTGAVEKTVTVGGSSTVVVVHTPLTTAVVQSQSTDEIKTVVTAR |
| | | PSTTTIVRDVCYNSVCSVATIVTGVTEKTITFSTGSITVVPTYVPLVESEEHQRTASTSETR |
| | | ATSVVVPTVVGQSSSASATSSIFPSVTIHEGVANTVKNSMISGAVALLFNALFL |
| Adhesin domain | SEQ ID NO: | DESGNGDESDTAYGCDITSNAFDGFDATIYEYNANDLKLIRDPVFMSTGYLGRNVLNKIS |
| only of Flo5-2 | 309 | GVTVPGFNIWNPRSRTATVYGVQNVNYYNMVLELKGYFKAAVSGDYKLTLSNIDDSSM |
| from Komagataella | | LFFGKNTAFQCCDTGSIPVDQAPTDYSLFTIKPSNQVNSEVISSTQYLEAGKYYPVRIVFV |
| phaffii (without | | NALERALFNFKLTIPSGTVLDDFQDYIYQFGALDENSC |
| signal peptide or | | |
| extension + anchor | | |
| domains) | | |
| (secretion signal | SEQ ID NO: | Nucleotide sequence |
| (mini-alpha, alpha | 337 | ATGAGATTCCCATCTATTTTCACCGCTGTCTTGTTCGCTGCCTCCTCTGCATTGGCTGC |
| factor secretion | | CCCTGTTCAGACTACCACTGAAGACGAGCTTGAGGGTGATTTCGACGTCGCTGTTTTG |
| signal with | | CCTTTCTCTGCTTCCATTGCTGCTAAGGAAGAGGGTGTCTCTCTCGAGAAAAGAGAG |
| deletion and | | GCCGAAGCT |
| mutations | | |
| to eliminate | | |

EPS production))

| | | |
|---|---|---|
| (SUC2 sequence) | SEQ ID NO: | Nucleotide sequence |
| | 338 | TCTATGACGAATGAGACGTCAGACAGGCCACTGGTACATTTCACACCAAATAAAGGA |
| | | TGGATGAATGATCCCAACGGTCTGTGGTACGACGAGAAAGACGCTAAGTGGCATCTG |
| | | TACTTTCAATATAACCCCAATGACACTGTTTGGGGTACGCCCCTTTTCTGGGGCCATG |
| | | CAACCTCAGATGATCTGACTAACTGGGAAGACCAACCTATTGCCATCGCCCCCAAGC |
| | | GTAATGATTCAGGCGCCTTCTCTGGAAGTATGGTAGTCGACTACAACAACACGAGTG |
| | | GTTTTTTCAACGACACAATTGACCCAAGACAGAGATGTGTTGCCATTTGGACATATA |
| | | ATACACCTGAGTCAGAAGAACAATATATATCCTACTCTCTGGATGGAGGTTATACTTT |
| | | TACGGAGTATCAGAAAAACCCTGTTCTGGCAGCTAATTCCACCCAATTTCGTGACCC |
| | | AAAGGTGTTTTGGTATGAGCCATCACAGAAATGGATCATGACCGCCGCAAAGTCACA |
| | | GGACTATAAAATTGAGATATATTCATCAGACGACTTGAAATCCTGGAAGCTGGAGAG |
| | | TGCTTTCGCAAATGAAGGATTTTTGGGATACCAATACGAATGCCCTGGCCTGATCGA |
| | | AGTGCCCACTGAACAAGACCCATCAAAGTCTTACTGGGTGATGTTTATCTCTATAAAC |
| | | CCCGGCGCTCCCGCTGGAGGCTCCTTCAACCAATATTTCGTAGGTTCTTTTAACGGAA |
| | | CCCACTTCGAGGCATTCGATAACCAATCTAGGGTTGTCGATTTCGGAAAAGATTATTA |
| | | TGCACTACAAACCTTTTTTAATACGGACCCTACTTATGGATCAGCTTTAGGCATAGCC |
| | | TGGGCTTCTAACTGGGAGTACAGTGCCTTTGTTCCTACAAACCCATGGCGTTCCTCAA |
| | | TGAGTCTTGTCAGAAAATTCTCTCTGAATACGGAATATCAGGCTAACCCCGAGACAG |
| | | AACTAATAAACTTGAAAGCAGAGCCTATCTTGAATATAAGTAACGCTGGACCTTGGA |
| | | GTCGTTTTGCCACCAACACCACATTAACAAAAGCCAATTCCTATAACGTGGACCTTTC |
| | | CAACTCTACGGGAACACTGGAATTTGAACTGGTGTACGCCGTAAATACTACGCAAAC |
| | | AATTTCAAAGTCAGTCTTTGCTGACCTTAGTCTATGGTTCAAGGGTTTAGAGGACCCC |
| | | GAGGAGTACTTACGTATGGGTTTTGAGGTATCAGCATCTTCCTTTTTCCTGGATCGTG |
| | | GAAACTCCAAGGTGAAGTTTGTCAAGGAAAATCCTTATTTCACTAACAGGATGTCCG |
| | | TGAACAACCAGCCTTTCAAATCTGAGAACGATCTTTCCTATTACAAGGTCTACGGACT |
| | | TCTAGACCAAAACATATTGGAACTATACTTCAACGATGGAGATGTAGTTTCCACCAA |
| | | CACCTATTTTATGACCACAGGCAACGCCCTTGGATCAGTAAACATGACAACAGGTGT |
| | | TGATAACCTTTTTTACATAGACAAATTCCAAGTTAGAGAGGTAAAG |
| (flex linkers) | SEQ ID NO: | Nucleotide sequence |
| | 339 | GGTTCATCAGGGTCCTCAGGATCATCCGGTAGTAGTGGTTCATCCGGTTCATCCGGAT |
| | | CAAGTGGCTCCTCTGAAGCTGCAGCAAGGGAGGCTGCAGCCCGTGAGGCAGCCGCTA |
| | | GAGAAGCCGCCGCTAGGGGTGGTGGCGGCTCTGGCGGAGGCGGTTCCGGTGGCGGA |
| | | GGCTCT |
| (Tir4 anchors) | SEQ ID NO: | Nucleotide sequence |
| | 340 | CAAATCAACGAATTGAACGTTGTTTTAGATGATGTTAAGACCAACATTGCCGACTAC |
| | | ATCACCCTATCCTACACTCCAAATTCAGGTTTTTCCTTGGACCAAATGCCAGCTGGTA |
| | | TTATGGATATTGCTGCGCAATTGGTTGCAAATCCAAGTGATGACTCCTACACCACTTT |
| | | GTACTCTGAAGTGGACTTTTCTGCTGTTGAGCATATGTTGACTATGGTCCCATGGTAC |
| | | TCTTCTAGACTGCTTCCAGAATTAGAAGCAATGGATGCTTCTCTAACTACCTCAAGTT |
| | | CTGCTGCCACATCTTCAAGTGAAGTTGCTAGCTCTTCTATTGCTTCATCCACTAGCTCT |
| | | TCTGTTGCACCATCCTCAAGTGAAGTTGTCAGCTCTTCCGTTGCTTCATCCTCAAGTG |
| | | AAGTTGCCAGCTCCTCTGTTGCGTCTACAAGCGAAGCTACTAGTTCTTCTGCTGTCAC |
| | | ATCTTCCTCCGCTGTTTCCTCTTCGACCGAGTCTGTTAGCTCTTCCTCTGTCAGTTCTT |
| | | CCTCAGCCGTTTCCTCTTCTGAAGCTGTCAGTTCCTCTCCAGTTTCCTCAGTTGTTTCA |
| | | TCTTCGGCCGGACCTGCTAGCTCAAGCGTTGCTCCTTACAACTCAACCATTGCTAGCT |
| | | CTTCTTCCACTGCCCAGACTTCTATCTCGACCATTGCTCCTTACAACTCCACAACCAC |
| | | CACCACCCCAGCTAGTTCTGCTTCCAGCGTTATTATCTCAACCAGAAACGGTACCACT |
| | | GTTACTGAAACTGACAACACTCTTGTCACCAAAGAAACCACTGTCTGTGACTACTCTT |
| | | CAACATCTGCCGTTCCAGCTTCCACCACCGGTTACAACAATTCTACTAAGGTTTCAAC |
| | | CGCTACTATCTGCAGTACATGCAAAGAAGGTACCTCTACTGCAACTGACTTCTCTACA |
| | | CTAAAGACTACAGTTACCGTATGTGACTCCGCCTGTCAAGCTAAGAAGTCTGCTACC |
| | | GTTGTTAGCGTTCAATCTAAAACTACCGGTATCGTTGAACAAACCGAAAACGGTGCT |
| | | GCCAAGGCTGTTATCGGTATGGGTGCCGGTGCTTTAGCTGCTGTTGCCGCCATGCTAC |
| | | TATGA |

TABLE 8
Terminator Sequences

| Sequence | Sequence | |
|---|---|---|
| Info | Info | Sequence Info |
| AOX1 | SEQ ID | TCAAGAGGATGTCAGAATGCCATTTGCCTGAGAGATGCAGGCTTCATTTTTGATACTTTTTTATT |
| terminator | NO: 310 | TGTAACCTATATAGTATAGGATTTTTTTTGTCATTTTGTTTCTTCTCGTACGAGCTTGCTCCTGAT |
| | | CAGCCTATCTCGCAGCAGATGAATATCTTGTGGTAGGGGTTTGGGAAAATCATTCGAGTTTGAT |
| | | GTTTTTCTTGGTATTTCCCACTCCTCTTCAGAGTACAGAAGATTAAGTGAAACCTTCGTTTGTGC |
| | | G |
| TDH3 | SEQ ID | TCGATTTGTATGTGAAATAGCTGAAATTCGAAAATTTCATTATGGCTGTATCTACTTTAGCGTAT |
| terminator | NO: 311 | TAGGCATTTGAGCATTGGCTTGAACAATGCGGGCTGTAGTGTGTCACCAAAGAAACCATTCGGG |
| | | TTCGGATCTGGAAGTCCTCATCACGTGATGCCGATCTCGTGTATTTTATTTTCAGATAACACCTG |
| | | AAGACTTT |
| RPS25A | SEQ ID | ATTAGTGTACATCTGATAATATAGTACTACCACGTATGATAATGTAGAGAATAGTCTTCCTTGTC |
| terminator | NO: 312 | GAGTGTGTTTGCAGTTTTCTTGAGTTTCAAGGTTTAAATGCTGGTATATTAGTTCATCGAAGGTT |
| | | TCAGCCAATAGCACCTTAAATCAATCAAACTAATTCGACTCTTACGAAAGAGCCTACTGTGTTTA |
| | | GTATCGAAGTCGTTTACCTTTCATGTTGAATAGCTTCCTCTCTGACCCTAACATTTCAAGATCCTC |
| | | CTAAAGTTACCCGGATTGTGAAATTCTAATGATCCACCTGCCCAATGCATTTTTTCTTTATTCAGT |
| | | TTACCTTTTTTACCTAATATACGAGCTTGTTAAAGTAAGTGGCACTGCAATACTAGGCTTATTGT |
| | | TGATATTATGATGAATCGTTTTCACAAACTTGATTTCCTGTGAACTCACCATGTACTAAGGAAAA |
| | | AAACATGCATCACCATCTGAATATTTGAC |
| RPL2A | SEQ ID | ACTATGTAACTAACGAAACAGCATGTACTAATAGAACCGTATCGAGAATATTTATTTAGGTGAG |
| terminator | NO: 313 | TAGTAGGAGTGAACCAGACAGTCAATTTAGTGAGCTGTCCCAGCTTTTGTGCATTCCAGAATTG |
| | | CCGGTCAAATTGGTTATGGGTTATGGGGCTTTTCCGATTGAGGTTCAGTTTCTGCGGTTATCTCTT |
| | | TCTTGACCTGGTCTTTTACAGGCTGTTCTTTCTCCCCATGATTATTCTTTAGCTGAAGATACCGCT |
| | | TAGCCTGATAATGTCGTCGTTTTGTAATCAAAATCTTTAGTTGGGCATCGTCTGAGGTTTCCTTTG |
| | | GCTTCTGGGGTTGTTAGTAGGAACGTAGGAACCATAGTAACTTTTACACATACATTCTTATGATT |
| | | GCGAAGTAAGCTGAGTCTGCTGCTTGGCTCCCGAAGTACTTTCTCTTTCTCTACCGGTTGATTCT |
| | | CCTTCTGGTGCTCCTAAACGATTGTGTTAGAAGGGATTGAC |
| (TDH3 | SEQ ID | GCGGCCGCTCGATTTGTATGTGAAATAGCTGAAATTCGAAAATTTCATTATGGCTGTATCTACTT |
| trans- | NO: 341 | TAGCGTATTAGGCATTTGAGCATTGGCTTGAACAATGCGGGCTGTAGTGTGTCACCAAAGAAAC |
| criptional | | CATTCGGGTTCGGATCTGGAAGTCCTCATCACGTGATGCCGATCTCGTGTATTTTATTTTCAGAT |
| terminator) | | AACACCTGAAGACTTT |

TABLE 9
Exemplary SUC surface display molecules (surface display proteins)

| Sequence | Sequence | |
|---|---|---|
| Info | Info | Sequence Info |
| Exemplary | SEQ ID | CAGGTGAACCCACCTAACTATTTTTAACTGGCATCCAGTGAGCTCGCTGGGTGAAAGCCAACCA |
| SUC2 | NO: 314 | TCTTTTGTTTCGGGGAACCGTGCTCGCCCCGTAAAGTTAATTTTTTTTTCCCGCGCAGCTTTAATC |
| surface | | TTTCGGCAGAGAAGGCGTTTTCATCGTAGCGTGGGAACAGAATAATCAGTTCATGTGCTATACA |
| display | | GGCACATGGCAGCAGTCACTATTTTGCTTTTTAACCTTAAAGTCGTTCATCAATCATTAACTGAC |
| nucleotide | | CAATCAGATTTTTTGCATTTGCCACTTATCTAAAAATACTTTTGTATCTCGCAGATACGTTCAGTG |
| sequence | | GTTTCCAGGACAACACCCAAAAAAAGGTATCAATGCCACTAGGCAGTCGGTTTTATTTTTGGTC |
| | | ACCCACGCAAAGAAGCACCCACCTCTTTTAGGTTTTAAGTTGTGGGAACAGTAACACCGCCTAG |
| | | AGCTTCAGGAAAAACCAGTACCTGTGACCGCAATTCACCATGATGCAGAATGTTAATTTAAACG |
| | | AGTGCCAAATCAAGATTTCAACAGACAAATCAATCGATCCATAGTTACCCATTCCAGCCTTTTCG |
| | | TCGTCGAGCCTGCTTCATTCCTGCCTCAGGTGCATAACTTTGCATGAAAAGTCCAGATTAGGGCA |
| | | GATTTTGAGTTTAAAATAGGAAATATAAACAAATATACCGCGAAAAAGGTTTGTTTATAGCTTT |
| | | TCGCCTGGTGCCGTACGGTATAAATACATACTCTCCTCCCCCCCCTGGTTCTCTTTTTCTTTTGTT |
| | | ACTTACATTTTACCGTTCCGTCACTCGCTTCACTCAACAACAAAAGAATTCCGAAACG (GCW14 |
| | | promoter) |
| | | ATGAGATTCCCATCTATTTTCACCGCTGTCTTGTTCGCTGCCTCCTCTGCATTGGCTGCCCCTGTT |
| | | CAGACTACCACTGAAGACGAGCTTGAGGGTGATTTCGACGTCGCTGTTTTGCCTTTCTCTGCTTC |
| | | CATTGCTGCTAAGGAAGAGGGTGTCTCTCTCGAGAAAAGAGAGGCCGAAGCT (secretion signal |
| | | (mini-alpha, alpha factor secretion signal with deletion and mutations to |
| | | eliminate EPS production)) |
| | | TCTATGACGAATGAGACGTCAGACAGGCCACTGGTACATTTCACACCAAATAAAGGATGGATGA |
| | | ATGATCCCAACGGTCTGTGGTACGACGAGAAAGACGCTAAGTGGCATCTGTACTTTCAATATAA |
| | | CCCCAATGACACTGTTTGGGGTACGCCCCTTTTCTGGGGCCATGCAACCTCAGATGATCTGACTA |
| | | ACTGGGAAGACCAACCTATTGCCATCGCCCCCAAGCGTAATGATTCAGGCGCCTTCTCTGGAAG |
| | | TATGGTAGTCGACTACAACAACACGAGTGGTTTTTTCAACGACACAATTGACCCAAGACAGAGA |
| | | TGTGTTGCCATTTGGACATATAATACACCTGAGTCAGAAGAACAATATATATCCTACTCTCTGGA |
| | | TGGAGGTTATACTTTTACGGAGTATCAGAAAAACCCTGTTCTGGCAGCTAATTCCACCCAATTTC |
| | | GTGACCCAAAGGTGTTTTGGTATGAGCCATCACAGAAATGGATCATGACCGCCGCAAAGTCACA |
| | | GGACTATAAAATTGAGATATATTCATCAGACGACTTGAAATCCTGGAAGCTGGAGAGTGCTTTC |
| | | GCAAATGAAGGATTTTTGGGATACCAATACGAATGCCCTGGCCTGATCGAAGTGCCCACTGAAC |
| | | AAGACCCATCAAAGTCTTACTGGGTGATGTTTATCTCTATAAACCCCGGCGCTCCCGCTGGAGG |
| | | CTCCTTCAACCAATATTTCGTAGGTTCTTTTAACGGAACCCACTTCGAGGCATTCGATAACCAAT |
| | | CTAGGGTTGTCGATTTCGGAAAAGATTATTATGCACTACAAACCTTTTTTAATACGGACCCTACT |
| | | TATGGATCAGCTTTAGGCATAGCCTGGGCTTCTAACTGGGAGTACAGTGCCTTTGTTCCTACAAA |
| | | CCCATGGCGTTCCTCAATGAGTCTTGTCAGAAAATTCTCTCTGAATACGGAATATCAGGCTAACC |
| | | CCGAGACAGAACTAATAAACTTGAAAGCAGAGCCTATCTTGAATATAAGTAACGCTGGACCTTG |
| | | GAGTCGTTTTGCCACCAACACCACATTAACAAAAGCCAATTCCTATAACGTGGACCTTTCCAACT |
| | | CTACGGGAACACTGGAATTTGAACTGGTGTACGCCGTAAATACTACGCAAACAATTTCAAAGTC |
| | | AGTCTTTGCTGACCTTAGTCTATGGTTCAAGGGTTTAGAGGACCCCGAGGAGTACTTACGTATGG |
| | | GTTTTGAGGTATCAGCATCTTCCTTTTTCCTGGATCGTGGAAACTCCAAGGTGAAGTTTGTCAAG |
| | | GAAAATCCTTATTTCACTAACAGGATGTCCGTGAACAACCAGCCTTTCAAATCTGAGAACGATC |
| | | TTTCCTATTACAAGGTCTACGGACTTCTAGACCAAAACATATTGGAACTATACTTCAACGATGGA |
| | | GATGTAGTTTCCACCAACACCTATTTTATGACCACAGGCAACGCCCTTGGATCAGTAAACATGA |
| | | CAACAGGTGTTGATAACCTTTTTTACATAGACAAATTCCAAGTTAGAGAGGTAAAG (SUC2 |
| | | sequence) |
| | | GGTTCATCAGGGTCCTCAGGATCATCCGGTAGTAGTGGTTCATCCGGTTCATCCGGATCAAGTG |
| | | GCTCCTCTGAAGCTGCAGCAAGGGAGGCTGCAGCCCGTGAGGCAGCCGCTAGAGAAGCCGCCG |
| | | CTAGGGGTGGTGGCGGCTCTGGCGGAGGCGGTTCCGGTGGCGGAGGCTCT (flex linkers) |
| | | CAAATCAACGAATTGAACGTTGTTTTAGATGATGTTAAGACCAACATTGCCGACTACATCACCC |
| | | TATCCTACACTCCAAATTCAGGTTTTTCCTTGGACCAAATGCCAGCTGGTATTATGGATATTGCT |
| | | GCGCAATTGGTTGCAAATCCAAGTGATGACTCCTACACCACTTTGTACTCTGAAGTGGACTTTTC |
| | | TGCTGTTGAGCATATGTTGACTATGGTCCCATGGTACTCTTCTAGACTGCTTCCAGAATTAGAAG |
| | | CAATGGATGCTTCTCTAACTACCTCAAGTTCTGCTGCCACATCTTCAAGTGAAGTTGCTAGCTCT |
| | | TCTATTGCTTCATCCACTAGCTCTTCTGTTGCACCATCCTCAAGTGAAGTTGTCAGCTCTTCCGTT |
| | | GCTTCATCCTCAAGTGAAGTTGCCAGCTCCTCTGTTGCGTCTACAAGCGAAGCTACTAGTTCTTC |
| | | TGCTGTCACATCTTCCTCCGCTGTTTCCTCTTCGACCGAGTCTGTTAGCTCTTCCTCTGTCAGTTC |
| | | TTCCTCAGCCGTTTCCTCTTCTGAAGCTGTCAGTTCCTCTCCAGTTTCCTCAGTTGTTTCATCTTCG |
| | | GCCGGACCTGCTAGCTCAAGCGTTGCTCCTTACAACTCAACCATTGCTAGCTCTTCTTCCACTGC |
| | | CCAGACTTCTATCTCGACCATTGCTCCTTACAACTCCACAACCACCACCACCCCAGCTAGTTCTG |
| | | CTTCCAGCGTTATTATCTCAACCAGAAACGGTACCACTGTTACTGAAACTGACAACACTCTTGTC |
| | | ACCAAAGAAACCACTGTCTGTGACTACTCTTCAACATCTGCCGTTCCAGCTTCCACCACCGGTTA |
| | | CAACAATTCTACTAAGGTTTCAACCGCTACTATCTGCAGTACATGCAAAGAAGGTACCTCTACT |
| | | GCAACTGACTTCTCTACACTAAAGACTACAGTTACCGTATGTGACTCCGCCTGTCAAGCTAAGA |
| | | AGTCTGCTACCGTTGTTAGCGTTCAATCTAAAACTACCGGTATCGTTGAACAAACCGAAAACGG |
| | | TGCTGCCAAGGCTGTTATCGGTATGGGTGCCGGTGCTTTAGCTGCTGTTGCCGCCATGCTACTAT |
| | | GA (Tir4 anchors) |
| | | GCGGCCGCTCGATTTGTATGTGAAATAGCTGAAATTCGAAAATTTCATTATGGCTGTATCTACTT |
| | | TAGCGTATTAGGCATTTGAGCATTGGCTTGAACAATGCGGGCTGTAGTGTGTCACCAAAGAAAC |
| | | CATTCGGGTTCGGATCTGGAAGTCCTCATCACGTGATGCCGATCTCGTGTATTTTATTTTCAGAT |
| | | AACACCTGAAGACTTT (TDH3 transcriptional terminator) |
| Exemplary | SEQ ID | SMTNETSDRPLVHFTPNKGWMNDPNGLWYDEKDAKWHLYFQYNPNDTVWGTPLFWGHATSDDL |
| SUC2 | NO: 315 | TNWEDQPIAIAPKRNDSGAFSGSMVVDYNNTSGFFNDTIDPRQRCVAIWTYNTPESEEQYISYSLDG |
| surface | | GYTFTEYQKNPVLAANSTQFRDPKVFWYEPSQKWIMTAAKSQDYKIEIYSSDDLKSWKLESAFANE |
| display | | GFLGYQYECPGLIEVPTEQDPSKSYWVMFISINPGAPAGGSFNQYFVGSFNGTHFEAFDNQSRVVDF |
| protein | | GKDYYALQTFFNTDPTYGSALGIAWASNWEYSAFVPTNPWRSSMSLVRKFSLNTEYQANPETELINL |
| sequence | | KAEPILNISNAGPWSRFATNTTLTKANSYNVDLSNSTGTLEFELVYAVNTTQTISKSVFADLSLWFKG |
| | | LEDPEEYLRMGFEVSASSFFLDRGNSKVKFVKENPYFTNRMSVNNQPFKSENDLSYYKVYGLLDQNI |
| | | LELYFNDGDVVSTNTYFMTTGNALGSVNMTTGVDNLFYIDKFQVREVK (SUC2 sequence) |
| | | GSSGSSGSSGSSGSSGSSGSSGSSEAAAREAAAREAAAREAAARGGGGSGGGGSGGGGS (linker |
| | | sequence) |
| | | QINELNVVLDDVKTNIADYITLSYTPNSGFSLDQMPAGIMDIAAQLVANPSDDSYTTLYSEVDFSAVE |
| | | HMLTMVPWYSSRLLPELEAMDASLTTSSSAATSSSEVASSSIASSTSSSVAPSSSEVVSSSVASSSSEV |
| | | ASSSVASTSEATSSSAVTSSSAVSSSTESVSSSSVSSSSAVSSSEAVSSSPVSSVVSSSAGPASSSVAPYN |
| | | STIASSSSTAQTSISTIAPYNSTTTTTPASSASSVIISTRNGTTVTETDNTLVTKETTVCDYSSTSAVPAST |
| | | TGYNNSTKVSTATICSTCKEGTSTATDFSTLKTTVTVCDSACQAKKSATVVSVQSKTTGIVEQTENG |
| | | AAKAVIGMGAGALAAVAAMLL (Tir4 anchor) |
| Exemplary | SEQ ID | SMTNETSDRPLVHFTPNKGWMNDPNGLWYDEKDAKWHLYFQYNPNDTVWGTPLFWGHATSDDL |
| SUC2 | NO: 332 | TNWEDQPIAIAPKRNDSGAFSGSMVVDYNNTSGFFNDTIDPRQRCVAIWTYNTPESEEQYISYSLDG |
| surface | | GYTFTEYQKNPVLAANSTQFRDPKVFWYEPSQKWIMTAAKSQDYKIEIYSSDDLKSWKLESAFANE |
| display | | GFLGYQYECPGLIEVPTEQDPSKSYWVMFISINPGAPAGGSFNQYFVGSFNGTHFEAFDNQSRVVDF |
| protein | | GKDYYALQTFFNTDPTYGSALGIAWASNWEYSAFVPTNPWRSSMSLVRKFSLNTEYQANPETELINL |
| sequence | | KAEPILNISNAGPWSRFATNTTLTKANSYNVDLSNSTGTLEFELVYAVNTTQTISKSVFADLSLWFKG |
| (without | | LEDPEEYLRMGFEVSASSFFLDRGNSKVKFVKENPYFTNRMSVNNQPFKSENDLSYYKVYGLLDQNI |
| C- | | LELYFNDGDVVSTNTYFMTTGNALGSVNMTTGVDNLFYIDKFQVREVK (SUC2 sequence) |
| terminus | | GSSGSSGSSGSSGSSGSSGSSGSSEAAAREAAAREAAAREAAARGGGGSGGGGSGGGGS (linker |
| of Tir4 | | sequence) |
| GPI | | QINELNVVLDDVKTNIADYITLSYTPNSGFSLDQMPAGIMDIAAQLVANPSDDSYTTLYSEVDFSAVE |
| anchor or | | HMLTMVPWYSSRLLPELEAMDASLTTSSSAATSSSEVASSSIASSTSSSVAPSSSEVVSSSVASSSSEV |
| signal | | ASSSVASTSEATSSSAVTSSSAVSSSTESVSSSSVSSSSAVSSSEAVSSSPVSSVVSSSAGPASSSVAPYN |
| peptide) | | STIASSSSTAQTSISTIAPYNSTTTTTPASSASSVIISTRNGTTVTETDNTLVTKETTVCDYSSTSAVPAST |
| | | TGYNNSTKVSTATICSTCKEGTSTATDFSTLKTTVTVCDSACQAKKSATVVSVQSKTTGIVEQTEN |
| Exemplary | SEQ ID | MRFPSIFTAVLFAASSALAAPVQTTTEDELEGDFDVAVLPFSASIAAKEEGVSLEKREAEASMTNETS |
| SUC2 | NO: 333 | DRPLVHFTPNKGWMNDPNGLWYDEKDAKWHLYFQYNPNDTVWGTPLFWGHATSDDLTNWEDQP |
| surface | | LAIAPKRNDSGAFSGSMVVDYNNTSGFFNDTIDPRQRCVAIWTYNTPESEEQYISYSLDGGYTFTEYQ |
| display | | KNPVLAANSTQFRDPKVFWYEPSQKWIMTAAKSQDYKIEIYSSDDLKSWKLESAFANEGFLGYQYE |
| protein | | CPGLIEVPTEQDPSKSYWVMFISINPGAPAGGSFNQYFVGSFNGTHFEAFDNQSRVVDFGKDYYALQ |
| sequence | | TFFNTDPTYGSALGIAWASNWEYSAFVPTNPWRSSMSLVRKFSLNTEYQANPETELINLKAEPILNIS |
| | | NAGPWSRFATNTTLTKANSYNVDLSNSTGTLEFELVYAVNTTQTISKSVFADLSLWFKGLEDPEEYL |
| | | RMGFEVSASSFFLDRGNSKVKFVKENPYFTNRMSVNNQPFKSENDLSYYKVYGLLDQNILELYFND |
| | | GDVVSTNTYFMTTGNALGSVNMTTGVDNLFYIDKFQVREVKGSSGSSGSSGSSGSSGSSGSSGSSEA |
| | | AAREAAAREAAAREAAARGGGGSGGGGSGGGGSQINELNVVLDDVKTNIADYITLSYTPNSGFSLD |
| | | QMPAGIMDIAAQLVANPSDDSYTTLYSEVDFSAVEHMLTMVPWYSSRLLPELEAMDASLTTSSSAA |
| | | TSSSEVASSSIASSTSSSVAPSSSEVVSSSVASSSSEVASSSVASTSEATSSSAVTSSSAVSSSTESVSSSS |
| | | VSSSSAVSSSEAVSSSPVSSVVSSSAGPASSSVAPYNSTIASSSSTAQTSISTIAPYNSTTTTTPASSASSV |
| | | IISTRNGTTVTETDNTLVTKETTVCDYSSTSAVPASTTGYNNSTKVSTATICSTCKEGTSTATDFSTLK |
| | | TTVTVCDSACQAKKSATVVSVQSKTTGIVEQTENGAAKAVIGMGAGALAAVAAMLL* |
| Exemplary | SEQ ID | SMTNETSDRPLVHFTPNKGWMNDPNGLWYDEKDAKWHLYFQYNPNDTVWGTPLFWGHATSDDL |
| SUC2 | NO: 334 | TNWEDQPIAIAPKRNDSGAFSGSMVVDYNNTSGFFNDTIDPRQRCVAIWTYNTPESEEQYISYSLDG |
| surface | | GYTFTEYQKNPVLAANSTQFRDPKVFWYEPSQKWIMTAAKSQDYKIEIYSSDDLKSWKLESAFANE |
| display | | GFLGYQYECPGLIEVPTEQDPSKSYWVMFISINPGAPAGGSFNQYFVGSFNGTHFEAFDNQSRVVDF |
| protein | | GKDYYALQTFFNTDPTYGSALGIAWASNWEYSAFVPTNPWRSSMSLVRKFSLNTEYQANPETELINL |
| sequence | | KAEPILNISNAGPWSRFATNTTLTKANSYNVDLSNSTGTLEFELVYAVNTTQTISKSVFADLSLWFKG |
| (without | | LEDPEEYLRMGFEVSASSFFLDRGNSKVKFVKENPYFTNRMSVNNQPFKSENDLSYYKVYGLLDQNI |
| extreme | | LELYFNDGDVVSTNTYFMTTGNALGSVNMTTGVDNLFYIDKFQVREVKGSSGSSGSSGSSGSSGSSG |
| C- | | SSGSSEAAAREAAAREAAAREAAARGGGGSGGGGSGGGGSQINELNVVLDDVKTNIADYITLSYTP |
| terminus | | NSGFSLDQMPAGIMDIAAQLVANPSDDSYTTLYSEVDFSAVEHMLTMVPWYSSRLLPELEAMDASL |
| of the Tir4 | | TTSSSAATSSSEVASSSIASSTSSSVAPSSSEVVSSSVASSSSEVASSSVASTSEATSSSAVTSSSAVSSST |
| GPI | | ESVSSSSVSSSSAVSSSEAVSSSPVSSVVSSSAGPASSSVAPYNSTIASSSSTAQTSISTIAPYNSTTTTTP |
| anchor or | | ASSASSVIISTRNGTTVTETDNTLVTKETTVCDYSSTSAVPASTTGYNNSTKVSTATICSTCKEGTSTA |
| signal | | TDFSTLKTTVTVCDSACQAKKSATVVSVQSKTTGIVEQTEN |
| peptide) | | |
| Exemplary | SEQ ID | MRFPSIFTAVLFAASSALAAPVQTTTEDELEGDFDVAVLPFSASIAAKEEGVSLEKREAEASMTNETS |
| SUC2 | NO: 335 | DRPLVHFTPNKGWMNDPNGLWYDEKDAKWHLYFQYNPNDTVWGTPLFWGHATSDDLTNWEDQP |
| surface | | LAIAPKRNDSGAFSGSMVVDYNNTSGFFNDTIDPRQRCVAIWTYNTPESEEQYISYSLDGGYTFTEYQ |
| display | | KNPVLAANSTQFRDPKVFWYEPSQKWIMTAAKSQDYKIEIYSSDDLKSWKLESAFANEGFLGYQYE |
| protein | | CPGLIEVPTEQDPSKSYWVMFISINPGAPAGGSFNQYFVGSFNGTHFEAFDNQSRVVDFGKDYYALQ |
| sequence | | TFFNTDPTYGSALGIAWASNWEYSAFVPTNPWRSSMSLVRKFSLNTEYQANPETELINLKAEPILNIS |
| (without | | NAGPWSRFATNTTLTKANSYNVDLSNSTGTLEFELVYAVNTTQTISKSVFADLSLWFKGLEDPEEYL |
| extreme | | RMGFEVSASSFFLDRGNSKVKFVKENPYFTNRMSVNNQPFKSENDLSYYKVYGLLDQNILELYFND |
| C- | | GDVVSTNTYFMTTGNALGSVNMTTGVDNLFYIDKFQVREVKGSSGSSGSSGSSGSSGSSGSSGSSEA |
| terminus | | AAREAAAREAAAREAAARGGGGSGGGGSGGGGSQINELNVVLDDVKTNIADYITLSYTPNSGFSLD |
| of the Tir4 | | QMPAGIMDIAAQLVANPSDDSYTTLYSEVDFSAVEHMLTMVPWYSSRLLPELEAMDASLTTSSSAA |
| GPI | | TSSSEVASSSIASSTSSSVAPSSSEVVSSSVASSSSEVASSSVASTSEATSSSAVTSSSAVSSSTESVSSSS |
| anchor) | | VSSSSAVSSSEAVSSSPVSSVVSSSAGPASSSVAPYNSTIASSSSTAQTSISTIAPYNSTTTTTPASSASSV |
| | | IISTRNGTTVTETDNTLVTKETTVCDYSSTSAVPASTTGYNNSTKVSTATICSTCKEGTSTATDFSTLK |
| | | TTVTVCDSACQAKKSATVVSVQSKTTGIVEQTEN |
| Exemplary | SEQ ID | EAEASMTNETSDRPLVHFTPNKGWMNDPNGLWYDEKDAKWHLYFQYNPNDTVWGTPLFWGHAT |
| SUC2 | NO: 342 | SDDLTNWEDQPIAIAPKRNDSGAFSGSMVVDYNNTSGFFNDTIDPRQRCVAIWTYNTPESEEQYISYS |
| surface | | LDGGYTFTEYQKNPVLAANSTQFRDPKVFWYEPSQKWIMTAAKSQDYKIEIYSSDDLKSWKLESAF |
| display | | ANEGFLGYQYECPGLIEVPTEQDPSKSYWVMFISINPGAPAGGSFNQYFVGSFNGTHFEAFDNQSRV |
| protein | | VDFGKDYYALQTFFNTDPTYGSALGIAWASNWEYSAFVPTNPWRSSMSLVRKFSLNTEYQANPETE |
| sequence | | LINLKAEPILNISNAGPWSRFATNTTLTKANSYNVDLSNSTGTLEFELVYAVNTTQTISKSVFADLSL |
| (Post- | | WFKGLEDPEEYLRMGFEVSASSFFLDRGNSKVKFVKENPYFTNRMSVNNQPFKSENDLSYYKVYGL |
| processing | | LDQNILELYFNDGDVVSTNTYFMTTGNALGSVNMTTGVDNLFYIDKFQVREVKGSSGSSGSSGSSGS |
| mature | | SGSSGSSGSSEAAAREAAAREAAAREAAARGGGGSGGGGSGGGGSQINELNVVLDDVKTNIADYIT |
| sequence | | LSYTPNSGFSLDQMPAGIMDIAAQLVANPSDDSYTTLYSEVDFSAVEHMLTMVPWYSSRLLPELEA |
| (with | | MDASLTTSSSAATSSSEVASSSIASSTSSSVAPSSSEVVSSSVASSSSEVASSSVASTSEATSSSAVTSSS |
| secretion | | AVSSSTESVSSSSVSSSSAVSSSEAVSSSPVSSVVSSSAGPASSSVAPYNSTIASSSSTAQTSISTIAPYNS |
| signal | | TTTTTPASSASSVIISTRNGTTVTETDNTLVTKETTVCDYSSTSAVPASTTGYNNSTKVSTATICSTCKE |
| cleaved | | GTSTATDFSTLKTTVTVCDSACQAKKSATVVSVQSKTTGIVEQTEN |
| off the N- | | |
| term and | | |
| propeptide | | |
| of Tir4 | | |
| cleaved | | |
| off the C- | | |
| term)) | | |

TABLE 10
Exemplary transporter proteins

| Sequence | Sequence | |
|---|---|---|
| Info | Info | Sequence |
| Saccharomyces | SEQ ID | MKNIISLVSKKKAASKNEDKNISESSRDIVNQQEVENTEDFEEGKKDSAFELDHLEFTTNSAQLGDS |
| cerevisiae | NO: 316 | DEDNENVINEMNATDDANEANSEEKSMTLKQALLKYPKAALWSILVSTTLVMEGYDTALLSALY |
| MAL11 or | | ALPVFQRKFGTLNGEGSYEITSQWQIGLNMCVLCGEMIGLQITTYMVEFMGNRYTMITALGLLTAY |
| AGT1- | | IFILYYCKSLAMIAVGQILSAIPWGCFQSLAVTYASEVCPLALRYYMTSYSNICWLFGQIFASGIMKN |
| sucrose | | SQENLGNSDLGYKLPFALQWIWPAPLMIGIFFAPESPWWLVRKDRVAEARKSLSRILSGKGAEKDI |
| permease | | QVDLTLKQIELTIEKERLLASKSGSFFNCFKGVNGRRTRLACLTWVAQNSSGAVLLGYSTYFFERA |
| UniProtKB- | | GMATDKAFTFSLIQYCLGLAGTLCSWVISGRVGRWTILTYGLAFQMVCLFIIGGMGFGSGSSASNG |
| P53048 | | AGGLLLALSFFYNAGIGAVVYCIVAEIPSAELRTKTIVLARICYNLMAVINAILTPYMLNVSDWNWG |
| (MAL11_YEAST) | | AKTGLYWGGFTAVTLAWVIIDLPETTGRTFSEINELFNQGVPARKFASTVVDPFGKGKTQHDSLAD |
| | | ESISQSSSIKQRELNAADKC |
| Pichia angusta | SEQ ID | MPEFVENIEKPEEAEVIPDITKKINTLSDSDDGSGAFNDYIARFVEISTNAQNNEHQEKHMSLKEGLK |
| MAL2- | NO: 317 | TFPKAACWSIVLSTAIIMEGYDTTLLNSLYSMQSFAKKYGKYYPEIDQYQVPAKWQTSLSMSTYVG |
| maltose | | EIVGLYIAGLVAEKWGYRRTLISFMAAVVGLIFILFFAVDVQMLLAGELLCGIVWGAFQTLTVSYA |
| transporter | | SEVCPVVLRIYLTTYVNACWVIGQLIAACLLRGTMTLTSEWSYKIPFAVQWIWPVPIMIGIYLAPESP |
| UniProtKB- | | WWLVKKNRDAEAKKSITRLLSPNTEVPDVAPLAEAMLNKMQLTIKEESARTSNVSYFDCFKHGNF |
| Q32SL4 | | RRTRIAAMIWLIQNITGSVLMGYSTYFYIQAGLDSSMSFTFSIIQYALGLLGTLASWLLSQKLGRFDI |
| (Q32SL4_PICAN) | | YFLGLSINTCILIIVGGLGFSSSTSASWAIGSLLLVFTFVYDSSIGPITYCTVAEIPSSTVRAKTVALAR |
| | | NWYNLSQIPLSIVTPYMLNPTAWNWKAKAALLWAGLSICSLIYIWFEFPETKGRTYAELDILFKNGT |
| | | SARKFRSTQVETFNPQEMLKKMNNEDIIQVVDGDLDAGAATAKV |

Expression or Secretion of a Protein of Interest in Host Cells with an Alternative Carbon Source

In some embodiments, the engineered host cell which expresses a surface-displayed enzyme that hydrolyses a disaccharide produces about the same amount of a protein of interest compared to a similar cell that does not express a surface-displayed enzyme that hydrolyses a disaccharide. In these embodiments, “about the same amount” includes from about 1% to about 10%—more or less—protein of interest production.

In some embodiments, the engineered host cell which expresses a surface-displayed enzyme that hydrolyses a disaccharide produces more of the protein of interest compared to a similar cell that does not express a surface-displayed enzyme that hydrolyses a disaccharide. In some embodiments, the engineered host cell which expresses a surface-displayed enzyme that hydrolyses a disaccharide produces about 1% to about 200% more of the protein of interest compared to a similar cell that does not express a surface-displayed enzyme that hydrolyses a disaccharide. In some embodiments, the engineered host cell which expresses a surface-displayed enzyme that hydrolyses a disaccharide produces about 1% to about 2%, about 1% to about 5%, about 1% to about 10%, about 1% to about 15%, about 1% to about 20%, about 1% to about 30%, about 1% to about 40%, about 1% to about 50%, about 1% to about 75%, about 1% to about 100%, about 1% to about 150%, about 1% to about 200%, about 2% to about 5%, about 2% to about 10%, about 2% to about 15%, about 2% to about 20%, about 2% to about 30%, about 2% to about 40%, about 2% to about 50%, about 2% to about 75%, about 2% to about 100%, about 2% to about 150%, about 2% to about 200%, about 5% to about 10%, about 5% to about 15%, about 5% to about 20%, about 5% to about 30%, about 5% to about 40%, about 5% to about 50%, about 5% to about 75%, about 5% to about 100%, about 5% to about 150%, about 5% to about 200%, about 10% to about 15%, about 10% to about 20%, about 10% to about 30%, about 10% to about 40%, about 10% to about 50%, about 10% to about 75%, about 10% to about 100%, about 10% to about 200%, about 15% to about 20%, about 15% to about 30%, about 15% to about 40%, about 15% to about 50%, about 15% to about 75%, about 15% to about 100%, about 15% to about 150%, about 15% to about 200%, about 20% to about 30%, about 20% to about 40%, about 20% to about 50%, about 20% to about 75%, about 20% to about 100%, about 20% to about 200%, about 30% to about 40%, about 30% to about 50%, about 30% to about 75%, about 30% to about 100%, about 30% to about 150%, about 30% to about 200%, about 40% to about 50%, about 40% to about 75%, about 40% to about 100%, about 40% to about 150%, about 40% to about 200%, about 50% to about 75%, about 50% to about 100%, about 50% to about 200%, about 75% to about 100%, about 75% to about 200%, or about 100% to about 200% more of the protein of interest compared to a similar cell that does not express a surface-displayed enzyme that hydrolyses a disaccharide. In some embodiments, the engineered host cell which expresses a surface-displayed enzyme that hydrolyses a disaccharide produces about 1%, about 2%, about 5%, about 10%, about 15%, about 20%, about 30%, about 40%, about 50%, about 75%, about 100%, about 150%, or about 200% more of the protein of interest compared to a similar cell that does not express a surface-displayed enzyme that hydrolyses a disaccharide. In some embodiments, the engineered host cell which expresses a surface-displayed enzyme that hydrolyses a disaccharide produces at least about 1%, about 2%, about 5%, about 10%, about 15%, about 20%, about 30%, about 40%, about 50%, about 75%, about 150%, or about 100% more of the protein of interest compared to a similar cell that does not express a surface-displayed enzyme that hydrolyses a disaccharide. In some embodiments, the engineered host cell which expresses a surface-displayed enzyme that hydrolyses a disaccharide produces at most about 2%, about 5%, about 10%, about 15%, about 20%, about 30%, about 40%, about 50%, about 75%, about 100%, about 150% or about 200% more of the protein of interest compared to a similar cell that does not express a surface-displayed enzyme that hydrolyses a disaccharide.

In some embodiments, the engineered host cell which expresses a surface-displayed enzyme that hydrolyses a disaccharide secretes the same amount of the protein of interest compared to a similar cell that does not express a surface-displayed enzyme that hydrolyses a disaccharide. In these embodiments, “about the same amount” includes from about 1% to about 10% —more or less—protein of interest secretion.

In some embodiments, the engineered host cell which expresses a surface-displayed enzyme that hydrolyses a disaccharide secretes more of the protein of interest compared to a similar cell that does not express a surface-displayed enzyme that hydrolyses a disaccharide. In some embodiments, the engineered host cell which expresses a surface-displayed enzyme that hydrolyses a disaccharide secretes about 1% to about 200% more of the protein of interest compared to a similar cell that does not express a surface-displayed enzyme that hydrolyses a disaccharide. In some embodiments, the engineered host cell which expresses a surface-displayed enzyme that hydrolyses a disaccharide secretes about 1% to about 2%, about 1% to about 5%, about 1% to about 10%, about 1% to about 15%, about 1% to about 20%, about 1% to about 30%, about 1% to about 40%, about 1% to about 50%, about 1% to about 75%, about 1% to about 100%, about 1% to about 150%, about 1% to about 200%, about 2% to about 5%, about 2% to about 10%, about 2% to about 15%, about 2% to about 20%, about 2% to about 30%, about 2% to about 40%, about 2% to about 50%, about 2% to about 75%, about 2% to about 100%, about 2% to about 150%, about 2% to about 200%, about 5% to about 10%, about 5% to about 15%, about 5% to about 20%, about 5% to about 30%, about 5% to about 40%, about 5% to about 50%, about 5% to about 75%, about 5% to about 100%, about 5% to about 150%, about 5% to about 200%, about 10% to about 15%, about 10% to about 20%, about 10% to about 30%, about 10% to about 40%, about 10% to about 50%, about 10% to about 75%, about 10% to about 100%, about 10% to about 200%, about 15% to about 20%, about 15% to about 30%, about 15% to about 40%, about 15% to about 50%, about 15% to about 75%, about 15% to about 100%, about 15% to about 150%, about 15% to about 200%, about 20% to about 30%, about 20% to about 40%, about 20% to about 50%, about 20% to about 75%, about 20% to about 100%, about 20% to about 200%, about 30% to about 40%, about 30% to about 50%, about 30% to about 75%, about 30% to about 100%, about 30% to about 150%, about 30% to about 200%, about 40% to about 50%, about 40% to about 75%, about 40% to about 100%, about 40% to about 150%, about 40% to about 200%, about 50% to about 75%, about 50% to about 100%, about 50% to about 200%, about 75% to about 100%, about 75% to about 200%, or about 100% to about 200% more of the protein of interest compared to a similar cell that does not express a surface-displayed enzyme that hydrolyses a disaccharide. In some embodiments, the engineered host cell which expresses a surface-displayed enzyme that hydrolyses a disaccharide secretes about 1%, about 2%, about 5%, about 10%, about 15%, about 20%, about 30%, about 40%, about 50%, about 75%, about 100%, about 150%, or about 200% more of the protein of interest compared to a similar cell that does not express a surface-displayed enzyme that hydrolyses a disaccharide. In some embodiments, the engineered host cell which expresses a surface-displayed enzyme that hydrolyses a disaccharide secretes at least about 1%, about 2%, about 5%, about 10%, about 15%, about 20%, about 30%, about 40%, about 50%, about 75%, about 150%, or about 100% more of the protein of interest compared to a similar cell that does not express a surface-displayed enzyme that hydrolyses a disaccharide. In some embodiments, the engineered host cell which expresses a surface-displayed enzyme that hydrolyses a disaccharide secretes at most about 2%, about 5%, about 10%, about 15%, about 20%, about 30%, about 40%, about 50%, about 75%, about 100%, about 150% or about 200% more of the protein of interest compared to a similar cell that does not express a surface-displayed enzyme that hydrolyses a disaccharide.

In some embodiments, the engineered host cell which expresses a surface-displayed enzyme that hydrolyses a disaccharide produces about the same amount of a protein of interest compared to a similar cell that does not express a surface-displayed enzyme that hydrolyses a disaccharide when each are fed a growth medium comprising glucose as its carbon source. In these embodiments, “about the same amount” includes from about 1% to about 10% —more or less—protein of interest production.

In some embodiments, the engineered host cell which expresses a surface-displayed enzyme that hydrolyses a disaccharide produces more of the protein of interest compared to a similar cell that does not express a surface-displayed enzyme that hydrolyses a disaccharide when each are fed a growth medium comprising glucose as its carbon source. In some embodiments, the engineered host cell which expresses a surface-displayed enzyme that hydrolyses a disaccharide produces about 1% to about 200% more of the protein of interest compared to a similar cell that does not express a surface-displayed enzyme that hydrolyses a disaccharide when each are fed a growth medium comprising glucose as its carbon source. In some embodiments, the engineered host cell which expresses a surface-displayed enzyme that hydrolyses a disaccharide produces about 1% to about 2%, about 1% to about 5%, about 1% to about 10%, about 1% to about 15%, about 1% to about 20%, about 1% to about 30%, about 1% to about 40%, about 1% to about 50%, about 1% to about 75%, about 1% to about 100%, about 1% to about 150%, about 1% to about 200%, about 2% to about 5%, about 2% to about 10%, about 2% to about 15%, about 2% to about 20%, about 2% to about 30%, about 2% to about 40%, about 2% to about 50%, about 2% to about 75%, about 2% to about 100%, about 2% to about 150%, about 2% to about 200%, about 5% to about 10%, about 5% to about 15%, about 5% to about 20%, about 5% to about 30%, about 5% to about 40%, about 5% to about 50%, about 5% to about 75%, about 5% to about 100%, about 5% to about 150%, about 5% to about 200%, about 10% to about 15%, about 10% to about 20%, about 10% to about 30%, about 10% to about 40%, about 10% to about 50%, about 10% to about 75%, about 10% to about 100%, about 10% to about 200%, about 15% to about 20%, about 15% to about 30%, about 15% to about 40%, about 15% to about 50%, about 15% to about 75%, about 15% to about 100%, about 15% to about 150%, about 15% to about 200%, about 20% to about 30%, about 20% to about 40%, about 20% to about 50%, about 20% to about 75%, about 20% to about 100%, about 20% to about 200%, about 30% to about 40%, about 30% to about 50%, about 30% to about 75%, about 30% to about 100%, about 30% to about 150%, about 30% to about 200%, about 40% to about 50%, about 40% to about 75%, about 40% to about 100%, about 40% to about 150%, about 40% to about 200%, about 50% to about 75%, about 50% to about 100%, about 50% to about 200%, about 75% to about 100%, about 75% to about 200%, or about 100% to about 200% more of the protein of interest compared to a similar cell that does not express a surface-displayed enzyme that hydrolyses a disaccharide when each are fed a growth medium comprising glucose as its carbon source. In some embodiments, the engineered host cell which expresses a surface-displayed enzyme that hydrolyses a disaccharide produces about 1%, about 2%, about 5%, about 10%, about 15%, about 20%, about 30%, about 40%, about 50%, about 75%, about 100%, about 150%, or about 200% more of the protein of interest compared to a similar cell that does not express a surface-displayed enzyme that hydrolyses a disaccharide when each are fed a growth medium comprising glucose as its carbon source. In some embodiments, the engineered host cell which expresses a surface-displayed enzyme that hydrolyses a disaccharide produces at least about 1%, about 2%, about 5%, about 10%, about 15%, about 20%, about 30%, about 40%, about 50%, about 75%, about 150%, or about 100% more of the protein of interest compared to a similar cell that does not express a surface-displayed enzyme that hydrolyses a disaccharide when each are fed a growth medium comprising glucose as its carbon source. In some embodiments, the engineered host cell which expresses a surface-displayed enzyme that hydrolyses a disaccharide produces at most about 2%, about 5%, about 10%, about 15%, about 20%, about 30%, about 40%, about 50%, about 75%, about 100%, about 150% or about 200% more of the protein of interest compared to a similar cell that does not express a surface-displayed enzyme that hydrolyses a disaccharide when each are fed a growth medium comprising glucose as its carbon source.

In some embodiments, the engineered host cell which expresses a surface-displayed enzyme that hydrolyses a disaccharide secretes the same amount of the protein of interest compared to a similar cell that does not express a surface-displayed enzyme that hydrolyses a disaccharide when each are fed a growth medium comprising glucose as its carbon source. In these embodiments, “about the same amount” includes from about 1% to about 10% —more or less—protein of interest secretion.

In some embodiments, the engineered host cell which expresses a surface-displayed enzyme that hydrolyses a disaccharide secretes more of the protein of interest compared to a similar cell that does not express a surface-displayed enzyme that hydrolyses a disaccharide when each are fed a growth medium comprising glucose as its carbon source. In some embodiments, the engineered host cell which expresses a surface-displayed enzyme that hydrolyses a disaccharide secretes about 1% to about 200% more of the protein of interest compared to a similar cell that does not express a surface-displayed enzyme that hydrolyses a disaccharide when each are fed a growth medium comprising glucose as its carbon source. In some embodiments, the engineered host cell which expresses a surface-displayed enzyme that hydrolyses a disaccharide secretes about 1% to about 2%, about 1% to about 5%, about 1% to about 10%, about 1% to about 15%, about 1% to about 20%, about 1% to about 30%, about 1% to about 40%, about 1% to about 50%, about 1% to about 75%, about 1% to about 100%, about 1% to about 150%, about 1% to about 200%, about 2% to about 5%, about 2% to about 10%, about 2% to about 15%, about 2% to about 20%, about 2% to about 30%, about 2% to about 40%, about 2% to about 50%, about 2% to about 75%, about 2% to about 100%, about 2% to about 150%, about 2% to about 200%, about 5% to about 10%, about 5% to about 15%, about 5% to about 20%, about 5% to about 30%, about 5% to about 40%, about 5% to about 50%, about 5% to about 75%, about 5% to about 100%, about 5% to about 150%, about 5% to about 200%, about 10% to about 15%, about 10% to about 20%, about 10% to about 30%, about 10% to about 40%, about 10% to about 50%, about 10% to about 75%, about 10% to about 100%, about 10% to about 200%, about 15% to about 20%, about 15% to about 30%, about 15% to about 40%, about 15% to about 50%, about 15% to about 75%, about 15% to about 100%, about 15% to about 150%, about 15% to about 200%, about 20% to about 30%, about 20% to about 40%, about 20% to about 50%, about 20% to about 75%, about 20% to about 100%, about 20% to about 200%, about 30% to about 40%, about 30% to about 50%, about 30% to about 75%, about 30% to about 100%, about 30% to about 150%, about 30% to about 200%, about 40% to about 50%, about 40% to about 75%, about 40% to about 100%, about 40% to about 150%, about 40% to about 200%, about 50% to about 75%, about 50% to about 100%, about 50% to about 200%, about 75% to about 100%, about 75% to about 200%, or about 100% to about 200% more of the protein of interest compared to a similar cell that does not express a surface-displayed enzyme that hydrolyses a disaccharide when each are fed a growth medium comprising glucose as its carbon source. In some embodiments, the engineered host cell which expresses a surface-displayed enzyme that hydrolyses a disaccharide secretes about 1%, about 2%, about 5%, about 10%, about 15%, about 20%, about 30%, about 40%, about 50%, about 75%, about 100%, about 150%, or about 200% more of the protein of interest compared to a similar cell that does not express a surface-displayed enzyme that hydrolyses a disaccharide when each are fed a growth medium comprising glucose as its carbon source. In some embodiments, the engineered host cell which expresses a surface-displayed enzyme that hydrolyses a disaccharide secretes at least about 1%, about 2%, about 5%, about 10%, about 15%, about 20%, about 30%, about 40%, about 50%, about 75%, about 150%, or about 100% more of the protein of interest compared to a similar cell that does not express a surface-displayed enzyme that hydrolyses a disaccharide when each are fed a growth medium comprising glucose as its carbon source. In some embodiments, the engineered host cell which expresses a surface-displayed enzyme that hydrolyses a disaccharide secretes at most about 2%, about 5%, about 10%, about 15%, about 20%, about 30%, about 40%, about 50%, about 75%, about 100%, about 150% or about 200% more of the protein of interest compared to a similar cell that does not express a surface-displayed enzyme that hydrolyses a disaccharide when each are fed a growth medium comprising glucose as its carbon source.

In some embodiments, the engineered host cell which expresses a surface-displayed enzyme that hydrolyses a disaccharide produces more of the protein of interest compared to a similar cell that does not express a surface-displayed enzyme that hydrolyses a disaccharide when each are fed a growth medium comprising a disaccharide, e.g., sucrose, as its carbon source. In some embodiments, the engineered host cell which expresses a surface-displayed enzyme that hydrolyses a disaccharide produces about 10% to about 2000% more of the protein of interest compared to a similar cell that does not express a surface-displayed enzyme that hydrolyses a disaccharide when each are fed a growth medium comprising a disaccharide, e.g., sucrose, as its carbon source. In some embodiments, the engineered host cell which expresses a surface-displayed enzyme that hydrolyses a disaccharide produces about 10% to about 20%, about 10% to about 50%, about 10% to about 100%, about 10% to about 150%, about 10% to about 200%, about 10% to about 300%, about 10% to about 400%, about 10% to about 500%, about 10% to about 750%, about 10% to about 1000%, about 10% to about 1500%, about 10% to about 2000%, about 20% to about 50%, about 20% to about 100%, about 20% to about 150%, about 20% to about 200%, about 20% to about 300%, about 20% to about 400%, about 20% to about 500%, about 20% to about 750%, about 20% to about 1000%, about 20% to about 1500%, about 20% to about 2000%, about 50% to about 100%, about 50% to about 150%, about 50% to about 200%, about 50% to about 300%, about 50% to about 400%, about 50% to about 500%, about 50% to about 750%, about 50% to about 1000%, about 50% to about 1500%, about 50% to about 2000%, about 100% to about 150%, about 100% to about 200%, about 100% to about 300%, about 100% to about 400%, about 100% to about 500%, about 100% to about 750%, about 100% to about 1000%, about 100% to about 2000%, about 150% to about 200%, about 150% to about 300%, about 150% to about 400%, about 150% to about 500%, about 150% to about 750%, about 150% to about 1000%, about 150% to about 1500%, about 150% to about 2000%, about 200% to about 300%, about 200% to about 400%, about 200% to about 500%, about 200% to about 750%, about 200% to about 1000%, about 200% to about 2000%, about 300% to about 400%, about 300% to about 500%, about 300% to about 750%, about 300% to about 1000%, about 300% to about 1500%, about 300% to about 2000%, about 400% to about 500%, about 400% to about 750%, about 400% to about 1000%, about 400% to about 1500%, about 400% to about 2000%, about 500% to about 750%, about 500% to about 1000%, about 500% to about 2000%, about 750% to about 1000%, about 750% to about 2000%, or about 1000% to about 2000% more of the protein of interest compared to a similar cell that does not express a surface-displayed enzyme that hydrolyses a disaccharide when each are fed a growth medium comprising a disaccharide, e.g., sucrose, as its carbon source. In some embodiments, the engineered host cell which expresses a surface-displayed enzyme that hydrolyses a disaccharide produces at least about 10%, at least about 20%, at least about 50%, at least about 100%, at least about 150%, at least about 200%, at least about 300%, at least about 400%, at least about 500%, at least about 750%, at least about 1000%, at least about 1500%, or at least about 2000%, at least about 3000%, at least about 4000%, at least about 5000%, at least about 7500%, at least about 10000% more of the protein of interest compared to a similar cell that does not express a surface-displayed enzyme that hydrolyses a disaccharide when each are fed a growth medium comprising a disaccharide, e.g., sucrose, as its carbon source. In some embodiments, the engineered host cell which expresses a surface-displayed enzyme that hydrolyses a disaccharide produces about 10%, about 20%, about 50%, about 100%, about 150%, about 200%, about 300%, about 400%, about 500%, about 750%, about 1000%, about 1500%, or about 2000%, about 3000%, about 4000%, about 5000%, about 7500%, about 10000% more of the protein of interest compared to a similar cell that does not express a surface-displayed enzyme that hydrolyses a disaccharide when each are fed a growth medium comprising a disaccharide, e.g., sucrose, as its carbon source. In some embodiments, the engineered host cell which expresses a surface-displayed enzyme that hydrolyses a disaccharide produces at most about 20%, about 50%, about 100%, about 150%, about 200%, about 300%, about 400%, about 500%, about 750%, about 1000%, about 1500%, or about 2000%, about 3000%, about 4000%, about 5000%, about 7500%, about 10000% more of the protein of interest compared to a similar cell that does not express a surface-displayed enzyme that hydrolyses a disaccharide when each are fed a growth medium comprising a disaccharide, e.g., sucrose, as its carbon source.

In some embodiments, the engineered host cell which expresses a surface-displayed enzyme that hydrolyses a disaccharide secretes more of the protein of interest compared to a similar cell that does not express a surface-displayed enzyme that hydrolyses a disaccharide when each are fed a growth medium comprising a disaccharide, e.g., sucrose, as its carbon source. In some embodiments, the engineered host cell which expresses a surface-displayed enzyme that hydrolyses a disaccharide secretes about 10% to about 2000% more of the protein of interest compared to a similar cell that does not express a surface-displayed enzyme that hydrolyses a disaccharide when each are fed a growth medium comprising a disaccharide, e.g., sucrose, as its carbon source. In some embodiments, the engineered host cell which expresses a surface-displayed enzyme that hydrolyses a disaccharide secretes about 10% to about 20%, about 10% to about 50%, about 10% to about 100%, about 10% to about 150%, about 10% to about 200%, about 10% to about 300%, about 10% to about 400%, about 10% to about 500%, about 10% to about 750%, about 10% to about 1000%, about 10% to about 1500%, about 10% to about 2000%, about 20% to about 50%, about 20% to about 100%, about 20% to about 150%, about 20% to about 200%, about 20% to about 300%, about 20% to about 400%, about 20% to about 500%, about 20% to about 750%, about 20% to about 1000%, about 20% to about 1500%, about 20% to about 2000%, about 50% to about 100%, about 50% to about 150%, about 50% to about 200%, about 50% to about 300%, about 50% to about 400%, about 50% to about 500%, about 50% to about 750%, about 50% to about 1000%, about 50% to about 1500%, about 50% to about 2000%, about 100% to about 150%, about 100% to about 200%, about 100% to about 300%, about 100% to about 400%, about 100% to about 500%, about 100% to about 750%, about 100% to about 1000%, about 100% to about 2000%, about 150% to about 200%, about 150% to about 300%, about 150% to about 400%, about 150% to about 500%, about 150% to about 750%, about 150% to about 1000%, about 150% to about 1500%, about 150% to about 2000%, about 200% to about 300%, about 200% to about 400%, about 200% to about 500%, about 200% to about 750%, about 200% to about 1000%, about 200% to about 2000%, about 300% to about 400%, about 300% to about 500%, about 300% to about 750%, about 300% to about 1000%, about 300% to about 1500%, about 300% to about 2000%, about 400% to about 500%, about 400% to about 750%, about 400% to about 1000%, about 400% to about 1500%, about 400% to about 2000%, about 500% to about 750%, about 500% to about 1000%, about 500% to about 2000%, about 750% to about 1000%, about 750% to about 2000%, or about 1000% to about 2000% more of the protein of interest compared to a similar cell that does not express a surface-displayed enzyme that hydrolyses a disaccharide when each are fed a growth medium comprising a disaccharide, e.g., sucrose, as its carbon source. In some embodiments, the engineered host cell which expresses a surface-displayed enzyme that hydrolyses a disaccharide secretes about 10%, about 20%, about 50%, about 100%, about 150%, about 200%, about 300%, about 400%, about 500%, about 750%, about 1000%, about 1500%, or about 2000%, about 3000%, about 4000%, about 5000%, about 7500%, about 10000% more of the protein of interest compared to a similar cell that does not express a surface-displayed enzyme that hydrolyses a disaccharide when each are fed a growth medium comprising a disaccharide, e.g., sucrose, as its carbon source. In some embodiments, the engineered host cell which expresses a surface-displayed enzyme that hydrolyses a disaccharide secretes at least about 10%, at least about 20%, at least about 50%, at least about 100%, at least about 150%, at least about 200%, at least about 300%, at least about 400%, at least about 500%, at least about 750%, at least about 1000%, at least about 1500%, or at least about 2000%, at least about 3000%, at least about 4000%, at least about 5000%, at least about 7500%, at least about 10000% more of the protein of interest compared to a similar cell that does not express a surface-displayed enzyme that hydrolyses a disaccharide when each are fed a growth medium comprising a disaccharide, e.g., sucrose, as its carbon source. In some embodiments, the engineered host cell which expresses a surface-displayed enzyme that hydrolyses a disaccharide secretes at most about 20%, about 50%, about 100%, about 150%, about 200%, about 300%, about 400%, about 500%, about 750%, about 1000%, about 1500%, or about 2000%, about 3000%, about 4000%, about 5000%, about 7500%, about 10000% more of the protein of interest compared to a similar cell that does not express a surface-displayed enzyme that hydrolyses a disaccharide when each are fed a growth medium comprising a disaccharide, e.g., sucrose, as its carbon source.

In some embodiments, the engineered host cell which expresses a surface-displayed enzyme that hydrolyses a disaccharide produces more of the protein of interest compared to a similar cell that does not express a surface-displayed enzyme that hydrolyses a disaccharide when the engineered host cell is fed a growth medium comprising a disaccharide, e.g., sucrose, as its carbon source and the similar cell is fed a growth medium comprising glucose. In some embodiments, the engineered host cell which expresses a surface-displayed enzyme that hydrolyses a disaccharide produces about 10% to about 2000% more of the protein of interest compared to a similar cell that does not express a surface-displayed enzyme that hydrolyses a disaccharide when the engineered host cell is fed a growth medium comprising a disaccharide, e.g., sucrose, as its carbon source and the similar cell is fed a growth medium comprising glucose. In some embodiments, the engineered host cell which expresses a surface-displayed enzyme that hydrolyses a disaccharide produces about 10% to about 20%, about 10% to about 50%, about 10% to about 100%, about 10% to about 150%, about 10% to about 200%, about 10% to about 300%, about 10% to about 400%, about 10% to about 500%, about 10% to about 750%, about 10% to about 1000%, about 10% to about 1500%, about 10% to about 2000%, about 20% to about 50%, about 20% to about 100%, about 20% to about 150%, about 20% to about 200%, about 20% to about 300%, about 20% to about 400%, about 20% to about 500%, about 20% to about 750%, about 20% to about 1000%, about 20% to about 1500%, about 20% to about 2000%, about 50% to about 100%, about 50% to about 150%, about 50% to about 200%, about 50% to about 300%, about 50% to about 400%, about 50% to about 500%, about 50% to about 750%, about 50% to about 1000%, about 50% to about 1500%, about 50% to about 2000%, about 100% to about 150%, about 100% to about 200%, about 100% to about 300%, about 100% to about 400%, about 100% to about 500%, about 100% to about 750%, about 100% to about 1000%, about 100% to about 2000%, about 150% to about 200%, about 150% to about 300%, about 150% to about 400%, about 150% to about 500%, about 150% to about 750%, about 150% to about 1000%, about 150% to about 1500%, about 150% to about 2000%, about 200% to about 300%, about 200% to about 400%, about 200% to about 500%, about 200% to about 750%, about 200% to about 1000%, about 200% to about 2000%, about 300% to about 400%, about 300% to about 500%, about 300% to about 750%, about 300% to about 1000%, about 300% to about 1500%, about 300% to about 2000%, about 400% to about 500%, about 400% to about 750%, about 400% to about 1000%, about 400% to about 1500%, about 400% to about 2000%, about 500% to about 750%, about 500% to about 1000%, about 500% to about 2000%, about 750% to about 1000%, about 750% to about 2000%, or about 1000% to about 2000% more of the protein of interest compared to a similar cell that does not express a surface-displayed enzyme that hydrolyses a disaccharide when the engineered host cell is fed a growth medium comprising a disaccharide, e.g., sucrose, as its carbon source and the similar cell is fed a growth medium comprising glucose. In some embodiments, the engineered host cell which expresses a surface-displayed enzyme that hydrolyses a disaccharide produces at least about 10%, at least about 20%, at least about 50%, at least about 100%, at least about 150%, at least about 200%, at least about 300%, at least about 400%, at least about 500%, at least about 750%, at least about 1000%, at least about 1500%, or at least about 2000%, at least about 3000%, at least about 4000%, at least about 5000%, at least about 7500%, at least about 10000% more of the protein of interest compared to a similar cell that does not express a surface-displayed enzyme that hydrolyses a disaccharide when the engineered host cell is fed a growth medium comprising a disaccharide, e.g., sucrose, as its carbon source and the similar cell is fed a growth medium comprising glucose. In some embodiments, the engineered host cell which expresses a surface-displayed enzyme that hydrolyses a disaccharide produces about 10%, about 20%, about 50%, about 100%, about 150%, about 200%, about 300%, about 400%, about 500%, about 750%, about 1000%, about 1500%, or about 2000%, about 3000%, about 4000%, about 5000%, about 7500%, about 10000% more of the protein of interest compared to a similar cell that does not express a surface-displayed enzyme that hydrolyses a disaccharide when the engineered host cell is fed a growth medium comprising a disaccharide, e.g., sucrose, as its carbon source and the similar cell is fed a growth medium comprising glucose. In some embodiments, the engineered host cell which expresses a surface-displayed enzyme that hydrolyses a disaccharide produces at most about 20%, about 50%, about 100%, about 150%, about 200%, about 300%, about 400%, about 500%, about 750%, about 1000%, about 1500%, or about 2000%, about 3000%, about 4000%, about 5000%, about 7500%, about 10000% more of the protein of interest compared to a similar cell that does not express a surface-displayed enzyme that hydrolyses a disaccharide when the engineered host cell is fed a growth medium comprising a disaccharide, e.g., sucrose, as its carbon source and the similar cell is fed a growth medium comprising glucose.

In some embodiments, the engineered host cell which expresses a surface-displayed enzyme that hydrolyses a disaccharide secretes more of the protein of interest compared to a similar cell that does not express a surface-displayed enzyme that hydrolyses a disaccharide when the engineered host cell is fed a growth medium comprising a disaccharide, e.g., sucrose, as its carbon source and the similar cell is fed a growth medium comprising glucose. In some embodiments, the engineered host cell which expresses a surface-displayed enzyme that hydrolyses a disaccharide secretes about 10% to about 2000% more of the protein of interest compared to a similar cell that does not express a surface-displayed enzyme that hydrolyses a disaccharide when the engineered host cell is fed a growth medium comprising a disaccharide, e.g., sucrose, as its carbon source and the similar cell is fed a growth medium comprising glucose. In some embodiments, the engineered host cell which expresses a surface-displayed enzyme that hydrolyses a disaccharide secretes about 10% to about 20%, about 10% to about 50%, about 10% to about 100%, about 10% to about 150%, about 10% to about 200%, about 10% to about 300%, about 10% to about 400%, about 10% to about 500%, about 10% to about 750%, about 10% to about 1000%, about 10% to about 1500%, about 10% to about 2000%, about 20% to about 50%, about 20% to about 100%, about 20% to about 150%, about 20% to about 200%, about 20% to about 300%, about 20% to about 400%, about 20% to about 500%, about 20% to about 750%, about 20% to about 1000%, about 20% to about 1500%, about 20% to about 2000%, about 50% to about 100%, about 50% to about 150%, about 50% to about 200%, about 50% to about 300%, about 50% to about 400%, about 50% to about 500%, about 50% to about 750%, about 50% to about 1000%, about 50% to about 1500%, about 50% to about 2000%, about 100% to about 150%, about 100% to about 200%, about 100% to about 300%, about 100% to about 400%, about 100% to about 500%, about 100% to about 750%, about 100% to about 1000%, about 100% to about 2000%, about 150% to about 200%, about 150% to about 300%, about 150% to about 400%, about 150% to about 500%, about 150% to about 750%, about 150% to about 1000%, about 150% to about 1500%, about 150% to about 2000%, about 200% to about 300%, about 200% to about 400%, about 200% to about 500%, about 200% to about 750%, about 200% to about 1000%, about 200% to about 2000%, about 300% to about 400%, about 300% to about 500%, about 300% to about 750%, about 300% to about 1000%, about 300% to about 1500%, about 300% to about 2000%, about 400% to about 500%, about 400% to about 750%, about 400% to about 1000%, about 400% to about 1500%, about 400% to about 2000%, about 500% to about 750%, about 500% to about 1000%, about 500% to about 2000%, about 750% to about 1000%, about 750% to about 2000%, or about 1000% to about 2000% more of the protein of interest compared to a similar cell that does not express a surface-displayed enzyme that hydrolyses a disaccharide when the engineered host cell is fed a growth medium comprising a disaccharide, e.g., sucrose, as its carbon source and the similar cell is fed a growth medium comprising glucose. In some embodiments, the engineered host cell which expresses a surface-displayed enzyme that hydrolyses a disaccharide secretes about 10%, about 20%, about 50%, about 100%, about 150%, about 200%, about 300%, about 400%, about 500%, about 750%, about 1000%, about 1500%, or about 2000%, about 3000%, about 4000%, about 5000%, about 7500%, about 10000% more of the protein of interest compared to a similar cell that does not express a surface-displayed enzyme that hydrolyses a disaccharide when the engineered host cell is fed a growth medium comprising a disaccharide, e.g., sucrose, as its carbon source and the similar cell is fed a growth medium comprising glucose. In some embodiments, the engineered host cell which expresses a surface-displayed enzyme that hydrolyses a disaccharide secretes at least about 10%, at least about 20%, at least about 50%, at least about 100%, at least about 150%, at least about 200%, at least about 300%, at least about 400%, at least about 500%, at least about 750%, at least about 1000%, at least about 1500%, or at least about 2000%, at least about 3000%, at least about 4000%, at least about 5000%, at least about 7500%, at least about 10000% more of the protein of interest compared to a similar cell that does not express a surface-displayed enzyme that hydrolyses a disaccharide when the engineered host cell is fed a growth medium comprising a disaccharide, e.g., sucrose, as its carbon source and the similar cell is fed a growth medium comprising glucose. In some embodiments, the engineered host cell which expresses a surface-displayed enzyme that hydrolyses a disaccharide secretes at most about 20%, about 50%, about 100%, about 150%, about 200%, about 300%, about 400%, about 500%, about 750%, about 1000%, about 1500%, or about 2000%, about 3000%, about 4000%, about 5000%, about 7500%, about 10000% more of the protein of interest compared to a similar cell that does not express a surface-displayed enzyme that hydrolyses a disaccharide when the engineered host cell is fed a growth medium comprising a disaccharide, e.g., sucrose, as its carbon source and the similar cell is fed a growth medium comprising glucose.

Cell Growth in Host Cells with an Alternative Carbon Source

In some embodiments, the engineered host cell which expresses a surface-displayed enzyme that hydrolyses a disaccharide provides about the same amount cellular proliferation and/or cellular growth compared to a similar cell that does not express a surface-displayed enzyme that hydrolyses a disaccharide. In these embodiments, “about the same amount” includes from about 1% to about 10%—more or less—cellular proliferation and/or cellular growth.

In some embodiments, the engineered host cell which expresses a surface-displayed enzyme that hydrolyses a disaccharide provides more cellular proliferation and/or cellular growth compared to a similar cell that does not express a surface-displayed enzyme that hydrolyses a disaccharide. In some embodiments, the engineered host cell which expresses a surface-displayed enzyme that hydrolyses a disaccharide provides about 1% to about 200% more cellular proliferation and/or cellular growth compared to a similar cell that does not express a surface-displayed enzyme that hydrolyses a disaccharide. In some embodiments, the engineered host cell which expresses a surface-displayed enzyme that hydrolyses a disaccharide provides about 1% to about 2%, about 1% to about 5%, about 1% to about 10%, about 1% to about 15%, about 1% to about 20%, about 1% to about 30%, about 1% to about 40%, about 1% to about 50%, about 1% to about 75%, about 1% to about 100%, about 1% to about 150%, about 1% to about 200%, about 2% to about 5%, about 2% to about 10%, about 2% to about 15%, about 2% to about 20%, about 2% to about 30%, about 2% to about 40%, about 2% to about 50%, about 2% to about 75%, about 2% to about 100%, about 2% to about 150%, about 2% to about 200%, about 5% to about 10%, about 5% to about 15%, about 5% to about 20%, about 5% to about 30%, about 5% to about 40%, about 5% to about 50%, about 5% to about 75%, about 5% to about 100%, about 5% to about 150%, about 5% to about 200%, about 10% to about 15%, about 10% to about 20%, about 10% to about 30%, about 10% to about 40%, about 10% to about 50%, about 10% to about 75%, about 10% to about 100%, about 10% to about 200%, about 15% to about 20%, about 15% to about 30%, about 15% to about 40%, about 15% to about 50%, about 15% to about 75%, about 15% to about 100%, about 15% to about 150%, about 15% to about 200%, about 20% to about 30%, about 20% to about 40%, about 20% to about 50%, about 20% to about 75%, about 20% to about 100%, about 20% to about 200%, about 30% to about 40%, about 30% to about 50%, about 30% to about 75%, about 30% to about 100%, about 30% to about 150%, about 30% to about 200%, about 40% to about 50%, about 40% to about 75%, about 40% to about 100%, about 40% to about 150%, about 40% to about 200%, about 50% to about 75%, about 50% to about 100%, about 50% to about 200%, about 75% to about 100%, about 75% to about 200%, or about 100% to about 200% more cellular proliferation and/or cellular growth compared to a similar cell that does not express a surface-displayed enzyme that hydrolyses a disaccharide. In some embodiments, the engineered host cell which expresses a surface-displayed enzyme that hydrolyses a disaccharide provides about 1%, about 2%, about 5%, about 10%, about 15%, about 20%, about 30%, about 40%, about 50%, about 75%, about 100%, about 150%, or about 200% more cellular proliferation and/or cellular growth compared to a similar cell that does not express a surface-displayed enzyme that hydrolyses a disaccharide. In some embodiments, the engineered host cell which expresses a surface-displayed enzyme that hydrolyses a disaccharide provides at least about 1%, about 2%, about 5%, about 10%, about 15%, about 20%, about 30%, about 40%, about 50%, about 75%, about 150%, or about 100% more cellular proliferation and/or cellular growth compared to a similar cell that does not express a surface-displayed enzyme that hydrolyses a disaccharide. In some embodiments, the engineered host cell which expresses a surface-displayed enzyme that hydrolyses a disaccharide provides at most about 20%, about 50%, about 100%, about 150%, about 200%, about 300%, about 400%, about 500%, about 750%, about 1000%, about 1500%, or about 2000%, about 3000%, about 4000%, about 5000%, about 7500%, about 10000% more cellular proliferation and/or cellular growth compared to a similar cell that does not express a surface-displayed enzyme that hydrolyses a disaccharide.

In some embodiments, the engineered host cell which expresses a surface-displayed enzyme that hydrolyses a disaccharide provides about the same amount cellular proliferation and/or cellular growth compared to a similar cell that does not express a surface-displayed enzyme that hydrolyses a disaccharide when each are fed a growth medium comprising glucose as its carbon source. In these embodiments, “about the same amount” includes from about 1% to about 10%—more or less—cellular proliferation and/or cellular growth.

In some embodiments, the engineered host cell which expresses a surface-displayed enzyme that hydrolyses a disaccharide provides more cellular proliferation and/or cellular growth compared to a similar cell that does not express a surface-displayed enzyme that hydrolyses a disaccharide when each are fed a growth medium comprising glucose as its carbon source. In some embodiments, the engineered host cell which expresses a surface-displayed enzyme that hydrolyses a disaccharide provides about 1% to about 200% more cellular proliferation and/or cellular growth compared to a similar cell that does not express a surface-displayed enzyme that hydrolyses a disaccharide when each are fed a growth medium comprising glucose as its carbon source. In some embodiments, the engineered host cell which expresses a surface-displayed enzyme that hydrolyses a disaccharide provides about 1% to about 2%, about 1% to about 5%, about 1% to about 10%, about 1% to about 15%, about 1% to about 20%, about 1% to about 30%, about 1% to about 40%, about 1% to about 50%, about 1% to about 75%, about 1% to about 100%, about 1% to about 150%, about 1% to about 200%, about 2% to about 5%, about 2% to about 10%, about 2% to about 15%, about 2% to about 20%, about 2% to about 30%, about 2% to about 40%, about 2% to about 50%, about 2% to about 75%, about 2% to about 100%, about 2% to about 150%, about 2% to about 200%, about 5% to about 10%, about 5% to about 15%, about 5% to about 20%, about 5% to about 30%, about 5% to about 40%, about 5% to about 50%, about 5% to about 75%, about 5% to about 100%, about 5% to about 150%, about 5% to about 200%, about 10% to about 15%, about 10% to about 20%, about 10% to about 30%, about 10% to about 40%, about 10% to about 50%, about 10% to about 75%, about 10% to about 100%, about 10% to about 200%, about 15% to about 20%, about 15% to about 30%, about 15% to about 40%, about 15% to about 50%, about 15% to about 75%, about 15% to about 100%, about 15% to about 150%, about 15% to about 200%, about 20% to about 30%, about 20% to about 40%, about 20% to about 50%, about 20% to about 75%, about 20% to about 100%, about 20% to about 200%, about 30% to about 40%, about 30% to about 50%, about 30% to about 75%, about 30% to about 100%, about 30% to about 150%, about 30% to about 200%, about 40% to about 50%, about 40% to about 75%, about 40% to about 100%, about 40% to about 150%, about 40% to about 200%, about 50% to about 75%, about 50% to about 100%, about 50% to about 200%, about 75% to about 100%, about 75% to about 200%, or about 100% to about 200% more cellular proliferation and/or cellular growth compared to a similar cell that does not express a surface-displayed enzyme that hydrolyses a disaccharide when each are fed a growth medium comprising glucose as its carbon source. In some embodiments, the engineered host cell which expresses a surface-displayed enzyme that hydrolyses a disaccharide provides about 1%, about 2%, about 5%, about 10%, about 15%, about 20%, about 30%, about 40%, about 50%, about 75%, about 100%, about 150%, or about 200% more cellular proliferation and/or cellular growth compared to a similar cell that does not express a surface-displayed enzyme that hydrolyses a disaccharide when each are fed a growth medium comprising glucose as its carbon source. In some embodiments, the engineered host cell which expresses a surface-displayed enzyme that hydrolyses a disaccharide provides at least about 1%, about 2%, about 5%, about 10%, about 15%, about 20%, about 30%, about 40%, about 50%, about 75%, about 150%, or about 100% more cellular proliferation and/or cellular growth compared to a similar cell that does not express a surface-displayed enzyme that hydrolyses a disaccharide when each are fed a growth medium comprising glucose as its carbon source. In some embodiments, the engineered host cell which expresses a surface-displayed enzyme that hydrolyses a disaccharide provides at most about 20%, about 50%, about 100%, about 150%, about 200%, about 300%, about 400%, about 500%, about 750%, about 1000%, about 1500%, or about 2000%, about 3000%, about 4000%, about 5000%, about 7500%, about 10000% more cellular proliferation and/or cellular growth compared to a similar cell that does not express a surface-displayed enzyme that hydrolyses a disaccharide when each are fed a growth medium comprising glucose as its carbon source.

In some embodiments, the engineered host cell which expresses a surface-displayed enzyme that hydrolyses a disaccharide provides more cellular proliferation and/or cellular growth compared to a similar cell that does not express a surface-displayed enzyme that hydrolyses a disaccharide when each are fed a growth medium comprising a disaccharide, e.g., sucrose, as its carbon source. In some embodiments, the engineered host cell which expresses a surface-displayed enzyme that hydrolyses a disaccharide provides about 10% to about 2000% more cellular proliferation and/or cellular growth compared to a similar cell that does not express a surface-displayed enzyme that hydrolyses a disaccharide when each are fed a growth medium comprising a disaccharide, e.g., sucrose, as its carbon source. In some embodiments, the engineered host cell which expresses a surface-displayed enzyme that hydrolyses a disaccharide provides about 10% to about 20%, about 10% to about 50%, about 10% to about 100%, about 10% to about 150%, about 10% to about 200%, about 10% to about 300%, about 10% to about 400%, about 10% to about 500%, about 10% to about 750%, about 10% to about 1000%, about 10% to about 1500%, about 10% to about 2000%, about 20% to about 50%, about 20% to about 100%, about 20% to about 150%, about 20% to about 200%, about 20% to about 300%, about 20% to about 400%, about 20% to about 500%, about 20% to about 750%, about 20% to about 1000%, about 20% to about 1500%, about 20% to about 2000%, about 50% to about 100%, about 50% to about 150%, about 50% to about 200%, about 50% to about 300%, about 50% to about 400%, about 50% to about 500%, about 50% to about 750%, about 50% to about 1000%, about 50% to about 1500%, about 50% to about 2000%, about 100% to about 150%, about 100% to about 200%, about 100% to about 300%, about 100% to about 400%, about 100% to about 500%, about 100% to about 750%, about 100% to about 1000%, about 100% to about 2000%, about 150% to about 200%, about 150% to about 300%, about 150% to about 400%, about 150% to about 500%, about 150% to about 750%, about 150% to about 1000%, about 150% to about 1500%, about 150% to about 2000%, about 200% to about 300%, about 200% to about 400%, about 200% to about 500%, about 200% to about 750%, about 200% to about 1000%, about 200% to about 2000%, about 300% to about 400%, about 300% to about 500%, about 300% to about 750%, about 300% to about 1000%, about 300% to about 1500%, about 300% to about 2000%, about 400% to about 500%, about 400% to about 750%, about 400% to about 1000%, about 400% to about 1500%, about 400% to about 2000%, about 500% to about 750%, about 500% to about 1000%, about 500% to about 2000%, about 750% to about 1000%, about 750% to about 2000%, or about 1000% to about 2000% more cellular proliferation and/or cellular growth compared to a similar cell that does not express a surface-displayed enzyme that hydrolyses a disaccharide when each are fed a growth medium comprising a disaccharide, e.g., sucrose, as its carbon source. In some embodiments, the engineered host cell which expresses a surface-displayed enzyme that hydrolyses a disaccharide provides at least about 10%, at least about 20%, at least about 50%, at least about 100%, at least about 150%, at least about 200%, at least about 300%, at least about 400%, at least about 500%, at least about 750%, at least about 1000%, at least about 1500%, or at least about 2000%, at least about 3000%, at least about 4000%, at least about 5000%, at least about 7500%, at least about 10000% more cellular proliferation and/or cellular growth compared to a similar cell that does not express a surface-displayed enzyme that hydrolyses a disaccharide when each are fed a growth medium comprising a disaccharide, e.g., sucrose, as its carbon source. In some embodiments, the engineered host cell which expresses a surface-displayed enzyme that hydrolyses a disaccharide provides about 10%, about 20%, about 50%, about 100%, about 150%, about 200%, about 300%, about 400%, about 500%, about 750%, about 1000%, about 1500%, or about 2000%, about 3000%, about 4000%, about 5000%, about 7500%, about 10000% more cellular proliferation and/or cellular growth compared to a similar cell that does not express a surface-displayed enzyme that hydrolyses a disaccharide when each are fed a growth medium comprising a disaccharide, e.g., sucrose, as its carbon source. In some embodiments, the engineered host cell which expresses a surface-displayed enzyme that hydrolyses a disaccharide provides at most about 20%, about 50%, about 100%, about 150%, about 200%, about 300%, about 400%, about 500%, about 750%, about 1000%, about 1500%, or about 2000%, about 3000%, about 4000%, about 5000%, about 7500%, about 10000% more cellular proliferation and/or cellular growth compared to a similar cell that does not express a surface-displayed enzyme that hydrolyses a disaccharide when each are fed a growth medium comprising a disaccharide, e.g., sucrose, as its carbon source.

In some embodiments, the engineered host cell which expresses a surface-displayed enzyme that hydrolyses a disaccharide provides more cellular proliferation and/or cellular growth compared to a similar cell that does not express a surface-displayed enzyme that hydrolyses a disaccharide when the engineered host cell is fed a growth medium comprising a disaccharide, e.g., sucrose, as its carbon source and the similar cell is fed a growth medium comprising glucose. In some embodiments, the engineered host cell which expresses a surface-displayed enzyme that hydrolyses a disaccharide provides about 10% to about 2000% more cellular proliferation and/or cellular growth compared to a similar cell that does not express a surface-displayed enzyme that hydrolyses a disaccharide when the engineered host cell is fed a growth medium comprising a disaccharide, e.g., sucrose, as its carbon source and the similar cell is fed a growth medium comprising glucose. In some embodiments, the engineered host cell which expresses a surface-displayed enzyme that hydrolyses a disaccharide provides about 10% to about 20%, about 10% to about 50%, about 10% to about 100%, about 10% to about 150%, about 10% to about 200%, about 10% to about 300%, about 10% to about 400%, about 10% to about 500%, about 10% to about 750%, about 10% to about 1000%, about 10% to about 1500%, about 10% to about 2000%, about 20% to about 50%, about 20% to about 100%, about 20% to about 150%, about 20% to about 200%, about 20% to about 300%, about 20% to about 400%, about 20% to about 500%, about 20% to about 750%, about 20% to about 1000%, about 20% to about 1500%, about 20% to about 2000%, about 50% to about 100%, about 50% to about 150%, about 50% to about 200%, about 50% to about 300%, about 50% to about 400%, about 50% to about 500%, about 50% to about 750%, about 50% to about 1000%, about 50% to about 1500%, about 50% to about 2000%, about 100% to about 150%, about 100% to about 200%, about 100% to about 300%, about 100% to about 400%, about 100% to about 500%, about 100% to about 750%, about 100% to about 1000%, about 100% to about 2000%, about 150% to about 200%, about 150% to about 300%, about 150% to about 400%, about 150% to about 500%, about 150% to about 750%, about 150% to about 1000%, about 150% to about 1500%, about 150% to about 2000%, about 200% to about 300%, about 200% to about 400%, about 200% to about 500%, about 200% to about 750%, about 200% to about 1000%, about 200% to about 2000%, about 300% to about 400%, about 300% to about 500%, about 300% to about 750%, about 300% to about 1000%, about 300% to about 1500%, about 300% to about 2000%, about 400% to about 500%, about 400% to about 750%, about 400% to about 1000%, about 400% to about 1500%, about 400% to about 2000%, about 500% to about 750%, about 500% to about 1000%, about 500% to about 2000%, about 750% to about 1000%, about 750% to about 2000%, or about 1000% to about 2000% more cellular proliferation and/or cellular growth compared to a similar cell that does not express a surface-displayed enzyme that hydrolyses a disaccharide when the engineered host cell is fed a growth medium comprising a disaccharide, e.g., sucrose, as its carbon source and the similar cell is fed a growth medium comprising glucose. In some embodiments, the engineered host cell which expresses a surface-displayed enzyme that hydrolyses a disaccharide provides at least about 10%, at least about 20%, at least about 50%, at least about 100%, at least about 150%, at least about 200%, at least about 300%, at least about 400%, at least about 500%, at least about 750%, at least about 1000%, at least about 1500%, or at least about 2000%, at least about 3000%, at least about 4000%, at least about 5000%, at least about 7500%, at least about 10000% more cellular proliferation and/or cellular growth compared to a similar cell that does not express a surface-displayed enzyme that hydrolyses a disaccharide when the engineered host cell is fed a growth medium comprising a disaccharide, e.g., sucrose, as its carbon source and the similar cell is fed a growth medium comprising glucose. In some embodiments, the engineered host cell which expresses a surface-displayed enzyme that hydrolyses a disaccharide provides about 10%, about 20%, about 50%, about 100%, about 150%, about 200%, about 300%, about 400%, about 500%, about 750%, about 1000%, about 1500%, or about 2000%, about 3000%, about 4000%, about 5000%, about 7500%, about 10000% more cellular proliferation and/or cellular growth compared to a similar cell that does not express a surface-displayed enzyme that hydrolyses a disaccharide when the engineered host cell is fed a growth medium comprising a disaccharide, e.g., sucrose, as its carbon source and the similar cell is fed a growth medium comprising glucose. In some embodiments, the engineered host cell which expresses a surface-displayed enzyme that hydrolyses a disaccharide provides at most about 20%, about 50%, about 100%, about 150%, about 200%, about 300%, about 400%, about 500%, about 750%, about 1000%, about 1500%, or about 2000%, about 3000%, about 4000%, about 5000%, about 7500%, about 10000% more cellular proliferation and/or cellular growth compared to a similar cell that does not express a surface-displayed enzyme that hydrolyses a disaccharide when the engineered host cell is fed a growth medium comprising a disaccharide, e.g., sucrose, as its carbon source and the similar cell is fed a growth medium comprising glucose.

Any aspect or embodiment described herein can be combined with any other aspect or embodiment as disclosed herein.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used in the specification and claims, the singular forms “a”, “an” and “the” include plural references unless the context clearly dictates otherwise.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” mean A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “substantially” is meant to be a significant extent, for the most part; or essentially. In other words, the term substantially may mean nearly exact to the desired attribute or slightly different from the exact attribute. Substantially may be indistinguishable from the desired attribute. Substantially may be distinguishable from the desired attribute but the difference is unimportant or negligible.

The terms “comprise”, “comprising”, “contain,” “containing,” “including”, “includes”, “having”, “has”, “with”, or variants thereof as used in either the present disclosure and/or in the claims, are intended to be inclusive in a manner similar to the term “comprising.”

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The terms “increased”, “increasing”, or “increase” are used herein to generally mean an increase by a statically significant amount relative to a reference level. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease in a value relative to a reference level. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level.

As used herein, “engineered” host cells are host cells which have been manipulated using genetic engineering, i.e., by human intervention. When a host cell is “engineered to underexpress” a given protein, the host cell is manipulated such that the host cell has no longer the capability to express the protein described or a functional homologue thereof such as a non-engineered host cell.

“Prior to engineering” when used in the context of host cells of the present invention means that such host cells are not engineered such that a polynucleotide encoding a recombinant protein or functional homologue thereof is not expressed.

A nucleic acid is “operably linked” when it is placed into a functional relationship with another nucleic acid sequence on the same nucleic acid molecule. For example, a promoter is operably linked with a coding sequence of a recombinant gene when it is capable of effecting the expression of that coding sequence.

For the purpose of the present invention the term “protein” is also meant to encompass functional homologues of the proteins described.

Sequence identity, such as for the purpose of assessing percent complementarity, may be measured by any suitable alignment algorithm, including but not limited to the Needleman-Wunsch algorithm (see e.g., the EMBOSS Needle aligner available at the World Wide Web at ebi.ac.uk/Tools/psa/emboss_needle/nucleotide.html, optionally with default settings), the BLAST algorithm (see e.g., the BLAST alignment tool available at blast.ncbi.nlm.nih.gov/Blast.cgi, optionally with default settings), and the Smith-Waterman algorithm (see e.g., the EMBOSS Water aligner available at the World Wide Web at ebi.ac.uk/Tools/psa/emboss_water/nucleotide.html, optionally with default settings). Optimal alignment may be assessed using any suitable parameters of a chosen algorithm, including default parameters.

The term “bird” includes both domesticated birds and non-domesticated birds such as wildlife and the like. Birds include, but are not limited to, poultry, fowl, waterfowl, game bird, ratite (e.g., flightless bird), chicken (Gallus Gallus, Gallus domesticus, or Gallus Gallus domesticus), quail, turkey, duck, ostrich (Struthio camelus), Somali ostrich (Struthio molybdophanes), goose, gull, guineafowl, pheasant, emu (Dromaius novaehollandiae), American rhea (Rhea americana), Darwin's rhea (Rhea pennata), and kiwi. Tissues, cells, and their progeny of a biological entity obtained in vivo or cultured in vitro are also encompassed. A bird may lay eggs.

### ADDITIONAL EMBODIMENTS

Embodiment 1: An engineered host cell comprising: an integrated coding sequence of a fusion protein comprising a catalytic domain of a heterologous glycosyl hydrolase; and an integrated coding sequence of a heterologous protein of interest (POI). In this embodiment, the engineered host cell does not endogenously express the glycosyl hydrolase and the POI; and the glycosyl hydrolase is anchored on the surface of the engineered host cell.

Embodiment 2: A method of growing/culturing the engineered host cell of Embodiment 1, wherein the method comprises culturing the engineered host cell with a carbon source that is not naturally utilized by the host cell in the absence of the glycosyl hydrolase.

Embodiment 3: A method for growing/culturing a host cell with a carbon source that is not naturally utilized by the host cell, the method comprising: (a) recombinantly producing in the host cell a fusion protein comprising a catalytic domain of a glycosyl hydrolase capable of digesting sucrose; optionally, wherein the glycosyl hydrolase capable of digesting sucrose is an invertase; and (b) recombinantly producing in the host cell a heterologous protein of interest (POI). In this embodiment, the host cell does not express the glycosyl hydrolase endogenously and the engineered host cell prior to step (a) does not utilize sucrose as a carbon source as efficiently as glucose, and wherein the glycosyl hydrolase is expressed on the surface of the engineered host cell.

Embodiment 4: A method for manufacturing a host cell capable of utilizing a carbon source that is not naturally utilized by the host cell, the method comprising: (a) obtaining a host cell that recombinantly expresses a fusion protein comprising a catalytic domain of a glycosyl hydrolase capable of digesting sucrose; optionally, wherein the glycosyl hydrolase capable of digesting sucrose is an invertase; and (b) genetically modifying the host cell to express a heterologous protein of interest (POI). In this embodiment, the host cell does not utilize sucrose as a carbon source as efficiently as glucose in the absence of the glycosyl hydrolase.

Embodiment 5: A method for manufacturing a host cell capable of utilizing a carbon source that is not naturally utilized by the host cell, the method comprising: (a) obtaining a host cell that recombinantly expresses a heterologous protein of interest (POI); and (b) genetically modifying the host cell to express a fusion protein comprising a catalytic domain of a glycosyl hydrolase capable of digesting sucrose, optionally, the glycosyl hydrolase capable of digesting sucrose is an invertase. In this embodiment, the host cell prior to step (b) does not utilize sucrose as a carbon source as efficiently as glucose.

Embodiment 6: The engineered host cell of Embodiment 1 or the method of Embodiment 2, wherein the glycosyl hydrolase is an invertase from S. cerevisiae.

Embodiment 7: The engineered host cell or the method of Embodiment 3, wherein the invertase is encoded by the SUC2 gene.

Embodiment 8: The engineered host cell or the method of Embodiment 3, wherein the invertase is encoded by the MAL1 gene.

Embodiment 9: The engineered host cell or the method of any one of the previous claims, wherein the fusion protein is surface-displayed on the engineered host cell; wherein the surface-displayed fusion protein comprises a catalytic domain of the glycosyl hydrolase and an anchoring domain of a glycosylphosphatidylinositol (GPI)-anchored protein, wherein the anchoring domain comprises at least about 200 amino acids and/or at least about 30% of the residues in the anchoring domain are serines or threonines.

Embodiment 10: The engineered host cell or the method of Embodiment 9, wherein the anchoring domain comprises at least about 225 amino acids, at least about 250 amino acids, at least about 275 amino acids, at least about 300 amino acids, at least about 325 amino acids, at least about 350 amino acids, at least about 375 amino acids, or at least about 400 amino acids.

Embodiment 11: The engineered host cell or the method of Embodiment 9 or Embodiment 10, wherein at least about 35% of the residues in the anchoring domain are serines or threonines, at least about 40% of the residues in the anchoring domain are serines or threonines, at least about 45% of the residues in the anchoring domain are serines or threonines, or at least about 50% of the residues in the anchoring domain are serines or threonines.

Embodiment 12: The engineered host cell or the method of Embodiment 11, wherein the serines or threonines in the anchoring domain are capable of being O-mannosylated.

Embodiment 13: The engineered host cell or the method of any one of the preceding claims, wherein a fusion protein having an anchoring domain comprising at least about 325 amino acids provides greater glycosyl hydrolase activity relative to a fusion protein having an anchoring domain comprising less than about 300 amino acids.

Embodiment 14: The engineered host cell or the method of any one of the preceding claims, wherein a fusion protein having an anchoring domain comprising at least about 300 amino acids provides greater glycosyl hydrolase activity relative to a fusion protein having an anchoring domain comprising less than about 250 amino acids.

Embodiment 15: The engineered host cell or the method of any one of the preceding claims, wherein the fusion protein comprises the anchoring domain of the GPI anchored protein.

Embodiment 16: The engineered host cell or the method of any one of the preceding claims, wherein the fusion protein comprises the GPI anchored protein without its native signal peptide or native secretory signal.

Embodiment 17: The engineered host cell or the method of any one of the preceding claims, wherein the GPI anchored protein is not native to the engineered host cell.

Embodiment 18: The engineered host cell or the method of any one of the preceding claims, wherein the GPI anchored protein is naturally expressed by a S. cerevisiae cell and the engineered host cell is not a S. cerevisiae cell.

Embodiment 19: The engineered host cell or the method of any one of the preceding claims, wherein the GPI anchored protein is selected from Tir4, Dan1, or Sed1.

Embodiment 20: The engineered host cell or the method of Embodiment 19, wherein an anchoring domain of the GPI anchored protein comprises an amino acid sequence that is at least 70% identical, at least 75% identical, at least 80% identical, at least 85% identical, at least 90% identical, or at least 95% identical, to one of SEQ ID NO: 1 to SEQ ID NO: 14.

Embodiment 21: The engineered host cell or the method of Embodiment 19 or Embodiment 20, wherein the anchoring domain of the GPI anchored protein comprises an amino acid sequence of one of SEQ ID NO: 1 to SEQ ID NO: 14.

Embodiment 22: The engineered host cell or the method of any one of the preceding claims, wherein the engineered host cell is a yeast cell.

Embodiment 23: The engineered host cell or the method of any one of the preceding claims, wherein the engineered host cell is a Pichia species.

Embodiment 24: The engineered host cell or the method of Embodiment 23, wherein the Pichia species is Pichia pastoris.

Embodiment 25: The engineered host cell or the method of any one of the preceding claims, wherein the engineered host cell comprises a genomic modification that expresses the fusion.

Embodiment 26: The engineered host cell or the method of any one of the preceding claims, wherein the fusion protein comprises a portion of the glycosyl hydrolase in addition to its catalytic domain.

Embodiment 27: The engineered host cell or the method of any one of the preceding claims, wherein the fusion protein comprises substantially the entire amino acid sequence of the glycosyl hydrolase.

Embodiment 28: The engineered host cell or the method of any one of Embodiments 20-27, wherein in the fusion protein, the catalytic domain is N-terminal to the anchoring domain.

Embodiment 29: The engineered host cell or the method of any one of Embodiments 20-27, wherein in the fusion protein, the catalytic domain is C-terminal to the anchoring domain.

Embodiment 30: The engineered host cell or the method of any one of the preceding claims, wherein the fusion protein comprises a linker between the catalytic domain and the anchoring domain.

Embodiment 31: The engineered host cell or the method of any one of the preceding claims, wherein, upon translation, the fusion protein comprises a signal peptide and/or a secretory signal.

Embodiment 32: The engineered host cell or the method of any one of the preceding claims, wherein a growth rate of the engineered host cell in a media containing sucrose as a primary carbon source is higher than a growth rate of a control host cell, wherein the control host cell is identical to the engineered host cell, except the control cell does not express the glycosyl hydrolase.

Embodiment 33: The engineered eukaryotic cell of any one of the preceding claims, wherein the engineered eukaryotic cell comprises a genomic modification that overexpresses a secreted recombinant protein and/or comprises an extrachromosomal modification that overexpresses a secreted recombinant protein.

Embodiment 34: The engineered eukaryotic cell of Embodiment 33, wherein the secreted recombinant protein is an animal protein.

Embodiment 35: The engineered eukaryotic cell of Embodiment 34, wherein the animal protein is an egg protein.

Embodiment 36: The engineered eukaryotic cell of Embodiment 35, wherein the egg protein is selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

Embodiment 37: The engineered eukaryotic cell of any one of Embodiments 33 to 36, wherein the genomic modification and/or the extrachromosomal modification that overexpresses the secreted recombinant protein comprises an inducible promoter.

Embodiment 38: The engineered eukaryotic cell of Embodiment 37, wherein the inducible promoter is an AOX1, DAK2, PEX11, FLD1, FGH1, DAS1, DAS2, CAT1, MDH3, HAC1, BiP, RAD30, RVS161-2, MPP10, THP3, TLR, GBP2, PMP20, SHB17, PEX8, PEX4, or TKL3 promoter.

Embodiment 39: The engineered eukaryotic cell of any one of Embodiments 33 to 38, wherein the genomic modification and/or the extrachromosomal modification that overexpresses a secreted recombinant protein comprises an AOX1, TDH3, MOX, RPS25A, or RPL2A terminator.

Embodiment 40: The engineered eukaryotic cell of any one of Embodiments 33 to 39, wherein the genomic modification and/or the extrachromosomal modification that overexpresses a secreted recombinant protein encodes a signal peptide and/or a secretory signal.

Embodiment 41: The engineered eukaryotic cell of any one of Embodiments 33 to 40, wherein the genomic modification and/or the extrachromosomal modification that overexpresses a secreted recombinant protein comprises codons that are optimized for the species of the engineered eukaryotic cell.

Embodiment 42: The engineered eukaryotic cell of any one of Embodiments 33 to 41, wherein the secreted recombinant protein is designed to be secreted from the cell and/or is capable of being secreted from the cell.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Growth of P. Pastoris on Carbon Sources Prior to Engineering

A background strain (strain 1) was used as a test strain. The genetic modifications present in strain 1 are deletion of AOX1 and AOX2. No target protein cassettes were present in this strain. strain 1 was plated on minimal nutrient plates containing Glucose, Fructose, or Sucrose.

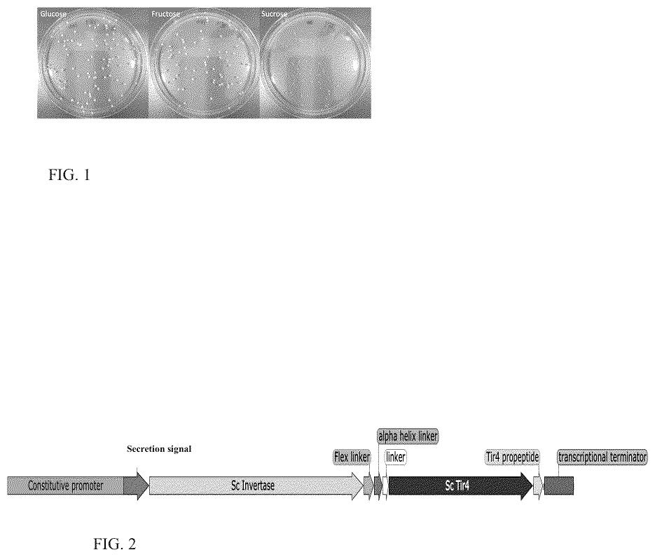

As shown in FIG. 1 the strain was able to grow on glucose and fructose at similar rates and had similar colony sizes. The strain grew to pinprick sized colonies on sucrose and stops. Without wishing to be bound by theory, it appears that sucrose source may naturally contain a small amount of hydrolyzed material, which produces separated glucose and fructose molecules.

### Example 2: Expression Constructs, Transformation, and Processing

A surface displayed invertase (suc2) from Saccharomyces cerevisiae was transformed into a high performing strain (strain 2; parent strain) previously transformed to express recombinant ovalbumin (rOVA). Strains 3 and Strain 4 are considered a “high-performing strain”. The fusion protein was driven by PGCW14, a highly expressed constitutive promoter. The DNA sequence for the expression cassette and the amino acid sequence for the fusion protein are disclosed herein respectively as SEQ ID NO: 314 and SEQ ID NO: 315. The DNA sequence encoded a secretion signal between the promoter and the SUC2 sequence, thereby permitting the invertase to become displayed on the outer surface of the cell.

In high throughput screening, those transformants which successfully expressed rOVA protein when fed sucrose, i.e., those transformants that expressed rOVA and the surface displayed invertase, were able to achieve a 50% or more increase in productivity when compared to the same strains when fed glucose alone. Candidate strains were picked into sucrose-containing media and grown for 24 hours. The starter cultures were divided equally and inoculated either sucrose-containing media or glucose-containing media for high throughput screening. Data from eight high performing candidate strains, showing growth and productivity comparisons when fed different carbon sources is shown below in Table 11. The parent strain strain 2 is unable to grow and express recombinant protein when fed sucrose, therefore all strain 2 comparisons below are made relative to its performance in glucose.

TABLE 11

| | | | | | Supernatant protein | Supernatant protein | | |
|---|---|---|---|---|---|---|---|---|
| | | | Supernatant protein | | concentration in | concentration in | Productivity in | Productivity in |
| | OD* in | OD in | concentration in | Productivity in | sucrose vs strain | glucose vs strain 2 | sucrose vs strain | glucose vs strain |
| Strain | sucrose | glucose | sucrose vs glucose | sucrose vs glucose | 2in glucose | in glucose | 2in glucose | 2in glucose |
| 1 | 16.76 | 14.02 | 0.81 | 0.68 | 1.09 | 1.34 | 0.77 | 1.13 |
| 2 | 17.16 | 14.2 | 0.92 | 0.76 | 1.04 | 1.13 | 0.71 | 0.93 |
| 3 | 15.8 | 13.37 | 0.79 | 0.67 | 0.99 | 1.25 | 0.74 | 1.10 |
| 4 | 16.41 | 14.29 | 1.15 | 1.00 | 0.98 | 0.85 | 0.71 | 0.70 |
| 5 | 19.29 | 17.66 | 1.15 | 1.05 | 0.87 | 0.76 | 0.53 | 0.50 |
| 6 | 16.66 | 14.59 | 0.76 | 0.66 | 0.87 | 1.14 | 0.61 | 0.92 |
| 7 | 17.04 | 13.67 | 0.67 | 0.54 | 0.75 | 1.12 | 0.52 | 0.96 |
| 8 | 16.14 | 14.45 | 0.61 | 0.55 | 0.68 | 1.11 | 0.49 | 0.90 |

In Table 11, above, optical density (OD) is an indirect measure of cell density in culture, thus reflecting cell growth. For reference, strain 2 achieved OD's of 1.14 in sucrose (practically no growth) and 11.76 in glucose. The columns of Table 11 reciting “vs. strain 2” show a relative comparison of protein production of a candidate strain using sucrose or glucose as a food source compared to strain 2 using glucose as a food source. Numbers shown in columns 3-8 show relative ratios of protein production. The ratios shown in Table 11 are described below:

The column entitled: “Supernatant protein concentration in sucrose vs glucose” in Table 11 shows ratios of the concentration of recombinantly-expressed protein measured in the culture supernatant when comparing sucrose-fed cultures to glucose-fed cultures.

The column entitled: “Productivity in sucrose vs glucose” in Table 11 shows ratios comparing sucrose-fed cultures to glucose-fed cultures. Productivity was measured by protein concentration in supernatant divided by OD; by dividing by the culture's OD, a “per-cell” protein productivity was determined.

The column entitled: “Supernatant protein concentration in sucrose vs strain 2 in glucose” in Table 11 shows ratios of protein concentration measured in the culture supernatant when comparing sucrose-fed cultures of each candidate strain to glucose-fed cultures of the parent strain strain 2.

The column entitled: “Supernatant protein concentration in glucose vs strain 2 in glucose” in Table 11 shows ratios of protein concentration measured in the culture supernatant when comparing glucose-fed cultures of each candidate strain to glucose-fed cultures of the parent strain strain 2.

The column entitled: “Productivity in sucrose vs strain 2 in glucose” in Table 11 shows ratios of per cell productivity comparing sucrose-fed cultures of each candidate strain to glucose-fed cultures of the parent strain strain 2.

The column entitled: “Productivity in glucose vs strain 2 in glucose” in Table 11 shows ratios of per cell productivity comparing glucose-fed cultures of each candidate strain to glucose-fed cultures of the parent strain strain 2.

All candidate strains grew more cell mass when fed sucrose when compared to their cell mass when fed glucose. When considering protein concentration and productivity by the candidate strains when fed sucrose in comparison to the strain 2 strain when fed glucose, candidate strains 1 to 4 each performed well, with similar supernatant protein concentration to parent and from about 71% to 77% productivity. The data herein show that candidate strains that were fed sucrose were as efficient as making protein as the strain 2 parent strain fed with glucose.

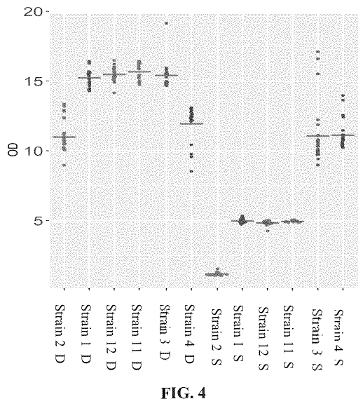

FIG. 4 illustrates the comparison of growth on glucose (G) (shown as “_D in FIG. 4) vs sucrose (S) (shown as “_S” in FIG. 4) of various background strains and the candidate strains which were engineered to display invertase. Strain 2, strain 1, and strain 11 are background strains which express rOVA, strain 12 is a “wild-type” P. pastoris strain, and strain 3 and strain 4 were engineered express the Suc2 construct (strain 2+Suc2-Tir4, i.e., the surface displayed invertase fusion protein). Although each strain achieved OD600 values of 10 or higher when grown in glucose-containing media, only the strains which were engineered to express the surface displayed invertase fusion protein could achieve such levels with sucrose was the main carbon source in a media. All other media components were the same, final concentrations of sugar (either sucrose or glucose) in the media were 0.5%. OD600 measures the amount turbidity of a culture, which is related to the amount of cells present in the culture and is an indicator of cell proliferation/cell growth.

### Example 3: Growth of Engineered P. pastoris Using Sucrose as a Carbon Source

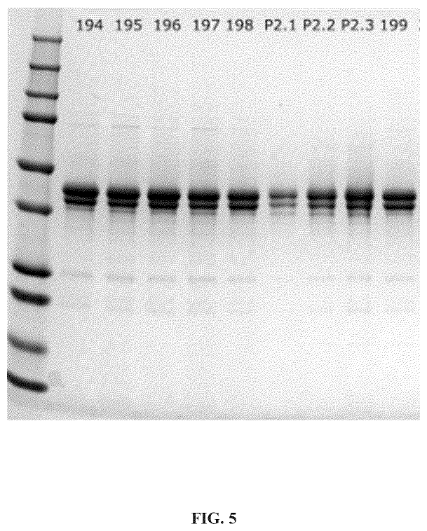

A surface displayed invertase (suc2) from Saccharomyces cerevisiae was transformed into a P2 strain (strain 5) which was previously transformed to express recombinant ovalbumin (rOVA). Performance of the suc2-expressing strain, referred to herein is strain 6, was evaluated in a 250 mL bioreactor. The strain 6 strain produced rOVA at a similar titer and quality as the strain 5 when fed either glucose or sucrose, as measured qualitatively by SDS-PAGE (FIG. 5) and quantitatively by HPLC (Table 12). The strain 6 strain and the control strain 5 strain (which expressed rOVA but did not express suc2) were run in bioreactors in parallel to undergo similar fermentation processes. Inclusion of either glucose or sucrose as the carbon source in a culturing media was the only variable. Strain 6 was further evaluated in a 50:50 glucose:fructose feed (not shown). The strain performed similarly in the 50:50 feed compared to sucrose feed, suggesting that its metabolism when fed sucrose is not rate limited by the sucrose hydrolysis step carried out by SUC2.

In FIG. 5 and Table 12: 194 and 195 are data for parent strain (strain 5) grown on glucose, 196 and 197 are data for a surface displayed suc2-expressing strain strain 6 grown on glucose; and 198 and 199 are data for a suc2-expressing strain 6 grown on sucrose. P2.1-P2-3 are data the standard strain 5 sample loaded as a reference. P2.1-P2.3 are a protein standard (not generated by strain 5) of known concentration loaded for reference. The standard sample was generated using an in-house strain expressing P2 and the protein was column purified to be used as an internal protein standard.

The performance measured by HPLC (Table 12) represents the broth titer of fermentation normalized to the average of the control (strain 5 that lacks suc2, fed glucose as the carbon source, run on Bay 194 and Bay 195).

TABLE 12

| | | Carbon | Performance* normalized |
|---|---|---|---|
| Sample | Strain | source | average of control |
| Bay 194 | strain 5 control | Glucose | 1.03 |
| Bay 195 | strain 5 control | Glucose | 0.97 |
| Bay 196 | strain 6 | Glucose | 1.02 |
| Bay 197 | strain 6 | Glucose | 1.01 |
| Bay 198 | strain 6 | Sucrose | 0.99 |
| Bay 199 | strain 6 | Sucrose | 0.99 |

*Broth titer of fermentation

To determine if hydrolysis of sucrose into glucose and fructose by the surface displayed invertase fusion protein affects cell growth and/or recombinant protein expression amounts, the strain 6 strain was a fed a media comprising equal parts of glucose and fructose and compared to the strain 6 strain fed a medium comprising an equivalent amount of sucrose. The strain 6 strain performed similarly when the two conditions were compared as shown in Table 12; suggesting that the extra step of hydrolyzing sucrose is not rate limiting to the cell growth and protein expression processes.

1. An engineered host cell comprising:
an integrated coding sequence of a fusion protein comprising a catalytic domain of a heterologous glycosyl hydrolase; and
an integrated coding sequence of a heterologous protein of interest (POI);
wherein the engineered host cell does not endogenously express the glycosyl hydrolase and the POI; and
wherein the glycosyl hydrolase is anchored on the surface of the engineered host cell.

2. The engineered host cell of claim 1, wherein the glycosyl hydrolase is an invertase selected from: S. cerevisiae, Kluyveromyces lactis, Cyberlindnera jadinii, Oryza sativa japonica (rice), Oryza sativa japonica (rice), Arabidopsis thaliana, Arabidopsis thaliana, Arabidopsis thaliana, Rattus norvegicus (rat), Oryctolagus cuniculus (Rabbit), and Homo sapiens.

3-4. (canceled)

5. The engineered host cell of claim 1, wherein the invertase is encoded by a gene selected from: SUC2, MAL1, invertase (INV1), cytosolic invertase 1 (CINV1), CIN2, CINV1, INVA, INVE, and sucrase-isomaltase (SI) gene.

6. The engineered host cell of claim 1, wherein the fusion protein is surface-displayed on the engineered host cell; wherein the surface-displayed fusion protein comprises a catalytic domain of the glycosyl hydrolase and an anchoring domain of a glycosylphosphatidylinositol (GPI)-anchored protein, wherein the anchoring domain comprises at least about 200 amino acids and/or at least about 30% of the residues in the anchoring domain are serines or threonines.

7-8. (canceled)

9. The engineered host cell of claim 8, wherein the serines or threonines in the anchoring domain are capable of being O-mannosylated.

10. The engineered host cell of claim 6, wherein a fusion protein having an anchoring domain comprising at least about 325 amino acids provides greater glycosyl hydrolase activity relative to a fusion protein having an anchoring domain comprising less than about 300 amino acids or less than about 250 amino acids.

11-12. (canceled)

13. The engineered host cell of claim 1, wherein the fusion protein comprises the GPI anchored protein without its native signal peptide or native secretory signal to the engineering host cell.

14. (canceled)

15. The engineered host cell of claim 1, wherein the GPI anchored protein is naturally expressed by a S. cerevisiae cell and the engineered host cell is not a S. cerevisiae cell.

16. The engineered host cell of claim 13, wherein the GPI anchored protein is selected from Tir4, Dan1, or Sed1.

17. The engineered host cell of claim 1, wherein an anchoring domain of the GPI anchored protein comprises an amino acid sequence that is at least 70% identical to one of SEQ ID NO: 1 to SEQ ID NO: 14.

18. (canceled)

19. The engineered host cell of claim 1, wherein the engineered host cell is a yeast cell or a Pichia species.

20. (canceled)

21. The engineered host cell of claim 19, wherein the Pichia species is Pichia pastoris.

22. The engineered host cell of claim 1, wherein the engineered host cell comprises a genomic modification that expresses the fusion or a portion of the glycosyl hydrolase in addition to its catalytic domain.

23-24. (canceled)

25. The engineered host cell of claim 1, wherein in the fusion protein, the catalytic domain is N-terminal to the anchoring domain, or wherein in the fusion protein, the catalytic domain is C-terminal to the anchoring domain.

26. (canceled)

27. The engineered host cell of claim 1, wherein the fusion protein comprises a linker between the catalytic domain and the anchoring domain.

28. (canceled)

29. The engineered host cell of claim 1, wherein a growth rate of the engineered host cell in a media containing sucrose as a primary carbon source is higher than a growth rate of a control host cell, wherein the control host cell is identical to the engineered host cell, except the control cell does not express the glycosyl hydrolase.

30. The engineered eukaryotic cell of claim 1, wherein the engineered eukaryotic cell comprises a genomic modification that overexpresses a secreted recombinant protein and/or comprises an extrachromosomal modification that overexpresses a secreted recombinant protein.

31. The engineered eukaryotic cell of claim 30, wherein the secreted recombinant protein is an egg protein.

32. (canceled)

33. The engineered eukaryotic cell of claim 31, wherein the egg protein is selected from the group consisting of ovalbumin, ovomucoid, lysozyme ovoglobulin G2, ovoglobulin G3, α-ovomucin, β-ovomucin, ovotransferrin, ovoinhibitor, ovoglycoprotein, flavoprotein, ovomacroglobulin, ovostatin, cystatin, avidin, ovalbumin related protein X, and ovalbumin related protein Y.

34. The engineered eukaryotic cell of claim 30, wherein the genomic modification and/or the extrachromosomal modification that overexpresses the secreted recombinant protein comprises an inducible promoter selected from an A0X1, DAK2, PEX11, FLD1, FGH1, DAS1, DAS2, CAT1, MDH3, HAC1, BiP, RAD30, RVS161-2, MPP10, THP3, TLR, GBP2, PMP20, SHB17, PEX8, PEX4, or TKL3 promoter, and/or a terminator selected from an AOX1, TDH3, MOX, RPS25A, or RPL2A terminator.

35-36. (canceled)

37. The engineered eukaryotic cell of claim 30, wherein the genomic modification and/or the extrachromosomal modification that overexpresses a secreted recombinant protein encodes a signal peptide, a secretory signal, and/or codons that are optimized for the species of the engineered eukaryotic cell.

38. (canceled)

39. The engineered eukaryotic cell of claim 30, wherein the secreted recombinant protein is designed to be secreted from the cell and/or is capable of being secreted from the cell.

40. The engineered eukaryotic cell of claim 1, wherein the fusion protein comprises an amino acid sequence that is at least 80%, at least 85%, at least 90%, at least 95%, at least 96%, at least 97%, at least 98%, at least 99%, or 100% identical to the amino acid sequence selected from SEQ ID NOs: 315, 332-335, and 342.

41. The engineered eukaryotic cell of claim 1, wherein the fusion protein comprises a nucleotide sequence that is at least 80%, at least 85%, at least 90%, at least 95%, at least 96%, at least 97%, at least 98%, at least 99%, or 100% identical to the nucleotide sequence of SEQ ID ON: 314.

42. A method of growing/culturing the engineered host cell of claim 1, wherein the method comprises culturing the engineered host cell with a carbon source that is not naturally utilized by the host cell in the absence of the glycosyl hydrolase.

43. A method for growing/culturing a host cell with a carbon source that is not naturally utilized by the host cell, the method comprising:
(a) recombinantly producing in the host cell, a fusion protein comprising a catalytic domain of a glycosyl hydrolase capable of digesting sucrose; optionally, wherein the glycosyl hydrolase capable of digesting sucrose is an invertase;
(b) recombinantly producing in the host cell a heterologous protein of interest (POI);
wherein the host cell does not express the glycosyl hydrolase endogenously;
wherein the engineered host cell prior to step (a) does not utilize sucrose as a carbon source as efficiently as glucose, and wherein the glycosyl hydrolase is expressed on the surface of the engineered host cell.

44. A method for manufacturing a host cell capable of utilizing a carbon source that is not naturally utilized by the host cell, the method comprising:
(a) obtaining a host cell that recombinantly expresses a fusion protein comprising a catalytic domain of a glycosyl hydrolase capable of digesting sucrose,
wherein the glycosyl hydrolase capable of digesting sucrose is an invertase; and
(b) genetically modifying the host cell to express a heterologous protein of interest (POI);
wherein the host cell does not utilize sucrose as a carbon source as efficiently as glucose in the absence of the glycosyl hydrolase.

45. A method for manufacturing a host cell capable of utilizing a carbon source that is not naturally utilized by the host cell, the method comprising:
(a) obtaining a host cell that recombinantly expresses a heterologous protein of interest (POI); and
(b) genetically modifying the host cell to express a fusion protein comprising a catalytic domain of a glycosyl hydrolase capable of digesting sucrose;
wherein the glycosyl hydrolase capable of digesting sucrose is an invertase;
wherein the host cell prior to step (b) does not utilize sucrose as a carbon source as efficiently as glucose.


SEQUENCE LISTING (344 sequences)

SEQ ID NO: 1 - protein, 368 aa, Saccharomyces cerevisiae
QINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVASSSSEVASSSVASTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTENGAAKAVIGMGAGALAAVAAMLL

SEQ ID NO: 2 - protein, 390 aa, Saccharomyces cerevisiae
MAYSKITLLAALAAIAYAQTQAQINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVASSSSEVASSSVASTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTENGAAKAVIGMGAGALAAVAAMLL

SEQ ID NO: 3 - protein, 465 aa, Saccharomyces cerevisiae
QINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVAPSSSEVVSSSVAPSSSEVVSSSVASSSSEVASSSVAPSSSEVVSSSVASSSSEVASSSVAPSSSEVVSSSVAPSSSEVVSSSVASSSSEVASSSVAPSSSEVVSSSVASSTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTENGAAKAVIGMGAGALAAVAAMLL

SEQ ID NO: 4 - protein, 487 aa, Saccharomyces cerevisiae
MAYSKITLLAALAAIAYAQTQAQINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVAPSSSEVVSSSVAPSSSEVVSSSVASSSSEVASSSVAPSSSEVVSSSVASSSSEVASSSVAPSSSEVVSSSVAPSSSEVVSSSVASSSSEVASSSVAPSSSEVVSSSVASSTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTENGAAKAVIGMGAGALAAVAAMLL

SEQ ID NO: 5 - protein, 279 aa, Saccharomyces cerevisiae
ASVTTTLSPYDERVNLIELAVYVSDIGAHLSEYYAFQALHKTETYPPEIAKAVFAGGDFTTMLTGISGDEVTRMITGVPWYSTRLMGAISEALANEGIATAVPASTTEASSTSTSEASSAATESSSSSESSAETSSNAASTQATVSSESSSAASTIASSAESSVASSVASSVASSASFANTTAPVSSTSSISVTPVVQNGTDSTVTKTQASTVETTITSCSNNVCSTVTKPVSSKAQSTATSVTSSASRVIDVTTNGANKFNNGVFGAAAIAGAAALLL

SEQ ID NO: 6 - protein, 298 aa, Saccharomyces cerevisiae
MSRISILAVAAALVASATAASVTTTLSPYDERVNLIELAVYVSDIGAHLSEYYAFQALHKTETYPPEIAKAVFAGGDFTTMLTGISGDEVTRMITGVPWYSTRLMGAISEALANEGIATAVPASTTEASSTSTSEASSAATESSSSSESSAETSSNAASTQATVSSESSSAASTIASSAESSVASSVASSVASSASFANTTAPVSSTSSISVTPVVQNGTDSTVTKTQASTVETTITSCSNNVCSTVTKPVSSKAQSTATSVTSSASRVIDVTTNGANKFNNGVFGAAAIAGAAALLL

SEQ ID NO: 7 - protein, 1142 aa, Saccharomyces cerevisiae
ITATTTLSPYDERVNLIELAVYVSDIRAHIFQYYSFRNHHKTETYPSEIAAAVFDYGDFTTRLTGISGDEVTRMITGVPWYSTRLKPAISSALSKDGIYTAIPTSTSTTTTKSSTSTTPTTTITSTTSTTSTTPTTSTTSTTPTTSTTSTTPTTSTTSTTPTTSTTSTTPTTSTTSTTPTTSTTSTTPTTSTTSTTPTTSTTSTTPTTSTTPTTSTTSTTSQTSTKSTTPTTSSTSTTPTTSTTPTTSTTSTAPTTSTTSTTSTTSTISTAPTTSTTSSTFSTSSASASSVISTTATTSTTFASLTTPATSTASTDHTTSSVSTTNAFTTSATTTTTSDTYISSSSPSQVTSSAEPTTVSEVTSSVEPTRSSQVTSSAEPTTVSEFTSSVEPTRSSQVTSSAEPTTVSEFTSSVEPTRSSQVTSSAEPTTVSEFTSSVEPTRSSQVTSSAEPTTVSEFTSSVEPTRSSQVTSSAEPTTVSEFTSSVEPIRSSQVTSSAEPTTVSEVTSSVEPIRSSQVTTTEPVSSFGSTFSEITSSAEPLSFSKATTSAESISSNQITISSELIVSSVITSSSEIPSSIEVLTSSGISSSVEPTSLVGPSSDESISSTESLSATSTFTSAVVSSSKAADFFTRSTVSAKSDVSGNSSTQSTTFFATPSTPLAVSSTVVTSSTDSVSPNIPFSEISSSPESSTAITSTSTSFIAERTSSLYLSSSNMSSFTLSTFTVSQSIVSSFSMEPTSSVASFASSSPLLVSSRSNCSDARSSNTISSGLFSTIENVRNATSTFTNLSTDEIVITSCKSSCTNEDSVLTKTQVSTVETTITSCSGGICTTLMSPVTTINAKANTLTTTETSTVETTITTCPGGVCSTLTVPVTTITSEATTTATISCEDNEEDITSTETELLTLETTITSCSGGICTTLMSPVTTINAKANTLTTTETSTVETTITTCSGGVCSTLTVPVTTITSEATTTATISCEDNEEDVASTKTELLTMETTITSCSGGICTTLMSPVSSFNSKATTSNNAESTIPKAIKVSCSAGACTTLTTVDAGISMFTRTGLSITQTTVTNCSGGTCTMLTAPIATATSKVISPIPKASSATSIAHSSASYTVSINTNGAYNFDKDNIFGTAIVAVVALLLL

SEQ ID NO: 8 - protein, 1161 aa, Saccharomyces cerevisiae
MVNISIVAGIVALATSAAAITATTTLSPYDERVNLIELAVYVSDIRAHIFQYYSFRNHHKTETYPSEIAAAVFDYGDFTTRLTGISGDEVTRMITGVPWYSTRLKPAISSALSKDGIYTAIPTSTSTTTTKSSTSTTPTTTITSTTSTTSTTPTTSTTSTTPTTSTTSTTPTTSTTSTTPTTSTTSTTPTTSTTSTTPTTSTTSTTPTTSTTSTTPTTSTTSTTPTTSTTPTTSTTSTTSQTSTKSTTPTTSSTSTTPTTSTTPTTSTTSTAPTTSTTSTTSTTSTISTAPTTSTTSSTFSTSSASASSVISTTATTSTTFASLTTPATSTASTDHTTSSVSTTNAFTTSATTTTTSDTYISSSSPSQVTSSAEPTTVSEVTSSVEPTRSSQVTSSAEPTTVSEFTSSVEPTRSSQVTSSAEPTTVSEFTSSVEPTRSSQVTSSAEPTTVSEFTSSVEPTRSSQVTSSAEPTTVSEFTSSVEPTRSSQVTSSAEPTTVSEFTSSVEPIRSSQVTSSAEPTTVSEVTSSVEPIRSSQVTTTEPVSSFGSTFSEITSSAEPLSFSKATTSAESISSNQITISSELIVSSVITSSSEIPSSIEVLTSSGISSSVEPTSLVGPSSDESISSTESLSATSTFTSAVVSSSKAADFFTRSTVSAKSDVSGNSSTQSTTFFATPSTPLAVSSTVVTSSTDSVSPNIPFSEISSSPESSTAITSTSTSFIAERTSSLYLSSSNMSSFTLSTFTVSQSIVSSFSMEPTSSVASFASSSPLLVSSRSNCSDARSSNTISSGLFSTIENVRNATSTFTNLSTDEIVITSCKSSCTNEDSVLTKTQVSTVETTITSCSGGICTTLMSPVTTINAKANTLTTTETSTVETTITTCPGGVCSTLTVPVTTITSEATTTATISCEDNEEDITSTETELLTLETTITSCSGGICTTLMSPVTTINAKANTLTTTETSTVETTITTCSGGVCSTLTVPVTTITSEATTTATISCEDNEEDVASTKTELLTMETTITSCSGGICTTLMSPVSSFNSKATTSNNAESTIPKAIKVSCSAGACTTLTTVDAGISMFTRTGLSITQTTVTNCSGGTCTMLTAPIATATSKVISPIPKASSATSIAHSSASYTVSINTNGAYNFDKDNIFGTAIVAVVALLLL

SEQ ID NO: 9 - protein, 631 aa, Saccharomyces cerevisiae
ININDITFSNLEITPLTANKQPDQGWTATFDFSIADASSIREGDEFTLSMPHVYRIKLLNSSQTATISLADGTEAFKCYVSQQAAYLYENTTFTCTAQNDLSSYNTIDGSITFSLNFSDGGSSYEYELENAKFFKSGPMLVKLGNQMSDVVNFDPAAFTENVFHSGRSTGYGSFESYHLGMYCPNGYFLGGTEKIDYDSSNNNVDLDCSSVQVYSSNDFNDWWFPQSYNDTNADVTCFGSNLWITLDEKLYDGEMLWVNALQSLPANVNTIDHALEFQYTCLDTIANTTYATQFSTTREFIVYQGRNLGTASAKSSFISTTTTDLTSINTSAYSTGSISTVETGNRTTSEVISHVVTTSTKLSPTATTSLTIAQTSIYSTDSNITVGTDIHTTSEVISDVETISRETASTVVAAPTSTTGWTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 10 - protein, 650 aa, Saccharomyces cerevisiae
MFTFLKIILWLFSLALASAININDITFSNLEITPLTANKQPDQGWTATFDFSIADASSIREGDEFTLSMPHVYRIKLLNSSQTATISLADGTEAFKCYVSQQAAYLYENTTFTCTAQNDLSSYNTIDGSITFSLNFSDGGSSYEYELENAKFFKSGPMLVKLGNQMSDVVNFDPAAFTENVFHSGRSTGYGSFESYHLGMYCPNGYFLGGTEKIDYDSSNNNVDLDCSSVQVYSSNDFNDWWFPQSYNDTNADVTCFGSNLWITLDEKLYDGEMLWVNALQSLPANVNTIDHALEFQYTCLDTIANTTYATQFSTTREFIVYQGRNLGTASAKSSFISTTTTDLTSINTSAYSTGSISTVETGNRTTSEVISHVVTTSTKLSPTATTSLTIAQTSIYSTDSNITVGTDIHTTSEVISDVETISRETASTVVAAPTSTTGWTGAMNTYISQFTSSSFATINSTPIISSSAVFETSDASIVNVHTENITNTAAVPSEEPTFVNATRNSLNSFCSSKQPSSPSSYTSSPLVSSLSVSKTLLSTSFTPSVPTSNTYIKTKNTGYFEHTALTTSSVGLNSFSETAVSSQGTKIDTFLVSSLIAYPSSASGSQLSGIQQNFTSTSLMISTYEGKASIFFSAELGSIIFLLLSYLLF

SEQ ID NO: 11 - protein, 1587 aa, Saccharomyces cerevisiae
QIVFYQNSSTSLPVPTLVSTSIADFHESSSTGEVQYSSSYSYVQPSIDSFTSSSFLTSFEAPTETSSSYAVSSSLITSDTFSSYSDIFDEETSSLISTSAASSEKASSTLSSTAQPHRTSHSSSSFELPVTAPSSSSLPSSTSLTFTSVNPSQSWTSFNSEKSSALSSTIDFTSSEISGSTSPKSLESFDTTGTITSSYSPSPSSKNSNQTSLLSPLEPLSSSSGDLILSSTIQATTNDQTSKTIPTLVDATSSLPPTLRSSSMAPTSGSDSISHNFTSPPSKTSGNYDVLTSNSIDPSLFTTTSEYSSTQLSSLNRASKSETVNFTASIASTPFGTDSATSLIDPISSVGSTASSFVGISTANFSTQGNSNYVPESTASGSSQYQDWSSSSLPLSQTTWVVINTTNTQGSVTSTTSPAYVSTATKTVDGVITEYVTWCPLTQTKSQAIGVSSSISSVPQASSFSGSSILSSNSSTLAASNNVPESTASGSSQYQDWSSSSLPLSQTTWVVINTTNTQGSVTSTTSPAYVSTATKTVDGVITEYVTWCPLTQTKSQAIGISSSTISATQTSKPSSILTLGISTLQLSDATFKGTETINTHLMTESTSITEPTYFSGTSDSFYLCTSEVNLASSLSSYPNFSSSEGSTATITNSTVTFGSTSKYPSTSVSNPTEASQHVSSSVNSLTDFTSNSTETIAVISNIHKTSSNKDYSLTTTQLKTSGMQTLVLSTVTTTVNGAATEYTTWCPASSIAYTTSISYKTLVLTTEVCSHSECTPTVITSVTATSSTIPLLSTSSSTVLSSTVSEGAKNPAASEVTINTQVSATSEATSTSTQVSATSATATASESSTTSQVSTASETISTLGTQNFTTTGSLLFPALSTEMINTTVVSRKTLIISTEVCSHSKCVPTVITEVVTSKGTPSNGHSSQTLQTEAVEVTLSSHQTVTMSTEVCSNSICTPTVITSVQMRSTPFPYLTSSTSSSSLASTKKSSLEASSEMSTFSVSTQSLPLAFTSSEKRSTTSVSQWSNTVLTNTIMSSSSNVISTNEKPSSTTSPYNFSSGYSLPSSSTPSQYSLSTATTTINGIKTVYTTWCPLAEKSTVAASSQSSRSVDRFVSSSKPSSSLSQTSIQYTLSTATTTISGLKTVYTTWCPLTSKSTLGATTQTSSTAKVRITSASSATSTSISLSTSTESESSSGYLSKGVCSGTECTQDVPTQSSSPASTLAYSPSVSTSSSSSFSTTTASTLTSTHTSVPLLPSSSSISASSPSSTSLLSTSLPSPAFTSSTLPTATAVSSSTFIASSLPLSSKSSLSLSPVSSSILMSQFSSSSSSSSSLASLPSLSISPTVDTVSVLQPTTSIATLTCTDSQCQQEVSTICNGSNCDDVTSTATTPPSTVTDTMTCTGSECQKTTSSSCDGYSCKVSETYKSSATISACSGEGCQASATSELNSQYVTMTSVITPSAITTTSVEVHSTESTISITTVKPVTYTSSDTNGELITITSSSQTVIPSVTTIITRTKVAITSAPKPTTTTYVEQRLSSSGIATSFVAAASSTWITTPIVSTYAGSASKFLCSKFFMIMVMVINFI

SEQ ID NO: 12 - protein, 1609 aa, Saccharomyces cerevisiae
MNSFASLGLIYSVVNLLTRVEAQIVFYQNSSTSLPVPTLVSTSIADFHESSSTGEVQYSSSYSYVQPSIDSFTSSSFLTSFEAPTETSSSYAVSSSLITSDTFSSYSDIFDEETSSLISTSAASSEKASSTLSSTAQPHRTSHSSSSFELPVTAPSSSSLPSSTSLTFTSVNPSQSWTSFNSEKSSALSSTIDFTSSEISGSTSPKSLESFDTTGTITSSYSPSPSSKNSNQTSLLSPLEPLSSSSGDLILSSTIQATTNDQTSKTIPTLVDATSSLPPTLRSSSMAPTSGSDSISHNFTSPPSKTSGNYDVLTSNSIDPSLFTTTSEYSSTQLSSLNRASKSETVNFTASIASTPFGTDSATSLIDPISSVGSTASSFVGISTANFSTQGNSNYVPESTASGSSQYQDWSSSSLPLSQTTWVVINTTNTQGSVTSTTSPAYVSTATKTVDGVITEYVTWCPLTQTKSQAIGVSSSISSVPQASSFSGSSILSSNSSTLAASNNVPESTASGSSQYQDWSSSSLPLSQTTWVVINTTNTQGSVTSTTSPAYVSTATKTVDGVITEYVTWCPLTQTKSQAIGISSSTISATQTSKPSSILTLGISTLQLSDATFKGTETINTHLMTESTSITEPTYFSGTSDSFYLCTSEVNLASSLSSYPNFSSSEGSTATITNSTVTFGSTSKYPSTSVSNPTEASQHVSSSVNSLTDFTSNSTETIAVISNIHKTSSNKDYSLTTTQLKTSGMQTLVLSTVTTTVNGAATEYTTWCPASSIAYTTSISYKTLVLTTEVCSHSECTPTVITSVTATSSTIPLLSTSSSTVLSSTVSEGAKNPAASEVTINTQVSATSEATSTSTQVSATSATATASESSTTSQVSTASETISTLGTQNFTTTGSLLFPALSTEMINTTVVSRKTLIISTEVCSHSKCVPTVITEVVTSKGTPSNGHSSQTLQTEAVEVTLSSHQTVTMSTEVCSNSICTPTVITSVQMRSTPFPYLTSSTSSSSLASTKKSSLEASSEMSTFSVSTQSLPLAFTSSEKRSTTSVSQWSNTVLTNTIMSSSSNVISTNEKPSSTTSPYNFSSGYSLPSSSTPSQYSLSTATTTINGIKTVYTTWCPLAEKSTVAASSQSSRSVDRFVSSSKPSSSLSQTSIQYTLSTATTTISGLKTVYTTWCPLTSKSTLGATTQTSSTAKVRITSASSATSTSISLSTSTESESSSGYLSKGVCSGTECTQDVPTQSSSPASTLAYSPSVSTSSSSSFSTTTASTLTSTHTSVPLLPSSSSISASSPSSTSLLSTSLPSPAFTSSTLPTATAVSSSTFIASSLPLSSKSSLSLSPVSSSILMSQFSSSSSSSSSLASLPSLSISPTVDTVSVLQPTTSIATLTCTDSQCQQEVSTICNGSNCDDVTSTATTPPSTVTDTMTCTGSECQKTTSSSCDGYSCKVSETYKSSATISACSGEGCQASATSELNSQYVTMTSVITPSAITTTSVEVHSTESTISITTVKPVTYTSSDTNGELITITSSSQTVIPSVTTIITRTKVAITSAPKPTTTTYVEQRLSSSGIATSFVAAASSTWITTPIVSTYAGSASKFLCSKFFMIMVMVINFI

SEQ ID NO: 13 - protein, 320 aa, Saccharomyces cerevisiae
QFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 14 - protein, 338 aa, Saccharomyces cerevisiae
MKLSTVLLSAGLASTTLAQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 15 - protein, 513 aa, Saccharomyces cerevisiae
SMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 16 - protein, 532 aa, Saccharomyces cerevisiae
MLLQAFLFLLAGFAAKISASMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 17 - protein, 564 aa, unidentified
MTIESQEPWWKSAVVYQVWPASFKDSNGDGIGDLNGITSELDHIKSLGTDVIWLSPHYASPLDDMGYDISDYNAINPQFGTMEDMDRLLAEIKKRDMRLILDLVINHTSSEHAWFKESRSSRDNPKRDWYIWKDNANNWLSFFSGSAWSYDEKTKQYYLRLFAETQPDLNWENPKTREAIYKSALEFWYEKGVSGFRIDTAGLYSKVQTFEDAPVTFPGEKYQPAGPLINSGPRIHEFHKEMYEKVTSRYDAMTVGEVGHCSKADALKYVSAKEKEMNMMFLFDTVDVGSDKSDRFRYKGFTLTDFKDAIINQSNFIFDDETGELNDAWSTVFIENHDQPRCVTRFGNTSNKLFWSRSAKMLALLQTTLTGTLFVYQGQEIGMTNVSPKWDISEYLDINTINYWNAFNETEHSDEEKAELLKIINLLARDNARTPVQWDSSENGGFGGKPWMRINDNYKDINVASQKEDPDSVLNFYRNAIKTRKHYSETLIFGRFEVQDYDNQEIFYYTKTSNKGQKKMAVVLNFTDREVEYPIPQGKLLLSNIANNITGKLQPYEGRLIEVN

SEQ ID NO: 18 - skipped

SEQ ID NO: 19 - protein, 4 aa, synthetic construct
EAEA

SEQ ID NO: 20 - protein, 5 aa, synthetic construct
GGGGS

SEQ ID NO: 21 - skipped

SEQ ID NO: 22 - protein, 20 aa, synthetic construct
EAAAREAAAREAAAREAAAR

SEQ ID NO: 23 - protein, 59 aa, synthetic construct
GSSGSSGSSGSSGSSGSSGSSGSSEAAAREAAAREAAAREAAARGGGGSGGGGSGGGGS

SEQ ID NO: 24 - protein, 24 aa, synthetic construct
GSSGSSGSSGSSGSSGSSGSSGSS

SEQ ID NO: 25 - protein, 15 aa, synthetic construct
GGGGSGGGGSGGGGS

SEQ ID NO: 26 - DNA, 941 bp, synthetic construct
  source 1..941: mol_type = other DNA
gatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattggatcccga

SEQ ID NO: 27 - DNA, 559 bp, synthetic construct
  source 1..559: mol_type = other DNA
aaataagcatgtttgtttcagatcaaagattagcgtttcaaagttgtggaaaagtgaccatgcaacaatatgcaacacattcggattatctgataagtttcaaagctactaagtaagcccgtttcaagtctccagaccgacatctgccatccagtgattttcttagtcctgaaaaatacgatgtgtaaacataaaccacaaagatcggcctccgaggttgaacccttacgaaagagacatctggtagcgccaatgccaaaaaaaaatcacaccagaaggacaattcccttcccccccagcccattaaagcttaccatttcctattccaatacgttccatagagggcatcgctcggctcattttcgcgtgggtcatactagagcggctagctagtcggctgtttgagctctctaatcgaggggtaaggatgtctaatatgtcataatggctcactatataaagaacccgcttgctcaaccttcgactcctttcccgatcctttgcttgttgcttcttcttttataacaggaaacaaaggaatttatacactttaagaatt

SEQ ID NO: 28 - DNA, 750 bp, synthetic construct
  source 1..750: mol_type = other DNA
cttccccatttcactgacagtttgtagaaatagggcaacaattgatgcaaatcgattttcaacgcattggttttgatagcattgatgatcttggagctgtaaaagtccggctggataagctcaatgaaataggttggttgatctggatcttcttttgggtcattttgttcgctctgtatttcacaaattgccagaatctctgccaaccacagtggtaggtccaacttggtgttctgaatcacaggcttccccgggttgttctctaaataaccgaggcccggcacagaaatcgtaaaccgacacggtatcttttgtccgtccgccagtatctcatcaaggtcgtagtagcccatgatgagtatcaaaggggatttggttatgcgatgcaacgagagattgtttatcccagatgctgatgtaaaaaccttaaccagcgtgacagtagaaataagacacgttaaaattacccgcgcttccctaacaattggctctgcctttcggcaagtttctaactgccctcccctctcacatgcaccacgaacttaccgttcgctcctagcagaaccaccccaaagtttaatcaggaccgcattttagcctattgctgtagaaccccacaacataacctggtccagagccagccctttatatatggtaaatcccgtttgaacttcgaagtggaatcggaatttttacatcaaagaaactgatactgaaacttttggcttcgacttggactttctcttaatc

SEQ ID NO: 29 - DNA, 1004 bp, synthetic construct
  source 1..1004: mol_type = other DNA
aaatcagccattaatctcacctcagtttttgaatcagtagaattttcaatgaaacaaacggttggtatattatttgatagggtagccaaatttccaaaaatgaacttttcatcaggtaatatcttgaataccgtaatgtagtgactattggaagaaactgctatcaaattatatttcggatagaaatccaaaccccagactgatctcttgagtctcaactctaagtcagccgcgactctaattatctgtggattaggagttagtgtggacaaagcatcagtatagtataactttacggttccattatcagacgctattgcaagaacttcctttccattgatctctccaattcgacagtaattgatatcataaggtaggtctggaaacacactggcgcttgtatcccattctgcaggaatttctggaacggtggtaatggtagttatccaacggagttggggtagttggtatatctggatatgccgcctataggataaaaacaggagagagtgaaccttgcttacggctactagattgttcttgtactcggaattgtcgttatcggaaactagactaatctcatctgtgtgttgcagtactattgagtcgttgtagtatctaccaggagggcattccatgaactagtgagacaaatgagttggattttctcaatagacatatgcaagaatgctacacaacggatgtcgcactctttttcttagttgataatatcatccaatcagaagacacgggctagaaggacttgctcccgaaggataatccactgctactatctcccttcctcacatatagtcttgcagggctcatgcccctttctccttcgaactgcccgatgaggaagtctttagcctatcaaggaattcgggaccatcatcaatttttagagccttacctgatcgcaatcaggatttcactactcatataaatacatcactcaaactccaactttgcttgttcatacaattcttgatattcacaggatc

SEQ ID NO: 30 - DNA, 758 bp, synthetic construct
  source 1..758: mol_type = other DNA
gtgaatttgtcacggaattgaccaagaggtcagacgatcctgtatcccattgagccgttatgctttgtgggggaaaccctatttctatcgtactaagaaaaccaatggtgaactcatattcggtatcaatggcgacgattccagcatagcctgtagacagtaacaacactagggcaacagcaactaacatatcttcattgatgaaacgttgtgatcggtgtgacttttatagtaaaagctacaactgtttgaaataccaagatatcattgtgaatggctcaaaagggtaatacatctgaaaaacctgaagtgtggaaaattccgatggagccaactcatgataacgcagaagtcccattttgccatcttctcttggtatgaaacggtagaaaatgatccgagtatgccaattgatactcttgattcatgccctatagtttgcgtagggtttaattgatctcctggtctatcgatctgggacgcaatgtagaccccattagtggaaacactgaaagggatccaacactctaggcggacccgctcacagtcatttcaggacaatcaccacaggaatcaactacttctcccagtcttccttgcgtgaagcttcaagcctacaacataacacttcttacttaatctttgattctcgaattgtttacccaatcttgacaacttagcctaagcaatactctggggttatatatagcaattgctcttcctcgctgtagcgttcattccatctttctagaattcgt

SEQ ID NO: 31 - DNA, 991 bp, synthetic construct
  source 1..991: mol_type = other DNA
cctgttgataagacgcattctagagttgtttcatgaaagggttacgggtgttgattggtttgagatatgccagaggacagatcaatctgtggtttgctaaactggaagtctggtaaggactctagcaagtccgttactcaaaaagtcataccaagtaagattacgtaacacctgggcatgactttctaagttagcaagtcaccaagagggtcctatttaacgtttggcggtatctgaaacacaagacttgcctatcccatagtacatcatattacctgtcaagctatgctaccccacagaaataccccaaaagttgaagtgaaaaaatgaaaattactggtaacttcaccccataacaaacttaataatttctgtagccaatgaaagtaaaccccattcaatgttccgagatttagtatacttgcccctataagaaacgaaggatttcagcttccttaccccatgaacagaaatcttccatttaccccccactggagagatccgcccaaacgaacagataatagaaaaaagaaattcggacaaatagaacactttctcagccaattaaagtcattccatgcactccctttagctgccgttccatccctttgttgagcaacaccatcgttagccagtacgaaagaggaaacttaaccgataccttggagaaatctaaggcgcgaatgagtttagcctagatatccttagtgaagggttgttccgatacttctccacattcagtcatagatgggcagctttgttatcatgaagagacggaaacgggcattaagggttaaccgccaaattatataaagacaacatgtccccagtttaaagtttttctttcctattcttgtatcctgagtgaccgttgtgtttaatataacaagttcgttttaacttaagaccaaaaccagttacaacaaattataacccctctaaacactaaagttcactcttatcaaactatcaaacatcaaaagaattcgcg

SEQ ID NO: 32 - DNA, 500 bp, synthetic construct
  source 1..500: mol_type = other DNA
taatcgaactccgaatgcggttctcctgtaaccttaattgtagcatagatcacttaaataaactcatggcctgacatctgtacacgttcttattggtcttttagcaatcttgaagtctttctattgttccggtcggcattacctaataaattcgaatcgagattgctagtacctgatatcatatgaagtaatcatcacatgcaagttccatgataccctctactaatggaattgaacaaagtttaagcttctcgcacgagaccgaatccatactatgcacccctcaaagttgggattagtcaggaaagctgagcaattaacttccctcgattggcctggacttttcgcttagcctgccgcaatcggtaagtttcattatcccagcggggtgatagcctctgttgctcatcaggccaaaatcatatataagctgtagacccagcacttcaattacttgaaattcaccataacacttgctctagtcaagacttacaattaaa

SEQ ID NO: 33 - DNA, 975 bp, synthetic construct
  source 1..975: mol_type = other DNA
tagcttgggtaggacttgacaagtacggcttccgtggtcataccaaacgcctttgttaccgttggctatacctaatgaccaaggcatttgtggattataacggtatcgtagttgaaaaatatgacgtaaccactggtactagcccccacaaggttgatgctgaatacgggaatcaaggtgccgattttaaaggagtagccactgaagggtttggctgggtcaatgcctcttttattttgggattaacctacttagatgtccaaggcatccgtgcgataggcgccgttacgtcccctgatgtatttttcaggaagctcaaaccttgggaacgcgcaagttatggcctaaggccatgtaacgagatagtcaagtcaaactagaagtatacggtttccccgcagaaatagcagaaataggcgacaaatacatacaacattttcattgtgatagggggcggcggttcctaggagggacaacccccagaaaccttgtagactacgttttcacgacgatgggttattactgtaaaggaagaatatactacccaccagttgaatgtttgaacggatcaaaggtcgaagggagtacacggcccaaccaacgtagctaccggagaaagcaagactttcccaaaccaaatagctccgggtttcttctccggcaacccgtcagtttttgtgtggccggacaaaaattcgcaccctcagtctaattgaaaggtcgggctccgagctctaggcgtttgcgcatgtaatattgcatcccctcccatagataatactgcgcgaacacagggtgcaaattatgatgaccacacatgccagtgaccaaaacagttttttagtctttaaaaaccctcggaacttctgagtatataaaggcttctcatttcctacaagcaaacaaagaagaaacttccactttctaactttttatctatagactttagagttacaaccaacgaacaataacaaa

SEQ ID NO: 34 - DNA, 960 bp, synthetic construct
  source 1..960: mol_type = other DNA
tgaagcttatctgctgagcaagttgtttgaccaaacttgagtcaacagtggttaactatatcctctattattttagatgggagcacatcaagtgtacgggaacaatgcaatcgacaacctgtagcctgacatacatagccatcttgaattgacaaaacttagaatgtcttgaatgtgatagatatgagttcccaaaaatctcttttacgatttcccagttgcggtgtactattacacagaggatatcatagcagacttacaatcctcaggcataaaacgagctttcttatcaaagtgtattcaaatggaccatttgattgcaccaaggcattagccccaaaccataccacacagtaacttgatattctcagcatgcatggaaattccactcataacgcgctattcaccgcgaatacttatctatgaaactgggttctttagtattctttgccaaatttcaccgattagaaattattaggtaatataatttctttggggaaccccttcccgttacgcccgctgcggctttgtggttcttttccagtcttgagcaaattacatctggtctagacagttcttccgtgccccagtatgcgagcgcaaactttcaatcaaacctcgtagcaaattggtacttgaacttcgtatttaaccgctattaaatgtactgactcttacattatgaaaaattttgataaagattttatatttcatctcagttaatctcctaataataatagtctgcataactcaaacggtacttccttttcggaacgcgaagagtagtctctatgtcattctcacactatccgcagcgcaatagagaacgagcatgttacccgactcatcccttgtcgattcggaaacgatttataaatacaattagatcgccaccgatcttcttttgtcaatattataaaaatagtacagattttccttagtcgaatcagatcgcagaaa

SEQ ID NO: 35 - DNA, 876 bp, synthetic construct
  source 1..876: mol_type = other DNA
agatctgagggtgtatacgatgtatcgtgccgaacacatgcacttgacggcacagcaaatggtattcaagaagaccactttagaatgggagttaatagggatggtttcatggaggttaaaacacttcaaggaggcatctgaagcattcaagtatgcactaggtctgaggttttcggtcaaggcatgcaagaaattaattgtattctatctgaacgaacgctccagaatgaaccagccagaaacctcaattgccctcaacaacttaaatcaatccacattatccatccaagagattctcaagtatcgttcgttcctcgatatcaacctaatttcaaacttggtcaaactaggagtttggaatcaccgctggtatgctgagttttctccaaaactcatagaaagccttgcggttgttgtggagaacggagggcttatcaaggtagaaaacgaggttaaggctacctatttcgattcacaagatggagtttacgacttgatgaacgaggtattcaagttcatgaagcattacgattatcctgggactgacaactaagagctcctagtgaagacttgagatggacatgataaacaattatagtgaaaatagaaaccataatacaatattctaatagaggaaccgtttacctgtggttcctattgtggcctactgttactagctagtgtaatacacccttgcctcagctttgcaagttgacaactcagccaaatgatctttgaatgcgcgaaacctcaaggtccatcgaattttctcgaattttcagtgttttcatacagcgtgtcatcttctttcgcgtacttattaaaatcgtacccagatcccttcttcttccttaatttcaattccaacactcaaga

SEQ ID NO: 36 - DNA, 727 bp, synthetic construct
  source 1..727: mol_type = other DNA
agatcttgcaaaatacctttccagctttccagcttcctagcactcatcttgaagatatcaaatattctccattcaaaccaacatcaaaaaatagaataattataatcagtttgaagagcaagagtaattttaaaggaaacacattcatggtcagctagaaggttgactgaagagtcgcaagatatctgagaataaaaaagagcatagctaacaagatgagtaaacacggcaaacagatttaggaacaggtgaagggtttctggctcttcaatgtatatcctgctagccacccattcagaaataacacaaagtaggaccctactgaaaaataaatttaatacatcttcatcctctcattaaaccaccgaccactcaaaccataccagccttgtccaattccatgcatcgtgctatccgtcagaattttcagtgttaatcgaatcggtcattatagctccgtctggggcgacaacttgtcatcacagaatagcacaattatgcgttggaatcgtcaaaaaatcacctccaggtctgtatacatacagaactggttgtaacgacaaccttgtttgattgaggtgactggaaggtggaaagaaagggaggaaataaatattgcaaggaaagaaaaaaaaattgttcacagtcacctcttcaccttcgcgatttcatgtttctttcatgtgctaactgatcccagggcttctccagcgcccttatctgttag

SEQ ID NO: 37 - DNA, 872 bp, synthetic construct
  source 1..872: mol_type = other DNA
ctgcccatctatgactgaatgtggagaagtatcggaacaacccttcactaaggatatctaggctaaactcattcgcgccttagatttctccaaggtatcggttaagtttcctctttcgtactggctaacgatggtgttgctcaacaaagggatggaacggcagctaaagggagtgcatggaatgactttaattggctgagaaagtgttctatttgtccgaatttcttttttctattatctgttcgtttgggcggatctctccagtggggggtaaatggaagatttctgttcatggggtaaggaagctgaaatccttcgtttcttataggggcaagtatactaaatctcggaacattgaatggggtttactttcattggctacagaaattattaagtttgttatggggtgaagttaccagtaattttcattttttcacttcaacttttggggtatttctgtggggtagcatagcttgacaggtaatatgatgtactatgggataggcaagtcttgtgtttcagataccgccaaacgttaaataggaccctcttggtgacttgctaacttagaaagtcatgcccaggtgttacgtaatcttacttggtatgactttttgagtaacggacttgctagagtccttaccagacttccagtttagcaaaccacagattgatctgtcctctggcatatctcaaaccaatcaacacccgtaaccctttcatgaaacaactctagaatgcgtcttatcaacaggattgcccaaaacagtaattggggcggtggaatctacatgggagttccatcgttgtctcggtttttctccctataagctactctggagacgaagtaactaacaccctcaaatatcatt

SEQ ID NO: 38 - DNA, 684 bp, synthetic construct
  source 1..684: mol_type = other DNA
tctgaatccgacctcctctaatctaccactgaagagaagcagtgtattgttcgtctacgtaaatttgaatgtgtaaatggcaaacatggcttcggggatgatttggcatatatattattgtagcatcgtctgtggctctatgagttgtgtggcggatgatgaaaagtttcgtgctgatcccacaatgcggcatttaccaaatggggaaagaccagatttcttcgctgcgccagctagggacagcataatgttccaagaagaagcgattacaggtggattacaaagcgttcgtctgcagttgatgttctacgtgatgggtatgagttgtagtgctacgctccatgaatacttctaatttgtcgttgacaatccatgaataatttaagtttgcttcccaagagtctattgcgaagggtgagccgaatctcttggcgtatgcacccgactcgtcggcttttgtgcgttccttgcaaagctcggtagcaatccgttggtgggagaaatttgtctcacgaatttcagttgggagtagctgttcctggtagcaagttcgaggggatctgtgctcataaaacgtgctcacgccaaaaatattcttacaaaatcttcgcggggtgtttgtcttacataatcgattggatattttcttcaaatttttttttcttactgaagtcccctatagag

SEQ ID NO: 39 - DNA, 842 bp, synthetic construct
  source 1..842: mol_type = other DNA
tcttgccagttgtctcctaagatgtcatcggagtaggctcggctaaagagtagtaatgcatcaagaccaaccaaaacaccttccacgagttcagatgaaccttttaataacttcaggtcactttgatgccggcacaactgggcgagtttcgtatagttaactctgatcttgcactccagaacgggaataggattgactttttgcttccgagaaacgatttgctctctcttcgtctggcttttcactttatatcgcacggaatcaatggatggaactcctaaagctcctaacttcgatgatttgctagccatgactctgtgggacattttcttgcatctcgtttgtaacctgtctgttcctacactaagtttatgagaggctactttggattctagcctcggtggtaaagtgggagataacaacggcataaggcaagaaccagaagtaccataacggtctggtaaagttggtgataacttaattggaagagtgtaagtaagacgtggcttgtaataaggctttccatcaaaaaggttctccgggttggagtttgtgaggctcacatctttgatcagtctttcaatataaattggtaacgttgatgacaatgccggaggtaatttctgtagttgttgatatacgcagataacagattcaaatctccattggttttcatcattgtggcttaaattagatcagaacatggtagtatttaaaaatggatctctttgcagatttactcaatatagcgaaaaaaggagacattcgttacaaaatatgaagataattcgcctcataactcgattaatcaaaacagacggtccagttcttcttttggtagt

SEQ ID NO: 40 - DNA, 689 bp, synthetic construct
  source 1..689: mol_type = other DNA
atctgtactggtactgacaaaggttatccagaatccgagacatttcaacaacagagattccaggcttcaaaacatccattttatcaccaatatctagtaatgcttgcaacaattctggatacttcttctgtgtaaccaaatctcttataaactgaacagctttctgtacgttgtcgtcagtagttggatcaacctcagtggtgacctggcctatcggttttccaaaagacttgtttatcacgtccgaaagctcccatttttgcagatgcgcaactttaaaaggcctggcttgaacatttgcatctcttgttgtgtgttctttgagaaaatattcatcgatctgggtgcttccaacgacagaagatactcttctgagaccagaaagtccccagccatgcttcctaattacaaaatatttgtaggaagatccctgattaggacaaagttgtcttctcatgagttcaactgaaactggggctcaaacggattatgaaaggggtgattaaaggttttcctagccttactttccaaatgtcgaccgagacgaacatttaaaatcctaacatcagaaatttctatccttaatctcattgatggttagtacacttcgcagagtctccacatttgcagaccctcctggataaccaaagcttatctaacagcggcattggacctttgaaaagaccctc

SEQ ID NO: 41 - DNA, 1048 bp, synthetic construct
  source 1..1048: mol_type = other DNA
aaatctgaacacgatgaaacctccccgtagattccaccgccccgttacttttttgggcaatcccgttgataagatccattttagagttgtttctgaaaggattacaggcgttgaagggtcagagagatgccagagaacagaccaattggtagtttgctaaagtggacgtctggcaggtgctctatcgtgttctttatttagggcgttacacttagtaggattacgtaacaatttggcttaaccttctaagttagaaagaaaccaagaggggtcctctttaacgttcagcagtatctaaaacacaaaacctgccctcataatacatcattctatctgtcaagctgtgctaccccacagaaatacccccaagagttaaagtgaaaagaaaagctaaatctgttagacttcaccccataacaaacttgatagttcctgtagccaatgaaagttaaccccattcaatgttccgagatctagtatgcttgctcctataaggaacgaagggttccagcttccttaccccatcaatggaaatctcctatttaccccccactggaaagatccgtccgaacgaacggataatagaaaaaagaaattcggacaaaatagaacacttatttagccaatgaaatccatttccagcatctccttcaactgccgttccatcccctttgttgagctacaccatcgtcagccagtaccgaataggaaacttaaccgatatcttggagaattctaatgcgcgaatgagtttagcctagatatccttagtgaagggttgttccgatacttctccacattcagtcatttcagatgggcagcattgttatcatgaagaaacggaaacgggcagtaagggttaaccgccaaattatataaagacaacatgtccccagtttaaagtttttctttcctattcttgtatcctgagtgaccgttgtgtttaaaataacaagttcgttttaacttaagaccaaaaccagttacaacaaattattccccaactaaacactaaagttcactcttatcaaactatcaaacatcaaag

SEQ ID NO: 42 - DNA, 758 bp, synthetic construct
  source 1..758: mol_type = other DNA
cttccccatttcactgacagtttgtagaaatagggcaacaattgatgcaaatcgattttcaacgcattggttttgatagcattgatgatcttggagctgtaaaagtccggctggataagctcaatgaaataggttggttgatctggatcttcttttgggtcattttgttcgctctgtatttcacaaattgccagaatctctgccaaccacagtggtaggtccaacttggtgttctgaatcacaggcttccccgggttgttctctaaataaccgaggcccggcacagaaatcgtaaaccgacacggtatcttttgtccgtccgccagtatctcatcaaggtcgtagtagcccatgatgagtatcaaaggggatttggttatgcgatgcaacgagagattgtttatcccagatgctgatgtaaaaaccttaaccagcgtgacagtagaaataagacacgttaaaattacccgcgcttccctaacaattggctctgcctttcggcaagtttctaactgccctcccctctcacatgcaccacgaacttaccgttcgctcctagcagaaccaccccaaagtttaatcaggaccgcattttagcctattgctgtagaaccccacaacataacctggtccagagccagccctttatatatggtaaatcccgtttgaacttcgaagtggaatcggaatttttacatcaaagaaactgatactgaaacttttggcttcgacttggactttctcttaatcgaattcgt

SEQ ID NO: 43 - DNA, 798 bp, synthetic construct
  source 1..798: mol_type = other DNA
caggtgaacccacctaactatttttaactggcatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttatagcttttcgcctggtgccgtacggtataaatacatactctcctcccccccctggttctctttttcttttgttacttacattttaccgttccgt

SEQ ID NO: 44 - DNA, 1009 bp, synthetic construct
  source 1..1009: mol_type = other DNA
aaataaatggcagaaggatcagcctggacgaagcaaccagttccaactgctaagtaaagaagatgctagacgaaggagacttcagaggtgaaaagtttgcaagaagagagctgcgggaaataaattttcaatttaaggacttgagtgcgtccatattcgtgtacgtgtccaactgttttccattacctaagaaaaacataaagattaaaaagataaacccaatcgggaaactttagcgtgccgtttcggattccgaaaaacttttggagcgccagatgactatggaaagaggagtgtaccaaaatggcaagtcgggggctactcaccggatagccaatacattctctaggaaccagggatgaatccaggtttttgttgtcacggtaggtcaagcattcacttcttaggaatatctcgttgaaagctacttgaaatcccattgggtgcggaaccagcttctaattaaatagttcgatgatgttctctaagtgggactctacggctcaaacttctacacagcatcatcttagtagtcccttcccaaaacaccattctaggtttcggaacgtaacgaaacaatgttcctctcttcacattgggccgttactctagccttccgaagaaccaataaaagggaccggctgaaacgggtgtggaaactcctgtccagtttatggcaaaggctacagaaatcccaatcttgtcgggatgttgctcctcccaaacgccatattgtactgcagttggtgcgcattttagggaaaatttaccccagatgtcctgattttcgagggctacccccaactccctgtgcttatacttagtctaattctattcagtgtgctgacctacacgtaatgatgtcgtaacccagttaaatggccgaaaaactatttaagtaagtttatttctcctccagatgagactctccttcttttctccgctagttatcaaactataaacctattttacctcaaatacctccaacatcacccacttaaacagaatt

SEQ ID NO: 45 - DNA, 686 bp, synthetic construct
  source 1..686: mol_type = other DNA
tgcttaagtaattgaaaacagtgttgtgattatataagcatggtatttgaatagaactactggggttaacttatctagtaggatggaagttgagggagatcaagatgcttaaagaaaaggattggccaatatgaaagccataattagcaatacttatttaatcagataattgtggggcattgtgacttgacttttaccaggacttcaaacctcaaccatttaaacagttatagaagacgtaccgtcacttttgcttttaatgtgatctaaatgtgatcacatgaactcaaactaaaatgatatcttttactggacaaaaatgttatcctgcaaacagaaagctttcttctattctaagaagaacatttacattggtgggaaacctgaaaacagaaaataaatactccccagtgaccctatgagcaggatttttgcatccctattgtaggcctttcaaactcacacctaatatttcccgccactcacactatcaatgatcacttcccagttctcttcttcccctattcgtaccatgcaacccttacacgccttttccatttcggttcggatgcgacttccagtctgtggggtacgtagcctattctcttagccggtatttaaacatacaaattcacccaaattctaccttgataaggtaattgattaatttcataaatgaattcgcg

SEQ ID NO: 46 - DNA, 477 bp, synthetic construct
  source 1..477: mol_type = other DNA
tttttgtagaaatgtcttggtgtcctcgtccaatcaggtagccatctctgaaatatctggctccgttgcaactccgaacgacctgctggcaacgtaaaattctccggggtaaaacttaaatgtggagtaatggaaccagaaacgtctcttcccttctctctccttccaccgcccgttaccgtccctaggaaattttactctgctggagagcttcttctacggcccccttgcagcaatgctcttcccagcattacgttgcgggtaaaacggaggtcgtgtacccgacctagcagcccagggatggaaaagtcccggccgtcgctggcaataatagcgggcggacgcatgtcatgagattattggaaaccaccagaatcgaatataaaaggcgaacacctttcccaattttggtttctcctgacccaaagactttaaatttaatttatttgtccctatttcaatcaattgaacaactat

SEQ ID NO: 47 - DNA, 1018 bp, synthetic construct
  source 1..1018: mol_type = other DNA
aaatagcagtttgcggtttcttgatttcatggggggaacaaacaatagtgttgccttaattctaattggcattgttgcttggaatcgaaattgggggataacgtcatatctgaaaagtaaacaacttcgggaaatcaggctgtttgaatggcttggaagcgagatagaaaggggatagcgagatagagggggcggagtagacgaagggtgttaaactgctgaaatctctcaatctggaagaaacggaataaattaactccttgcgataataaaatccgagtccgttatgaccccacaccgtgttgaccacggcataccccatggaatctggtacaaagcgtcagtcttgaagacaccatcacgtgtaggagactgattgtctgaccgtccagcaaaaagggcattataaatcttgctgttaaaggggtgaggggagatgcaggttgttcttttattcgccttgaactttttaattttcccggggttgcggagcgtgaacagttagcccgatctgatagcttgcaagattcaacagtttatccactacaggtcagagagatcgccgcagaagaaatgctcgtctcgtgttccagcacacatactggtgaagtcgttattttgccgaagggggggtaataaggttatgcaccccctctccacaccccagaatcattttttagctgggttcaaggcattagactttgcacatttttcccttaaacacccttgaaacgcggataaacagttgcatgtgcatcctaaaactaggtgagatgcgtactccgtgctccgataataacagtggtgttggggttgctgctagctcacgcactccgttcttttttttcaaccagcaaaattcgatggggagaaacttggggtactttgccgactcctccaccatgctggtatataaataatactcgcccacttttcgtttgctgcttttatatttcatagactgaaaaagactcttcttctactttttcataatatatctcagatatcactactatag

SEQ ID NO: 48 - DNA, 508 bp, synthetic construct
  source 1..508: mol_type = other DNA
gcgatttaaattcgcgaaagaacagcctaataaactccgaagcatgatggcctctatccggaaaacgttaagagatgtggcaacaggagggcacatagaatttttaaagacgctgaagaatgctatcatagtccgtaaaaatgtgatagtactttgtttagtgcgtacgccacttattcggggccaatagctaaacccaggtttgctggcagcaaattcaactgtagattgaatctctctaacaataatggtgttcaatcccctggctggtcacggggaggactatcttgcgtgatccgcttggaaaatgttgtgtatccctttctcaattgcggaaagcatctgctacttcccataggcaccagttacccaattgatatttccaaaaaagattaccatatgttcatctagaagtataaatacaagtggacattcaatgaatatttcattcaattagtcattgacactttcatcaacttactacgtcttattcaacaatgaattcgcg

SEQ ID NO: 49 - DNA, 507 bp, synthetic construct
  source 1..507: mol_type = other DNA
acacagttattattcatttaaatgtcaaaacagtagtgataaaaggctatgaaggaggttgtctaggggctcgcggaggaaagtgattcaaacagacctgccaaaaagagaaaaaagagggaatccctgttctttccaatggaaatgacgtaactttaacttgaaaaataccccaaccagaagggttcaaactcaacaaggattgcgtaattcctacaagtagcttagagctgggggagagacaactgaaggcagcttaacgataacgcggggggattggtgcacgactcgaaaggaggtatcttagtcttgtaacctcttttttccagaggctattcaagattcataggcgatatcgatgtggagaagggtgaacaatataaaaggctggagagatgtcaatgaagcagctggatagatttcaaattttctagatttcagagtaatcgcacaaaacgaaggaatcccaccaagacaaaaaaaaaaattctaaggaattccgaaacg

SEQ ID NO: 50 - DNA, 759 bp, synthetic construct
  source 1..759: mol_type = other DNA
aaattctttttacgtggtgcgcatactggacagaggcagagtctcaatttcttcttttgagacaggctactacagcctgtgattcctcttggtacttggatttgcttttatctggctccgttgggaactgtgcctgggttttgaagtatcttgtggatgtgtttctaacactttttcaatcttcttggagtgagaatgcaggactttgaacatcgtctagctcgttggtaggtgaaccgttttaccttgcatgtggttaggagttttctggagtaaccaagaccgtcttatcatcgccgtaaaatcgctcttactgtcgctaataatcccgctggaagagaagttcgaacagaagtagcacgcaaagctcttgtcaaatgagaattgttaatcgtttgacaggtcacactcgtgggctatgtacgatcaacttgccggctgttgctggagagatgacaccagttgtggcatggccaattggtattcagccgtaccactgtatggaaaatgagattatcttgttcttgatctagtttcttgccattttagagttgccacattcgtaggtttcagtaccaataatggtaacttccaaacttccaacgcagataccagagatctgccgatccttccccaacaataggagcttactacgccatacatatagcctatctattttcactttcgcgtgggtgcttctatataaacggttccccatcttccgtttcatactacttgaattttaagcactaaagaatt

SEQ ID NO: 51 - DNA, 260 bp, synthetic construct
  source 1..260: mol_type = other DNA
aaattaaccagtgttttcttatctatttgtctttttacactaaagtgaagtacgaatccatgcgattgattcctcctcagatatcagctgaattcttgcttatgtaatacttgcgcgaactacatgtgaacttaggattcgataaggctggggggtcaaccaaccccacttcaaagagccgacccgtataaatagcctctgcgtcctcagatcaacaagacgaagcaatttttttttacctatcttcaggtgcctgttag

SEQ ID NO: 52 - DNA, 223 bp, synthetic construct
  source 1..223: mol_type = other DNA
agggaggcaattagttgtccttgtggaatcaaaagagcacaagaaacctgtgattgaaagtctgggctgtctggggttggcaagaaaatcataaagtttatatagtacatttgttagttgcttctttgaatgacaccttgatctacatgttgttcttcccagttcccaccgcgaagtttctctaactctcaatctctctttccccacttgataatccaaagaa

SEQ ID NO: 53 - DNA, 425 bp, synthetic construct
  source 1..425: mol_type = other DNA
gtcgaggaaagggtcgtttcggggagttaaatatttttggctatgtagcagacatgtttcgacgctggcgtcgcgtcgatcggaaaatattaccccaggaacaagcacttgcttgggttagccaccaccctgcgcaagcctttttgccggctctacacagggccaatgaaatctgggcggaatctgaaaccgatgaaacggacgacactggcaacaagctcactgcactattttttttttctagtgaaatagcctatcctcgtctcgctcccctcatacctgtaaaggggtgcaatttagcctcgttccagccattcacgggccactcaacaacacgtcggctaccatggggtgcttgggcaccaaaaggcctataaataggcccccatccgtctgctacacagtcatctctgtcttttcttccc

SEQ ID NO: 54-55 - skipped (2 entries)

SEQ ID NO: 56 - protein, 42 aa, synthetic construct
MFTPVRRRVRTAALALSAAAALVLGSTAASGASATPSPAPAP

SEQ ID NO: 57 - protein, 18 aa, synthetic construct
MKLSTVLLSAGLASTTLA

SEQ ID NO: 58 - protein, 19 aa, synthetic construct
MRFPSIFTAVLFAASSALA

SEQ ID NO: 59 - protein, 22 aa, synthetic construct
MVSLRSIFTSSILAAGLTRAHG

SEQ ID NO: 60 - protein, 21 aa, synthetic construct
MKFPVPLLFLLQLFFIIATQG

SEQ ID NO: 61 - protein, 18 aa, synthetic construct
MQVKSIVNLLLACSLAVA

SEQ ID NO: 62 - protein, 22 aa, synthetic construct
MQFNWNIKTVASILSALTLAQA

SEQ ID NO: 63 - protein, 59 aa, synthetic construct
MYRNLIIATALTCGAYSAYVPSEPWSTLTPDASLESALKDYSQTFGIAIKSLDADKIKR

SEQ ID NO: 64 - protein, 22 aa, synthetic construct
MNLYLITLLFASLCSAITLPKR

SEQ ID NO: 65 - protein, 25 aa, synthetic construct
MFEKSKFVVSFLLLLQLFCVLGVHG

SEQ ID NO: 66 - protein, 21 aa, synthetic construct
MQFNSVVISQLLLTLASVSMG

SEQ ID NO: 67 - protein, 31 aa, synthetic construct
MKSQLIFMALASLVASAPLEHQQQHHKHEKR

SEQ ID NO: 68 - protein, 19 aa, synthetic construct
MKFAISTLLIILQAAAVFA

SEQ ID NO: 69 - protein, 23 aa, synthetic construct
MKLLNFLLSFVTLFGLLSGSVFA

SEQ ID NO: 70 - protein, 20 aa, synthetic construct
MIFNLKTLAAVAISISQVSA

SEQ ID NO: 71 - protein, 40 aa, synthetic construct
MKISALTACAVTLAGLAIAAPAPKPEDCTTTVQKRHQHKR

SEQ ID NO: 72 - protein, 18 aa, synthetic construct
MSYLKISALLSVLSVALA

SEQ ID NO: 73 - protein, 20 aa, synthetic construct
MLSTILNIFILLLFIQASLQ

SEQ ID NO: 74 - protein, 36 aa, synthetic construct
MKLSTNLILAIAAASAVVSAAPVAPAEEAANHLHKR

SEQ ID NO: 75 - protein, 19 aa, synthetic construct
MFKSLCMLIGSCLLSSVLA

SEQ ID NO: 76 - protein, 19 aa, synthetic construct
MKLAALSTIALTILPVALA

SEQ ID NO: 77 - protein, 23 aa, synthetic construct
MSFSSNVPQLFLLLVLLTNIVSG

SEQ ID NO: 78 - protein, 46 aa, synthetic construct
MQLQYLAVLCALLLNVQSKNVVDFSRFGDAKISPDDTDLESRERKR

SEQ ID NO: 79 - protein, 19 aa, synthetic construct
MKIHSLLLWNLFFIPSILG

SEQ ID NO: 80 - protein, 19 aa, synthetic construct
MSTLTLLAVLLSLQNSALA

SEQ ID NO: 81 - protein, 38 aa, synthetic construct
MINLNSFLILTVTLLSPALALPKNVLEEQQAKDDLAKR

SEQ ID NO: 82 - protein, 16 aa, synthetic construct
MFSLAVGALLLTQAFG

SEQ ID NO: 83 - protein, 16 aa, synthetic construct
MKILSALLLLFTLAFA

SEQ ID NO: 84 - protein, 20 aa, synthetic construct
MKVSTTKFLAVFLLVRLVCA

SEQ ID NO: 85 - protein, 19 aa, synthetic construct
MQFGKVLFAISALAVTALG

SEQ ID NO: 86 - protein, 18 aa, synthetic construct
MWSLFISGLLIFYPLVLG

SEQ ID NO: 87 - protein, 21 aa, synthetic construct
MRNHLNDLVVLFLLLTVAAQA

SEQ ID NO: 88 - protein, 18 aa, synthetic construct
MFLKSLLSFASILTLCKA

SEQ ID NO: 89 - protein, 20 aa, synthetic construct
MFVFEPVLLAVLVASTCVTA

SEQ ID NO: 90 - protein, 22 aa, synthetic construct
MFSPILSLEIILALATLQSVFA

SEQ ID NO: 91 - protein, 16 aa, synthetic construct
MIINHLVLTALSIALA

SEQ ID NO: 92 - protein, 19 aa, synthetic construct
MLALVRISTLLLLALTASA

SEQ ID NO: 93 - protein, 17 aa, synthetic construct
MRPVLSLLLLLASSVLA

SEQ ID NO: 94 - protein, 24 aa, synthetic construct
MVLIQNFLPLFAYTLFFNQRAALA

SEQ ID NO: 95 - protein, 20 aa, synthetic construct
MVSLTRLLITGIATALQVNA

SEQ ID NO: 96 - protein, 24 aa, synthetic construct
MIFDGTTMSIAIGLLSTLGIGAEA

SEQ ID NO: 97 - protein, 31 aa, synthetic construct
MVLVGLLTRLVPLVLLAGTVLLLVFVVLSGG

SEQ ID NO: 98 - protein, 16 aa, synthetic construct
MLSILSALTLLGLSCA

SEQ ID NO: 99 - protein, 20 aa, synthetic construct
MRLLHISLLSIISVLTKANA

SEQ ID NO: 100 - protein, 89 aa, synthetic construct
MRFPSIFTAVLFAASSALAAPVNTTTEDETAQIPAEAVIGYLDLEGDFDVAVLPFSNSTNNGLLFINTTIASIAAKEEGVSLDKREAEA

SEQ ID NO: 101 - protein, 17 aa, synthetic construct
MFKSVVYSILAASLANA

SEQ ID NO: 102 - protein, 19 aa, synthetic construct
MLLQAFLFLLAGFAAKISA

SEQ ID NO: 103 - protein, 21 aa, synthetic construct
MASSNLLSLALFLVLLTHANS

SEQ ID NO: 104 - protein, 29 aa, synthetic construct
MNIFYIFLFLLSFVQGLEHTHRRGSLVKR

SEQ ID NO: 105 - protein, 48 aa, synthetic construct
MLIIVLLFLATLANSLDCSGDVFFGYTRGDKTDVHKSQALTAVKNIKR

SEQ ID NO: 106 - protein, 49 aa, synthetic construct
MESVSSLFNIFSTIMVNYKSLVLALLSVSNLKYARGMPTSERQQGLEER

SEQ ID NO: 107 - protein, 18 aa, synthetic construct
MFAFYFLTACISLKGVFG

SEQ ID NO: 108 - protein, 23 aa, synthetic construct
MRFSTTLATAATALFFTASQVSA

SEQ ID NO: 109 - protein, 23 aa, synthetic construct
MKFAYSLLLPLAGVSASVINYKR

SEQ ID NO: 110 - protein, 22 aa, synthetic construct
MKFFAIAALFAAAAVAQPLEDR

SEQ ID NO: 111 - protein, 15 aa, synthetic construct
MQFFAVALFATSALA

SEQ ID NO: 112 - protein, 24 aa, synthetic construct
MKWVTFISLLFLFSSAYSRGVFRR

SEQ ID NO: 113 - protein, 18 aa, synthetic construct
MRSLLILVLCFLPLAALG

SEQ ID NO: 114 - protein, 15 aa, synthetic construct
MKVLILACLVALALA

SEQ ID NO: 115 - protein, 18 aa, synthetic construct
MFNLKTILISTLASIAVA

SEQ ID NO: 116 - protein, 20 aa, synthetic construct
MYRKLAVISAFLATARAQSA

SEQ ID NO: 117 - protein, 85 aa, synthetic construct
MRFPSIFTAVLFAASSALAAPVNTTTEDETAQIPAEAVIGYLDLEGDFDVAVLPFSNSTNNGLLFINTTIASIAAKEEGVQLDKR

SEQ ID NO: 118 - protein, 85 aa, synthetic construct
MRFPPIFTAALFAASSALAAPANTTTEDETAQIPAEAVIGYLDSEGDSDVAVLPFSNSTNNGLSFINTTIASIAAKEEGVQLDKR

SEQ ID NO: 119 - protein, 85 aa, synthetic construct
MRFPSIFTAVLFAASSALAAPANTTTEDETAQIPAEAVISYSDLEGDFDAAALPLSNSTNNGLSSTNTTIASIAAKEEGVQLDKR

SEQ ID NO: 120 - protein, 85 aa, synthetic construct
MRPPSIFTAVLFAASSALAAPANTTTEDETTQIPAEAVATYLDLEGDVDVAVLPFSSSTNNGLSFINTTIASIAAKEEGVQLDKR

SEQ ID NO: 121 - protein, 85 aa, synthetic construct
MRFPSIFTAALFAASSALAAPANTTTEGETAQTPAEAVIGYRDLEGDFDVAVLPFPNSTNNGLLFTNTTTASIAAKEEGVQLDKR

SEQ ID NO: 122 - protein, 85 aa, synthetic construct
MRFPSIFTAVLLAAPSALAAPANATTEDEAAQIPAEAVIGYLDLEGDFDAAVLPFSNSTNNGLLSINTTIASIAAKEEGVQLDKR

SEQ ID NO: 123 - protein, 85 aa, synthetic construct
MRFPSIFTAVVFAASSALAAPANTTAEDETAQIPAEAVIGYLGLEGDSDVAALPLSDSTNNGSLSTNTTIASIAAKEEGVQLDKR

SEQ ID NO: 124 - protein, 85 aa, synthetic construct
MRLPSIFTAAVFAASSALAAPANTTTEDETAQIPAEAAIGYLDLEGDSDVAVLPLSNSTNNGLLFINTTIASIAAKEEGVQLDKR

SEQ ID NO: 125 - protein, 85 aa, synthetic construct
MRFPSIFTAVLFAASSALAAPANTTTEDETAQIPAEAVIGYLDLEGDFDVAVLPFSNSTNDGLSFINTTTASIAAKEEGVQLDKR

SEQ ID NO: 126 - protein, 35 aa, synthetic construct
MRFPSIFTAVLFAASSALAAPVNTTTEDETAQIPA

SEQ ID NO: 127 - protein, 37 aa, synthetic construct
MLSTILNIFILLLFIQASLQAPIPVVTKYVTEGIAVV

SEQ ID NO: 128 - protein, 36 aa, synthetic construct
MSFSSNVPQLFLLLVLLTNIVSGAVISVWSTSKVTK

SEQ ID NO: 129 - protein, 35 aa, synthetic construct
MNLYLITLLFASLCSAITLPKRDIIWDYSSEKIMG

SEQ ID NO: 130 - protein, 35 aa, synthetic construct; identical to SEQ ID NO: 126

SEQ ID NO: 131 - protein, 35 aa, synthetic construct
MLSTILNIFILLLFIQASLQEFDYKDDDDKMVSKG

SEQ ID NO: 132 - protein, 35 aa, synthetic construct
MSFSSNVPQLFLLLVLLTNIVSGEFDYKDDDDKMV

SEQ ID NO: 133 - protein, 35 aa, synthetic construct
MNLYLITLLFASLCSAEFDYKDDDDKMVSKGEELF

SEQ ID NO: 134 - protein, 35 aa, synthetic construct; identical to SEQ ID NO: 126

SEQ ID NO: 135 - protein, 35 aa, synthetic construct
MLSTILNIFILLLFIQASLQEFLPSGSDPAFSQPK

SEQ ID NO: 136 - protein, 35 aa, synthetic construct
MSFSSNVPQLFLLLVLLTNIVSGEFLPSGSDPAFS

SEQ ID NO: 137 - protein, 35 aa, synthetic construct
MNLYLITLLFASLCSAEFLPSGSDPAFSQPKSVLD

SEQ ID NO: 138 - protein, 206 aa, synthetic construct
MVAWWSLFLYGLQVAAPALAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 139 - protein, 243 aa, synthetic construct
MRFPSIFTAVLFAASSALAAPVNTTTEDELEGDFDVAVLPFSASIAAKEEGVSLEKRAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 140 - protein, 205 aa, synthetic construct
MRFPSIFTAVLFAASSALAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 141 - protein, 204 aa, synthetic construct
MSFRSLLALSGLVCSGLAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 142-143 - skipped (2 entries)

SEQ ID NO: 144 - protein, 24 aa, synthetic construct
MAMAGVFVLFSFVLCGFLPDAAFG

SEQ ID NO: 145 - protein, 18 aa, synthetic construct
MRSLLILVLCFLPLAALG

SEQ ID NO: 146 - protein, 89 aa, synthetic construct
MRFPSIFTAVLFAASSALAAPVNTTTEDETAQIPAEAVIGYSDLEGDFDVAVLPFSNSTNNGLLFINTTIASIAAKEEGVSLDKREAEA

SEQ ID NO: 147 - protein, 19 aa, synthetic construct
MKLILCTVLSLGIAAVCFA

SEQ ID NO: 148 - protein, 19 aa, synthetic construct
MKLFVPALLSLGALGLCLA

SEQ ID NO: 149 - protein, 19 aa, synthetic construct
MKLFIPALLFLGTLGLCLA

SEQ ID NO: 150 - protein, 23 aa, synthetic construct
MESKALLLLALSVWLQSLTVSHG

SEQ ID NO: 151 - protein, 16 aa, synthetic construct
MLLIWTLSLLLGAVLG

SEQ ID NO: 152 - protein, 186 aa, synthetic construct
AEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 153 - protein, 186 aa, synthetic construct
AEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 154 - protein, 186 aa, synthetic construct
AEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYMNKCNACNAVVESNGTLTLSHFGKC

SEQ ID NO: 155 - protein, 210 aa, synthetic construct
MAMAGVFVLFSFVLCGFLPDAAFGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 156 - protein, 210 aa, synthetic construct
MAMAGVFVLFSFVLCGFLPDAVFGAEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 157 - protein, 208 aa, synthetic construct
MAMAGVFVLFSFVLCGFLPDAAFGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVDCSEYPKPDCTAEDRPLCGSDNKTYGNKCNFCNAVVESNGTLTLSHFGKC

SEQ ID NO: 158 - protein, 186 aa, synthetic construct
AEVDCSRFPNATDKEGKDVLVCNKDLRPICGTDGVTYNNECLLCAYSIEFGTNISKEHDGECKETVPMNCSSYANTTSEDGKVMVLCNRAFNPVCGTDGVTYDNECLLCAHKVEQGASVDKRHDGECRKELAAVSVDCSEYPKPDCTAEDRPLCGSDNKTYGNKCNFCNAVVESNGTLTLSHFGKC

SEQ ID NO: 159 - protein, 208 aa, synthetic construct
MAMAGVFVLFSFALCGFLPDAAFGVEVDCSRFPNATNEEGKDVLVCTEDLRPICGTDGVTYSNDCLLCAYNIEYGTNISKEHDGECREAVPVDCSRYPNMTSEEGKVLILCNKAFNPVCGTDGVTYDNECLLCAHNVEQGTSVGKKHDGECRKELAAVDCSEYPKPACTMEYRPLCGSDNKTYDNKCNFCNAVVESNGTLTLSHFGKC

SEQ ID NO: 160 - protein, 239 aa, synthetic construct
MQTITWRQPQGDHLRSRAPAATCRAGQYLTMAMAGIFVLFSFALCGFLPDAAFGVEVDCSRFPNTTNEEGKDVLVCTEDLRPICGTDGVTHSECLLCAYNIEYGTNISKEHDGECREAVPMDCSRYPNTTNEEGKVMILCNKALNPVCGTDGVTYDNECVLCAHNLEQGTSVGKKHDGGCRKELAAVSVDCSEYPKPACTLEYRPLCGSDNKTYGNKCNFCNAVVESNGTLTLSHFGKC

SEQ ID NO: 161 - protein, 185 aa, synthetic construct
VEVDCSRFPNTTNEEGKDVLVCTEDLRPICGTDGVTHSECLLCAYNIEYGTNISKEHDGECREAVPMDCSRYPNTTSEEGKVMILCNKALNPVCGTDGVTYDNECVLCAHNLEQGTSVGKKHDGECRKELAAVSVDCSEYPKPACTLEYRPLCGSDNKTYGNKCNFCNAVVESNGTLTLSHFGKC

SEQ ID NO: 162 - protein, 237 aa, synthetic construct
MQTITWRQPQGDHLRSRAPAATCRAGQYLTMAMAGIFVLFSFALCGFLPDAAFGVEVDCSRFPNTTNEEGKDVLVCTEDLRPICGTDGVTHSECLLCAYNIEYGTNISKEHDGECREAVPMDCSRYPNTTNEEGKVMILCNKALNPVCGTDGVTYDNECVLCAHNLEQGTSVGKKHDGGCRKELAAVDCSEYPKPACTLEYRPLCGSDNKTYGNKCNFCNAVVESNGTLTLSHFGKC

SEQ ID NO: 163 - protein, 128 aa, synthetic construct
EYGTNISIKHNGECKETVPMDCSRYANMTNEEGKVMMPCDRTYNPVCGTDGVTYDNECQLCAHNVEQGTSVDKKHDGVCGKELAAVSVDCSEYPKPECTAEERPICGSDNKTYGNKCNFCNAVVYVQP

SEQ ID NO: 164 - protein, 195 aa, synthetic construct
VDCSRFPNTTNEEGKDVLACTKELHPICGTDGVTYSNECLLCYYNIEYGTNISKEHDGECTEAVPVDCSRYPNTTSEEGKVLIPCNRDFNPVCGSDGVTYENECLLCAHNVEQGTSVGKKHDGGCRKEFAAVSVDCSEYPKPDCTLEYRPLCGSDNKTYASKCNFCNAVVIWEQEKNTRHHASHSVFFISARLVC

SEQ ID NO: 165 - protein, 339 aa, synthetic construct
MLPLGLREYGTNTSKEHDGECTEAVPVDCSRYPNTTSEEGKVRILCKKDINPVCGTDGVTYDNECLLCSHSVGQGASIDKKHDGGCRKEFAAVSVDCSEYPKPACMSEYRPLCGSDNKTYVNKCNFCNAVVYVQPWLHSRCRLPPTGTSFLGSEGRETSLLTSRATDLQVAGCTAISAMEATRAAALLGLVLLSSFCELSHLCFSQASCDVYRLSGSRNLACPRIFQPVCGTDNVTYPNECSLCRQMLRSRAVYKKHDGRCVKVDCTGYMRATGGLGTACSQQYSPLYATNGVIYSNKCTFCSAVANGEDIDLLAVKYPEEESWISVSPTPWRMLSAGA

SEQ ID NO: 166 - protein, 238 aa, synthetic construct
MSWWGIKPALERPSQEQSTSGQPVDSGSTSTTTMAGIFVLLSLVLCCFPDAAFGVEVDCSRFPNTTNEEGKEVLLCTKDLSPICGTDGVTYSNECLLCAYNIEYGTNISKDHDGECKEAVPVDCSTYPNMTNEEGKVMLVCNKMFSPVCGTDGVTYDNECMLCAHNVEQGTSVGKKYDGKCKKEVATVDCSDYPKPACTVEYMPLCGSDNKTYDNKCNFCNAVVDSNGTLTLSHFGKC

SEQ ID NO: 167 - protein, 284 aa, synthetic construct
MSSQNQLHRRRRPLPGGQDLNKYYWPHCTSDRFSWLLHVTAEQFRHCVCIYLQPALERPSQEQSTSGQPVDSGSTSTTTMAGIFVLLSLVLCCFPDAAFGVEVDCSRFPNTTNEEGKEVLLCTKDLSPICGTDGVTYSNECLLCAYNIEYGTNISKDHDGECKEAVPVDCSTYPNMTNEEGKVMLVCNKMFSPVCGTDGVTYDNECMLCAHNVEQGTSVGKKYDGKCKKEVATVDCSDYPKPACTVEYMPLCGSDNKTYDNKCNFCNAVVDSNGTLTLSHFGKC

SEQ ID NO: 168 - protein, 186 aa, synthetic construct
VEVDCSRFPNTTNEEGKDEVVCPDELRLICGTDGVTYNHECMLCFYNKEYGTNISKEQDGECGETVPMDCSRYPNTTSEDGKVTILCTKDFSFVCGTDGVTYDNECMLCAHNVVQGTSVGKKHDGECRKELAAVSVDCSEYPKPACPKDYRPVCGSDNKTYSNKCNFCNAVVESNGTLTLNHFGKC

SEQ ID NO: 169 - protein, 210 aa, synthetic construct
MAMAGVFLLFSFALCGFLPDAAFGVEVDCSRFPNTTNEEGKDEVVCPDELRLICGTDGVTYNHECMLCFYNKEYGTNISKEQDGECGETVPMDCSRYPNTTSEDGKVTILCTKDFSFVCGTDGVTYDNECMLCAHNIVQGTSVGKKHDGECRKELAAVSVDCSEYPKPACPKDYRPVCGSDNKTYSNKCNFCNAVVESNGTLTLNHFGKC

SEQ ID NO: 170 - protein, 205 aa, synthetic construct
MAGVFVLLSLVLCCFPDAAFGVEVDCSRFPNTTNEEGKDVLLCTKELSPVCGTDGVTYSNECLLCAYNIEYGTNISKDHDGECKEAVPADCSMYPNMTNEEGKMTLLCNKMFSPVCGTDGVTYDNECMLCAHNVEQGTSVGKKYDGKCKKEVATVDCSGYPKPACTMEYMPLCGSDNKTYGNKCNFCNAVVDSNGTLTLSHFGEC

SEQ ID NO: 171 - protein, 171 aa, synthetic construct
QVDCSRFPNTTNEEGKEVLLCTKELSPVCGTDGVTYSNECLLCAYNIEYGTNISKDHDGECKEAVPADCSMYPNMTNEEGKMTLLCNKMFSPVCGTDGVTYDNECMLCAHNVEQGTSVGKKYDGKCKKEVATVSVDCSGYPKPACTMEYMPLCGSDNKTYGNKCNFCNAVV

SEQ ID NO: 172 - protein, 207 aa, synthetic construct
MTMPGAFVVLSFVLCCFPDATFGVEVDCSTYPNTTNEEGKEVLVCSKILSPICGTDGVTYSNECLLCANNIEYGTNISKYHDGECKEFVPVNCSRYPNTTNEEGKVMLICNKDLSPVCGTDGVTYDNECLLCAHNLEPGTSVGKKYDGECKKEIATVDCSDYPKPVCSLESMPLCGSDNKTYSNKCNFCNAVVDSNETLTLSHFGKC

SEQ ID NO: 173 - protein, 207 aa, synthetic construct
MTMAGVFVLLSFALCCFPDAAFGVEVDCSTYPNTTNEEGKEVLVCTKILSPICGTDGVTYSNECLLCAYNIEYGTNVSKDHDGECKEVVPVDCSRYPNSTNEEGKVVMLCSKDLNPVCGTDGVTYDNECVLCAHNVESGTSVGKKYDGECKKETATVDCSDYPKPACTLEYMPFCGSDSKTYSNKCNFCNAVVDSNGTLTLSHFGKC

SEQ ID NO: 174 - protein, 207 aa, synthetic construct
MTTAGVFVLLSFALCSFPDAAFGVEVDCSTYPNTTNEEGKEVLVCTKILSPICGTDGVTYSNECLLCAYNIEYGTNVSKDHDGECKEFVPVDCSRYPNTTNEDGKVVLLCNKDLSPICGTDGVTYDNECLLCARNLEPGTSVGKKYDGECKKEIATVDCSDYPKPVCSLEYMPLCGSDSKTYSNKCNFCNAVVDSNGTLTLSHFGKC

SEQ ID NO: 175 - protein, 207 aa, synthetic construct
MTTAGVFVLLSFTLCSFPDAAFGVEVDCSPYPNTTNEEGKEVLVCNKILSPICGTDGVTYSNECLLCAYNLEYGTNISKDYDGECKEVAPVDCSRHPNTTNEEGKVELLCNKDLNPICGTNGVTYDNECLLCARNLESGTSIGKKYDGECKKEIATVDCSDYPKPVCTLEEMPLCGSDNKTYGNKCNFCNAVVDSNGTLTLSHFGKC

SEQ ID NO: 176 - protein, 207 aa, synthetic construct
MTTAVVFVLLSFALCCFPDAAFGVEVDCSTYPNSTNEEGKDVLVCPKILGPICGTDGVTYSNECLLCAYNIQYGTNVSKDHDGECKEIVPVDCSRYPNTTNEEGKVVFLCNKNFDPVCGTDGDTYDNECMLCARSLEPGTTVGKKHDGECKREIATVDCSDYPKPTCSAEDMPLCGSDSKTYSNKCNFCNAVVDSNGTLTLSRFGKC

SEQ ID NO: 177 - protein, 207 aa, synthetic construct
MTMTGVFVLLSFAICCFPDAAFGVEVDCSTYPNTTNEEGKEVLVCTKILSPICGTDGVTYSNECLLCAYNIEYGTNVSKDHDGECKEVVPVDCSKYPNTTNEEGKVVLLCSKDLSPVCGTDGVTYDNECLLCARNLEPGSSVGKKYDGECKKEIATIDCSDYPKPVCSLEYMPLCGSDSKTYDNKCNFCNAVVDSNGTLTLSHFGKC

SEQ ID NO: 178 - protein, 207 aa, synthetic construct
MTTAGVFVLLSFVLCCFPDAVFGVEVDCSTYPNTTNEEGKEVLVCTKILSPICGTDGVTYSNECLLCAYNIEYGTNVSKDHDGECKEVVPVNCSRYPNTTNEEGKVVLRCSKDLSPVCGTDGVTYDNECLMCARNLEPGAVVGKNYDGECKKEIATVDCSDYPKPVCSLEYMPLCGSDSKTYSNKCNFCNAVVDSNGTLTLSHFGKC

SEQ ID NO: 179 - protein, 207 aa, synthetic construct
MTTAGVFVLLSIALCCFPDAAFGVEVDCSAYSNTTSEEGKEVLSCTKILSPICGTDGVTYSNECLLCAYNIEYGTNISKDHDGECKEVVSVDCSRYPNTTNEEGKAVLLCNKDLSPVCGTDGVTYDNECLLCAHNLEPGTSVGKKYDGACKKEIATVDCSDYPKPVCTLEYLPLCGSDSKTYSNKCDFCNAVVDSNGTLTLSHFGKC

SEQ ID NO: 180 - protein, 206 aa, synthetic construct
MTTAGVFVLLSFALCCFPDAAFGVEVDCSTYPNTTNEEGKEVLVCTKILSPICGTDGTTYSNECLLCAYNIEYGTNVSKDHDGECKVVPVDCSKYPNTTNEDGKVVLLCNKALSPICGTDRVTYDNECLMCAHNLEPGTSVGKKHDGECQKEVATVDCSDYPKPVCSLEYMPLCGSDGKTYSNKCNFCNAVVNSNGTLTLSHFEKC

SEQ ID NO: 181 - protein, 213 aa, synthetic construct
MTTAGVFVLLSFVLCCFFPDAAFGVEVDCSTYPNTTNEEGKEVLVCAKILSPVCGTDGVTYSNECLLCAHNIENGTNVGKDHDGKCKEAVPVDCSRYPNTTDEEGKVVLLCNKDVSPVCGTDGVTYDNECLLCAHNLEAGTSVDKKNDSECKTEDTTLAAVSVDCSDYPKPVCTLEYLPLCGSDNKTYSNKCRFCNAVVDSNGTLTLSRFGKC

SEQ ID NO: 182 - protein, 207 aa, synthetic construct
MTTAGVFVLLSFALCCSPDAAFGVEVDCSTYPNTTNEEGKEVLACTKILSPICGTDGVTYSNECLLCAYNMEYGTNVSKDHDGKCKEVVPVDCSRYPNTTNEEGKVVLLCNKDLSPVCGTDGVTYDNECLLCARNLEPGASVGKKYDGECKKEIATVDCSDYPKPVCSLEHMPLCGSDSKTYSNKCTFCNAVVDSNGTLTLSHFGKC

SEQ ID NO: 183 - protein, 207 aa, synthetic construct
MTTAGVFVLLSFALCCFPDAAFGVEVDCSTYPNTTNEEGREVLVCTKILSPICGTDGVTYSNECLLCAYNIEYGTNVSKDHDGECKEVAPVGCSRYPNTTNEEGKVVLLCNKDLSPVCGTDGVTYDNECLLCARHLEPGTSVGKKYDGECKKEIATVDCSDYPKPVCSLEYMPLCGSDSKTYSNKCNFCNAVLDSNGTLTLSHFGKC

SEQ ID NO: 184 - protein, 207 aa, synthetic construct
MTTAGVFVLLSFALCCFPDAVFGVEVDCSTYPNTTNEEGKEVLVCTKILSPICGTDGVTYSNECLLCAYNIEYGTNVSKDHDGECKEVVPVDCSRYPNTTNEEGKVVLRCNKDLSPVCGTDGVTYDNECLMCARNLEPGAIVGKKYDGECKKEIATVDCSDYPKPVCSLEYMPLCGSDSKTYSNKCNFCNAVVDSNGTLILSHFGKC

SEQ ID NO: 185 - protein, 207 aa, synthetic construct
MTTAGVFVLLSFVLCCFPDAVFGVEVDCSTYPNTTNEEGKEVLVCTKILSPICGTDGVTYSNECLLCAYNIEYGTNVSKDHDGECKEVVPVDCSRYPNTTNEEGKVVLRCSKDLSPVCGTDGVTYDNECLMCARNLEPGAVVGKNYDGECKKEIATVDCSDYPKPVCSLEYMPLCGSDSKTYSNKCNFCNAVVDSNGTLTLSHFGKC

SEQ ID NO: 186 - protein, 282 aa, synthetic construct
MSSQNQLPSRCRPLPGSQDLNKYYQPHCTGDRFCWLFYVTVEQFRHCICIYLQLALERPSHEQSGQPADSRNTSTMTTAGVFVLLSFALCCFPDAVFGVEVDCSTYPNTTNEEGKEVLVCTKILSPICGTDGVTYSNECLLCAYNIEYGTNVSKDHDGECKEVVPVDCSRYPNTTNEEGKVVLRCNKDLSPVCGTDGVTYDNECLMCARNLEPGAIVGKKYDGECKKEIATVDCSDYPKPVCSLEYMPLCGSDSKTYSNKCNFCNAVVDSNGTLILSHFGKC

SEQ ID NO: 187 - protein, 193 aa, synthetic construct
MTTAVVFVLLSFALCCFPDAAFGVEVDCSTYPNSTNEEGKDVLVCPKILGPICGTDGVTYSNECLLCAYNIQYGTNVSKDHDGECKEIVPVDCSRYPNTTNEEGKVVFLCNKNFDPVCGTDGDTYDNECMLCARSLEPGTTVGKKHDGECKREIATVDCSDYPKPTCSAEDMPLCGSDSKTYSNKCNFCNAVV

SEQ ID NO: 188 - protein, 190 aa, synthetic construct
EAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 189 - protein, 199 aa, synthetic construct
EEGVSLEKREAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 190 - protein, 275 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEAAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 191 - protein, 273 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEAVEVDCSTYPNTTNEEGKEVLVCTKILSPICGTDGVTYSNECLLCAYNIEYGTNVSKDHDGECKEFVPVDCSRYPNTTNEDGKVVLLCNKDLSPICGTDGVTYDNECLLCARNLEPGTSVGKKYDGECKKEIATVDCSDYPKPVCSLEYMPLCGSDSKTYSNKCNFCNAVVDSNGTLTLSHFGKC

SEQ ID NO: 192 - protein, 188 aa, synthetic construct
EAEAVEVDCSTYPNTTNEEGKEVLVCTKILSPICGTDGVTYSNECLLCAYNIEYGTNVSKDHDGECKEFVPVDCSRYPNTTNEDGKVVLLCNKDLSPICGTDGVTYDNECLLCARNLEPGTSVGKKYDGECKKEIATVDCSDYPKPVCSLEYMPLCGSDSKTYSNKCNFCNAVVDSNGTLTLSHFGKC

SEQ ID NO: 193 - protein, 206 aa, synthetic construct
MTMAGVFVLLSFILCCFPDTAFGVEVDCSIYPNTTSEEGKEVLVCTETLSPICGSDGVTYNNECQLCAYNVEYGTNVSKDHDGECKEIVPVDCSRYPNTTEEGRVVMLCNKALSPVCGTDGVTYDNECLLCARNLESGTSVGKKFDGECKKEIATVDCTDYPKPVCSLDYMPLCGSDSKTYSNKCNFCNAVMDSNGTLTLNHFGKC

SEQ ID NO: 194 - protein, 272 aa, synthetic construct
MRFPSIFTAVLFAASSALAAPVNTTTEDETAQIPAEAVIGYSDLEGDFDVAVLPFSNSTNNGLLFINTTIASIAAKEEGVSLDKREAEAVEVDCSIYPNTTSEEGKEVLVCTETLSPICGSDGVTYNNECQLCAYNVEYGTNVSKDHDGECKEIVPVDCSRYPNTTEEGRVVMLCNKALSPVCGTDGVTYDNECLLCARNLESGTSVGKKFDGECKKEIATVDCTDYPKPVCSLDYMPLCGSDSKTYSNKCNFCNAVMDSNGTLTLNHFGKC

SEQ ID NO: 195 - protein, 187 aa, synthetic construct
EAEAVEVDCSIYPNTTSEEGKEVLVCTETLSPICGSDGVTYNNECQLCAYNVEYGTNVSKDHDGECKEIVPVDCSRYPNTTEEGRVVMLCNKALSPVCGTDGVTYDNECLLCARNLESGTSVGKKFDGECKKEIATVDCTDYPKPVCSLDYMPLCGSDSKTYSNKCNFCNAVMDSNGTLTLNHFGKC

SEQ ID NO: 196 - protein, 402 aa, synthetic construct
MFFYNTDFRMGSISAANAEFCFDVFNELKVQHTNENILYSPLSIIVALAMVYMGARGNTEYQMEKALHFDSIAGLGGSTQTKVQKPKCGKSVNIHLLFKELLSDITASKANYSLRIANRLYAEKSRPILPIYLKCVKKLYRAGLETVNFKTASDQARQLINSWVEKQTEGQIKDLLVSSSTDLDTTLVLVNAIYFKGMWKTAFNAEDTREMPFHVTKEESKPVQMMCMNNSFNVATLPAEKMKILELPFASGDLSMLVLLPDEVSGLERIEKTINFEKLTEWTNPNTMEKRRVKVYLPQMKIEEKYNLTSVLMALGMTDLFIPSANLTGISSAESLKISQAVHGAFMELSEDGIEMAGSTGVIEDIKHSPELEQFRADHPFLFLIKHNPTNTIVYFGRYWSP

SEQ ID NO: 197 - protein, 388 aa, synthetic construct
MDSISVTNAKFCFDVFNEMKVHHVNENILYCPLSILTALAMVYLGARGNTESQMKKVLHFDSITGAGSTTDSQCGSSEYVHNLFKELLSEITRPNATYSLEIADKLYVDKTFSVLPEYLSCARKFYTGGVEEVNFKTAAEEARQLINSWVEKETNGQIKDLLVSSSIDFGTTMVFINTIYFKGIWKIAFNTEDTREMPFSMTKEESKPVQMMCMNNSFNVATLPAEKMKILELPYASGDLSMLVLLPDEVSGLERIEKTINFDKLREWTSTNAMAKKSMKVYLPRMKIEEKYNLTSILMALGMTDLFSRSANLTGISSVDNLMISDAVHGVFMEVNEEGTEATGSTGAIGNIKHSLELEEFRADHPFLFFIRYNPTNAILFFGRYWSP

SEQ ID NO: 198 - protein, 386 aa, synthetic construct
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 199 - protein, 474 aa, synthetic construct
MRFPSIFTAVLFAASSALAAPVNTTTEDETAQIPAEAVIGYSDLEGDFDVAVLPFSNSTNNGLLFINTTIASIAAKEEGVSLDKREAEAGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 200 - protein, 389 aa, synthetic construct
EAEAGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 201 - protein, 419 aa, synthetic construct
MRVPAQLLGLLLLWLPGARCGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPLEIKRAAAHHHHHH

SEQ ID NO: 202 - protein, 406 aa, synthetic construct
MTSGFANELGPRLMGKLTMGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKTFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPSR

SEQ ID NO: 203 - protein, 451 aa, synthetic construct
MGGRRVRWEVYISRAGYVNRQIAWRRHHRSLTMRVPAQLLGLLLLWLPGARCGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSPLEIKRAAAHHHHHH

SEQ ID NO: 204 - protein, 386 aa, synthetic construct
MGSIGAVSMEFCFDVFKELKVHHANENIFYSPFTIISALAMVYLGAKDSTRTQINKVVRFDKLPGFGDSVEAQCGTSVNVHSSLRDILNQITKPNDVYSFSLASRLYAEETYPILPEYLQCVKELYRGGLESINFQTAADQARGLINSWVESQTNGMIKNVLQPSSVDSQTAMVLVNAIVFKGLWEKAFKDEDTQAIPFRVTEQESKPVQMMYQIGLFKVASMASEKMKILELPFASGTMSMWVLLPDEVSGLEQLETTISFEKMTEWISSNIMEERRIKVYLPRMKMEEKYNLTSVLMAMGITDLFSSSANLSGISSAGSLKISQAVHAAYAEIYEAGREVIGSAEAGADATSVSEEFRVDHPFLYCIKHNLTNSILFFGRCISP

SEQ ID NO: 205 - protein, 386 aa, synthetic construct
MGSIGAVSMEFCFDVFKELKVHHANENIFYSPFTIISALAMVYLGAKDSTRTQINKVVRFDKLPGFGDSVEAQCGTSVNVHSSLRDILNQITKPNDVYSFSLASRLYAEETYPILPEYLQCVKELYRGGLESINFQTAADQARGLINSWVESQTNGMIKNVLQPSSVDSQTAMVLVNAIVFKGLWEKAFKDEDTQAIPFRVTEQESKPVQMMYQIGLFKVASMASEKMKILELPFASGTMSMWVLLPDEVSGLEQLETTISFEKMTEWISSNIMEERRIKVYLPRMKMEEKYNLTSVLMAMGITDLFSSSANLSGISSAGSLKISQAAHAAYAEIYEAGREVIGSAEAGADATSVSEEFRVDHPFLYCIKHNLTNSILFFGRCISP

SEQ ID NO: 206 - protein, 417 aa, synthetic construct
YYRVPCMVLCTAFHPYIFIVLLFALDNSEFTMGSIGAVSMEFCFDVFKELRVHHPNENIFFCPFAIMSAMAMVYLGAKDSTRTQINKVIRFDKLPGFGDSTEAQCGKSANVHSSLKDILNQITKPNDVYSFSLASRLYADETYSIQSEYLQCVNELYRGGLESINFQTAADQARELINSWVESQTNGIIRNVLQPSSVDSQTAMVLVNAIVFRGLWEKAFKDEDTQTMPFRVTEQESKPVQMMYQIGSFKVASMASEKMKILELPLASGTMSMLVLLPDEVSGLEQLETTISFEKLTEWTSSNVMEERKIKVYLPRMKMEEKYNLTSVLMAMGITDLFRSSANLSGISLAGNLKISQAVHAAHAEINEAGRKAVSSAEAGVDATSVSEEFRADRPFLFCIKHIATKVVFFFGRYTSP

SEQ ID NO: 207 - protein, 383 aa, synthetic construct
MGSIGAASMEFCFDVFKELKVHHANDNMLYSPFAILSTLAMVFLGAKDSTRTQINKVVHFDKLPGFGDSIEAQCGTSVNVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMYQIGSFKVASMASEKMKILELPFASGTMSMLVLLPDDVSGLEQLESIISFEKLTEWTSSSIMEERKVKVYLPRMKMEEKYNLTSLLMAMGITDLFSSSANLSGISSVGSLKISQAVHAAHAEINEAGRDVVGSAEAGVDATEEFRADHPFLFCVKHIETNAILLFGRCVSP

SEQ ID NO: 208 - protein, 386 aa, synthetic construct
MASIGAVSTEFCVDVYKELRVHHANENIFYSPFTIISTLAMVYLGAKDSTRTQINKVVRFDKLPGFGDSIEAQCGTSVNVHSSLRDILNQITKPNDVYSFSLASRLYAEETYPILPEYLQCVKELYRGGLESINFQTAADQARELINSWVESQTSGIIKNVLQPSSVNSQTAMVLVNAIYFKGLWERAFKDEDTQAIPFRVTEQESKPVQMMSQIGSFKVASVASEKVKILELPFVSGTMSMLVLLPDEVSGLEQLESTISTEKLTEWTSSSIMEERKIKVFLPRMRMEEKYNLTSVLMAMGMTDLFSSSANLSGISSAESLKISQAVHAAYAEIYEAGREVVSSAEAGVDATSVSEEFRVDHPFLLCIKHNPTNSILFFGRCISP

SEQ ID NO: 209 - protein, 411 aa, synthetic construct
MALCKAFHPYIFIVLLFDVDNSAFTMASIGAVSTEFCVDVYKELRVHHANENIFYSPFTIISTLAMVYLGAKDSTRTQINKVVRFDKLPGFGDSIEAQCGTSVNVHSSLRDILNQITKPNDVYSFSLASRLYAEETYPILPEYLQCVKELYRGGLESINFQTAADQARELINSWVESQTSGIIKNVLQPSSVNSQTAMVLVNAIYFKGLWERAFKDEDTQAIPFRVTEQESKPVQMMSQIGSFKVASVASEKVKILELPFVSGTMSMLVLLPDEVSGLEQLESTISTEKLTEWTSSSIMEERKIKVFLPRMRMEEKYNLTSVLMAMGMTDLFSSSANLSGISSAESLKISQAVHAAYAEIYEAGREVVSSAEAGVDATSVSEEFRVDHPFLLCIKHNPTNSILFFGRCISP

SEQ ID NO: 210 - protein, 383 aa, synthetic construct
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKISQAVHAAYAEINEAGRDVVGSAEAGVDATEEFRADHPFLFCVKHIETNAILLFGRCVSP

SEQ ID NO: 211 - protein, 408 aa, synthetic construct
MGLCTAFHPYIFIVLLFALDNSEFTMGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKISQAVHAAYAEINEAGRDVVGSAEAGVDATEEFRADHPFLFCVKHIETNAILLFGRCVSP

SEQ ID NO: 212 - protein, 383 aa, synthetic construct
MGSIGAASMEFCFDVFKELKVHHANDNMLYSPFAILSTLAMVFLGAKDSTRTQINKVVHFDKLPGFGDSIEAQCGTSANVHSSLRDILNQITKQNDAYSFSLASRLYAQETYTVVPEYLQCVKELYRGGLESVNFQTAADQARGLINAWVESQTNGIIRNILQPSSVDSQTAMVLVNAIAFKGLWEKAFKAEDTQTIPFRVTEQESKPVQMMHQIGSFKVASMASEKMKILELPFASGTMSMLVLLPDDVSGLEQLESTISFEKLTEWTSSSIMEERKVKVYLPRMKMEEKYNLTSLLMAMGITDLFSSSANLSGISSVGSLKIPQAVHAAYAEINEAGRDVVGSAEAGVDATEEFRADHPFLFCVKHIETNAILLFGRCVSP

SEQ ID NO: 213 - protein, 386 aa, synthetic construct
MGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 214 - protein, 386 aa, synthetic construct
MGSIGAASTEFCFDVFRELKVQHVNENIFYSPLSIISALAMVYLGARDNTRTQIDQVVHFDKIPGFGESMEAQCGTSVSVHSSLRDILTEITKPSDNFSLSFASRLYAEETYTILPEYLQCVKELYKGGLESISFQTAADQARELINSWVESQTNGIIKNILQPSSVDSQTTMVLVNAIYFKGMWEKAFKDEDTQTMPFRMTEQESKPVQMMYQVGSFKLATVTSEKVKILELPFASGMMSMCVLLPDEVSGLEQLETTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPSNSILFFGRWISP

SEQ ID NO: 215 - protein, 386 aa, synthetic construct
MGSIGAASTEFCFDVFKELKVQHVNENIFYSPLTIISALSMVYLGARENTRAQIDKVLHFDKMPGFGDTIESQCGTSVSIHTSLKDMFTQITKPSDNYSLSFASRLYAEETYPILPEYLQCVKELYKGGLETISFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITFEKLMAWTSSTTMEERKMKVYLPRMKIEEKYNLTSVLMALGVTDLFSSSANLSGISSAESLKISKAVHEAFVEIYEAGSEVVGSTEAGMEVTSVSEEFRADHPFLFLIKHNPTNSILFFGRCFSP

SEQ ID NO: 216 - protein, 386 aa, synthetic construct
MGSIGAASTEFCFDVFKELKVQHVNENIFYSPLTIISALSMVYLGARENTRTQIDKVLHFDKMTGFGDTVESQCGTSVSIHTSLKDIFTQITKPSDNYSLSLASRLYAEETYPILPEYLQCVKELYKGGLETVSFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITSEKLMEWTSSTTMEERKMKVYLPRMKIEEKYNLTSVLMALGVTDLFSSSADLSGISSAESLKISKAVHEAFVEIYEAGSEVVGSTEGGMEVTSVSEEFRADHPFLFLIKHKPTNSILFFGRCFSP

SEQ ID NO: 217 - protein, 386 aa, synthetic construct
MGSIGAASTEFCFDVFKELKVQHVNENIFYSPLTIISALSMVYLGARENTRTQIDKVLHFDKMTGFGDTVESQCGTSVSIHTSLKDIFTQITKPSDNYSLSLASRLYAEETYPILPEYLQCVKELYKGGLETVSFQTAAEQARELINSWVESQTNGMIKNILQPSSVDPQTKMVLVNAIYFKGVWEKAFKDEDTQEVPFRVTEQESKPVQMMYQIGSFKVAVMASEKMKILELPYASGQLSMLVLLPDDVSGLEQLESAITSEKLMEWTSSTTMEERKMKVYLPRMKIEEKYNLTSVLMALGVTDLFSSSADLSGISSAESLKISKAVHEAFVEIYEAGSEVVGSTEGGMEVTSFSEEFRADHPFLFLIKHKPTNSILFFGRCFSP

SEQ ID NO: 218 - protein, 386 aa, synthetic construct
MGSIGAASTEFCFDVFKELKVQHVNENIFYSPLSIISALSMVYLGARENTRAQIDKVVHFDKITGFGETIESQCGTSVSVHTSLKDMFTQITKPSDNYSLSFASRLYAEETYPILPEYLQCVKELYKGGLETTSFQTAADQARELINSWVESQTNGMIKNILQPGSVDPQTEMVLVNAIYFKGMWEKAFKDEDTQAVPFRMTEQESKTVQMMYQIGSFKVAVMASEKMKILELPYASGELSMLVMLPDDVSGLEQLETAITFEKLMEWTSSNMMEERKMKVYLPRMKMEEKYNLTSVLMALGVTDLFSSSANLSGISSAESLKMSEAVHEAFVEIYEAGSEVVGSTGAGMEVTSVSEEFRADHPFLFLIKHNPTNSILFFGRCFSP

SEQ ID NO: 219 - protein, 386 aa, synthetic construct
MGSIGAASTEFCFDVFKELRVQHVNENVCYSPLIIISALSLVYLGARENTRAQIDKVVHFDKITGFGESIESQCGTSVSVHTSLKDMFNQITKPSDNYSLSVASRLYAEERYPILPEYLQCVKELYKGGLESISFQTAADQAREAINSWVESQTNGMIKNILQPSSVDPQTEMVLVNAIYFKGMWQKAFKDEDTQAVPFRISEQESKPVQMMYQIGSFKVAVMAAEKMKILELPYASGELSMLVLLPDEVSGLEQLENAITVEKLMEWTSSSPMEERIMKVYLPRMKIEEKYNLTSVLMALGITDLFSSSANLSGISAEESLKMSEAVHQAFAEISEAGSEVVGSSEAGIDATSVSEEFRADHPFLFLIKHNATNSILFFGRCFSP

SEQ ID NO: 220 - protein, 386 aa, synthetic construct
MGSISAASTEFCFDVFKELKVQHVNENIFYSPLSIISALSMVYLGARENTRAQIEKVVHFDKITGFGESIESQCSTSVSVHTSLKDMFTQITKPSDNYSLSFASRFYAEETYPILPEYLQCVKELYKGGLETINFRTAADQARELINSWVESQTNGMIKNILQPGSVDPQTDMVLVNAIYFKGMWEKAFKDEDTQALPFRVTEQESKPVQMMYQIGSFKVAVLASEKVKILELPYASGQLSMLVLLPDDVSGLEQLETAITVEKLMEWTSSNNMEERKIKVYLPRIKIEEKYNLTSVLMALGITDLFSSSANLSGISSAESLKVSEAIHEAFVEIYEAGSEVAGSTEAGIEVTSVSEEFRADHPFLFLIKHNATNSILFFGRCFSP

SEQ ID NO: 221 - protein, 386 aa, synthetic construct
MVSIGAASTEFCFDVFKELKVQHVNENIFYSPLSIISALSMVYLGARENTRAQIDKVVHFDKITGFEETIESQCSTSVSVHTSLKDMFTQITKPSDNYSLSFASRLYAEETYPILPEYLQCVKELYKGGLETISFQTAADQARELINSWVESQTDGMIKNILQPGSVDPQTEMVLVNAIYFKGMWEKAFKDEDTQAVPFRMTEQESKPVQMMYQIGSFKVAVMASEKMKILELPYASGGMSMLVMLPDDVSGLEQLETAITFEKLMEWTSSNMMEERKMKVYLPRMKMEEKYNLTSVLMALGMTDLFSSSANLSGISSAESLKMSEAVHEAFVEIYEAGSEAVGSTGAGMEVTSVSEEFRADHPFLFLIKHNPTNSILFFGRCFSP

SEQ ID NO: 222 - protein, 386 aa, synthetic construct
MGSIGAASTEFCFDVFKELKVQHVNENIFYSPLSIISALSMVYLGARENTRAQIDKVVHFDKITGFGEPIESQCGISVSVHTSLKDMITQITKPSDNYSLSFASRLYAEETYPILPEYLQCVKELYKGGLETISFQTAADQARELINSWVENQTNGMIKNILQPGSVDPQTEMVLVNAVYFKGMWEKAFKDEDTQAVPFRMTEQESKPVQMMYQIGSFKVAVMASEKIKILELPYASGELSMLVLLPDDVSGLEQLETAITLDKLTEWTSSNAMEERKMKVYLPRMKIEKKYNLTSVLIALGMTDLFSSSANLSGISSAESLKMSEAIHEAFLEIYEAGSEVVGSTEAGMEVTSVSEEFRADHPFLFLIKHNPTNSILFFGRCLSP

SEQ ID NO: 223 - protein, 386 aa, synthetic construct
MGSIGAASTEFCFDVFKELKVQHVNENIFYSPLTIISALSMVYLGARENTRAQIDKVVHFDKIPGFGDTTESQCGTSVSVHTSLKDMFTQITKPSDNYSVSFASRLYAEETYPILPEFLECVKELYKGGLESISFQTAADQARELINSWVESQTNGMIKNILQPGSVDSQTEMVLVNAIYFKGMWEKAFKDEDTQTVPFRMTEQETKPVQMMYQIGTFKVAVMPSEKMKILELPYASGELCMLVMLPDDVSGLEELESSITVEKLMEWTSSNMMEERKMKVFLPRMKIEEKYNLTSVLMALGMTDLFSSSANLSGISSAEPLKMSEAVHEAFIEIYEAGSEVVGSTGAGMEITSVSEEFRADHPFLFLIKHNPTNSILFFGRCVSP

SEQ ID NO: 224 - protein, 386 aa, synthetic construct
MGSIGAVSTEFCFDVFKELKVQHVNENIFYSPLSIISALSMVYLGARENTRAQIDKVVHFDKITGSGETIEAQCGTSVSVHTSLKDMFTQITKPSENYSVGFASRLYADETYPIIPEYLQCVKELYKGGLEMISFQTAADQARELINSWVESQTNGMIKNILQPGSVDPQTEMILVNAIYFKGVWEKAFKDEDTQAVPFRMTEQESKPVQMMYQFGSFKVAAMAAEKMKILELPYASGALSMLVLLPDDVSGLEQLESAITFEKLMEWTSSNMMEEKKIKVYLPRMKMEEKYNFTSVLMALGMTDLFSSSANLSGISSADSLKMSEVVHEAFVEIYEAGSEVVGSTGSGMEAASVSEEFRADHPFLFLIKHNPTNSILFFGRCFSP

SEQ ID NO: 225 - protein, 391 aa, synthetic construct
MVSIGAASTEFCFDVFKELKVQHVNENIFYSPLSIISALSMVYLGARENTRAQIDKVVHFDKITGFEETIESQVQKKQCSTSVSVHTSLKDMFTQITKPSDNYSLSFASRLYAEETYPILPEYLQCVKELYKGGLETISFQTAADQARELINSWVESQTDGMIKNILQPGSVDPQTEMVLVNAIYFKGMWEKAFKDEDTQAVPFRMTEQESKPVQMMYQIGSFKVAVMASEKMKILELPYASGGMSMLVMLPDDVSGLEQLETAITFEKLMEWTSSNMMEERKMKVYLPRMKMEEKYNLTSVLMALGMTDLFSSSANLSGISSAESLKMSEAVHEAFVEIYEAGSEAVGSTGAGMEVTSVSEEFRADHPFLFLIKHNPTNSILFFGRCFSP

SEQ ID NO: 226 - protein, 386 aa, synthetic construct
MGSIGAASGEFCFDVFKELKVQHVNENIFYSPLSIISALSMVYLGARENTRAQIDKVVHFDKIIGFGESIESQCGTSVSVHTSLKDMFAQITKPSDNYSLSFASRLYAEETFPILPEYLQCVKELYKGGLETLSFQTAADQARELINSWVESQTNGMIKDILQPGSVDPQTEMVLVNAIYFKGVWEKAFKDEDTQTVPFRMTEQESKPVQMMYQIGSFKVAVVAAEKIKILELPYASGALSMLVLLPDDVSSLEQLETAITFEKLTEWTSSNIMEERKIKVYLPRMKIEEKYNLTSVLMDLGITDLFSSSANLSGISSAESLKVSEAIHEAIVDIYEAGSEVVGSSGAGLEGTSVSEEFRADHPFLFLIKHNPTSSILFFGRCFSP

SEQ ID NO: 227 - protein, 386 aa, synthetic construct
MGSIGAASTEFCFDVFKELKVQHVNENIFYSPLSIISALSMVYLGARENTRAQIDKVVHFDKITGSGEAIESQCGTSVSVHISLKDMFTQITKPSDNYSLSFASRLYAEETYPILPEYLQCVKELYKEGLATISFQTAADQAREFINSWVESQTNGMIKNILQPGSVDPQTQMVLVNAIYFKGVWEKAFKDEDTQAVPFRMTKQESKPVQMMYQIGSFKVAVMASEKMKILELPYASGQLSMLVMLPDDVSGLEQIENAITFEKLMEWTNPNMMEERKMKVYLPRMKMEEKYNLTSVLMALGMTDLFSSSANLSGISSAESLKMSEAVHEAFVEIYEAGSEVVGSTGAGIEVTSVSEEFRADHPFLFLIKHNPTNSILFFGRCFSP

SEQ ID NO: 228 - protein, 386 aa, synthetic construct
MGSIGEASTEFCIDVFRELKVQHVNENIFYSPLSIISALSMVYLGARENTRAQIDQVVHFDKITGFGDTVESQCGSSLSVHSSLKDIFAQITQPKDNYSLNFASRLYAEETYPILPEYLQCVKELYKGGLETISFQTAADQARELINSWVESQTNGMIKNILQPSSVDPQTEMVLVNAIYFKGVWEKAFKDEETQAVPFRITEQENRPVQIMYQFGSFKVAVVASEKIKILELPYASGQLSMLVLLPDEVSGLEQLENAITFEKLTEWTSSDIMEEKKIKVFLPRMKIEEKYNLTSVLVALGIADLFSSSANLSGISSAESLKMSEAVHEAFVEIYEAGSEVVGSSGAGIEAASDSEEFRADHPFLFLIKHKPTNSILFFGRCFSP

SEQ ID NO: 229 - protein, 386 aa, synthetic construct
MGSIGAASTEFCFDIFNELKVQHVNENIFYSPLSIISALSMVYLGARENTKAQIDKVVHFDKITGFGESIESQCSTSASVHTSFKDMFTQITKPSDNYSLSFASRLYAEETYPILPEYSQCVKELYKGGLESISFQTAADQARELINSWVESQTNGMIKNILQPGSVDPQTELVLVNAIYFKGTWEKAFKDKDTQAVPFRVTEQESKPVQMMYQIGSYKVAVIASEKMKILELPYASGELSMLVLLPDDVSGLEQLETAITFEKLMEWTSSNMMEERKVKVYLPRMKIEEKYNLTSVLMALGMTDLFSPSANLSGISSAESLKMSEAIHEAFVEIYEAGSEVVGSTEAGMEVTSVSEEFRADHPFLFLIKCNLTNSILFFGRCFSP

SEQ ID NO: 230 - protein, 385 aa, synthetic construct
MGSISTASTEFCFDVFKELKVQHVNENIFYSPLSIISALSMVYLGARENTRAQIEKVVHFDKITGFGESIESQCGTSVSVHTSLKDMLIQISKPSDNYSLSFASKLYAEETYPILPEYLQCVKELYKGGLESINFQTAADQARQLINSWVESQTNGMIKDILQPSSVDPQTEMVLVNAIYFKGIWEKAFKDEDTQEVPFRITEQESKPVQMMYQIGSFKVAVIASEKIKILELPYASGELSMLIVLPDDVSGLEQLETAITFEKLIEWTSPSIMEERKTKVYLPRMKIEEKYNLTSVLMALGMTDLFSPSANLSGISSAESLKMSEAIHEAFVEIYEAGSEVVGSAEAGMEATSVSEFRVDHPFLFLIKHNPANIILFFGRCVSP

SEQ ID NO: 231 - protein, 386 aa, synthetic construct
MGSIGAASTEFCFDVFKELKVQHVNENIFYSPLTIISALSLVYLGARENTRAQIDKVFHFDKISGFGETTESQCGTSVSVHTSLKEMFTQITKPSDNYSVSFASRLYAEDTYPILPEYLQCVKELYKGGLETISFQTAADQAREVINSWVESQTNGMIKNILQPGSVDSQTEMVLVNAIYFKGMWEKAFKDEDTQTMPFRITEQERKPVQMMYQAGSFKVAVMASEKMKILELPYASGEFCMLIMLPDDVSGLEQLENSFSFEKLMEWTTSNMMEERKMKVYIPRMKMEEKYNLTSVLMALGMTDLFSSSANLSGISSAETLKMSEAVHEAFMEIYEAGSEVVGSTGSGAEVTGVYEEFRADHPFLFLVKHKPTNSILFFGRCVSP

SEQ ID NO: 232 - protein, 386 aa, synthetic construct
MGSIGAASTEFCFDIFNELKVQHVNENIFYSPLSIISALSMVYLGARENTKAQIDKVVHFDKITGFGETIESQCSTSVSVHTSLKDTFTQITKPSDNYSLSFASRLYAEETYPILPEYSQCVKELYKGGLETISFQTAADQARELINSWVESQTNGMIKNILQPGSVDPQTELVLVNAIYFKGTWEKAFKDKDTQAVPFRVTEQESKPVQMMYQIGSYKVAVIASEKMKILELPYASRELSMLVLLPDDVSGLEQLETAITFEKLMEWTSSNMMEERKVKVYLPRMKIEEKYNLTSVLMALGMTDLFSPSANLSGISSAESLKMSEAVHEAFVEIYEAGSEVVGSTGAGMEVTSVSEEFRADHPFLFLIKCNPTNSILFFGRCFSP

SEQ ID NO: 233 - protein, 386 aa, synthetic construct
MGSISAASAEFCLDVFKELKVQHVNENIFYSPLSIISALSMVYLGARENTRAQIDKVVHFDKITGSGETIEFQCGTSANIHPSLKDMFTQITRLSDNYSLSFASRLYAEERYPILPEYLQCVKELYKGGLETISFQTAADQARELINSWVESQTNGMIKNILQPGSVNPQTEMVLVNAIYFKGLWEKAFKDEDTQTVPFRMTEQESKPVQMMYQVGSFKVAVMASDKIKILELPYASGELSMLVLLPDDVTGLEQLETSITFEKLMEWTSSNVMEERTMKVYLPHMRMEEKYNLTSVLMALGVTDLFSSSANLSGISSAESLKMSEAVHEAFVEIYESGSQVVGSTGAGTEVTSVSEEFRVDHPFLFLIKHNPTNSILFFGRCFSP

SEQ ID NO: 234 - protein, 385 aa, synthetic construct
MGSIGAASVEFCFDVFKELKVQHVNENIFYSPLSIISALSMVYLGARENTKAQIDKVVHFDKIAGFGEAIESQCVTSASIHSLKDMFTQITKPSDNYSLSFASRLYAEEAYSILPEYLQCVKELYKGGLETISFQTAADQARDLINSWVESQTNGMIKNILQPGAVDLETEMVLVNAIYFKGMWEKAFKDEDTQTVPFRMTEQESKPVQMMYQVGSFKVAVMASDKIKILELPYASGQLSMVVVLPDDVSGLEQLEASITSEKLMEWTSSSIMEEKKIKVYFPHMKIEEKYNLTSVLMALGMTDLFSSSANLSGISSAEKLKVSEAVHEAFVEISEAGSEVVGSTEAGTEVTSVSEEFKADHPFLFLIKHNPTNSILFFGRCFSP

SEQ ID NO: 235 - protein, 386 aa, synthetic construct
MGSIGAASSEFCFDIFKELKVQHVNENIFYSPLSIISALSMVYLGARENTRAQIDKVVPFDKITASGESIESQCSTSVSVHTSLKDIFTQITKSSDNHSLSFASRLYAEETYPILPEYLQCVKELYEGGLETISFQTAADQARELINSWIESQTNGRIKNILQPGSVDPQTEMVLVNAIYFKGMWEKAFKDEDTQAVPFRMTEQESKPVQVMHQIGSFKVAVLASEKIKILELPYASGELSMLVLLPDDVSGLEQLETAITFEKLMEWTSPNIMEERKIKVFLPRMKIEEKYNLTSVLMALGITDLFSPLANLSGISSAESLKMSEAIHEAFVEISEAGSEVIGSTEAEVEVTNDPEEFRADHPFLFLIKHNPTNSILFFGRCFSP

SEQ ID NO: 236 - protein, 386 aa, synthetic construct
MGSIGAASTEFCFDVFKELKAQYVNENIFYSPMTIITALSMVYLGSKENTRAQIAKVAHFDKITGFGESIESQCGASASIQFSLKDLFTQITKPSGNHSLSVASRIYAEETYPILPEYLECMKELYKGGLETINFQTAANQARELINSWVERQTSGMIKNILQPSSVDSQTEMVLVNAIYFRGLWEKAFKVEDTQATPFRITEQESKPVQMMHQIGSFKVAVVASEKIKILELPYASGRLTMLVVLPDDVSGLKQLETTITFEKLMEWTTSNIMEERKIKVYLPRMKIEEKYNLTSVLMALGLTDLFSSSANLSGISSAESLKMSEAVHEAFVEIYEAGSEVVASAEAGMDATSVSEEFRADHPFLFLIKDNTSNSILFFGRCFSP

SEQ ID NO: 237 - protein, 386 aa, synthetic construct
MGSIGAASTEFCFDVFKELKGQHVNENIFFCPLSIVSALSMVYLGARENTRAQIVKVAHFDKIAGFAESIESQCGTSVSIHTSLKDMFTQITKPSDNYSLNFASRLYAEETYPIIPEYLQCVKELYKGGLETISFQTAADQAREIINSWVESQTNGMIKNILRPSSVHPQTELVLVNAVYFKGTWEKAFKDEDTQAVPFRITEQESKPVQMMYQIGSFKVAAVTSEKMKILEVPYASGELSMLVLLPDDVSGLEQLETAITAEKLIEWTSSTVMEERKLKVYLPRMKIEEKYNLTTVLTALGVTDLFSSSANLSGISSAQGLKMSNAVHEAFVEIYEAGSEVVGSKGEGTEVSSVSDEFKADHPFLFLIKHNPTNSIVFFGRCFSP

SEQ ID NO: 238 - protein, 386 aa, synthetic construct
MGSIGAASTEFCFDVFKELKVHHVNENILYSPLAIISALSMVYLGAKENTRDQIDKVVHFDKITGIGESIESQCSTAVSVHTSLKDVFDQITRPSDNYSLAFASRLYAEKTYPILPEYLQCVKELYKGGLETIDFQTAADQARQLINSWVEDETNGMIKNILRPSSVNPQTKIILVNAIYFKGMWEKAFKDEDTQEVPFRITEQETKSVQMMYQIGSFKVAEVVSDKMKILELPYASGKLSMLVLLPDDVYGLEQLETVITVEKLKEWTSSIVMEERITKVYLPRMKIMEKYNLTSVLTAFGITDLFSPSANLSGISSTESLKVSEAVHEAFVEIHEAGSEVVGSAGAGIEATSVSEEFKADHPFLFLIKHNPTNSILFFGRCFSP

SEQ ID NO: 239 - protein, 386 aa, synthetic construct
MGSIGAASTEFCLDVFKELKVQHVNENIFYSPLSIISALSMVYLGARENTRAQIDKVVHFDKITGFEDSIESQCGTSVSVHTSLKDMFTQITKPSDNYSVGFASRLYAAETYQILPEYSQCVKELYKGGLETINFQKAADQATELINSWVESQTNGMIKNILQPSSVDPQTQIFLVNAIYFKGMWQRAFKEEDTQAVPFRISEKESKPVQMMYQIGSFKVAVIPSEKIKILELPYASGLLSMLVILPDDVSGLEQLENAITLEKLMQWTSSNMMEERKIKVYLPRMRMEEKYNLTSVFMALGITDLFSSSANLSGISSAESLKMSDAVHEASVEIHEAGSEVVGSTGSGTEASSVSEEFRADHPYLFLIKHNPTDSIVFFGRCFSP

SEQ ID NO: 240 - protein, 386 aa, synthetic construct
MGSIGAASTEFCFDVFKELKFQHVDENIFYSPLTIISALSMVYLGARENTRAQIDKVVHFDKIAGFEETVESQCGTSVSVHTSLKDMFAQITKPSDNYSLSFASRLYAEETYPILPEYLQCVKELYKGGLETISFQTAADQARDLINSWVESQTNGMIKNILQPSSVGPQTELILVNAIYFKGMWQKAFKDEDTQEVPFRMTEQQSKPVQMMYQTGSFKVAVVASEKMKILALPYASGQLSLLVMLPDDVSGLKQLESAITSEKLIEWTSPSMMEERKIKVYLPRMKIEEKYNLTSVLMALGITDLFSPSANLSGISSAESLKMSQAVHEAFVEIYEAGSEVVGSTGAGMEDSSDSEEFRVDHPFLFFIKHNPTNSILFFGRCFSP

SEQ ID NO: 241 - protein, 386 aa, synthetic construct
MGSIGPLSVEFCCDVFKELRIQHPRENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPINFQTAAEQARELINSWVESQTNGMIKNILQPSSVNPETDMVLVNAIYFKGLWEKAFKDEDIQTVPFRITEQESKPVQMMFQIGSFRVAEITSEKIRILELPYASGQLSLWVLLPDDISGLEQLETAITFENLKEWTSSTKMEERKIKVYLPRMKIEEKYNLTSVLTSLGITDLFSSSANLSGISSAESLKVSSAFHEASVEIYEAGSKVVGSTGAEVEDTSVSEEFRADHPFLFLIKHNPSNSIFFFGRCFSP

SEQ ID NO: 242 - protein, 386 aa, synthetic construct
MGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 243 - protein, 386 aa, synthetic construct
MGSIGAVSTEFSCDVFKELRIHHVQENIFYSPVTIISALSMIYLGARDSTKAQIEKAVHFDKIPGFGESIESQCGTSLSIHTSIKDMFTKITKASDNYSIGIASRLYAEEKYPILPEYLQCVKELYKGGLESISFQTAAEQAREIINSWVESQTNGMIKNILQPSSVDPQTDIVLVNAIYFKGLWEKAFRDEDTQTVPFKITEQESKPVQMMYQIGSFKVAEITSEKIKILEVPYASGQLSLWVLLPDDISGLEKLETAITFENLKEWTSSTKMEERKIKVYLPRMKIEEKYNLTSVLTALGITDLFSSSANLSGISSAESLKVSEAFHEAIVEISEAGSKVVGSVGAGVDDTSVSEEFRADHPFLFLIKHNPTSSIFFFGRCFSP

SEQ ID NO: 244 - protein, 381 aa, synthetic construct
MGSIGAASTEFCFDVFKELKVQHVNENIFYSPLSIISALSMVYLGARENTRAQIDKVVHFDKIAGFGESTESQCGTSVSAHTSLKDMSNQITKLSDNYSLSFASRLYAEETYPILPEYSQCVKELYKGGLESISFQTAAYQARELINAWVESQTNGMIKDILQPGSVDSQTKMVLVNAIYFKGIWEKAFKDEDTQEVPFRMTEQETKPVQMMYQIGSFKVAVIAAEKIKILELPYASGQLSMLVILPDDVSGLEQLETAITFEKLTEWTSASVMEERKIKVYLPRMSIEEKYNLTSVLIALGVTDLFSSSANLSGISSAESLRMSEAIHEAFVETYEAGSTESGTEVTSASEEFRVDHPFLFLIKHKPTNSILFFGRCFSP

SEQ ID NO: 245 - protein, 391 aa, synthetic construct
MGSIGAASSEFCFDIFKELKVQHVNENIFYSPLSIISALSMVYLGARENTRAQIDKVVPFDKITASGESIESQVQKIQCSTSVSVHTSLKDIFTQITKSSDNHSLSFASRLYAEETYPILPEYLQCVKELYEGGLETISFQTAADQARELINSWIESQTNGRIKNILQPGSVDPQTEMVLVNAIYFKGMWEKAFKDEDTQAVPFRMTEQESKPVQVMHQIGSFKVAVLASEKIKILELPYASGELSMLVLLPDDVSGLEQLETAITFEKLMEWTSPNIMEERKIKVFLPRMKIEEKYNLTSVLMALGITDLFSPLANLSGISSAESLKMSEAIHEAFVEISEAGSEVIGSTEAEVEVTNDPEEFRADHPFLFLIKHNPTNSILFFGRCFSP

SEQ ID NO: 246 - protein, 383 aa, synthetic construct
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGIIKNILQPSSVNPETDMVLVNAIYFKGLWEKAFKDEGTQTVPFRITEQESKPVQMMFQIGSFRVAEIASEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEINEAGSKVVGAGVDDTSVSEEFRVDRPFLFLIKHNPSNSIFFFGRCFSP

SEQ ID NO: 247 - protein, 386 aa, synthetic construct
MGSIGAASTEFCFDMFKELKVHHVNENIIYSPLSIISILSMVFLGARENTKTQMEKVIHFDKITGFGESLESQCGTSVSVHASLKDILSEITKPSDNYSLSLASKLYAEETYPVLPEYLQCIKELYKGSLETVSFQTAADQARELINSWVETQTNGVIKNFLQPGSVDPQTEMVLVDAIYFKGTWEKAFKDEDTQEVPFRITEQESKPVQMMYQAGSFKVATVAAEKMKILELPYASGELSMFVLLPDDISGLEQLETTISIEKLSEWTSSNMMEDRKMKVYLPHMKIEEKYNLTSVLVALGMTDLFSPSANLSGISTAQTLKMSEAIHGAYVEIYEAGSEMATSTGVLVEAASVSEEFRVDHPFLFLIKHNPSNSILFFGRCIFP

SEQ ID NO: 248 - protein, 392 aa, synthetic construct
MGSIGAASTEFCFDMFKELKVHHVNENIIYSPLSIISILSMVFLGARENTKTQMEKVIHFDKITGFGESLESQCGTSVSVHASLKDILSEITKPSDNYSLSLASKLYAEETYPVLPEYLQCIKELYKGSLETVSFQTAADQARELINSWVETQTNGVIKNFLQPGSVDPQTEMVLVDAIYFKGTWEKAFKDEDTQEVPFRITEQESKPVQMMYQAGSFKVATVAAEKMKILELPYASGELSMFVLLPDDISGLEQLETTISIEKLSEWTSSNMMEDRKMKVYLPHMKIEEKYNLTSVLVALGMTDLFSPSANLSGISTAQTLKMSEAIHGAYVEIYEAGSEMATSTGVLVEAASVSEEFRVDHPFLFLIKHNPSNSILFFGRCIFPHHHHHH

SEQ ID NO: 249 - protein, 386 aa, synthetic construct
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGMIKNILQPSAVNPETDMVLVNAIYFKGLWEKAFKDEGTQTVPFRITEQESKPVQMMFQIGSFRVAEITSEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEIYEAGSKVVGSTGAGVDDTSVSEEFRVDRPFLFLIKHNPSNSIFFFGRCFSP

SEQ ID NO: 250 - protein, 395 aa, synthetic construct
  VARIANT 99: note = Any amino acid
  VARIANT 233: note = Any amino acid
  VARIANT 341: note = Any amino acid
  VARIANT 379: note = Any amino acid
MEDQRGNTGFTMGSIGAASTEFCIDVFRELRVQHVNENIFYSPLTIISALSMVYLGARENTRAQIDQVVHFDKIAGFGDTVESQCGSSPSVHNSLKTVXAQITQPRDNYSLNLASRLYAEESYPILPEYLQCVKELYNGGLETVSFQTAADQARELINSWVESQTNGIIKNILQPSSVDPQTEMVLVNAIYFKGLWEKAFKDEETQAVPFRITEQENRPVQMMYQFGSFKVAXVASEKIKILELPYASGQLSMLVLLPDEVSGLEQNAITFEKLTEWTSSDLMEERKIKVFFPRVKIEEKYNLTAVLVSLGITDLFSSSANLSGISSAENLKMSEAVHEAXVEIYEAGSEVAGSSGAGIEVASDSEEFRVDHPFLFLIXHNPTNSILFFGRCFSP

SEQ ID NO: 251 - protein, 386 aa, synthetic construct
MGSIGAASTEFCIDVFRELRVQHVNENIFYSPLSIISALSMVYLGARENTRAQIDEVFHFDKIAGFGDTVDPQCGASLSVHKSLQNVFAQITQPKDNYSLNLASRLYAEESYPILPEYLQCVKELYNEGLETVSFQTGADQARELINSWVENQTNGVIKNILQPSSVDPQTEMVLVNAIYFKGLWQKAFKDEETQAVPFRITEQENRPVQMMYQFGSFKVAVVASEKVKILELPYASGQLSMWVLLPDEVSGLEQLENAITFEKLTEWTSSDLTEERKIKVFLPRVKIEEKYNLTAVLMALGVTDLFSSSANFSGISAAENLKMSEAVHEAFVEIYEAGSEVVGSSGAGIEAPSDSEEFRADHPFLFLIKHNPTNSILFFGRCFSP

SEQ ID NO: 252 - protein, 386 aa, synthetic construct
MGSIGPLSVEFCCDVFKELRIQHARDNIFYSPVTIISALSMVYLGARDNTKAQIEKAVHFDKIPGFGESIESQCGTSLSVHTSLKDIFTQITKPRENYTVGIASRLYAEEKYPILPEYLQCIKELYKGGLEPISFQTAAEQARELINSWVESQTNGMIKNILQPSSVNPETDMVLVNAIYFKGLWKKAFKDEGTQTVPFRITEQESKPVQMMFQIGSFRVAEITSEKIRILELPYASGQLSLWVLLPDDISGLEQLESAITFENLKEWTSSTKMEERKIKVYLPRMKIEEKYNLTSVLTSLGITDLFSSSANLSGISSAEKLKVSSAFHEASMEIYEAGNKVVGSTGAGVDDTSVSEEFRVDRPFLFLIKHNPSNSIFFFGRCFSP

SEQ ID NO: 253 - protein, 385 aa, synthetic construct
MGSIGAASAEFCVDVFKELKDQHVNNIVFSPLMIISALSMVNIGAREDTRAQIDKVVHFDKITGYGESIESQCGTSIGIYFSLKDAFTQITKPSDNYSLSFASKLYAEETYPILPEYLKCVKELYKGGLETISFQTAADQARELINSWVESQTNGMIKNILQPSSVDPQTEMVLVNAIYFKGLWEKAFKDEDTQAVPFRITEQESKPVQMMYQIGSFKVAVIASEKIKILELPYASGQLSLLVLLPDDVSGLEQLESAITSEKLLEWTNPNIMEERKTKVYLPRMKIEEKYNLTSVLVALGITDLFSSSANLSGISSAEGLKLSDAVHEAFVEIYEAGREVVGSSEAGVEDSSVSEEFKADRPFIFLIKHNPTNGILYFGRYISP

SEQ ID NO: 254 - protein, 386 aa, synthetic construct
MGSIGAANTDFCFDVFKELKVHHANENIFYSPLSIVSALAMVYLGARENTRAQIDKALHFDKILGFGETVESQCDTSVSVHTSLKDMLIQITKPSDNYSFSFASKIYTEETYPILPEYLQCVKELYKGGVETISFQTAADQAREVINSWVESHTNGMIKNILQPGSVDPQTKMVLVNAVYFKGIWEKAFKEEDTQEMPFRINEQESKPVQMMYQIGSFKLTVAASENLKILEFPYASGQLSMMVILPDEVSGLKQLETSITSEKLIKWTSSNTMEERKIRVYLPRMKIEEKYNLKSVLMALGITDLFSSSANLSGISSAESLKMSEAVHEAFVEIYEAGSEVTSSTGTEMEAENVSEEFKADHPFLFLIKHNPTDSIVFFGRCMSP

SEQ ID NO: 255 - protein, 383 aa, synthetic construct
MGSIGPLSVEFCCDVFKELRIQHARENIFYSPVTIISALSMVYLGARDNTKAQIEKAVHFDKIPGFGESIESQCGTSLSIHTSLKDIFTQITKPSDNYTVGIASRLYAEEKYPILPEYLQCIKELYKGGLEPISFQTAAEQARELINSWVESQTNGMIKNILQPSSVNPETDMVLVNAIYFKGLWEKAFKDESTQTVPFRITEQESKPVQMMFQIGSFRVAEIASEKIRILELPYASGQLSLWVLLPDDISGLEQLETAITFENLKEWTSSTKMEERKIKVYLPRMKIEEKYNLTSVLTSLGITDLFSSSANLSGISSAERLKVSSAFHEASMEIYEAGSRVVEAGVDDTSVSEEFRVDRPFLFLIKHNPSNSIFFFGRCFSP

SEQ ID NO: 256 - protein, 386 aa, synthetic construct
MGSIGPVSTEFCCDIFKELRIQHARENIIYSPVTIISALSMVYLGARDNTKAQIEKAVHFDKIPGFGESIESQCGTSLSIHTSLKDILTQITKPSDNYTVGIASRLYAEEKYPILSEYLQCIKELYKGGLEPISFQTAAEQARELINSWVESQTNGMIKNILQPSSVNPETDMVLVNAIYFKGLWEKAFKDEGTQTVPFRITEQESKPVQMMFQIGSFKVAEITSEKIRILELPYASGKLSLWVLLPDDISGLEQLETAITFENLKEWTSSTRMEERKIKVYLPRMKIEEKYNLTSVLTSLGITDLFSSSANLSGISSAERLKVSSAFHEVFVEIYEAGSKVEGSTGAGVDDTSVSEEFRADHPFLFLVKHNPSNSIIFFGRCYLP

SEQ ID NO: 257 - protein, 386 aa, synthetic construct
MGSTGAASMEFCFALFRELKVQHVNENIFFSPVTIISALSMVYLGARENTRAQLDKVAPFDKITGFGETIGSQCSTSASSHTSLKDVFTQITKASDNYSLSFASRLYAEETYPILPEYLQCVKELYKGGLESISFQTAADQARELINSWVESQTNGMIKDILRPSSVDPQTKIILITAIYFKGMWEKAFKEEDTQAVPFRMTEQESKPVQMMYQIGSFKVAVIPSEKLKILELPYASGQLSMLVILPDDVSGLEQLETAITTEKLKEWTSPSMMKERKMKVYFPRMRIEEKYNLTSVLMALGITDLFSPSANLSGISSAESLKVSEAVHEASVDIDEAGSEVIGSTGVGTEVTSVSEEIRADHPFLFLIKHKPTNSILFFGRCFSP

SEQ ID NO: 258 - protein, 2174 aa, synthetic construct
MEHAQLTQLVNSNMTSNTCHEADEFENIDFRMDSISVTNTKFCFDVFNEMKVHHVNENILYSPLSILTALAMVYLGARGNTESQMKKALHFDSITGAGSTTDSQCGSSEYIHNLFKEFLTEITRTNATYSLEIADKLYVDKTFTVLPEYINCARKFYTGGVEEVNFKTAAEEARQLINSWVEKETNGQIKDLLVPSSVDFGTMMVFINTIYFKGIWKTAFNTEDTREMPFSMTKQESKPVQMMCLNDTFNMATLPAEKMRILELPYASGELSMLVLLPDEVSGLEQIEKAINFEKLREWTSTNAMEKKSMKVYLPRMKIEEKYNLTSTLMALGMTDLFSRSANLTGISSVENLMISDAVHGAFMEVNEEGTEAAGSTGAIGNIKHSVEFEEFRADHPFLFLIRYNPTNVILFFDNSEFTMGSIGAVSTEFCFDVFKELRVHHANENIFYSPFTVISALAMVYLGAKDSTRTQINKVVRFDKLPGFGDSIEAQCGTSANVHSSLRDILNQITKPNDIYSFSLASRLYADETYTILPEYLQCVKELYRGGLESINFQTAADQARELINSWVESQTSGIIRNVLQPSSVDSQTAMVLVNAIYFKGLWEKGFKDEDTQAMPFRVTEQENKSVQMMYQIGTFKVASVASEKMKILELPFASGTMSMWVLLPDEVSGLEQLETTISIEKLTEWTSSSVMEERKIKVFLPRMKMEEKYNLTSVLMAMGMTDLFSSSANLSGISSTLQKKGFRSQELGDKYAKPMLESPALTPQVTAWDNSWIVAHPAAIEPDLCYQIMEQKWKPFDWPDFRLPMRVSCRFRTMEALNKANTSFALDFFKHECQEDDDENILFSPFSISSALATVYLGAKGNTADQMAKTEIGKSGNIHAGFKALDLEINQPTKNYLLNSVNQLYGEKSLPFSKEYLQLAKKYYSAEPQSVDFLGKANEIRREINSRVEHQTEGKIKNLLPPGSIDSLTRLVLVNALYFKGNWATKFEAEDTRHRPFRINMHTTKQVPMMYLRDKFNWTYVESVQTDVLELPYVNNDLSMFILLPRDITGLQKLINELTFEKLSAWTSPELMEKMKMEVYLPRFTVEKKYDMKSTLSKMGIEDAFTKVDSCGVTNVDEITTHIVSSKCLELKHIQINKKLKCNKAVAMEQVSASIGNFTIDLFNKLNETSRDKNIFFSPWSVSSALALTSLAAKGNTAREMAEDPENEQAENIHSGFKELMTALNKPRNTYSLKSANRIYVEKNYPLLPTYIQLSKKYYKAEPYKVNFKTAPEQSRKEINNWVEKQTERKIKNFLSSDDVKNSTKSILVNAIYFKAEWEEKFQAGNTDMQPFRMSKNKSKLVKMMYMRHTFPVLIMEKLNFKMIELPYVKRELSMFILLPDDIKDSTTGLEQLERELTYEKLSEWADSKKMSVTLVDLHLPKFSMEDRYDLKDALKSMGMASAFNSNADFSGMTGFQAVPMESLSASTNSFTLDLYKKLDETSKGQNIFFASWSIATALAMVHLGAKGDTATQVAKGPEYEETENIHSGFKELLSAINKPRNTYLMKSANRLFGDKTYPLLPKFLELVARYYQAKPQAVNFKTDAEQARAQINSWVENETESKIQNLLPAGSIDSHTVLVLVNAIYFKGNWEKRFLEKDTSKMPFRLSKTETKPVQMMFLKDTFLIHHERTMKFKIIELPYVGNELSAFVLLPDDISDNTTGLELVERELTYEKLAEWSNSASMMKAKVELYLPKLKMEENYDLKSVLSDMGIRSAFDPAQADFTRMSEKKDLFISKVIHKAFVEVNEEDRIVQLASGRLTGRCRTLANKELSEKNRTKNLFFSPFSISSALSMILLGSKGNTEAQIAKVLSLSKAEDAHNGYQSLLSEINNPDTKYILRTANRLYGEKTFEFLSSFIDSSQKFYHAGLEQTDFKNASEDSRKQINGWVEEKTEGKIQKLLSEGIINSMTKLVLVNAIYFKGNWQEKFDKETTKEMPFKINKNETKPVQMMFRKGKYNMTYIGDLETTVLEIPYVDNELSMIILLPDSIQDESTGLEKLERELTYEKLMDWINPNMMDSTEVRVSLPRFKLEENYELKPTLSTMGMPDAFDLRTADFSGISSGNELVLSEVVHKSFVEVNEEGTEAAAATAGIMLLRCAMIVANFTADHPFLFFIRHNKTNSILFCGRFCSP

SEQ ID NO: 259 - protein, 386 aa, synthetic construct
MGSIGTASTEFCFDMFKEMKVQHANQNIIFSPLTIISALSMVYLGARDNTKAQMEKVIHFDKITGFGESVESQCGTSVSIHTSLKDMLSEITKPSDNYSLSLASRLYAEETYPILPEYLQCMKELYKGGLETVSFQTAADQARELINSWVESQTNGVIKNFLQPGSVDPQTEMVLVNAIYFKGMWEKAFKDEDTQEVPFRITEQESKPVQMMYQVGSFKVATVAAEKMKILEIPYTHRELSMFVLLPDDISGLEQLETTISFEKLTEWTSSNMMEERKVKVYLPHMKIEEKYNLTSVLMALGMTDLFSPSANLSGISTAQTLMMSEAIHGAYVEIYEAGREMASSTGVQVEVTSVLEEVRADKPFLFFIRHNPTNSMVVFGRYMSP

SEQ ID NO: 260 - protein, 2104 aa, synthetic construct
MTSNTCHEADEFENIDFRMDSISVTNTKFCFDVFNEMKVHHVNENILYSPLSILTALAMVYLGARGNTESQMKKALHFDSITGGGSTTDSQCGSSEYIHNLFKEFLTEITRTNATYSLEIADKLYVDKTFTVLPEYINCARKFYTGGVEEVNFKTAAEEARQLMNSWVEKETNGQIKDLLVPSSVDFGTMMVFINTIYFKGIWKTAFNTEDTREMPFSMTKQESKPVQMMCLNDTFNMVTLPAEKMRILELPYASGELSMLVLLPDEVSGLERIEKAINFEKLREWTSTNAMEKKSMKVYLPRMKIEEKYNLTSTLMALGMTDLFSRSANLTGISSVDNLMISDAVHGAFMEVNEEGTEAAGSTGAIGNIKHSVEFEEFRADHPFLFLIRYNPTNVILFFDNSEFTMGSIGAVSTEFCFDVFKELRVHHANENIFYSPFTIISALAMVYLGAKDSTRTQINKVVRFDKLPGFGDSIEAQCGTSANVHSSLRDILNQITKPNDIYSFSLASRLYADETYTILPEYLQCVKELYRGGLESINFQTAADQARELINSWVESQTSGIIRNVLQPSSVDSQTAMVLVNAIYFKGLWEKGFKDEDTQAIPFRVTEQENKSVQMMYQIGTFKVASVASEKMKILELPFASGTMSMWVLLPDEVSGLEQLETTISIEKLTEWTSSSVMEERKIKVFLPRMKMEEKYNLTSVLMAMGMTDLFSSSANLSGISSTLQKKGFRSQELGDKYAKPMLESPALTPQATAWDNSWIVAHPPAIEPDLYYQIMEQKWKPFDWPDFRLPMRVSCRFRTMEALNKANTSFALDFFKHECQEDDSENILFSPFSISSALATVYLGAKGNTADQMAKVLHFNEAEGARNVTTTIRMQVYSRTDQQRLNRRACFQKTEIGKSGNIHAGFKGLNLEINQPTKNYLLNSVNQLYGEKSLPFSKEYLQLAKKYYSAEPQSVDFVGTANEIRREINSRVEHQTEGKIKNLLPPGSIDSLTRLVLVNALYFKGNWATKFEAEDTRHRPFRINTHTTKQVPMMYLSDKFNWTYVESVQTDVLELPYVNNDLSMFILLPRDITGLQKLINELTFEKLSAWTSPELMEKMKMEVYLPRFTVEKKYDMKSTLSKMGIEDAFTKVDNCGVTNVDEITIHVVPSKCLELKHIQINKELKCNKAVAMEQVSASIGNFTIDLFNKLNETSRDKNIFFSPWSVSSALALTSLAAKGNTAREMAEDPENEQAENIHSGFNELLTALNKPRNTYSLKSANRIYVEKNYPLLPTYIQLSKKYYKAEPHKVNFKTAPEQSRKEINNWVEKQTERKIKNFLSSDDVKNSTKLILVNAIYFKAEWEEKFQAGNTDMQPFRMSKNKSKLVKMMYMRHTFPVLIMEKLNFKMIELPYVKRELSMFILLPDDIKDSTTGLEQLERELTYEKLSEWADSKKMSVTLVDLHLPKFSMEDRYDLKDALRSMGMASAFNSNADFSGMTGERDLVISKVCHQSFVAVDEKGTEAAAATAVIAEAVPMESLSASTNSFTLDLYKKLDETSKGQNIFFASWSIATALTMVHLGAKGDTATQVAKGPEYEETENIHSGFKELLSALNKPRNTYSMKSANRLFGDKTYPLLPTKTKPVQMMFLKDTFLIHHERTMKFKIIELPYMGNELSAFVLLPDDISDNTTGLELVERELTYEKLAEWSNSASMMKVKVELYLPKLKMEENYDLKSALSDMGIRSAFDPAQADFTRMSEKKDLFISKVIHKAFVEVNEEDRIVQLASGRLTGNTEAQIAKVLSLSKAEDAHNGYQSLLSEINNPDTKYILRTANRLYGEKTFEFLSSFIDSSQKFYHAGLEQTDFKNASEDSRKQINGWVEEKTEGKIQKLLSEGIINSMTKLVLVNAIYFKGNWQEKFDKETTKEMPFKINKNETKPVQMMFRKGKYNMTYIGDLETTVLEIPYVDNELSMIILLPDSIQDESTGLEKLERELTYEKLMDWINPNMMDSTEVRVSLPRFKLEENYELKPTLSTMGMPDAFDLRTADFSGISSGNELVLSEVVHKSFVEVNEEGTEAAAATAGIMLLRCAMIVANFTADHPFLFFIRHNKTNSILFCGRFCSP

SEQ ID NO: 261 - protein, 386 aa, synthetic construct
MASIGAASTEFCFDVFKELKTQHVKENIFYSPMAIISALSMVYIGARENTRAEIDKVVHFDKITGFGNAVESQCGPSVSVHSSLKDLITQISKRSDNYSLSYASRIYAEETYPILPEYLQCVKEVYKGGLESISFQTAADQARENINAWVESQTNGMIKNILQPSSVNPQTEMVLVNAIYLKGMWEKAFKDEDTQTMPFRVTQQESKPVQMMYQIGSFKVAVIASEKMKILELPYTSGQLSMLVLLPDDVSGLEQVESAITAEKLMEWTSPSIMEERTMKVYLPRMKMVEKYNLTSVLMALGMTDLFTSVANLSGISSAQGLKMSQAIHEAFVEIYEAGSEAVGSTGVGMEITSVSEEFKADLSFLFLIRHNPTNSIIFFGRCISP

SEQ ID NO: 262 - protein, 420 aa, synthetic construct
MGSIGAASTEFCFDVFRELRVQHVNENIFYSPFSIISALAMVYLGARDNTRTQIDKISQFQALSDEHLVLCIQQLGEFFVCTNRERREVTRYSEQTEDKTQDQNTGQIHKIVDTCMLRQDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 263 - protein, 386 aa, synthetic construct
MGSIGAASAEFCLDIFKELKVQHVNENIIFSPMTIISALSLVYLGAKEDTRAQIEKVVPFDKIPGFGEIVESQCPKSASVHSSIQDIFNQIIKRSDNYSLSLASRLYAEESYPIRPEYLQCVKELDKEGLETISFQTAADQARQLINSWVESQTNGMIKNILQPSSVNSQTEMVLVNAIYFRGLWQKAFKDEDTQAVPFRITEQESKPVQMMQQIGSFKVAEIASEKMKILELPYASGQLSMLVLLPDDVSGLEKLESSITVEKLIEWTSSNLTEERNVKVYLPRLKIEEKYNLTSVLAALGITDLFSSSANLSGISTAESLKLSRAVHESFVEIQEAGHEVEGPKEAGIEVTSALDEFRVDRPFLFVTKHNPTNSILFLGRCLSP

SEQ ID NO: 264 - protein, 386 aa, synthetic construct
MGSISAASGEFCLDIFKELKVQHVNENIFYSPMVIVSALSLVYLGARENTRAQIDKVIPFDKITGSSEAVESQCGTPVGAHISLKDVFAQIAKRSDNYSLSFVNRLYAEETYPILPEYLQCVKELYKGGLETISFQTAADQAREIINSWVESQTDGKIKNILQPSSVDPQTKMVLVSAIYFKGLWEKSFKDEDTQAVPFRVTEQESKPVQMMYQIGSFKVAAIAAEKIKILELPYASEQLSMLVLLPDDVSGLEQLEKKISYEKLTEWTSSSVMEEKKIKVYLPRMKIEEKYNLTSILMSLGITDLFSSSANLSGISSTKSLKMSEAVHEASVEIYEAGSEASGITGDGMEATSVFGEFKVDHPFLFMIKHKPTNSILFFGRCISP

SEQ ID NO: 265 - protein, 386 aa, synthetic construct
MGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIARRLYAEEKYPILPEYIQCVKELYKGGLESISFQTAAEKSRELINSWVESQTNGTIKNILQPSSVSSQTDMVLVSAIYFKGLWEKAFKEEDTQTIPFRITEQESKPVQMMSQIGTFKVAEIPSEKCRILELPYASGRLSLWVLLPDDISGLEQLETAITFENLKEWTSSSKMEERKIRVYLPRMKIEEKYNLTSVLKSLGITDLFSSSANLSGISSAESLKVSAAFHEASVEIYEAGSKGVGSSEAGVDGTSVSEEIRADHPFLFLIKHNPSDSILFFGRCFSP

SEQ ID NO: 266 - protein, 386 aa, synthetic construct
MGSIGAASTEFCFDVFKELKVQHVNENIIISPLSIISALSMVYLGAREDTRAQIDKVVHFDKITGFGEAIESQCPTSESVHASLKETFSQLTKPSDNYSLAFASRLYAEETYPILPEYLQCVKELYKGGLETINFQTAAEQARQVINSWVESQTDGMIKSLLQPSSVDPQTEMILVNAIYFRGLWERAFKDEDTQELPFRITEQESKPVQMMSQIGSFKVAVVASEKVKILELPYASGQLSMLVLLPDDVSGLEQLESSITVEKLIEWISSNTKEERNIKVYLPRMKIEEKYNLTSVLVALGITDLFSSSANLSGISSAESLKISEAVHEAFVEIQEAGSEVVGSPGPEVEVTSVSEEWKADRPFLFLIKHNPTNSILFFGRYISP

SEQ ID NO: 267 - protein, 386 aa, synthetic construct
MGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIARRLYAEEKYPILQEYIQCVKELYKGGLESISFQTAAEKSRELINSWVESQTNGTIKNILQPSSVSSQTDMVLVSAIYFKGLWEKAFKEEDTQTIPFRITEQESKPVQMMSQIGTFKVAEIPSEKCRILELPYASGRLSLWVLLPDDISGLEQLETSITFENLKEWTSSSKMEERKIRVYLPRMKIEEKYNLTSVLKSLGITDLFSSSANLSGISSAESLKVSAVFHEASVEIYEAGSKGVGSSEAGVDGTSVSEEIRADHPFLFLIKHNPSDSILFFGRCFSP

SEQ ID NO: 268 - protein, 399 aa, synthetic construct
MLNLMHPKQFCCTMGSIGPVSTEVCCDIFRELRSQSVQENVCYSPLLIISTLSMVYIGAKDNTKAQIEKAIHFDKIPGFGESTESQCGTSVSIHTSLKDIFTQITKPSDNYSISIASRLYAEEKYPILPEYIQCVKELYKGGLESISFQTAAEKSRELINSWVESQTNGTIKNILQPSSVSSQTDMVLVSAIYFKGLWEKAFKEEDTQTVPFRITEQESKPVQMMSQIGTFKVAEIPSEKCRILELPYASGRLSLWVLLPDDISGLEQLETAITSENLKEWTSSSKMEERKIKVYLPRMKIEEKYNLTSVLKSLGITDLFSSSANLSGISSAESLKVSGAFHEAFVEIYEAGSKAVGSSGAGVEDTSVSEEIRADHPFLFFIKHNPSDSILFFGRCFSP

SEQ ID NO: 269 - protein, 389 aa, synthetic construct
EAEAGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 270 - protein, 474 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEAGSIGTASAEFCFDVFKELKVHHVNENIFYSPLSIISALSMVYLGARENTKTQMEKVIHFDKITGLGESMESQCGTGVSIHTALKDMLSEITKPSDNYSLSLASRLYAEQTYAILPEYLQCIKELYKESLETVSFQTAADQARELINSWIESQTNGVIKNFLQPGSVDSQTELVLVNAIYFKGMWEKAFKDEDTQEVPFRITEQESRPVQMMYQAGSFKVATVAAEKIKILELPYASGELSMLVLLPDDISGLEQLETTISFEKLTEWTSSNMMEDRNMKVYLPRMKIEEKYNLTSVLIALGMTDLFSPAANLSGISAAESLKMSEAIHAAYVEIYEADSEIVSSAGVQVEVTSDSEEFRVDHPFLFLIKHNPTNSVLFFGRCISP

SEQ ID NO: 271 - protein, 389 aa, synthetic construct
EAEAGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 272 - protein, 474 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEAGSIGAASTEFCFDVFRELRVQHVNENIFYSPFSIISALAMVYLGARDNTRTQIDKVVHFDKLPGFGESMEAQCGTSVSVHSSLRDILTQITKPSDNFSLSFASRLYAEETYAILPEYLQCVKELYKGGLESISFQTAADQARELINSWVESQTNGIIKNILQPSSVDSQTTMVLVNAIYFKGMWEKAFKDEDTQAMPFRMTEQESKPVQMMYQVGSFKVAMVTSEKMKILELPFASGMMSMFVLLPDEVSGLEQLESTISFEKLTEWTSSTMMEERRMKVYLPRMKMEEKYNLTSVFMALGMTDLFSSSANMSGISSTVSLKMSEAVHAACVEIFEAGRDVVGSAEAGMDVTSVSEEFRADHPFLFFIKHNPTNSILFFGRWMSP

SEQ ID NO: 273 - protein, 426 aa, synthetic construct
TRAPDCGGILTPLGLSYLAEVSKPHAEVVLRQDLMAQRASDLFLGSMEPSRNRITSVKVADLWLSVIPEAGLRLGIEVELRIAPLHAVPMPVRISIRADLHVDMGPDGNLQLLTSACRPTVQAQSTREAESKSSRSILDKVVDVDKLCLDVSKLLLFPNEQLMSLTALFPVTPNCQLQYLPLAAPVFSKQGIALSLQTTFQVAGAVVPVPVSPVPFSMPELASTSTSHLILALSEHFYTSLYFTLERAGAFNMTIPSMLTTATLAQKITQVGSLYHEDLPITLSAALRSSPRVVLEEGRAALKLFLTVHIGAGSPDFQSFLSVSADVTAGLQLSVSDTRMMISTAVIEDAELSLAASNVGLVRAALLEELFLAPVCQQVPAWMDDVLREGVHLPHLSHFTYTDVNVVVHKDYVLVPCKLKLRSTMA

SEQ ID NO: 274 - protein, 388 aa, synthetic construct; identical to SEQ ID NO: 197

SEQ ID NO: 275 - protein, 1185 aa, synthetic construct
CSTWGGGHFSTFDKYQYDFTGTCNYIFATVCDESSPDFNIQFRRGLDKKIARIIIELGPSVIIVEKDSISVRSVGVIKLPYASNGIQIAPYGRSVRLVAKLMEMELVVMWNNEDYLMVLTEKKYMGKTCGMCGNYDGYELNDFVSEGKLLDTYKFAALQKMDDPSEICLSEEISIPAIPHKKYAVICSQLLNLVSPTCSVPKDGFVTRCQLDMQDCSEPGQKNCTCSTLSEYSRQCAMSHQVVFNWRTENFCSVGKCSANQIYEECGSPCIKTCSNPEYSCSSHCTYGCFCPEGTVLDDISKNRTCVHLEQCPCTLNGETYAPGDTMKAACRTCKCTMGQWNCKELPCPGRCSLEGGSFVTTFDSRSYRFHGVCTYILMKSSSLPHNGTLMAIYEKSGYSHSETSLSAIIYLSTKDKIVISQNELLTDDDELKRLPYKSGDITIFKQSSMFIQMHTEFGLELVVQTSPVFQAYVKVSAQFQGRTLGLCGNYNGDTTDDFMTSMDITEGTASLFVDSWRAGNCLPAMERETDPCALSQLNKISAETHCSILTKKGTVFETCHAVVNPTPFYKRCVYQACNYEETFPYICSALGSYARTCSSMGLILENWRNSMDNCTITCTGNQTFSYNTQACERTCLSLSNPTLECHPTDIPIEGCNCPKGMYLNHKNECVRKSHCPCYLEDRKYILPDQSTMTGGITCYCVNGRLSCTGKLQNPAESCKAPKKYISCSDSLENKYGATCAPTCQMLATGIECIPTKCESGCVCADGLYENLDGRCVPPEECPCEYGGLSYGKGEQIQTECEICTCRKGKWKCVQKSRCSSTCNLYGEGHITTFDGQRFVFDGNCEYILAMDGCNVNRPLSSFKIVTENVICGKSGVTCSRSISIYLGNLTIILRDETYSISGKNLQVKYNVKKNALHLMFDIIIPGKYNMTLIWNKHMNFFIKISRETQETICGLCGNYNGNMKDDFETRSKYVASNELEFVNSWKENPLCGDVYFVVDPCSKNPYRKAWAEKTCSIINSQVFSACHNKVNRMPYYEACVRDSCGCDIGGDCECMCDAIAVYAMACLDKGICIDWRTPEFCPVYCEYYNSHRKTGSGGAYSYGSSVNCTWHYRPCNCPNQYYKYVNIEGCYNCSHDEYFDYEKEKCMPCAMQPTSVTLPTATQPTSPSTSSASTVLTETTNPPV

SEQ ID NO: 276 - protein, 129 aa, synthetic construct [sequence omitted]

SEQ ID NO: 277 - protein, 129 aa, synthetic construct
KVFGRCELAAAMKRHGLDNYRGYSLGNWVCVAKFESNFNTQATNRNTDGSTDYGILQINSRWWCNDGRTPGSRNLCNIPCSALLSSDITASVNCAKKIVSDGNGMSAWVAWRNRCKGTDVQAWIRGCRL

SEQ ID NO: 278 - protein, 130 aa, synthetic construct
KVFERCELARTLKRLGMDGYRGISLANWMCLAKWESGYNTRATNYNAGDRSTDYGIFQINSRYWCNDGKTPGAVNACHLSCSALLQDNIADAVACAKRVVRDPQGIRAWVAWRNRCQNRDVRQYVQGCGV

SEQ ID NO: 279 - protein, 129 aa, synthetic construct
KVFERCELARTLKKLGLDGYKGVSLANWLCLTKWESSYNTKATNYNPSSESTDYGIFQINSKWWCNDGKTPNAVDGCHVSCRELMENDIAKAVACAKHIVSEQGITAWVAWKSHCRDHDVSSYVEGCTL

SEQ ID NO: 280 - protein, 449 aa, synthetic construct
IEVNCSLYASGIGKDGTSWVACPRNLKPVCGTDGSTYSNECGICLYNREHGANVEKEYDGECRPKHVMIDCSPYLQVVRDGNTMVACPRILKPVCGSDSFTYDNECGICAYNAEHHTNISKLHDGECKLEIGSVDCSKYPSTVSKDGRTLVACPRILSPVCGTDGFTYDNECGICAHNAEQRTHVSKKHDGKCRQEIPEIDCDQYPTRKTTGGKLLVRCPRILLPVCGTDGFTYDNECGICAHNAQHGTEVKKSHDGRCKERSTPLDCTQYLSNTQNGEAITACPFILQEVCGTDGVTYSNDCSLCAHNIELGTSVAKKHDGRCREEVPELDCSKYKTSTLKDGRQVVACTMIYDPVCATNGVTYASECTLCAHNLEQRTNLGKRKNGRCEEDITKEHCREFQKVSPICTMEYVPHCGSDGVTYSNRCFFCNAYVQSNRTLNLVSMAAC

SEQ ID NO: 281 - protein, 139 aa, synthetic construct
MAGARGCVVLLAAALMLVGAVLGSEDRSRLLGAPVPVDENDEGLQRALQFAMAEYNRASNDKYSSRVVRVISAKRQLVSGIKYILQVEIGRTTCPKSSGDLQSCEFHDEPEMAKYTTCTFVVYSIPWLNQIKLLESKCQ

SEQ ID NO: 282 - protein, 450 aa, synthetic construct
SEVCFPRLGCFSDDAPWAGIVQRPLKILPWSPKDVDTRFLLYTNQNQNNYQELVADPSTITNSNFRMDRKTRFIIHGFIDKGEEDWLSNICKNLFKVESVNCICVDWKGGSRTGYTQASQNIRIVGAEVAYFVEVLKSSLGYSPSNVHVIGHSLGSHAAGEAGRRTNGTIERITGLDPAEPCFQGTPELVRLDPSDAKFVDVIHTDAAPIIPNLGFGMSQTVGHLDFFPNGGKQMPGCQKNILSQIVDIDGIWEGTRDFVACNHLRSYKYYADSILNPDGFAGFPCDSYNVFTANKCFPCPSEGCPQMGHYADRFPGKTNGVSQVFYLNTGDASNFARWRYKVSVTLSGKKVTGHILVSLFGNEGNSRQYEIYKGTLQPDNTHSDEFDSDVEVGDLQKVKFIWYNNNVINPTLPRVGASKITVERNDGKVYDFCSQETVREEVLLTLNPC

SEQ ID NO: 283 - protein, 455 aa, synthetic construct
GLVAADRITGGKDFRDIESKFALRTPEDTAEDTCHLIPGVTESVANCHFNHSSKTFVVIHGWTVTGMYESWVPKLVAALYKREPDSNVIVVDWLSRAQQHYPVSAGYTKLVGQDVAKFMNWMADEFNYPLGNVHLLGYSLGAHAAGIAGSLTSKKVNRITGLDPAGPNFEYAEAPSRLSPDDADFVDVLHTFTRGSPGRSIGIQKPVGHVDIYPNGGTFQPGCNIGEALRVIAERGLGDVDQLVKCSHERSVHLFIDSLLNEENPSKAYRCNSKEAFEKGLCLSCRKNRCNNMGYEINKVRAKRSSKMYLKTRSQMPYKVFHYQVKIHFSGTESNTYTNQAFEISLYGTVAESENIPFTLPEVSTNKTYSFLLYTEVDIGELLMLKLKWISDSYFSWSNWWSSPGFDIGKIRVKAGETQKKVIFCSREKMSYLQKGKSPVIFVKCHDKSLNRKSG

SEQ ID NO: 284 - protein, 685 aa, synthetic construct
APKKGVRWCVISTAEYSKCRQWQSKIRRTNPMFCIRRASPTDCIRAIAAKRADAVTLDGGLVFEADQYKLRPVAAEIYGTEENPQTYYYAVAVVKKGFNFQLNQLQGRKSCHTGLGRSAGWNIPIGLLRRFLDWAGPPEPLQKAVAKFFSQSCVPCADGNAYPNLCQLCIGKGKDKCACSSQEPYFGYSGAFNCLHKGIGDVAFVKESTVFENLPQKADRDKYELLCPDNTRKPVEAFRECHLARVPSHAVVARSVNGKENSIWELLYQSQKKFGKSNPQEFQLFGSPGQQKDLLFRDATIGFLKIPSKIDSKLYLGLPYLTAIQGLRETAAEVEARQAKVVWCAVGPEELRKCRQWSSQSSQNLNCSLASTTEDCIVQVLKGEADAMSLDGGFIYTAGKCGLVPVLAENQKSRQSSSSDCVHRPTQGYFAVAVVRKANGGITWNSVRGTKSCHTAVDRTAGWNIPMGLLVNQTGSCKFDEFFSQSCAPGSQPGSNLCALCVGNDQGVDKCVPNSNERYYGYTGAFRCLAENAGDVAFVKDVTVLDNTNGQNTEEWARELRSDDFELLCLDGTRKPVTEAQNCHLAVAPSHAVVSRKEKAAQVEQVLLTEQAQFGRYGKDCPDKFCLFRSETKNLLFNDNTEVLAQLQGKTTYEKYLGSEYVTAIANLKQCSVSPLLEACAFMMR

SEQ ID NO: 285 - protein, 689 aa, synthetic construct
APRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 286 - protein, 471 aa, synthetic construct
MFEKSKFVVSFLLLLQLFCVLGVHGQESGNGTTSDTAYACDIGATPFDGFNATIYQYQASDDNSIQDPVFMSTGYLQRNQLHSTTGVTNPGFNIFTAGVATTTLYGIPNVNYQNMLLELKGYFRADASGNYGLSLRNIDDSAILFFGRETAFECCNENLIPLDEAPTDYSLFTIKEGEASTNPDSYTYTQYLEAGRYYPVRTFFANIRTRAVFNFTMTLPDGSELTDFQNYIFQFGALNQQQCQAEIVTRENYTTTTEPWTGTFEATTTVIPSGTEPGTVIVQTPYSTIDSTSTWTGTFTTFTTDADGSTIAVVPSSTIDDHFASTETVLTDTAISTTVITVTSCGTSKCTKTTALTGVTQRTLTIDDRTTVVTTYCPLPTDVATIKTASVSGSEVVQTIYTAKHSQAVSYVHPSTVTITREVCDAQTCTQATIVTGEILQTTVVDSGSTTVVPKYVPVETHEPTFELSTL

SEQ ID NO: 287 - protein, 233 aa, synthetic construct
MQFTFASTSVVVSLIAALAKPAVATPPACLLACAAEVVKESSDCDALNNIQCICENEGSAIHACLESTCPDGLSSTALQSFEDVCESVGTEANLDESSSSQSSSSSSSSESSSSSVSSSSSSASSSSETSSSVTSSSVTSSSTAVSSSTESSSSVEPSTSHSSSHSSSEVSSTVAPTTSVAPTTSSITTSSTSLTSATTSSVTISIEPTSDAADKVIIPGLAGLVGALAVGLI

SEQ ID NO: 288 - protein, 203 aa, synthetic construct
MQYRSLFLGSALLAAANAAVYNTTVTDVVSELETTVLTITSCAEDKCITSKSTGLITTSTLTKHGVVTVVTTVCDLPSTTKSYVPPAKTTTIPPPEKTTTTVPPPAKTTTTVPPPAKTTSTVPPPAKTSSHHESTITVTVPSSTSTKKIETESTTYHFVTQTTTARNITPPAITTQSHGAAGMNAANFVGLGAAAVAAAALVL

SEQ ID NO: 289 - protein, 439 aa, synthetic construct
MSLLLFLVLGAFLLSSVKAADIGAFRLRVYTPGRFTNGALNFNNWGYQYLDASSSNGQLFAGYATVTSVTTFLAPDDEGFVWGSSLGGYPGFLGIGAGATAFHLTGIPGDALSWYIEDNILKTSSPTYVCSRNDGDVVVGIEANTRWLAMHDTSQLPPNYYCFQADYEIVALWYIPDTTSTWTGTETSTTTDDDGSVIELVPTPLPDTTSTWTGTFTTFTTDDDGSVIELVPTPLPDSTSTWTGTYTTFTTDEDGSTIAVVPSSTIDSTSTWTGTYTTFTTDEDGSTIAVVPSSTIDSTSTWTGTYTTFTTDEDGSTIAVYHHLLSTPHPPGLVLTPRSLPMRMEVLLLWYHHLLSTLHPPGLVLTPRSLPMRMEVLLLWYHRLLSTPHPGLVLTPRSLPMRMEVLLLYHHLLSTPHPPGLVLTPRSLPMRMEVLLLWY

SEQ ID NO: 290 - protein, 2140 aa, synthetic construct
MKLQLQSFVFFLLSAVNVLADDSYGCSIATSPRSTGFVANLYEFPNMAISNAELKTYVRYRYKEGRLYDTISNIISPYFYYQGQGANSAYGTLYGRPNVYLYNFSMELKGYFRPPITGQYTIDFNGANVDDAAMVFFGKAGAFDCCNSDYILPEQSAEYSLYSVYPHTATDQILSATIYLEAGKYYPLRVTYTNIGNIGSLDLRVVLPSGASITSLGAFVYQFPNNLSPGTCTPDVEYFTTTTQAWTGTYETTYTVPPSGTQPGTVIIETPESYVTTTQPWTGTYETTYTVPPTGTEPGTVIIETPESYVTTTQPWTGTYETTYTVPPSGTEPGTVIIETPESYVTTTQPWTGTYETTYTVPPSGTEPGTVIIETPESYVTTTQPWTGTYETTYTVPPSGTEPGIVIIETPESYVTTTQPWTGTYETTYTVPPSGTEPGTVVIETPEITDCEAVCCGAVPTSDPLRRRDVCDCETFCCPGDTNCETYVTTTQPWTGTYETTYTVPPSGTEPGTVIIETPESYVTTTQPWTGTYETTYTVPPSGTEPGIVIIETPESYVTTTQPWTGTYETTYTVPPTGTEPGTVIIETPESYVTTTQPWTGTYETTYTVPPSGTEPGIVIIETPESYVTTTQPWTGTYETTYTVPPSGTEPGTVIIETPESYVTTTQPWTGTYETTYTVPPTGTEPGTVIIETPESYVTTTQPWTGTYETTYTVPPSGTEPGIVIIETPESYVTTTQPWTGTYETTYTVPPTGTEPGTVIIETPESYVTTTQPWTGTYETTYTVPPTGTEPGTVIIETPESYVTTTQPWTGTYETTYTVPPSGTEPGTVIIETPESYVTTTQPWTGTYETTYTVPPSGTEPGTVVIETPEITDCEAVCCGAVPTSDPLRRRDVCDCETFCCPGDTNCETYVTTTQPWTGTYETTYTVPPSGTEPGTVIIETPESYVTTTQPWTGTYETTYTVPPTGTEPGTVIIETPESYVTTTQPWTGTYETTYTVPPSGTQPGTVIIETPESYVTTTQPWTGTYETTYTVPPTGTEPGTVIIETPESYVTTTQPWTGTYETTYTVPPSGTEPGTVIIETPESYVTTTQPWTGTYETTYTVPPSGTQPGTVIIETPESYVTTTQPWTGTYETTYTVPPTGTEPGTVIIETPESYVTTTQPWTGTYETTYTVPPSGTEPGIVIIETPESYVTTTQPWTGTYETTYTVPPTGTEPGTVIIETPESYVTTTQPWTGTYETTYTVPPTGTEPGTVIIETPESYVTTTQPWTGTYETTYTVPPSGTEPGTVIIETPESYVTTTQPWTGTYETTYTVPPSGTEPGTVVIETPEITDCEAVCCGAVPTSDPLRRRDVCDCETFCCPGDTNCETYVTTTQPWTGTYETTYTVPPSGTEPGTVIIETPESYVTTTQPWTGTYETTYTVPPTGTEPGTVIIETPESYVTTTQPWTGTYETTYTVPPSGTQPGTVIIETPESYVTTTQPWTGTYETTYTVPPTGTEPGTVIIETPESYVTTTQPWTGTYETTYTVPPSGTEPGTVIIETPESYVTTTQPWTGTYETTYTVPPSGTQPGTVIIETPESYVTTTQPWTGTYETTYTVPPTGTEPGTVIIETPESYVTTTQPWTGTYETTYTVPPSGTEPGIVIIETPESYVTTTQPWTGTYETTYTVPPTGTEPGTVIIETPESYVTTTQPWTGTYETTYTVPPSGTEPGTVIIETPESYVTTTQPWTGTYETTYTVPPSGTQPGTVIIETPESYVTTTQPWTGTYETTYTVPPSGTEPGTVIVETPDVPGSYVTTTQPWTGTYETTHTVPPTGTEPGTVVVETPDVPGSYVTTTQPWTGTYETTHTVPPTGTEPGTVVVETPDVPGSYVTTTQPWTGTYETTYTVPPSGTEPGTVIVETPDVPGSYVTTTQPWTGTYETTHTVPPTGTEPGTVVVETPDVPGSYVTTTQPWTGVYKTTYTVPPSGTIPGTVIIETPFGYFNTSSISTKTDKRTITSVVPCSQCSESKTQYITPTGPGDVTVIISQPPSKITLSSPEDKTKTDFITSTGSIGGGSPPSHPNDKPGIITTPTQPIGGGNPSDIPSAISSVSSGGNSRASVPSFSTSSAISVQVSSLYDENSGSTFEVSLLFSVVSGFFLTLMV

SEQ ID NO: 291 - protein, 1149 aa, synthetic construct
MKFPVPLLFLLQLFFIIATQGDESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSCYETTVSKITEWTTYTTPWTGTFETTRTITPTGTEGTVVIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPSGTEPGTVVIETPEIVDCEAYCCASVAIKKRELCQCENFCCSWDQSCQTYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIINCEAVCCGPFLTAFSFRKREECQCENICCPGDTNCETYVTTTQPWTGTYETTYTVPPTGTEPGTVIIETPESYVTTTQPWTGTYETTYTVPSTGTEPGTVIIETPESYVTTTQPWTGTYETTFTVPPTGTEPGTVVIETPESYVTTTQPWTGTYETTYSVPPSGTEPGTVVIETPEASTARTKFTTVTSSWTGVFTTTKTLPASGTEPATIVIQTPTGYFNTSSLVSTRTKTNVDTVTRVIPCPICTAPKTITVVPEEPNESVSVIISQPQSSSTDTTLSKPDSVRVISQPETASQMDTSLSKTDSAVISTETAGNNIIPLAGSHSYNTIVTTVTDSPQVAQSTTATSSSNVHLTISTQTTTPSLVYSSSLSTVHQVSPSNGGFRSSITVHPLLSVIGAIFGALFM

SEQ ID NO: 292 - protein, 715 aa, synthetic construct
MTKFTILLLVLLKFYSILAIEVDGSANGQPLAHPIVVEVHEATKWITHTSPWTGTPEAIRTVTGETPYEQKIARYDEFNPRLANREIIDCVAFCCGDATSSPSITEPESTATELPESYVTINRPWSLSWIPDVPPGSPYWSTSTIPPSGTEPGTVIIYFYLYDDARKRREINFGSTQPYHGRPKLLGSIEKRELCQCDAVCCLGDLSCEVYVTTTQPWTGTYETTYTITPTGSEPGTVIIETPELYVTTTQPWTGTYETTYTITPTGSEPGTVIIETPESYVTTTQPWTGTYETTYTITPTGSEPGTVIIETPESYVTTTQPWTGTYETTYTITPTGSEPGTVIIETPESYVTTTQPWTGTYETTYTVPPSGTEPGAVIIETPELYVTTTQPWTGTYETTYTITPTGSEPGTVIIETPESYVTTTQPWTGTYETTYTVPPSGTEPGTVIIETPELYVTTTQPWTGTYETTYTITPTGSEPGTVIVEIPVSYVNSTQISTSTYDTTDTVLSSGVEPGTIAIETPIVYLNTSVSAFSRPWTKIDTVTQFSSCAVCSKPETITVTPENPIDTVTIIISQPQSTSQSNTPTSFKANSTSAFSRFDEDSIPVFGSYSYEITVNIDVNTEDDTTTNLNADTTIIIGSLSAIRTVAGSSSNYHASNISPTINSQKTASSVVVHSDSSATVYQFSPSNGAPWLSVQISTLLSVVGTLLAAVLL

SEQ ID NO: 293 - protein, 2498 aa, synthetic construct
MNFRYLLILPIYASIVLGQVGDFQLLLNAKEPIRNSPSLLSSNYGNLTLPAMANGALESHFDYGNAYVGDDQITVVYHLPDEHGQINAYRQDTDEYIGYLGLVTDDYGEYTYLSVIMPGVQYDQTTSVNWYIENEELKSTSINVQPLLGCYYKNPPQYSWYWASIDEPGNIASSNFVCEPCKVYVDFVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADATSVWTGDHTTWTTDDDGNVIEQIPTPSADITSMWTGSETSWTTDADGTVIELVPTPSADITSMWTGSETSWTTDADGTVIELVPTPSADITSMWTGSETSWTTDADGTVIELVPTPSADTTSVWTGSYTTWTTDEDGTVIEQVPTPSADTPSADTTSVWTGSYTTWTTDEDGTVIEQVPTPSADTTSVWTGSYTTWTTDEDGTVIEQVPTPSADTPSADTTSVWTGSYTTWTTEVGDGGSSTVVELVPTESSTSTNVMQTPVPSSGVSDGVSVFNGFNVEVFHYPADNYELANEISFLSYGYENLGLVTTVTGVSDINFDTDSNWPYYIDRDALGNTGSYVNATIEYEGFFRAPVDGEYVFSFSSTDYNSILFVGSPAAADQALQKREVQFLKPETSPDYVLLFNNTRDLGKTVSTTQYLLADQYYPLRVVIAAISQHALLDFQIKLPNGASLTQYQGYVYNFALEGSESTTVIGDKTSTWTGSYTTWTTDSDGSTIVVVPPATITADKTSTWTGSYTTWTTDSDGSTVVICPSITSDHNDKPSESTLTDSSISTTVVTVTSCDIEKCTKTTALTGVRETTLTTGGTTTVVTTYCPLPTDIVTVKTTSIDGSEVLQTIYTAKPNHVVPDVQTSTVTITREVCDAFTCTHATIVTGEILKTTTLADTHYTTVVPVYVPLETYQPAVELSTLETVLKSSDLASGPVVTAGSVQPSYQSGGVAESSLTVSEFEAHSTSDTVSQPSTISLQTGEANALKWSSFFGAALVPLVNVFFV

SEQ ID NO: 294 - protein, 2812 aa, synthetic construct
MQNTNDKLIIRTFYSISTIHGLLSINIFSDTRVYKFAIYSTDAVSLEPRTKNNMSLVTVLACFIIFAAHAFGQDTFYMLKVRTLTPNGYPLADSLSNPMQYWDLYYVPGGPRRLESSFVNWQPTTAAPINQFYCRLGTDGHMTGYNRVTGSVIGKLSFGTNAATALAFGSYDGDPSYPPQAFSISSSVSGTMTYLNVHYVNARSITWYSTTTATGETNVYINVASTGYTGDRTTYQAELWVEPFVPNIPVDTTTSIWTGSQTSYTTEVGENGGSTVIELIPTPPADATSTWTGTYTTRTTDADGSVIEQIPTPSADTTSVWTGTYTTWTTDADGSVIEQIPTPSADTTSVWTGTYTTWTTDADGSVIEQIPTPSADTTATWTGTETSYTTDVGEDGSSTVIELVPTPSADTTATWTGTETSYTTDVGEDGSSTVVELVPTPSADTTATWTGTETSYTTDVGEDGSSTVIELVPTPSADTTATWTGTETSYTTDVGEDGSSTVIELVPTPSADTTATWTGTETSYTTDVGEDGSSTVVELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPTPSADTTATWTGTETSYTTDVGEDGSSTVIELVPTPSADTTATWTGTETSYTTDVGEDGSSTVIELVPTPSADTTATWTGTETSYTTDVGEDGSSTVVELVPTPTPSADTTATWTGTETSYTTDVGEDGSSTVVELVPTPSADTTATWTGTETSYTTDVGEDGSSTVIELVPTPSADTTATWTGTETSYTTDVGEDGSSTVVELVPTPSADTTATWTGTETSYTTDVGEDGSSTVVELVPTPTADTTATWTGTETSYTTDVGEDGSSTVIELVPTPSADTTATWTGTETSYTTDVGEDGSSTVVELVPTPSADTTATWTGTETSYTTDVGEDGSSTVVELVPTPSADTTATWTGTETSYTTDVGEDGSSTVIELVPTPSADTTATWTGTETSYTTDVGEDGSSTVVELVPTPSADTTATWTGTETSYTTDVGEDGSSTVVELVPTPTADTTATWTGTETSYTTDVGEDGSSTVIELVPTPSADTTATWTGTETSYTTDVGEDGSSTVVELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPTPSADTTATWTGTETSYTTDVGEDGSSTVIELVPTPSADTTATWTGTETSYTTDVGEDGSSTVIELVPTPTPSADTTATWTGTETSYTTDVGEDGSSTVVELVPTPSADTTATWTGTETSYTTDVGEDGSSTVIELVPTPSADTTATWTGTETSYTTDVGEDGSSTVVELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVIELVPTPSADTTATWTGTETSYTTDVGEDGSSTVVELVPTPTPSADTTATWTGTETSYTTDVGEDGSSTVIELVPSDTETATNIVETPVPSSGVSDGVSVFDGFNVEVFHYPADNYELANEIGFLSYGYENLGLVTNATGVSDINFDTDSNWPYYIDRDALGNTGSYVNATIEYEGFFRAPVDGEYVFSFSNTDYNSILFVGSPAAAGQALQKRRVQFLKPETSPDHVLLFNNTRDLGQTISTTQYLLADQYYPLRVVIAAISQHALLDFQIKLPNGALLTQYQGYVYNFALEGSESTTVIGDKTSTWTGSYTTWTTDSDGSTVVVVPSATITADKTSTWTGSYTTWTTDSDGSTIVICPSITSDHNDKPSESTLTDGSISTTVVTVTSCDIEKCTKTTALTGVTETTLTTGGTTTVVTTYCPLPTDIVTVKTTSISGSEVLQTIYTAKPSHVVPNVHTLTVTITREVCDAFTCTQATIVTGEILKTTTLADTHSTTVVPVYVPLESYQSAVELSTLETVLKSSDFASGSAVTAGSAQPSYQSGGVAESSLTGSELEAHSTSDTVSQPSTISPQTGEANALRWSSFFGAALVPLVNVFFV

SEQ ID NO: 295 - protein, 860 aa, synthetic construct
MTKLTILLSVLLQLFSVLAEVPKKTEWSSHTTYWTSTLEALRTVTPTGTERAVIGEAPYEYKLIGNDQFDPGLNAKREIIDCEAVCCGAVPTSDPLKRRDVCECENVCCPGDDCETYVTTTQPWTGTYETTYTVPPSGTEPGTVVIETPEITDCEAVCCGAVPTSDPLRRRDVCECENVCCPGDDCETYVTTTQPWTGTYETTYTVPPSGTEPGTVVIETPEITDCEAVCCGAVPTSDPLRRRDVCECENVCCPGDDCETYVTTTQPWTGTYETTYTVPPTGTEPGTVVIETPVTYVTTTQPWTGTYETTYTVPPTGTEPGTVVIETPEITDCEAVCCGAVPTSDPLRRRDVCECENVCCPGDDCETYVTTTQPWTGTYETTYTVPPTGTEPGTVVIETPVTYVTTTQPWTGTYETTYTVPPTGTEPGTVVIETPVTYVTTTQPWTGTYETTYTVPPTGTEPGTVVIETPVTYVTTTQPWTGTYETTYTIPPTGTEPGTVVIETPEITDCEAVCCGAVPTSDPLRRRDVCECENVCCPGDDCETYVTTTQPWTGTYETTYTVPPTGTEPGTVVIETPVTYVTTTQPWTGTYETTYTVPPTGTEPGTVVIETPVTYVTTTQPWTGTYETTYTVPPTGTEPGTVVIETPVTYVTTTKPWTGTYETTHTVPASGTEPGTVIIETPIKYLNTSISASTSTWTKINTVTQFISCPVCTIPKTITVTPKISNETVTIIISQPHGTSSRTTTVVKTDGASVSSHSYKTALTTDVKPEEKTSTKLGTVTTVSGSHSAIDTVTGSLSDYHASSIPHTVKSEEKASSTVTHTISSSTVYQVSPSNGASWLSVRLNTALSIIGTLFAAVFI

SEQ ID NO: 296 - protein, 1436 aa, synthetic construct
MSKTKNGGSEFVHIAYVFHIEASTPSDYINMIQIVLFPHQAQITKRMNLVTLLVCNLLCVSLTLGQGVYRLKFPALVVTGRESVGTTVVNYDFLVGNTGQYGDLGEFFYDGEPYYCWNSTDSQPLSCSSSSSLLISTQNVTISHPDEDGTVYAYAERDGGLLGRFTVGSVSADWPQWAVIVYSTSSSAHPSSWYVDDNKLKLTSGLGPNNSTTLQACYFTQSSGRDRYAISLEGSPAYTGQVSCQATEFDLEFIPPSADTTSIWDGSYTTWTTDSNGIVVEQIPTPSADTTSIWTGSETSWTTDSDGTVIELVPTPSADATSIWTGDHTTWTTDSEGNVIEQIPTPSADTTSIWTGSETSWTTDSDGTVIELVPTPSADATSIWTGDHTTWTTDSEGNVIEQIPTPSADATSIWTGDHTTWTTDREGNVIEQIPTPSADTTSIWTGSETSWTTDSDGTVIELVPTPSADATSIWTGDHTTWTTDSEGNVIEQIPTPSADATSIWTGSETSWTTDSDGTVIELVPTPSADATSIWTGDHTTWTTDSEGNVIEQIPTPSADATSIWTGDHTTWTTDSEGNVIEQIPTPSADTTSIWTGSETSWTTDSDGTVIELVPTPSADATSIWTGDHTTWTTDSEGNVIEQIPTPSADATSIWTGDHTTWTTDSEGNVIEQIPTPSADTTSIWTGSETSWTTDSDGTVIELVPTPSADATSIWTGDHTTWTTDSEGNVIEQIPTPSADATSIWTGDHTTWTTDSEGNVIEQIPTPSADTTSIWTGSETSWTTDSDGTVIELVPTPSADATSIWTGDHTTWTTDSEGNVIEQIPTPSADATSIWTGSETSWTTDSDGTVIELVPTPSADATSIWTGDHTTWTTDSEGNVIEQIPTPSADATSIWTGDHTTWTTDSEGNVIEQIPTPSADTTSIWTGDHTTWTTEVGGDGSSIVVELVPSETGTATNVVQTPVPSSGISDGVSALDGFNVEVFHYPADNYELANEISFLSYGYENLGLVTTATGVSDINFDTDSNWPSYIDRNALGNTGSYVNATIKYEGFFRAPVDGDYEFSFSNIDYNSILFVGSAAADQALRKREAQFLKPETSPNHILFFNNSRDVGQTISTTQYLSADSYYPLRVVIAAVSQHALLDFQIKLPNGVSLTQFQGYVYNFALEGAESTTVIGDKTSTWTGTYTTWTTDSEGSTIVLCPSIISDHNGKPADTTLTDGSISTTVVTVTSCDIKKCTKTTALTGVTQKTLTVKGTTTVVTAYCPLPTDVATVKTISVGGSEVLQTVYTAKPSHIVPDVQTLTVTITREVCDALTCIPATIVTGEILKTTTLADTHSTTVIPVYVPLETHQPALDLITLETVLKSSDFANGPAITSVSVESLSHQSGVVVSEFDSDSTSGAVSQPSSAVSLQTGKASALKWSPFLGAAVISLFNVFFV

SEQ ID NO: 297 - protein, 2435 aa, synthetic construct
MNLFTILAWGFLYVPLVLGEGYYSLNFDARVPIALGILGSSYQKYTIMADRSLLGGSNIDLDVTFSGIIELLTNRVHIVVSLPDADGRVSVYDMYSGTSLGYLSFVCSLTTCEVHAVSSSSGATTWTLDGNQLIPTSPSTVYACYRSLVGLLAQYTLNDRTSITAQCEQTNLYVELAIPAFPETTAVWTGTYTTWTTDESGSVIEQMPTPSADTTTTWTGTYTTWTTDADGSVIEQIPTPPADTTSVWTGTYTTRTTDADGSVIEQIPTPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSTDTTLAPSADTTSIWTGTYTTWTTDADGSVIEQIPTPSADTTSIWTGTYTTWTTDADGSVIEQIPTPSADTTSVWTGTYTTWTTDADGSVIEQIPTPSTDTTLAPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSIWTGTYTTWTTDADGSVIEQIPTPSADTTSVWTGTYTTWTTDADGSVIEQIPTPSADTTLAPSADTTSIWTGTYTTWTTDADGSVIEQIPTPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSTDTTLAPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSTDTTLAPSADTTSIWTGTYTTWTTDADGSVIEQIPTPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQSPTPSAYTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQSPTPSAYTTSVWTGTYTTWTTDADGSVIEQIPTPSADTTLAPSADTTSIWTGTYTTWTTDADGSVIEQIPTPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSTDTTLAPSADTTSIWTGTYTTWTTDADGSVIEQIPTPSADTTSVWTGTYTTWTTDADGSVIEQIPTPSTDTTLAPSADTTSIWTGTYTTWTTDADGSVIEQIPTPSADTTSVWTGTYTTWTTDADGSVIEQIPTPSADTTSVWTGTYTTWTTDADGSVIEQIPTPSADTTSVWTGTYTTWTTDADGSVIEQIPTPSADTTLAPSADTTSIWTGTYTTWTTDADGSVIEQIPTPSADTTSVWTGTYTTWTTDAAGTVIEVIPSGTSISSDVIPTPLPTSGVDIDTIPYDAFNVAVYHYPADNYELANNLGFLTSGYEGLGQVTTATSVGNINFDTSSGWPYYIESNALGNTGSYVNATIEYVGFFQAPANGNYELSFSNIDYNAILFLGSPATDSSLAKREVQFLKPETSSEYVLFFDHGKDAGQTVSTTQYLSAGLYYPLRIVLAAVSERAQLDFQITLPDGRVLDQYQGYVYNFAHEGIESATSSAHETSWSRFTNSTIYSHSSTIGIITSSTDAPHSVINPTAIETTSTDTSISTVAVTTSICDTKDCVKTTVITPNSPLPTQTVSLTTTTIDRSEVVQTAHSAVPSQFAPDAHPSAVTITREQCDAYSCSQATIVSGKVLQTTTVSDSTTVVPLDTPQLSVEASTLETRLKSTQSSRAPTVTVQTSQSSRHSEDVTESSVHVSEFDAQSTSATSASALQAPSSISLQTGGANTLRLSAFLGTALLPMLNVLFI

SEQ ID NO: 298 - protein, 135 aa, synthetic construct
MQFSIVATLALAGSALAAYSNVTYTYETTITDVVTELTTYCPEPTTFVHKNKTITVTAPTTLTITDCPCTISKTTKITTDVPPTTHSTPHTTTTHVPSTSTPAPTHSVSTISHGGAAKAGVAGLAGVAAAAAYFL

SEQ ID NO: 299 - protein, 219 aa, synthetic construct
MLLTSLLQVFACCLVLPAQVTAFYYYTSGAERKCFHKELSKGTLFQATYKAQIYDDQLQNYRDAGAQDFGVLIDIEETFDDNHLVVHQKGSASGDLTFLASDSGEHKICIQPEAGGWLIKAKTKIDVEFQVGSDEKLDSKGKATIDILHAKVNVLNSKIGEIRREQKLMRDREATFRDASEAVNSRAMWWIVIQLIVLAVTCGWQMKHLGKFFVKQKIL

SEQ ID NO: 300 - protein, 215 aa, synthetic construct
MIKSTIALPSFFIVLILALVNSVAASSSYAPVAISLPAFSKECLYYDMVTEDDSLAVGYQVLTGGNFEIDFDITAPDGSVITSEKQKKYSDFLLKSFGVGKYTFCFSNNYGTALKKVEITLEKEKTLTDEHEADVNNDDIIANNAVEEIDRNLNKITKTLNYLRAREWRNMSTVNSTESRLTWLSILIIIIIAVISIAQVLLIQFLFTGRQKNYV

SEQ ID NO: 301 - protein, 203 aa, synthetic construct [sequence omitted]

SEQ ID NO: 302 - protein, 211 aa, synthetic construct [sequence omitted]

SEQ ID NO: 303 - protein, 225 aa, synthetic construct
MSNLCVLFFQFFFLAQFFAEASPLTFELNKGRKECLYTLTPEIDCTISYYFAVQQGESNDFDVNYEIFAPDDKNKPIIERSGERQGEWSFIGQHKGEYAICFYGGKAHDKIVDLDFKYNCERQDDIRNERRKARKAQRNLRDSKTDPLQDSVENSIDTIERQLHVLERNIQYYKSRNTRNHHTVCSTEHRIVMFSIYGILLIIGMSCAQIAILEFIFRESRKHNV

SEQ ID NO: 304 - protein, 212 aa, synthetic construct
MKYNIVHGICLLFAITQAVGAVHFYAKSGETKCFYEHLSRGNLLIGDLDLYVEKDGLFEEDPESSLTITVDETFDNDHRVLNQKNSHTGDVTFTALDTGEHRFCFTPFYSKKSATLRVFIELEIGNVEALDSKKKEDMNSLKGRVGQLTQRLSSIRKEQDAIREKEAEFRNQSESANSKIMTWSVFQLLILLGTCAFQLRYLKNFFVKQKVV

SEQ ID NO: 305 - protein, 1163 aa, synthetic construct
DESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSCYETTVSKITEWTTYTTPWTGTFETTRTITPTGTEGTVVIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPSGTEPGTVVIETPEIVDCEAYCCASVAIKKRELCQCENFCCSWDQSCQTYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIINCEAVCCGPFLTAFSFRKREECQCENICCPGDTNCETYVTTTQPWTGTYETTYTVPPTGTEPGTVIIETPESYVTTTQPWTGTYETTYTVPSTGTEPGTVIIETPESYVTTTQPWTGTYETTFTVPPTGTEPGTVVIETPESYVTTTQPWTGTYETTYSVPPSGTEPGTVVIETPESYVTTTQPWTGTYETTYSVPPSGTEPGTVVIETPEASTARTKFTTVTSSWTGVFTTTKTLPASGTEPATIVIQTPTGYFNTSSLVSTRTKTNVDTVTRVIPCPICTAPKTITVVPEEPNESVSVIISQPQSSSTDTTLSKPDSVRVISQPETASQMDTSLSKTDSAVISTETAGNNIIPLAGSHSYNTIVTTVTDSPQVAQSTTATSSSNVHLTISTQTTTPSLVYSSSLSTVHQVSPSNGGFRSSITVHPLLSVIGAIFGALFM

SEQ ID NO: 306 - protein, 1184 aa, synthetic construct
MKFPVPLLFLLQLFFIIATQGDESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSCYETTVSKITEWTTYTTPWTGTFETTRTITPTGTEGTVVIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPSGTEPGTVVIETPEIVDCEAYCCASVAIKKRELCQCENFCCSWDQSCQTYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIIDCEAVCCGPFLTAFSFRKREECQCENICCPGDTNCETYVTTTQPWTGTYETTYTVPPTGTEPGTVIIETPESYVTTTQPWTGTYETTYTVPPTGTEPGTVIIETPESYVTTTQPWTGTYETTYTVPPTGTEPGTVIIETPEIINCEAVCCGPFLTAFSFRKREECQCENICCPGDTNCETYVTTTQPWTGTYETTYTVPPTGTEPGTVIIETPESYVTTTQPWTGTYETTYTVPSTGTEPGTVIIETPESYVTTTQPWTGTYETTFTVPPTGTEPGTVVIETPESYVTTTQPWTGTYETTYSVPPSGTEPGTVVIETPESYVTTTQPWTGTYETTYSVPPSGTEPGTVVIETPEASTARTKFTTVTSSWTGVFTTTKTLPASGTEPATIVIQTPTGYFNTSSLVSTRTKTNVDTVTRVIPCPICTAPKTITVVPEEPNESVSVIISQPQSSSTDTTLSKPDSVRVISQPETASQMDTSLSKTDSAVISTETAGNNIIPLAGSHSYNTIVTTVTDSPQVAQSTTATSSSNVHLTISTQTTTPSLVYSSSLSTVHQVSPSNGGFRSSITVHPLLSVIGAIFGALFM

SEQ ID NO: 307 - protein, 1092 aa, synthetic construct
SSGKTCPTSEVSPACYANQWETTFPPSDIKITGATWVQDNIYDVTLSYEAESLELENLTELKIIGLNSPTGGTKLVWSLNSKVYDIDNPAKWTTTLRVYTKSSADDCYVEMYPFQIQVDWCEAGASTDGCSAWKWPKSYDYDIGCDNMQDGVSRKHHPVYKWPKKCSSNCGVEPTTSDEPEEPTTSEEPEEPTTSEEPEEPTSSDEEPTTSEEPEEPTTSDEPEEPTTSEEPEEPTTSEEPEEPTTSEEPTTSEEPEEPTSSDEEPTTSDEPEEPTTSDEPEEPTTSEEPTTSEEPEEPTTSSEEPTPSEEPEGPTCPTSEVSPACYADQWETTFPPSDIKITGATWVEDNIYDVTLSYEAESLELENLTELKIIGLNSPTGGTKVVWSLNSGIYDIDNPAKWTTTLRVYTKSSADDCYVEMYPFQIQVDWCEAGASTDGCSAWKWPKSYDYDIGCDNMQDGVSRKHHPVYKWPKKCSSDCGVEPTTSDEPEEPTTSEEPVEPTSSDEEPTTSEEPTTSEEPEEPTTSDEPEEPTTSEEPEEPTTSEEPEEPTTSEEPTTSEEPEEPTSSDEEPTTSDEPEEPTTSEEPEEPTTSEEPEEPTTSEEPEEPTTSDEPEEPTTSEEPEEPTTSEEPEEPTSSDEEPTTSEEPEEPTTSEEPEEPTTSEEPEEPTTSEEPEEPTSSDEEPTTSEEPEEPTTSDEPEEPTTSEEPEEPTTSEEPEEPTSSDEEPTTSEEPEEPTTSDEPEEPTTSEEPEEPTTSEEPEEPTTSEEPEEPTTSEEPEEPTSSDEEPTTSEEPEEPTTSDEPEEPTTSEEPEEPTTSEEPEEPTTSEEPEEPTTSDEEPGTTEEPLVPTTKTETDVSTTLLTVTDCGTKTCTKSLVITGVTKETVTTHGKTTVITTYCPLPTETVTPTPVTVTSTIYADESVTKTTVYTTGAVEKTVTVGGSSTVVVVHTPLTTAVVQSQSTDEIKTVVTARPSTTTIVRDVCYNSVCSVATIVTGVTEKTITFSTGSITVVPTYVPLVESEEHQRTASTSETRATSVVVPTVVGQSSSASATSSIFPSVTIHEGVANTVKNSMISGAVALLFNALFL

SEQ ID NO: 308 - protein, 1114 aa, synthetic construct
MVSLRSIFTSSILAAGLTRAHGSSGKTCPTSEVSPACYANQWETTFPPSDIKITGATWVQDNIYDVTLSYEAESLELENLTELKIIGLNSPTGGTKLVWSLNSKVYDIDNPAKWTTTLRVYTKSSADDCYVEMYPFQIQVDWCEAGASTDGCSAWKWPKSYDYDIGCDNMQDGVSRKHHPVYKWPKKCSSNCGVEPTTSDEPEEPTTSEEPEEPTTSEEPEEPTSSDEEPTTSEEPEEPTTSDEPEEPTTSEEPEEPTTSEEPEEPTTSEEPTTSEEPEEPTSSDEEPTTSDEPEEPTTSDEPEEPTTSEEPTTSEEPEEPTTSSEEPTPSEEPEGPTCPTSEVSPACYADQWETTFPPSDIKITGATWVEDNIYDVTLSYEAESLELENLTELKIIGLNSPTGGTKVVWSLNSGIYDIDNPAKWTTTLRVYTKSSADDCYVEMYPFQIQVDWCEAGASTDGCSAWKWPKSYDYDIGCDNMQDGVSRKHHPVYKWPKKCSSDCGVEPTTSDEPEEPTTSEEPVEPTSSDEEPTTSEEPTTSEEPEEPTTSDEPEEPTTSEEPEEPTTSEEPEEPTTSEEPTTSEEPEEPTSSDEEPTTSDEPEEPTTSEEPEEPTTSEEPEEPTTSEEPEEPTTSDEPEEPTTSEEPEEPTTSEEPEEPTSSDEEPTTSEEPEEPTTSEEPEEPTTSEEPEEPTTSEEPEEPTSSDEEPTTSEEPEEPTTSDEPEEPTTSEEPEEPTTSEEPEEPTSSDEEPTTSEEPEEPTTSDEPEEPTTSEEPEEPTTSEEPEEPTTSEEPEEPTTSEEPEEPTSSDEEPTTSEEPEEPTTSDEPEEPTTSEEPEEPTTSEEPEEPTTSEEPEEPTTSDEEPGTTEEPLVPTTKTETDVSTTLLTVTDCGTKTCTKSLVITGVTKETVTTHGKTTVITTYCPLPTETVTPTPVTVTSTIYADESVTKTTVYTTGAVEKTVTVGGSSTVVVVHTPLTTAVVQSQSTDEIKTVVTARPSTTTIVRDVCYNSVCSVATIVTGVTEKTITFSTGSITVVPTYVPLVESEEHQRTASTSETRATSVVVPTVVGQSSSASATSSIFPSVTIHEGVANTVKNSMISGAVALLFNALFL

SEQ ID NO: 309 - protein, 217 aa, synthetic construct
DESGNGDESDTAYGCDITSNAFDGFDATIYEYNANDLKLIRDPVFMSTGYLGRNVLNKISGVTVPGFNIWNPRSRTATVYGVQNVNYYNMVLELKGYFKAAVSGDYKLTLSNIDDSSMLFFGKNTAFQCCDTGSIPVDQAPTDYSLFTIKPSNQVNSEVISSTQYLEAGKYYPVRIVFVNALERALFNFKLTIPSGTVLDDFQDYIYQFGALDENSC

SEQ ID NO: 310 - DNA, 261 bp, synthetic construct
  source 1..261: mol_type = other DNA
tcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgaaaccttcgtttgtgcg

SEQ ID NO: 311 - DNA, 202 bp, synthetic construct
  source 1..202: mol_type = other DNA
tcgatttgtatgtgaaatagctgaaattcgaaaatttcattatggctgtatctactttagcgtattaggcatttgagcattggcttgaacaatgcgggctgtagtgtgtcaccaaagaaaccattcgggttcggatctggaagtcctcatcacgtgatgccgatctcgtgtattttattttcagataacacctgaagacttt

SEQ ID NO: 312 - DNA, 486 bp, synthetic construct
  source 1..486: mol_type = other DNA
attagtgtacatctgataatatagtactaccacgtatgataatgtagagaatagtcttccttgtcgagtgtgtttgcagttttcttgagtttcaaggtttaaatgctggtatattagttcatcgaaggtttcagccaatagcaccttaaatcaatcaaactaattcgactcttacgaaagagcctactgtgtttagtatcgaagtcgtttacctttcatgttgaatagcttcctctctgaccctaacatttcaagatcctcctaaagttacccggattgtgaaattctaatgatccacctgcccaatgcattttttctttattcagtttaccttttttacctaatatacgagcttgttaaagtaagtggcactgcaatactaggcttattgttgatattatgatgaatcgttttcacaaacttgatttcctgtgaactcaccatgtactaaggaaaaaaacatgcatcaccatctgaatatttgac

SEQ ID NO: 313 - DNA, 497 bp, synthetic construct
  source 1..497: mol_type = other DNA
actatgtaactaacgaaacagcatgtactaatagaaccgtatcgagaatatttatttaggtgagtagtaggagtgaaccagacagtcaatttagtgagctgtcccagcttttgtgcattccagaattgccggtcaaattggttatgggttatggggcttttccgattgaggttcagtttctgcggttatctctttcttgacctggtcttttacaggctgttctttctccccatgattattctttagctgaagataccgcttagcctgataatgtcgtcgttttgtaatcaaaatctttagttgggcatcgtctgaggtttcctttggcttctggggttgttagtaggaacgtaggaaccatagtaacttttacacatacattcttatgattgcgaagtaagctgagtctgctgcttggctcccgaagtactttctctttctctaccggttgattctccttctggtgctcctaaacgattgtgttagaagggattgac

SEQ ID NO: 314 - DNA, 4051 bp, synthetic construct
  source 1..4051: mol_type = other DNA
caggtgaacccacctaactatttttaactggcatccagtgagctcgctgggtgaaagccaaccatcttttgtttcggggaaccgtgctcgccccgtaaagttaatttttttttcccgcgcagctttaatctttcggcagagaaggcgttttcatcgtagcgtgggaacagaataatcagttcatgtgctatacaggcacatggcagcagtcactattttgctttttaaccttaaagtcgttcatcaatcattaactgaccaatcagattttttgcatttgccacttatctaaaaatacttttgtatctcgcagatacgttcagtggtttccaggacaacacccaaaaaaaggtatcaatgccactaggcagtcggttttatttttggtcacccacgcaaagaagcacccacctcttttaggttttaagttgtgggaacagtaacaccgcctagagcttcaggaaaaaccagtacctgtgaccgcaattcaccatgatgcagaatgttaatttaaacgagtgccaaatcaagatttcaacagacaaatcaatcgatccatagttacccattccagccttttcgtcgtcgagcctgcttcattcctgcctcaggtgcataactttgcatgaaaagtccagattagggcagattttgagtttaaaataggaaatataaacaaatataccgcgaaaaaggtttgtttatagcttttcgcctggtgccgtacggtataaatacatactctcctcccccccctggttctctttttcttttgttacttacattttaccgttccgtcactcgcttcactcaacaacaaaagaattccgaaacgatgagattcccatctattttcaccgctgtcttgttcgctgcctcctctgcattggctgcccctgttcagactaccactgaagacgagcttgagggtgatttcgacgtcgctgttttgcctttctctgcttccattgctgctaaggaagagggtgtctctctcgagaaaagagaggccgaagcttctatgacgaatgagacgtcagacaggccactggtacatttcacaccaaataaaggatggatgaatgatcccaacggtctgtggtacgacgagaaagacgctaagtggcatctgtactttcaatataaccccaatgacactgtttggggtacgccccttttctggggccatgcaacctcagatgatctgactaactgggaagaccaacctattgccatcgcccccaagcgtaatgattcaggcgccttctctggaagtatggtagtcgactacaacaacacgagtggttttttcaacgacacaattgacccaagacagagatgtgttgccatttggacatataatacacctgagtcagaagaacaatatatatcctactctctggatggaggttatacttttacggagtatcagaaaaaccctgttctggcagctaattccacccaatttcgtgacccaaaggtgttttggtatgagccatcacagaaatggatcatgaccgccgcaaagtcacaggactataaaattgagatatattcatcagacgacttgaaatcctggaagctggagagtgctttcgcaaatgaaggatttttgggataccaatacgaatgccctggcctgatcgaagtgcccactgaacaagacccatcaaagtcttactgggtgatgtttatctctataaaccccggcgctcccgctggaggctccttcaaccaatatttcgtaggttcttttaacggaacccacttcgaggcattcgataaccaatctagggttgtcgatttcggaaaagattattatgcactacaaaccttttttaatacggaccctacttatggatcagctttaggcatagcctgggcttctaactgggagtacagtgcctttgttcctacaaacccatggcgttcctcaatgagtcttgtcagaaaattctctctgaatacggaatatcaggctaaccccgagacagaactaataaacttgaaagcagagcctatcttgaatataagtaacgctggaccttggagtcgttttgccaccaacaccacattaacaaaagccaattcctataacgtggacctttccaactctacgggaacactggaatttgaactggtgtacgccgtaaatactacgcaaacaatttcaaagtcagtctttgctgaccttagtctatggttcaagggtttagaggaccccgaggagtacttacgtatgggttttgaggtatcagcatcttcctttttcctggatcgtggaaactccaaggtgaagtttgtcaaggaaaatccttatttcactaacaggatgtccgtgaacaaccagcctttcaaatctgagaacgatctttcctattacaaggtctacggacttctagaccaaaacatattggaactatacttcaacgatggagatgtagtttccaccaacacctattttatgaccacaggcaacgcccttggatcagtaaacatgacaacaggtgttgataaccttttttacatagacaaattccaagttagagaggtaaagggttcatcagggtcctcaggatcatccggtagtagtggttcatccggttcatccggatcaagtggctcctctgaagctgcagcaagggaggctgcagcccgtgaggcagccgctagagaagccgccgctaggggtggtggcggctctggcggaggcggttccggtggcggaggctctcaaatcaacgaattgaacgttgttttagatgatgttaagaccaacattgccgactacatcaccctatcctacactccaaattcaggtttttccttggaccaaatgccagctggtattatggatattgctgcgcaattggttgcaaatccaagtgatgactcctacaccactttgtactctgaagtggacttttctgctgttgagcatatgttgactatggtcccatggtactcttctagactgcttccagaattagaagcaatggatgcttctctaactacctcaagttctgctgccacatcttcaagtgaagttgctagctcttctattgcttcatccactagctcttctgttgcaccatcctcaagtgaagttgtcagctcttccgttgcttcatcctcaagtgaagttgccagctcctctgttgcgtctacaagcgaagctactagttcttctgctgtcacatcttcctccgctgtttcctcttcgaccgagtctgttagctcttcctctgtcagttcttcctcagccgtttcctcttctgaagctgtcagttcctctccagtttcctcagttgtttcatcttcggccggacctgctagctcaagcgttgctccttacaactcaaccattgctagctcttcttccactgcccagacttctatctcgaccattgctccttacaactccacaaccaccaccaccccagctagttctgcttccagcgttattatctcaaccagaaacggtaccactgttactgaaactgacaacactcttgtcaccaaagaaaccactgtctgtgactactcttcaacatctgccgttccagcttccaccaccggttacaacaattctactaaggtttcaaccgctactatctgcagtacatgcaaagaaggtacctctactgcaactgacttctctacactaaagactacagttaccgtatgtgactccgcctgtcaagctaagaagtctgctaccgttgttagcgttcaatctaaaactaccggtatcgttgaacaaaccgaaaacggtgctgccaaggctgttatcggtatgggtgccggtgctttagctgctgttgccgccatgctactatgagcggccgctcgatttgtatgtgaaatagctgaaattcgaaaatttcattatggctgtatctactttagcgtattaggcatttgagcattggcttgaacaatgcgggctgtagtgtgtcaccaaagaaaccattcgggttcggatctggaagtcctcatcacgtgatgccgatctcgtgtattttattttcagataacacctgaagacttt

SEQ ID NO: 315 - protein, 940 aa, synthetic construct
SMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVKGSSGSSGSSGSSGSSGSSGSSGSSEAAAREAAAREAAAREAAARGGGGSGGGGSGGGGSQINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVASSSSEVASSSVASTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTENGAAKAVIGMGAGALAAVAAMLL

SEQ ID NO: 316 - protein, 616 aa, Saccharomyces cerevisiae
MKNIISLVSKKKAASKNEDKNISESSRDIVNQQEVFNTEDFEEGKKDSAFELDHLEFTTNSAQLGDSDEDNENVINEMNATDDANEANSEEKSMTLKQALLKYPKAALWSILVSTTLVMEGYDTALLSALYALPVFQRKFGTLNGEGSYEITSQWQIGLNMCVLCGEMIGLQITTYMVEFMGNRYTMITALGLLTAYIFILYYCKSLAMIAVGQILSAIPWGCFQSLAVTYASEVCPLALRYYMTSYSNICWLFGQIFASGIMKNSQENLGNSDLGYKLPFALQWIWPAPLMIGIFFAPESPWWLVRKDRVAEARKSLSRILSGKGAEKDIQVDLTLKQIELTIEKERLLASKSGSFFNCFKGVNGRRTRLACLTWVAQNSSGAVLLGYSTYFFERAGMATDKAFTFSLIQYCLGLAGTLCSWVISGRVGRWTILTYGLAFQMVCLFIIGGMGFGSGSSASNGAGGLLLALSFFYNAGIGAVVYCIVAEIPSAELRTKTIVLARICYNLMAVINAILTPYMLNVSDWNWGAKTGLYWGGFTAVTLAWVIIDLPETTGRTFSEINELFNQGVPARKFASTVVDPFGKGKTQHDSLADESISQSSSIKQRELNAADKC

SEQ ID NO: 317 - protein, 582 aa, unidentified
MPEFVENIEKPEEAEVIPDITKKINTLSDSDDGSGAFNDYIARFVEISTNAQNNEHQEKHMSLKEGLKTFPKAACWSIVLSTAIIMEGYDTTLLNSLYSMQSFAKKYGKYYPEIDQYQVPAKWQTSLSMSTYVGEIVGLYIAGLVAEKWGYRRTLISFMAAVVGLIFILFFAVDVQMLLAGELLCGIVWGAFQTLTVSYASEVCPVVLRIYLTTYVNACWVIGQLIAACLLRGTMTLTSEWSYKIPFAVQWIWPVPIMIGIYLAPESPWWLVKKNRDAEAKKSITRLLSPNTEVPDVAPLAEAMLNKMQLTIKEESARTSNVSYFDCFKHGNFRRTRIAAMIWLIQNITGSVLMGYSTYFYIQAGLDSSMSFTFSIIQYALGLLGTLASWLLSQKLGRFDIYFLGLSINTCILIIVGGLGFSSSTSASWAIGSLLLVFTFVYDSSIGPITYCTVAEIPSSTVRAKTVALARNWYNLSQIPLSIVTPYMLNPTAWNWKAKAALLWAGLSICSLIYIWFEFPETKGRTYAELDILFKNGTSARKFRSTQVETFNPQEMLKKMNNEDIIQVVDGDLDAGAATAKV

SEQ ID NO: 318-319 - skipped (2 entries)

SEQ ID NO: 320 - protein, 346 aa, Saccharomyces cerevisiae
QINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVASSSSEVASSSVASTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTEN

SEQ ID NO: 321 - protein, 443 aa, Saccharomyces cerevisiae
QINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVAPSSSEVVSSSVAPSSSEVVSSSVASSSSEVASSSVAPSSSEVVSSSVASSSSEVASSSVAPSSSEVVSSSVAPSSSEVVSSSVASSSSEVASSSVAPSSSEVVSSSVASSTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTEN

SEQ ID NO: 322 - protein, 532 aa, Saccharomyces cerevisiae
MLLQAFLFLLAGFAAKISASMTNETSDRPLVHFTPNKGWMNDPNGLWYDAKEGKWHLYFQYNPNDTVWGLPLFWGHATSDDLTHWQDEPVAIAPKRKDSGAYSGSMVIDYNNTSGFFNDTIDPRQRCVAIWTYNTPESEEQYISYSLDGGYTFTEYQKNPVLAANSTQFRDPKVFWYEPSKKWIMTAAKSQDYKIEIYSSDDLKSWKLESAFANEGFLGYQYECPGLIEVPSEQDPSKSHWVMFISINPGAPAGGSFNQYFVGSFNGHHFEAFDNQSRVVDFGKDYYALQTFFNTDPTYGSALGIAWASNWEYSAFVPSNPWRSSMSLVRPFSLNTEYQANPETELINLKAEPILNISSAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 323 - protein, 609 aa, unidentified
MLKLLSLMVPLASAAVIHRRDANISAIASEWNSTSNSSSSLSLNRPAVHYSPEEGWMNDPNGLWYDAKEEDWHIYYQYYPDAPHWGLPLTWGHAVSKDLTVWDEQGVAFGPEFETAGAFSGSMVIDYNNTSGFFNSSTDPRQRVVAIWTLDYSGSETQQLSYSHDGGYTFTEYSDNPVLDIDSDAFRDPKVFWYQGEDSESEGNWVMTVAEADRFSVLIYSSPDLKNWTLESNFSREGYLGYNYECPGLVKVPYVKNTTYASAPGSNITSSGPLHPNSTVSFSNSSSIAWNASSVPLNITLSNSTLVDETSQLEEVGYAWVMIVSFNPGSILGGSGTEYFIGDFNGTHFEPLDKQTRFLDLGKDYYALQTFFNTPNEVDVLGIAWASNWQYANQVPTDPWRSSMSLVRNFTITEYNINSNTTALVLNSQPVLDFTSLRKNGTSYTLENLTLNSSSHEVLEFEDPTGVFEFSLEYSVNFTGIHNWVFTDLSLYFQGDKDSDEYLRLGYEANSKQFFLDRGHSNIPFVQENPFFTQRLSVSNPPSSNSSTFDVYGIVDRNIIELYFNNGTVTSTNTFFFSTGNNIGSIIVKSGVDDVYEIESLKVNQFYVD

SEQ ID NO: 324 - protein, 533 aa, unidentified
MSLTKDASEDQEDIKSLTMNTSLVDSSIYRPLVHLTPPVGWMNDPNGLFYDSSESTYHVYYQYNPNDTIWGLPLYWGHATSDDLLTWDHHAPAIGPENDDEGIYSGSIVIDYDNTSGFFDDSTRPEQRIVAIYTNNLPDVETQDIAYSTDGGYTFEKYENNPVIDVNSTQFRDPKVIWYEETEQWVMTVAKSQEYKIQIYTSDNLKDWSLASNFSTKGYVGYQYECPGLFEATIENPKSGDPEKKWVMVLAINPGSPLGGSINEYFVGDFNGTEFIPDDDATRFMDTGKDFYAFQAFFNAPENRSIGVAWSSNWQYSNQVPDPDGYRSSMSSIREYTLRYVSTNPESEQLILCQKPFFVNETDLKVVEEYKVSNSSLTVDHTFGSSFANSNTTGLLDFNMTFTVNGTTDVTQKDSVTFELRIKSNQSDEAIALGYDYNNEQFYINRATESYFQRTNQFFQERWSTYVQPLTITESGDKQYQLYGLVDNNILELYFNDGAFTSTNTFFLEKGKPSNVDIVASSSKEAYHRGPAD

SEQ ID NO: 325 - protein, 562 aa, Oryza sativa
MELAVGAGGMRRSASHTSLSESDDFDLSRLLNKPRINVERQRSFDDRSLSDVSYSGGGHGGTRGGFDGMYSPGGGLRSLVGTPASSALHSFEPHPIVGDAWEALRRSLVFFRGQPLGTIAAFDHASEEVLNYDQVFVRDFVPSALAFLMNGEPEIVRHFLLKTLLLQGWEKKVDRFKLGEGAMPASFKVLHDSKKGVDTLHADFGESAIGRVAPVDSGFWWIILLRAYTKSTGDLTLAETPECQKGMRLILSLCLSEGFDTFPTLLCADGCCMIDRRMGVYGYPIEIQALFFMALRCALQLLKHDNEGKEFVERIATRLHALSYHMRSYYWLDFQQLNDIYRYKTEEYSHTAVNKFNVIPDSIPDWLFDFMPCQGGFFIGNVSPARMDFRWFALGNMIAILSSLATPEQSTAIMDLIEERWEELIGEMPLKICYPAIENHEWRIVTGCDPKNTRWSYHNGGSWPVLLWLLTAACIKTGRPQIARRAIDLAERRLLKDGWPEYYDGKLGRYVGKQARKFQTWSIAGYLVAKMMLEDPSHLGMISLEEDKAMKPVLKRSASWTN

SEQ ID NO: 326 - protein, 551 aa, Arabidopsis thaliana
MEGVGLRAVGSHCSLSEMDDLDLTRALDKPRLKIERKRSFDERSMSELSTGYSRHDGIHDSPRGRSVLDTPLSSARNSFEPHPMMAEAWEALRRSMVFFRGQPVGTLAAVDNTTDEVLNYDQVFVRDFVPSALAFLMNGEPDIVKHFLLKTLQLQGWEKRVDRFKLGEGVMPASFKVLHDPIRETDNIVADFGESAIGRVAPVDSGFWWIILLRAYTKSTGDLTLSETPECQKGMKLILSLCLAEGFDTFPTLLCADGCSMIDRRMGVYGYPIEIQALFFMALRSALSMLKPDGDGREVIERIVKRLHALSFHMRNYFWLDHQNLNDIYRFKTEEYSHTAVNKFNVMPDSIPEWVFDFMPLRGGYFVGNVGPAHMDFRWFALGNCVSILSSLATPDQSMAIMDLLEHRWAELVGEMPLKICYPCLEGHEWRIVTGCDPKNTRWSYHNGGSWPVLLWQLTAACIKTGRPQIARRAVDLIESRLHRDCWPEYYDGKLGRYVGKQARKYQTWSIAGYLVAKMLLEDPSHIGMISLEEDKLMKPVIKRSASWPQL

SEQ ID NO: 327 - protein, 616 aa, Arabidopsis thaliana
MSAIYLLRKISTKTPSRFHRSLFFSTFSKDSPPDLSRTTSIRHLSSSQRFVSSSIYCFPQSKILPNRFSEKTTGISVRQFSTSVETNLSDKSFERIHVQSDAILERIHKNEEEVETVSIGSEKVVREESEAEKEAWRILENAVVRYCGSPVGTVAANDPGDKMPLNYDQVFIRDFVPSALAFLLKGEGDIVRNFLLHTLQLQSWEKTVDCYSPGQGLMPASFKVRTVALDENTTEEVLDPDFGESAIGRVAPVDSGLWWIILLRAYGKITGDFSLQERIDVQTGIKLIMNLCLADGFDMFPTLLVTDGSCMIDRRMGIHGHPLEIQSLFYSALRCSREMLSVNDSSKDLVRAINNRLSALSFHIREYYWVDIKKINEIYRYKTEEYSTDATNKFNIYPEQIPPWLMDWIPEQGGYLLGNLQPAHMDFRFFTLGNFWSIVSSLATPKQNEAILNLIEAKWDDIIGNMPLKICYPALEYDDWRIITGSDPKNTPWSYHNSGSWPTLLWQFTLACMKMGRPELAEKALAVAEKRLLADRWPEYYDTRSGKFIGKQSRLYQTWTVAGFLTSKLLLANPEMASLLFWEEDYELLDICACGLRKSDRKKCSRVAAKTQILVR

SEQ ID NO: 328 - protein, 617 aa, Arabidopsis thaliana
MAASETVLRVPLGSVSQSCYLASFFVNSTPNLSFKPVSRNRKTVRCTNSHEVSSVPKHSFHSSNSVLKGKKFVSTICKCQKHDVEESIRSTLLPSDGLSSELKSDLDEMPLPVNGSVSSNGNAQSVGTKSIEDEAWDLLRQSVVFYCGSPIGTIAANDPNSTSVLNYDQVFIRDFIPSGIAFLLKGEYDIVRNFILYTLQLQSWEKTMDCHSPGQGLMPCSFKVKTVPLDGDDSMTEEVLDPDFGEAAIGRVAPVDSGLWWIILLRAYGKCTGDLSVQERVDVQTGIKMILKLCLADGFDMFPTLLVTDGSCMIDRRMGIHGHPLEIQALFYSALVCAREMLTPEDGSADLIRALNNRLVALNFHIREYYWLDLKKINEIYRYQTEEYSYDAVNKFNIYPDQIPSWLVDFMPNRGGYLIGNLQPAHMDFRFFTLGNLWSIVSSLASNDQSHAILDFIEAKWAELVADMPLKICYPAMEGEEWRIITGSDPKNTPWSYHNGGAWPTLLWQLTVASIKMGRPEIAEKAVELAERRISLDKWPEYYDTKRARFIGKQARLYQTWSIAGYLVAKLLLANPAAAKFLTSEEDSDLRNAFSCMLSANPRRTRGPKKAQQPFIV

SEQ ID NO: 329 - protein, 598 aa, Oryza sativa
MGVLGSRVAWAWLVQLLLLQQLAGASHVVYDDLELQAAATTADGVPPSIVDSELRTGYHFQPPKNWINDPNAPMYYKGWYHLFYQYNPKGAVWGNIVWAHSVSRDLINWVALKPAIEPSIRADKYGCWSGSATMMADGTPVIMYTGVNRPDVNYQVQNVALPRNGSDPLLREWVKPGHNPVIVPEGGINATQFRDPTTAWRGADGHWRLLVGSLAGQSRGVAYVYRSRDFRRWTRAAQPLHSAPTGMWECPDFYPVTADGRREGVDTSSAVVDAAASARVKYVLKNSLDLRRYDYYTVGTYDRKAERYVPDDPAGDEHHIRYDYGNFYASKTFYDPAKRRRILWGWANESDTAADDVAKGWAGIQAIPRKVWLDPSGKQLLQWPIEEVERLRGKWPVILKDRVVKPGEHVEVTGLQTAQADVEVSFEVGSLEAAERLDPAMAYDAQRLCSARGADARGGVGPFGLWVLASAGLEEKTAVFFRVFRPAARGGGAGKPVVLMCTDPTKSSRNPNMYQPTFAGFVDTDITNGKISLRSLIDRSVVESFGAGGKACILSRVYPSLAIGKNARLYVFNNGKAEIKVSQLTAWEMKKPVMMNGA

SEQ ID NO: 330 - protein, 1841 aa, Rattus norvegicus
MAKKKFSALEISLIVLFIIVTAIAIALVTVLATKVPAVEEIKSPTPTSNSTPTSTPTSTSTPTSTSTPSPGKCPPEQGEPINERINCIPEQHPTKAICEERGCCWRPWNNTVIPWCFFADNHGYNAESITNENAGLKATLNRIPSPTLFGEDIKSVILTTQTQTGNRFRFKITDPNNKRYEVPHQFVKEETGIPAADTLYDVQVSENPFSIKVIRKSNNKVLCDTSVGPLLYSNQYLQISTRLPSEYIYGFGGHIHKRFRHDLYWKTWPIFTRDEIPGDNNHNLYGHQTFFMGIGDTSGKSYGVFLMNSNAMEVFIQPTPIITYRVTGGILDFYIFLGDTPEQVVQQYQEVHWRPAMPAYWNLGFQLSRWNYGSLDTVSEVVRRNREAGIPYDAQVTDIDYMEDHKEFTYDRVKFNGLPEFAQDLHNHGKYIIILDPAISINKRANGAEYQTYVRGNEKNVWVNESDGTTPLIGEVWPGLTVYPDFTNPQTIEWWANECNLFHQQVEYDGLWIDMNEVSSFIQGSLNLKGVLLIVLNYPPFTPGILDKVMYSKTLCMDAVQHWGKQYDVHSLYGYSMAIATEQAVERVFPNKRSFILTRSTFGGSGRHANHWLGDNTASWEQMEWSITGMLEFGIFGMPLVGATSCGFLADTTEELCRRWMQLGAFYPFSRNHNAEGYMEQDPAYFGQDSSRHYLTIRYTLLPFLYTLFYRAHMFGETVARPFLYEFYDDTNSWIEDTQFLWGPALLITPVLRPGVENVSAYIPNATWYDYETGIKRPWRKERINMYLPGDKIGLHLRGGYIIPTQEPDVTTTASRKNPLGLIVALDDNQAAKGELFWDDGESKDSIEKKMYILYTFSVSNNELVLNCTHSSYAEGTSLAFKTIKVLGLREDVRSITVGENDQQMATHTNFTFDSANKILSITALNFNLAGSFIVRWCRTFSDNEKFTCYPDVGTATEGTCTQRGCLWQPVSGLSNVPPYYFPPENNPYTLTSIQPLPTGITAELQLNPPNARIKLPSNPISTLRVGVKYHPNDMLQFKIYDAQHKRYEVPVPLNIPDTPTSSNERLYDVEIKENPFGIQVRRRSSGKLIWDSRLPGFGFNDQFIQISTRLPSNYLYGFGEVEHTAFKRDLNWHTWGMFTRDQPPGYKLNSYGFHPYYMALENEGNAHGVLLLNSNGMDVTFQPTPALTYRTIGGILDFYMFLGPTPEIATRQYHEVIGFPVMPPYWALGFQLCRYGYRNTSEIEQLYNDMVAANIPYDVQYTDINYMERQLDFTIGERFKTLPEFVDRIRKDGMKYIVILAPAISGNETQPYPAFERGIQKDVFVKWPNTNDICWPKVWPDLPNVTIDETITEDEAVNASRAHVAFPDFFRNSTLEWWAREIYDFYNEKMKFDGLWIDMNEPSSFGIQMGGKVLNECRRMMTLNYPPVFSPELRVKEGEGASISEAMCMETEHILIDGSSVLQYDVHNLYGWSQVKPTLDALQNTTGLRGIVISRSTYPTTGRWGGHWLGDNYTTWDNLEKSLIGMLELNLFGIPYIGADICGVFHDSGYPSLYFVGIQVGAFYPYPRESPTINFTRSQDPVSWMKLLLQMSKKVLEIRYTLLPYFYTQMHEAHAHGGTVIRPLMHEFFDDKETWEIYKQFLWGPAFMVTPVVEPFRTSVTGYVPKARWFDYHTGADIKLKGILHTFSAPFDTINLHVRGGYILPCQEPARNTHLSRQNYMKLIVAADDNQMAQGTLFGDDGESIDTYERGQYTSIQFNLNQTTLTSTVLANGYKNKQEMRLGSIHIWGKGTLRISNANLVYGGRKHQPPFTQEEAKETLIFDLKNMNVTLDEPIQITWS

SEQ ID NO: 331 - protein, 1827 aa, Oryctolagus cuniculus
MAKRKFSGLEITLIVLFVIVFIIAIALIAVLATKTPAVEEVNPSSSTPTTTSTTTSTSGSVSCPSELNEVVNERINCIPEQSPTQAICAQRNCCWRPWNNSDIPWCFFVDNHGYNVEGMTTTSTGLEARLNRKSTPTLFGNDINNVLLTTESQTANRLRFKLTDPNNKRYEVPHQFVTEFAGPAATETLYDVQVTENPFSIKVIRKSNNRILFDSSIGPLVYSDQYLQISTRLPSEYMYGFGEHVHKRFRHDLYWKTWPIFTRDQHTDDNNNNLYGHQTFFMCIEDTTGKSFGVFLMNSNAMEIFIQPTPIVTYRVIGGILDFYIFLGDTPEQVVQQYQELIGRPAMPAYWSLGFQLSRWNYNSLDVVKEVVRRNREALIPFDTQVSDIDYMEDKKDFTYDRVAYNGLPDFVQDLHDHGQKYVIILDPAISINRRASGEAYESYDRGNAQNVWVNESDGTTPIVGEVWPGDTVYPDFTSPNCIEWWANECNIFHQEVNYDGLWIDMNEVSSFVQGSNKGCNDNTLNYPPYIPDIVDKLMYSKTLCMDSVQYWGKQYDVHSLYGYSMAIATERAVERVFPNKRSFILTRSTFAGSGRHAAHWLGDNTATWEQMEWSITGMLEFGLFGMPLVGADICGFLAETTEELCRRWMQLGAFYPFSRNHNADGFEHQDPAFFGQDSLLVKSSRHYLNIRYTLLPFLYTLFYKAHAFGETVARPVLHEFYEDTNSWVEDREFLWGPALLITPVLTQGAETVSAYIPDAVWYDYETGAKRPWRKQRVEMSLPADKIGLHLRGGYIIPIQQPAVTTTASRMNPLGLIIALNDDNTAVGDFFWDDGETKDTVQNDNYILYTFAVSNNNLNITCTHELYSEGTTLAFQTIKILGVTETVTQVTVAENNQSMSTHSNFTYDPSNQVLLIENLNFNLGRNFRVQWDQTFLESEKITCYPDADIATQEKCTQRGCIWDTNTVNPRAPECYFPKTDNPYSVSSTQYSPTGITADLQLNPTRTRITLPSEPITNLRVEVKYHKNDMVQFKIFDPQNKRYEVPVPLDIPATPTSTQENRLYDVEIKENPFGIQIRRRSTGKVIWDSCLPGFAFNDQFIQISTRLPSEYIYGFGEAEHTAFKRDLNWHTWGMFTRDQPPGYKLNSYGFHPYYMALEDEGNAHGVLLLNSNAMDVTFMPTPALTYRVIGGILDFYMFLGPTPEVATQQYHEVIGHPVMPPYWSLGFQLCRYGYRNTSEIIELYEGMVAADIPYDVQYTDIDYMERQLDFTIDENFRELPQFVDRIRGEGMRYIIILDPAISGNETRPYPAFDRGEAKDVFVKWPNTSDICWAKVWPDLPNITIDESLTEDEAVNASRAHAAFPDFFRNSTAEWWTREILDFYNNYMKFDGLWIDMNEPSSFVNGTTTNVCRNTELNYPPYFPELTKRTDGLHFRTMCMETEHILSDGSSVLHYDVHNLYGWSQAKPTYDALQKTTGKRGIVISRSTYPTAGRWAGHWLGDNYARWDNMDKSIIGMMEFSLFGISYTGADICGFFNDSEYHLCTRWTQLGAFYPFARNHNIQFTRRQDPVSWNQTFVEMTRNVLNIRYTLLPYFYTQLHEIHAHGGTVIRPLMHEFFDDRTTWDIFLQFLWGPAFMVTPVLEPYTTVVRGYVPNARWFDYHTGEDIGIRGQVQDLTLLMNAINLHVRGGHILPCQEPARTTFLSRQKYMKLIVAADDNHMAQGSLFWDDGDTIDTYERDLYLSVQFNLNKTTLTSTLLKTGYINKTEIRLGYVHVWGIGNTLINEVNLMYNEINYPLIFNQTQAQEILNIDLTAHEVTLDDPIEISWS

SEQ ID NO: 332 - protein, 918 aa, synthetic construct
SMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVKGSSGSSGSSGSSGSSGSSGSSGSSEAAAREAAAREAAAREAAARGGGGSGGGGSGGGGSQINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVASSSSEVASSSVASTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTEN

SEQ ID NO: 333 - protein, 1001 aa, synthetic construct
MRFPSIFTAVLFAASSALAAPVQTTTEDELEGDFDVAVLPFSASIAAKEEGVSLEKREAEASMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVKGSSGSSGSSGSSGSSGSSGSSGSSEAAAREAAAREAAAREAAARGGGGSGGGGSGGGGSQINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVASSSSEVASSSVASTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTENGAAKAVIGMGAGALAAVAAMLL

SEQ ID NO: 334 - protein, 918 aa, synthetic construct; identical to SEQ ID NO: 332

SEQ ID NO: 335 - protein, 979 aa, synthetic construct
MRFPSIFTAVLFAASSALAAPVQTTTEDELEGDFDVAVLPFSASIAAKEEGVSLEKREAEASMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVKGSSGSSGSSGSSGSSGSSGSSGSSEAAAREAAAREAAAREAAARGGGGSGGGGSGGGGSQINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVASSSSEVASSSVASTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTEN

SEQ ID NO: 336 - protein, 21 aa, synthetic construct
GAAKAVIGMGAGALAAVAAML

SEQ ID NO: 337 - DNA, 183 bp, synthetic construct
  source 1..183: mol_type = other DNA
atgagattcccatctattttcaccgctgtcttgttcgctgcctcctctgcattggctgcccctgttcagactaccactgaagacgagcttgagggtgatttcgacgtcgctgttttgcctttctctgcttccattgctgctaaggaagagggtgtctctctcgagaaaagagaggccgaagct

SEQ ID NO: 338 - DNA, 1539 bp, synthetic construct
  source 1..1539: mol_type = other DNA
tctatgacgaatgagacgtcagacaggccactggtacatttcacaccaaataaaggatggatgaatgatcccaacggtctgtggtacgacgagaaagacgctaagtggcatctgtactttcaatataaccccaatgacactgtttggggtacgccccttttctggggccatgcaacctcagatgatctgactaactgggaagaccaacctattgccatcgcccccaagcgtaatgattcaggcgccttctctggaagtatggtagtcgactacaacaacacgagtggttttttcaacgacacaattgacccaagacagagatgtgttgccatttggacatataatacacctgagtcagaagaacaatatatatcctactctctggatggaggttatacttttacggagtatcagaaaaaccctgttctggcagctaattccacccaatttcgtgacccaaaggtgttttggtatgagccatcacagaaatggatcatgaccgccgcaaagtcacaggactataaaattgagatatattcatcagacgacttgaaatcctggaagctggagagtgctttcgcaaatgaaggatttttgggataccaatacgaatgccctggcctgatcgaagtgcccactgaacaagacccatcaaagtcttactgggtgatgtttatctctataaaccccggcgctcccgctggaggctccttcaaccaatatttcgtaggttcttttaacggaacccacttcgaggcattcgataaccaatctagggttgtcgatttcggaaaagattattatgcactacaaaccttttttaatacggaccctacttatggatcagctttaggcatagcctgggcttctaactgggagtacagtgcctttgttcctacaaacccatggcgttcctcaatgagtcttgtcagaaaattctctctgaatacggaatatcaggctaaccccgagacagaactaataaacttgaaagcagagcctatcttgaatataagtaacgctggaccttggagtcgttttgccaccaacaccacattaacaaaagccaattcctataacgtggacctttccaactctacgggaacactggaatttgaactggtgtacgccgtaaatactacgcaaacaatttcaaagtcagtctttgctgaccttagtctatggttcaagggtttagaggaccccgaggagtacttacgtatgggttttgaggtatcagcatcttcctttttcctggatcgtggaaactccaaggtgaagtttgtcaaggaaaatccttatttcactaacaggatgtccgtgaacaaccagcctttcaaatctgagaacgatctttcctattacaaggtctacggacttctagaccaaaacatattggaactatacttcaacgatggagatgtagtttccaccaacacctattttatgaccacaggcaacgcccttggatcagtaaacatgacaacaggtgttgataaccttttttacatagacaaattccaagttagagaggtaaag

SEQ ID NO: 339 - DNA, 177 bp, synthetic construct
  source 1..177: mol_type = other DNA
ggttcatcagggtcctcaggatcatccggtagtagtggttcatccggttcatccggatcaagtggctcctctgaagctgcagcaagggaggctgcagcccgtgaggcagccgctagagaagccgccgctaggggtggtggcggctctggcggaggcggttccggtggcggaggctct

SEQ ID NO: 340 - DNA, 1107 bp, synthetic construct
  source 1..1107: mol_type = other DNA
caaatcaacgaattgaacgttgttttagatgatgttaagaccaacattgccgactacatcaccctatcctacactccaaattcaggtttttccttggaccaaatgccagctggtattatggatattgctgcgcaattggttgcaaatccaagtgatgactcctacaccactttgtactctgaagtggacttttctgctgttgagcatatgttgactatggtcccatggtactcttctagactgcttccagaattagaagcaatggatgcttctctaactacctcaagttctgctgccacatcttcaagtgaagttgctagctcttctattgcttcatccactagctcttctgttgcaccatcctcaagtgaagttgtcagctcttccgttgcttcatcctcaagtgaagttgccagctcctctgttgcgtctacaagcgaagctactagttcttctgctgtcacatcttcctccgctgtttcctcttcgaccgagtctgttagctcttcctctgtcagttcttcctcagccgtttcctcttctgaagctgtcagttcctctccagtttcctcagttgtttcatcttcggccggacctgctagctcaagcgttgctccttacaactcaaccattgctagctcttcttccactgcccagacttctatctcgaccattgctccttacaactccacaaccaccaccaccccagctagttctgcttccagcgttattatctcaaccagaaacggtaccactgttactgaaactgacaacactcttgtcaccaaagaaaccactgtctgtgactactcttcaacatctgccgttccagcttccaccaccggttacaacaattctactaaggtttcaaccgctactatctgcagtacatgcaaagaaggtacctctactgcaactgacttctctacactaaagactacagttaccgtatgtgactccgcctgtcaagctaagaagtctgctaccgttgttagcgttcaatctaaaactaccggtatcgttgaacaaaccgaaaacggtgctgccaaggctgttatcggtatgggtgccggtgctttagctgctgttgccgccatgctactatga

SEQ ID NO: 341 - DNA, 210 bp, synthetic construct
  source 1..210: mol_type = other DNA
gcggccgctcgatttgtatgtgaaatagctgaaattcgaaaatttcattatggctgtatctactttagcgtattaggcatttgagcattggcttgaacaatgcgggctgtagtgtgtcaccaaagaaaccattcgggttcggatctggaagtcctcatcacgtgatgccgatctcgtgtattttattttcagataacacctgaagacttt

SEQ ID NO: 342 - protein, 922 aa, synthetic construct
EAEASMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVKGSSGSSGSSGSSGSSGSSGSSGSSEAAAREAAAREAAAREAAARGGGGSGGGGSGGGGSQINELNVVLDDVKTNIADYITLSYTPNSGFSLDQMPAGIMDIAAQLVANPSDDSYTTLYSEVDFSAVEHMLTMVPWYSSRLLPELEAMDASLTTSSSAATSSSEVASSSIASSTSSSVAPSSSEVVSSSVASSSSEVASSSVASTSEATSSSAVTSSSAVSSSTESVSSSSVSSSSAVSSSEAVSSSPVSSVVSSSAGPASSSVAPYNSTIASSSSTAQTSISTIAPYNSTTTTTPASSASSVIISTRNGTTVTETDNTLVTKETTVCDYSSTSAVPASTTGYNNSTKVSTATICSTCKEGTSTATDFSTLKTTVTVCDSACQAKKSATVVSVQSKTTGIVEQTEN

SEQ ID NO: 343 - protein, 1827 aa, Homo sapiens
MARKKFSGLEISLIVLFVIVTIIAIALIVVLATKTPAVDEISDSTSTPATTRVTTNPSDSGKCPNVLNDPVNVRINCIPEQFPTEGICAQRGCCWRPWNDSLIPWCFFVDNHGYNVQDMTTTSIGVEAKLNRIPSPTLFGNDINSVLFTTQNQTPNRFRFKITDPNNRRYEVPHQYVKEFTGPTVSDTLYDVKVAQNPFSIQVIRKSNGKTLFDTSIGPLVYSDQYLQISTRLPSDYIYGIGEQVHKRFRHDLSWKTWPIFTRDQLPGDNNNNLYGHQTFFMCIEDTSGKSFGVFLMNSNAMEIFIQPTPIVTYRVTGGILDFYILLGDTPEQVVQQYQQLVGLPAMPAYWNLGFQLSRWNYKSLDVVKEVVRRNREAGIPFDTQVTDIDYMEDKKDFTYDQVAFNGLPQFVQDLHDHGQKYVIILDPAISIGRRANGTTYATYERGNTQHVWINESDGSTPIIGEVWPGLTVYPDFTNPNCIDWWANECSIFHQEVQYDGLWIDMNEVSSFIQGSTKGCNVNKLNYPPFTPDILDKLMYSKTICMDAVQNWGKQYDVHSLYGYSMAIATEQAVQKVFPNKRSFILTRSTFAGSGRHAAHWLGDNTASWEQMEWSITGMLEFSLFGIPLVGADICGFVAETTEELCRRWMQLGAFYPFSRNHNSDGYEHQDPAFFGQNSLLVKSSRQYLTIRYTLLPFLYTLFYKAHVFGETVARPVLHEFYEDTNSWIEDTEFLWGPALLITPVLKQGADTVSAYIPDAIWYDYESGAKRPWRKQRVDMYLPADKIGLHLRGGYIIPIQEPDVTTTASRKNPLGLIVALGENNTAKGDFFWDDGETKDTIQNGNYILYTFSVSNNTLDIVCTHSSYQEGTTLAFQTVKILGLTDSVTEVRVAENNQPMNAHSNFTYDASNQVLLIADLKLNLGRNFSVQWNQIFSENERFNCYPDADLATEQKCTQRGCVWRTGSSLSKAPECYFPRQDNSYSVNSARYSSMGITADLQLNTANARIKLPSDPISTLRVEVKYHKNDMLQFKIYDPQKKRYEVPVPLNIPTTPISTYEDRLYDVEIKENPFGIQIRRRSSGRVIWDSWLPGFAFNDQFIQISTRLPSEYIYGFGEVEHTAFKRDLNWNTWGMFTRDQPPGYKLNSYGFHPYYMALEEEGNAHGVFLLNSNAMDVTFQPTPALTYRTVGGILDFYMFLGPTPEVATKQYHEVIGHPVMPAYWALGFQLCRYGYANTSEVRELYDAMVAANIPYDVQYTDIDYMERQLDFTIGEAFQDLPQFVDKIRGEGMRYIIILDPAISGNETKTYPAFERGQQNDVFVKWPNTNDICWAKVWPDLPNITIDKTLTEDEAVNASRAHVAFPDFFRTSTAEWWAREIVDFYNEKMKFDGLWIDMNEPSSFVNGTTTNQCRNDELNYPPYFPELTKRTDGLHFRTICMEAEQILSDGTSVLHYDVHNLYGWSQMKPTHDALQKTTGKRGIVISRSTYPTSGRWGGHWLGDNYARWDNMDKSIIGMMEFSLFGMSYTGADICGFFNNSEYHLCTRWMQLGAFYPYSRNHNIANTRRQDPASWNETFAEMSRNILNIRYTLLPYFYTQMHEIHANGGTVIRPLLHEFFDEKPTWDIFKQFLWGPAFMVTPVLEPYVQTVNAYVPNARWFDYHTGKDIGVRGQFQTFNASYDTINLHVRGGHILPCQEPAQNTFYSRQKHMKLIVAADDNQMAQGSLFWDDGESIDTYERDLYLSVQFNLNQTTLTSTILKRGYINKSETRLGSLHVWGKGTTPVNAVTLTYNGNKNSLPFNEDTTNMILRIDLTTHNVTLEEPIEINWS

SEQ ID NO: 344 - protein, 61 aa, synthetic construct
MRFPSIFTAVLFAASSALAAPVQTTTEDELEGDFDVAVLPFSASIAAKEEGVSLEKREAEA